(19) United States
(12) Patent Application Publication
(10) Pub. No.: US 2021/0093619 A1
(43) Pub. Date: Apr. 1, 2021

(54) Chemical Compounds as ATF-4 Pathway Inhibitors

(71) Applicant: GlaxoSmithKline Intellectual Property Development Limited, Brentford, Middlesex (GB)

(72) Inventors: Jeffrey Michael AXTEN, Collegeville, PA (US); Michael P. DEMARTINO, Collegeville, PA (US); Karen Anderson EVANS, Collegeville, PA (US); Biswajit KALITA, Yeshwanthpur, Bangalore (IN); Jeffrey M. RALPH, Collegeville, PA (US)

(21) Appl. No.: 16/619,985

(22) PCT Filed: Jun. 7, 2018
(86) PCT No.: PCT/IN2018/050375

§ 371 Date: Dec. 6, 2019

Related U.S. Application Data
(60) Provisional application No. 62/595,425, filed on Dec. 6, 2017.

(30) Foreign Application Priority Data
Jun. 7, 2017 (IB) PCT/IB2017/053373

(51) Int. Cl. A61K 31/4439; A61K 45/06; C07C 233/41; A61K 31/165; A01N 1/02; C07D 213/65; A61K 31/44; A61K 31/444; C07D 401/12; C07D 305/08; A61K 31/337; A61K 31/4166; C07D 233/36; C07D 207/12; A61K 31/40; C07D 401/04; A61K 31/397; C07D 205/04; A61K 31/343; C07D 307/85; C07D 311/58; A61K 31/353; A61K 31/4035; C07D 209/44; A61K 31/451; C07D 211/18; C07D 401/14; C07D 207/273; C07D 211/46; A61K 9/48; A61K 9/00; A61K 9/20

(52) U.S. Cl. CPC A61K 31/4439; A61K 9/2018; C07C 233/41; A61K 31/165; A01N 1/0226; C07D 213/65; A61K 31/44; A61K 31/444; C07D 401/12; C07D 305/08; A61K 31/337; A61K 31/4166; C07D 233/36; C07D 207/12; A61K 31/40; C07D 401/04; A61K 31/397; C07D 205/04; A61K 31/343; C07D 307/85; C07D 311/58; A61K 31/353; A61K 31/4035; C07D 209/44; A61K 31/451; C07D 211/18; C07D 401/14; C07D 207/273; C07D 211/46; A61K 9/485; A61K 9/4858; A61K 9/0019; A61K 9/2009; A61K 9/2059; A61K 9/2013; A61K 45/06

(57) ABSTRACT

The invention is directed to substituted bridged cycloalkane derivatives. Specifically, the invention is directed to compounds according to Formula IIIQ:

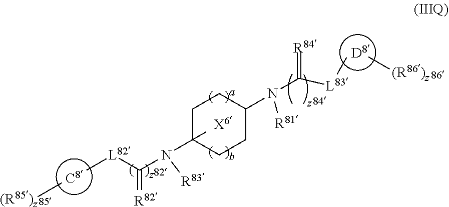

wherein X^{6′}, a, b, C^{8′}, D^{8′}, L^{82′}, L^{83′}, R^{81′}, R^{82′}, R^{83′}, R^{84′}, R^{85′}, R^{86′}, z^{82′}, z^{84′}, z^{85′}, and z^{86′} are as defined herein; or salts thereof.

The compounds of the invention are inhibitors of the ATF4 pathway. Accordingly, the invention is further directed to pharmaceutical compositions comprising a compound of the invention. The invention is still further directed to methods of inhibiting the ATF4 pathway and treatment of disorders associated therewith using a compound of the invention or a pharmaceutical composition comprising a compound of the invention.

### FIELD OF THE INVENTION

The present invention relates to substituted bridged cycloalkane derivatives that are inhibitors of the ATF4 pathway. The present invention also relates to pharmaceutical compositions comprising such compounds and methods of using such compounds in the treatment of diseases/injuries associated with activated unfolded protein response pathways, such as cancer, pre-cancerous syndromes, Alzheimer's disease, spinal cord injury, traumatic brain injury, ischemic stroke, stroke, diabetes, Parkinson disease, Huntington's disease, Creutzfeldt-Jakob Disease, and related prion diseases, progressive supranuclear palsy, amyotrophic lateral sclerosis, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementia, cognitive impairment, atherosclerosis, ocular diseases, neurological disorders, pain, arrhythmias, in organ transplantation and in the transportation of organs for transplantation.

### BACKGROUND OF THE INVENTION

In metazoa, diverse stress signals converge at a single phosphorylation event at serine 51 of a common effector, the translation initiation factor eIF2α. This step is carried out by four eIF2α kinases in mammalian cells: PERK, which responds to an accumulation of unfolded proteins in the endoplasmic reticulum (ER), GCN2 to amino acid starvation and UV light, PKR to viral infection, and HRI to heme deficiency. This collection of signaling pathways has been termed the “integrated stress response” (ISR), as they converge on the same molecular event. eIF2α phosphorylation results in an attenuation of translation with consequences that allow cells to cope with the varied stresses (1).

eIF2 (which is comprised of three subunits, α, β, and γ) binds GTP and the initiator Met-tRNA to form the ternary complex (eIF2-GTP-Met-tRNAi), which, in tum, associates with the 40S ribosomal subunit scanning the 5′UTR ofmRNAs to select the initiating AUG codon. Upon phosphorylation of its a-subunit, eIF2 becomes a competitive inhibitor of its GTP-exchange factor (GEF), eIF2B (2). The tight and nonproductive binding of phosphorylated eIF2 to eIF2B prevents loading of the eIF2 complex with GTP thus blocking ternary complex formation and reducing translation initiation (3). Because eIF2B is less abundant than eIF2, phosphorylation of only a small fraction of the total eIF2 has a dramatic impact on eIF2B activity in cells.

Paradoxically, under conditions of reduced protein synthesis, a small group of mRNAs that contain upstream open reading frames (uORFs) in their 5′UTR are translationally up-regulated (4, 5). These include mammalian ATF4 (a cAMP element binding (CREB) transcription factor) and CHOP (a pro-apoptotic transcription factor) (6-8). ATF4 regulates the expression of many genes involved in metabolism and nutrient uptake and additional transcription factors, such as CHOP, which is under both translational and transcriptional control (9). Phosphorylation of eIF2α thus leads to preferential translation of key regulatory molecules and directs diverse changes in the transcriptome of cells upon cellular stress.

One of the eIF2α kinases, PERK, lies at the intersection of the ISR and the unfolded protein response (UPR) that maintains homeostasis of protein folding rates in the ER (10). The UPR is activated by unfolded or misfolded proteins that accumulate in the ER lumen because of an imbalance between protein folding load and protein folding capacity, a condition known as “ER stress”. In mammals, the UPR is comprised of three signaling branches mediated by ER-localized transmembrane sensors, PERK, IRE1, and ATF6. These sensor proteins detect the accumulation of unfolded protein in the ER and transmit the information across the ER membrane, initiating unique signaling pathways that converge in the activation of an extensive transcriptional response, which ultimately results in ER expansion (11). The lumenal stress-sensing domains of PERK and IRE1 are homologous and likely activated in analogous ways by direct binding to unfolded peptides (12). This binding event leads to oligomerization and trans-autophosphorylation of their cytosolic kinase domains, and, for PERK, phosphorylation of its only known substrate, eIF2α. In this way, PERK activation results in a quick reduction in the load of newly synthesized proteins that are translocated into the ER-lumen (13).

Upon ER stress, both the transcription factor XBP 1 s, produced as the consequence of a non-conventional mRNA splicing reaction initiated by IRE1, and the transcription factor ATF6, produced by proteolysis and release from the ER membrane, collaborate with ATF4 to induce the vast UPR transcriptional response. Transcriptional targets of the UPR include the ER protein folding machinery, the ER-associated degradation machinery, and many other components functioning in the secretory pathway (14). Although the UPR initially mitigates ER stress and as such confers cytoprotection, persistent and severe ER stress leads to activation of apoptosis that eliminates damaged cells (15, 16).

Small-molecule therapeutics that inhibit the UPR and/or the Integrated Stress Response could be used in cancer as a single agent or in combination with other chemotherapeutics (17, 18, 19), for enhancement of long-term memory (24, 25), in neurodegenerative and prion associated diseases (20), in white matter disease (VWM) (23) and in biotechnology applications that would benefit from increased protein translation.

It is an object of the instant invention to provide novel compounds that prevent the translation of ATF4 or are inhibitors of the ATF4 pathway.

It is also an object of the present invention to provide pharmaceutical compositions that comprise a pharmaceutically acceptable excipient and compounds of Formula (IIIQ).

It is also an object of the present invention to provide a method for treating neurodegenerative diseases, cancer, and other diseases/injuries associated with activated unfolded protein response pathways such as: Alzheimer's disease, spinal cord injury, traumatic brain injury, ischemic stroke, stroke, diabetes, Parkinson disease, Huntington's disease, Creutzfeldt-Jakob Disease, and related prion diseases, amyotrophic lateral sclerosis, progressive supranuclear palsy, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementias, atherosclerosis, ocular diseases, neurological disorders, pain, arrhythmias, in organ transplantation and in the transportation of organs for transplantation that comprises administering novel inhibitors of the ATF4 pathway.

### SUMMARY OF THE INVENTION

The invention is directed to substituted bridged cycloalkane derivatives. Specifically, the invention is directed to compounds according to Formula IIIQ:

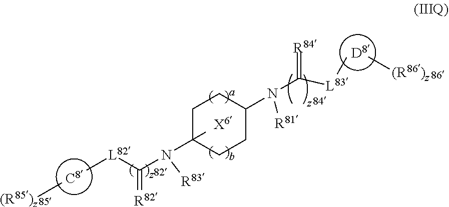

wherein X^{6′}, a, b, C^{8′}, D^{8′}, L^{82′}, L^{83′}, R^{81′}, R^{82′}, R^{83′}, R^{84′}, R^{85′}, R^{86′}, z^{82′}, z^{84′}, z^{85′}, and z^{86′} are as defined below; or a salt thereof including a pharmaceutically acceptable salt thereof.

The present invention also relates to the discovery that the compounds of Formula (IIIQ) are active as inhibitors of the ATF4 pathway.

The present invention also relates to the discovery that the compounds of Formula (IIIQ) prevent the translation of ATF4.

This invention also relates to a method of treating Alzheimer's disease, which comprises administering to a human in need thereof an effective amount of a compound of Formula (IIIQ) or a pharmaceutically acceptable salt thereof.

This invention also relates to a method of treating Parkinson's disease, which comprises administering to a human in need thereof an effective amount of a compound of Formula (IIIQ) or a pharmaceutically acceptable salt thereof.

This invention also relates to a method of treating amyotrophic lateral sclerosis, which comprises administering to a human in need thereof an effective amount of a compound of Formula (IIIQ) or a pharmaceutically acceptable salt thereof.

This invention also relates to a method of treating Huntington's disease, which comprises administering to a human in need thereof an effective amount of a compound of Formula (IIIQ) or a pharmaceutically acceptable salt thereof.

This invention also relates to a method of treating Creutzfeldt-Jakob Disease, which comprises administering to a human in need thereof an effective amount of a compound of Formula (IIIQ) or a pharmaceutically acceptable salt thereof.

This invention also relates to a method of treating progressive supranuclear palsy (PSP), which comprises administering to a human in need thereof an effective amount of a compound of Formula (IIIQ) or a pharmaceutically acceptable salt thereof.

This invention also relates to a method of treating dementia, which comprises administering to a human in need thereof an effective amount of a compound of Formula (IIIQ) or a pharmaceutically acceptable salt thereof.

This invention also relates to a method of treating spinal cord injury, which comprises administering to a human in need thereof an effective amount of a compound of Formula (IIIQ) or a pharmaceutically acceptable salt thereof.

This invention also relates to a method of treating traumatic brain injury, which comprises administering to a human in need thereof an effective amount of a compound of Formula (IIIQ) or a pharmaceutically acceptable salt thereof.

This invention also relates to a method of treating ischemic stroke, which comprises administering to a human in need thereof an effective amount of a compound of Formula (IIIQ) or a pharmaceutically acceptable salt thereof.

This invention also relates to a method of treating diabetes, which comprises administering to a human in need thereof an effective amount of a compound of Formula (IIIQ) or a pharmaceutically acceptable salt thereof.

This invention also relates to a method of treating a disease state selected from: myocardial infarction, cardiovascular disease, atherosclerosis, ocular diseases, and arrhythmias, which comprises administering to a human in need thereof an effective amount of a compound of Formula (IIIQ) or a pharmaceutically acceptable salt thereof.

This invention also relates to a method of treating an integrated stress response-associated disease in a patient in need of such treatment, which comprises administering a therapeutically effective amount of a compound of Formula (IIIQ) or a pharmaceutically acceptable salt thereof, to the patient.

This invention also relates to a method of treating a disease associated with phosphorylation of eIF2a in a patient in need of such treatment, which comprises administering a therapeutically effective amount of a compound of Formula (IIIQ), or a pharmaceutically acceptable salt thereof, to the patient.

This invention also relates to a method of treating a disease in a patient in need of such treatment, which comprises administering a therapeutically effective amount of a compound of Formula (IIIQ) or a pharmaceutically acceptable salt thereof, to the patient, wherein the disease is selected from the group consisting of cancer, a neurodegenerative disease, vanishing white matter disease, childhood ataxia with CNS hypomyelination, and an intellectual disability syndrome.

This invention also relates to a method of improving long-term memory in a patient, which comprises administering a therapeutically effective amount of a compound of Formula (IIIQ) or a pharmaceutically acceptable salt thereof, to the patient.

This invention also relates to a method of increasing protein expression of a cell or in vitro expression system, which comprises administering an effective amount of a compound of Formula (IIIQ) or a pharmaceutically acceptable salt thereof, to the cell or expression system.

This invention also relates to a method of treating an inflammatory disease in a patient in need of such treatment, which comprises administering a therapeutically effective amount of a compound of Formula (IIIQ), or a pharmaceutically acceptable salt thereof, to the patient.

This invention also relates to a method of using the compounds of Formula (IIIQ) in organ transplantation and in the transportation of organs for transplantation.

Also included in the present invention are methods of co-administering the presently invented compounds with further active ingredients.

Included in the present invention is a method for treating neurodegenerative diseases, cancer, and other diseases/injuries associated with activated unfolded protein response pathways, such as: Alzheimer's disease, spinal cord injury, traumatic brain injury, ischemic stroke, stroke, diabetes, Parkinson disease, Huntington's disease, Creutzfeldt-Jakob Disease, and related prion diseases, amyotrophic lateral sclerosis, progressive supranuclear palsy, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementias, atherosclerosis, ocular diseases, arrhythmias, in organ transplantation and in the transportation of organs for transplantation that comprises administering the compounds of Formula (IIIQ).

The invention also relates to a compound of Formula (IIIQ) or a pharmaceutically acceptable salt thereof for use in therapy.

The invention also relates to a compound of Formula (IIIQ) or a pharmaceutically acceptable salt thereof for use in the treatment of Alzheimer's disease.

The invention also relates to a compound of Formula (IIIQ) or a pharmaceutically acceptable salt thereof for use in the treatment of Parkinson's disease syndromes.

The invention also relates to a compound of Formula (IIIQ) or a pharmaceutically acceptable salt thereof for use in the treatment of amyotrophic lateral sclerosis.

The invention also relates to a compound of Formula (IIIQ) or a pharmaceutically acceptable salt thereof for use in the treatment of Huntington's disease.

The invention also relates to a compound of Formula (IIIQ) or a pharmaceutically acceptable salt thereof for use in the treatment of Creutzfeldt-Jakob Disease.

The invention also relates to a compound of Formula (IIIQ) or a pharmaceutically acceptable salt thereof for use in the treatment of progressive supranuclear palsy (PSP).

The invention also relates to a compound of Formula (IIIQ) or a pharmaceutically acceptable salt thereof for use in the treatment of dementia.

The invention also relates to a compound of Formula (IIIQ) or a pharmaceutically acceptable salt thereof for use in the treatment of spinal cord injury.

The invention also relates to a compound of Formula (IIIQ) or a pharmaceutically acceptable salt thereof for use in the treatment of traumatic brain injury.

The invention also relates to a compound of Formula (IIIQ) or a pharmaceutically acceptable salt thereof for use in the treatment of ischemic stroke.

The invention also relates to a compound of Formula (IIIQ) or a pharmaceutically acceptable salt thereof for use in the treatment of diabetes.

The invention also relates to a compound of Formula (IIIQ) or a pharmaceutically acceptable salt thereof for use in the treatment of a disease state selected from: myocardial infarction, cardiovascular disease, atherosclerosis, ocular diseases, and arrhythmias.

The invention also relates to the use of a compound of Formula (IIIQ) or a pharmaceutically acceptable salt thereof in the manufacture of a medicament for the treatment of an integrated stress response-associated disease.

The invention also relates to the use of a compound of Formula (IIIQ) or a pharmaceutically acceptable salt thereof in the manufacture of a medicament for the treatment of a disease associated with phosphorylation of eIF2a.

The invention also relates to the use of a compound of Formula (IIIQ) or a pharmaceutically acceptable salt thereof in the manufacture of a medicament for the treatment of a disease selected from the group consisting of: cancer, a neurodegenerative disease, vanishing white matter disease, childhood ataxia with CNS hypomyelination, and an intellectual disability syndrome.

The invention also relates to the use of a compound of Formula (IIIQ) or a pharmaceutically acceptable salt thereof in the manufacture of a medicament for improving long-term memory.

The invention also relates to the use of a compound of Formula (IIIQ) or a pharmaceutically acceptable salt thereof in the manufacture of a medicament for increasing protein expression of a cell or in vitro expression system.

The invention also relates to the use of a compound of Formula (IIIQ) or a pharmaceutically acceptable salt thereof in the manufacture of a medicament for the treatment of inflammatory disease.

The invention also relates to the use of a compound of Formula (IIIQ) or a pharmaceutically acceptable salt thereof in the manufacture of a medicament in organ transplantation and in the transportation of organs for transplantation.

The invention also relates to the use of a compound of Formula (IIIQ) or a pharmaceutically acceptable salt thereof in the manufacture of a medicament for the treatment of a disease state selected from: neurodegenerative diseases, cancer, and other diseases/injuries associated with activated unfolded protein response pathways such as: Alzheimer's disease, spinal cord injury, traumatic brain injury, ischemic stroke, stroke, diabetes, Parkinson disease, Huntington's disease, Creutzfeldt-Jakob Disease, and related prion diseases, amyotrophic lateral sclerosis, progressive supranuclear palsy, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementias, atherosclerosis, ocular diseases, neurological disorders, pain, arrhythmias, in organ transplantation and in the transportation of organs for transplantation.

Included in the present invention are pharmaceutical compositions that comprise a pharmaceutical excipient and a compound of Formula (IIIQ) or a pharmaceutically acceptable salt thereof.

The invention also relates to a pharmaceutical composition as defined above for use in therapy.

The invention also relates to a combination for use in therapy which comprises a therapeutically effective amount of (i) a compound of Formula (IIIQ) or a pharmaceutically acceptable salt thereof; and (ii) further active ingredients.

### DETAILED DESCRIPTION OF THE INVENTION

Included in the compounds of the invention and used in the methods of the invention are compounds of Formula (I):

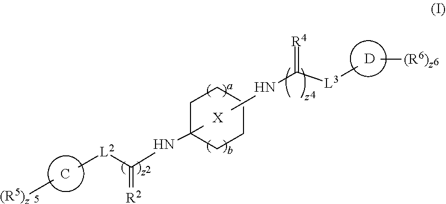

wherein:
   L² and L³ are independently a bond, —NH—, —O—, —S—, —S(O)—, —S(O)₂—, substituted or unsubstituted C₁₋₆alkylene or substituted or unsubstituted C₁₋₆heteroalkylene;
   R⁵ and R⁶ are each independently hydrogen, fluoro, chloro, bromo, iodo, —OCH₃, —OCH₂Ph, —C(O)Ph, —CH₃, —CF₃, —CN, —S(O)CH₃, —OH, —NH₂, —COOH, —CONH₂, —NO₂, —C(O)CH₃, —CH(CH₃)₂, —CCH, —CH₂CCH, —SO₃H, —SO₂NH₂, —NHC(O)NH₂, —NHC(O))H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R² and R⁴ are independently NR⁸, O, CH₂, or S;
   R⁸ is selected from: hydrogen, C₁₋₆alkyl and C₁₋₆alkyl substituted 1 to 6 times by fluoro;
   a and b are independently 0 or 1;
   C and D are independently phenyl or pyridine;
   X is C₁₋₃alkylene or C₁₋₃alkylene substituted 1 to 3 times by fluoro;
   Z² and z⁴ are independently 0 or 1; and
   Z⁵ and z⁶ are independently an integer from 0 to 5;
and salts thereof.

This invention also relates to pharmaceutically acceptable salts of the compounds of Formula (I).

Included in the compounds of the invention and used in the methods of the invention are compounds of Formula (II):

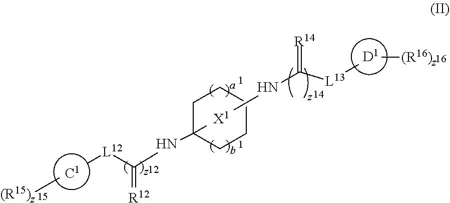

wherein:
L¹² and L¹³ are independently: —CH₂—O—, —O—CH₂—, —O—CH₂—CH₂—, and —CH₂—CH₂—O—;
R¹⁵ and R¹⁶ are independently hydrogen or chloro;
R¹² and R¹⁴ are O;
a¹ and b¹ areindependently 0 or 1;
C¹ and D¹ are independently phenyl or pyridine;
X¹ is selected from —CH₂— and —CH₂—CH₂—;
z¹² and z¹⁴ are independently 0 or 1; and
z¹⁵ and z¹⁶ are independently an integer from 0 to 5;
and salts thereof.

This invention also relates to pharmaceutically acceptable salts of the compounds of Formula (II).

Included in the compounds of the invention and used in the methods of the invention are compounds of Formula (III):

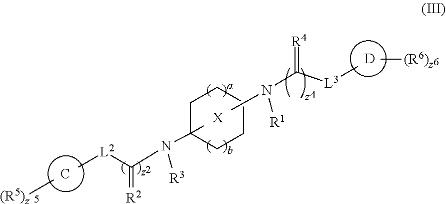

wherein:
   L² is selected from: a bond, —NH—, —O—, —S—,—S(O)—, —S(O)₂—, substituted or unsubstituted C₁₋₆alkylene or substituted or unsubstituted C₁₋₆heteroalkylene, or L² is further taken together with R³ to form heterocycloalkyl;
   L³ is selected from: a bond, —NH—, —O—, —S—, —S(O)—, —S(O)₂—, substituted or unsubstituted C₁₋₆alkylene or substituted or unsubstituted C₁₋₆heteroalkylene, or L³ is further taken together with R¹ to form heterocycloalkyl;
   R¹ is selected from: hydrogen, C₁₋₆alkyl, substituted C₁₋₆alkyl, heterocycloalkyl, or R¹ is taken together with L³ to form heterocycloalkyl;
   R³ is selected from: hydrogen, C₁₋₆alkyl, substituted C₁₋₆alkyl, heterocycloalkyl, or R³ is taken together with L² to form heterocycloalkyl;
   R⁵ and R⁶ are each independently hydrogen, fluoro, chloro, bromo, iodo, —OCH₃, —OCH₂Ph, —C(O)Ph, —CH₃, —CF₃, —CN, —S(O)CH₃, —OH, —NH₂, —COOH, —CONH₂, —NO₂, —C(O)CH₃, —CH(CH₃)₂, —C≡CH, —CH₂C≡CH, —SO₃H, —SO₂NH₂, —NHC(O)NH₂, —NHC(O)H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R² and R⁴ are independently NR⁸, O, CH₂, or S;
   R⁸ is selected from: hydrogen, C₁₋₆alkyl and C₁₋₆alkyl substituted 1 to 6 times by fluoro;
   a and b are independently 0 or 1;
   C and D are independently phenyl or pyridyl;
   X is C₁₋₃alkylene or C₁₋₃alkylene substituted 1 to 3 times by fluoro;
   z² and z⁴ are independently 0 or 1; and
   z⁵ and z⁶ are independently an integer from 0 to 5;
or a salt thereof including a pharmaceutically acceptable salt thereof.

This invention also relates to pharmaceutically acceptable salts of the compounds of Formula (III).

Included in the compounds of the invention and used in the methods of the invention are compounds of Formula (IV):

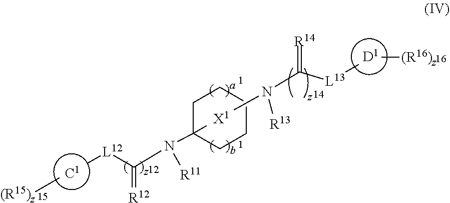

wherein:
   L¹² is selected from: a bond, —CH₂—, —NH—, CH₂—O—, —O—CH₂—, —O—CH₂—CH₂—, and —CH₂CH₂—O—, or L¹² is further taken together with R¹¹ to form imidazolidinyl;
   L¹³ is selected from: a bond, —CH₂—, —NH—, CH₂—O—, —O—CH₂—, —O—CH₂—CH₂—, and —CH₂—CH₂—O—, or L¹³ is further taken together with R¹³ to form imidazolidinyl;
   R¹¹ is selected from: hydrogen, C₁₋₆alkyl, substituted C₁₋₆alkyl, oxetanyl, or R¹¹ is taken together with L¹² to form imidazolidinyl;
   R¹³ is selected from: hydrogen, C₁₋₆alkyl, substituted C₁₋₆alkyl, oxetanyl, or R¹³ is taken together with L¹³ to form imidazolidinyl;
   R¹⁵ and R¹⁶ are independently hydrogen, methyl, or chloro;
   R¹² and R¹⁴ are O;
   a¹ and b¹ are independently 0 or 1;
   C¹ and D¹ areindependently phenyl or pyridyl;
   X¹ is selected from —CH₂— and —CH₂—CH₂—;
   Z¹² and z¹⁴ are independently 0 or 1; and
   Z¹⁵ and z¹⁶ are independently an integer from 0 to 5;
or a salt thereof including a pharmaceutically acceptable salt thereof.

This invention also relates to pharmaceutically acceptable salts of the compounds of Formula (IV).

Included in the compounds of the invention and used in the methods of the invention are compounds of Formula (IIIX):

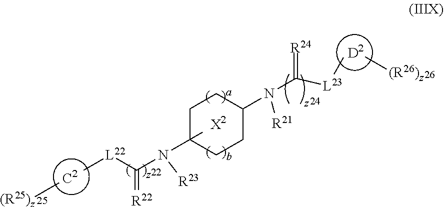

wherein:
   L²² is selected from: a bond, —NH—, —O—, —S—, —S(O)—, —S(O)₂—, substituted or unsubstituted C₁₋₆alkylene and substituted or unsubstituted C₁₋₆heteroalkylene, or L²² is taken together with R²³ to form heterocycloalkyl;
   L²³ and R²¹ are taken together to form heterocycloalkyl;
   R²³ is selected from: hydrogen, C₁₋₆alkyl, substituted C₁₋₆alkyl, heterocycloalkyl, or R²³ is taken together with L²² to form heterocycloalkyl;
   R²⁵ and R²⁶ are each independently hydrogen, fluoro, chloro, bromo, iodo, —OCH₃, —OCH₂Ph, —C(O)Ph, —CH₃, —CF₃, —CN, —S(O)CH₃, —OH, —NH₂, —COOH, —CONH₂, —NO₂, —C(O)CH₃, —CH(CH₃)₂, —C≡CH, —CH₂C≡CH, —SO₃H, —SO₂NH₂, —NHC(O)NH₂, —NHC(O)H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R²² and R²⁴ are independently NR²⁸, O, CH₂, or S;
   R²⁸ is selected from: hydrogen, C₁₋₆alkyl and C₁₋₆alkyl substituted 1 to 6 times by fluoro;
   a and b are independently 0 or 1;
   C² and D² are independently phenyl or pyridyl;
   X² is C₁₋₃alkylene or C₁₋₃alkylene substituted 1 to 3 times by fluoro;
   z²² and z²⁴ are independently 0 or 1; and
   z²⁵ and z²⁶ are independently an integer from 0 to 5;
or a salt thereof including a pharmaceutically acceptable salt thereof.

This invention also relates to pharmaceutically acceptable salts of the compounds of Formula (IIIX).

Included in the compounds of the invention and used in the methods of the invention are compounds of Formula (IVX):

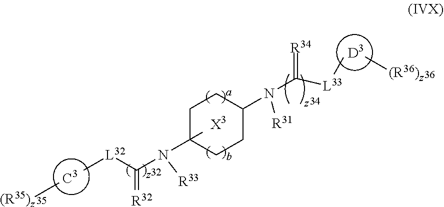

wherein:
   L³² is selected from: a bond, —NH—, —O—, —S—, —S(O)—, —S(O)₂—, substituted or unsubstituted C₁₋₆alkylene and substituted or unsubstituted C₁₋₆heteroalkylene;
   L³³ taken together with R³¹ to form heterocycloalkyl;
   R³³ is selected from: hydrogen, C₁₋₆alkyl, substituted C₁₋₆alkyl, and heterocycloalkyl;
   R³⁵ and R³⁶ are each independently hydrogen, fluoro, chloro, bromo, iodo, —OCH₃, —OCH₂Ph, —C(O)Ph, —CH₃, —CF₃, —CN, —S(O)CH₃, —OH, —NH₂, —COOH, —CONH₂, —NO₂, —C(O)CH₃, —CH(CH₃)₂, —C≡CH, —CH₂C≡CH, —SO₃H, —SO₂NH₂, —NHC(O)NH₂, —NHC(O)H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R³² and R³⁴ are independently NR³⁸, O, CH₂, or S;
   R³⁸ is selected from: hydrogen, C₁₋₆alkyl and C₁₋₆alkyl substituted 1 to 6 times by fluoro;
   a and b are independently 0 or 1;
   C³ and D³ are independently phenyl or pyridyl;
   X³ is C₁₋₃alkylene or C₁₋₃alkylene substituted 1 to 3 times by fluoro;
   z³² and z³⁴ are independently 0 or 1; and
   z³⁵ and z³⁶ are independently an integer from 0 to 5;
or a salt thereof including a pharmaceutically acceptable salt thereof.

This invention also relates to pharmaceutically acceptable salts of the compounds of Formula (IVX).

Included in the compounds of the invention and used in the methods of the invention are compounds of Formula (VX):

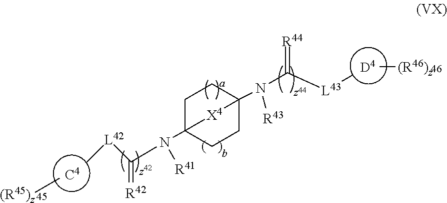

wherein:
   L⁴² is selected from: a bond, —CH₂—, —NH—, CH₂—O—, —O—CH₂—, —O—CH₂—CH₂—, and —CH₂—CH₂—O—, or L⁴² is taken together with R⁴¹ to form imidazolidinyl or pyrrolidinyl;
   L⁴³ is taken together with R⁴³ to form imidazolidinyl or pyrrolidinyl;
   R⁴¹ is selected from: hydrogen, C₁₋₆alkyl, substituted C₁₋₆alkyl, and oxetanyl, or R⁴¹ is taken together with L⁴² to form imidazolidinyl or pyrrolidinyl;
   R⁴⁵ and R⁴⁶ are independently hydrogen, methyl, or chloro;
   R⁴² and R⁴⁴ are O;
   a and b are independently 0 or 1;
   C⁴ and D⁴ are independently phenyl or pyridyl;
   X⁴ is selected from —CH₂- and —CH₂—CH₂—;
   Z⁴² and z⁴⁴ are independently 0 or 1; and
   Z⁴⁵ and z⁴⁶ are independently an integer from 0 to 5;
or a salt thereof including a pharmaceutically acceptable salt thereof.

This invention also relates to pharmaceutically acceptable salts of the compounds of Formula (VX).

Included in the compounds of the invention and used in the methods of the invention are compounds of Formula (VIX):

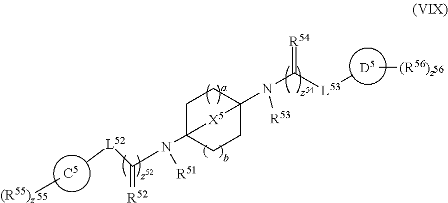

wherein:
   L⁵² is selected from: a bond, —CH₂—, —NH—, CH₂—O—, —O—CH₂—, —O—CH₂—CH₂—, and —CH₂—CH₂—O—;
   L⁵³ is taken together with R⁵³ to form imidazolidinyl or pyrrolidinyl;
   R⁵¹ is selected from: hydrogen, C₁₋₆alkyl, substituted C₁₋₆alkyl, and oxetanyl;
   R⁵⁵ and R⁵⁶ are independently hydrogen, methyl, or chloro;
   R⁵² and R⁵⁴ are 0;
   a and b are independently 0 or 1;
   C⁵ and D⁵ are independently phenyl or pyridyl;
   X⁵ is selected from —CH₂— and —CH₂—CH₂—;
   Z⁵² and z⁵⁴ are independently 0 or 1; and
   Z⁵⁵ and z⁵⁶ are independently an integer from 0 to 5;
or a salt thereof including a pharmaceutically acceptable salt thereof.

This invention also relates to pharmaceutically acceptable salts of the compounds of Formula (VIX).

Included in the compounds of the invention and used in the methods of the invention are compounds of Formula (VIIX):

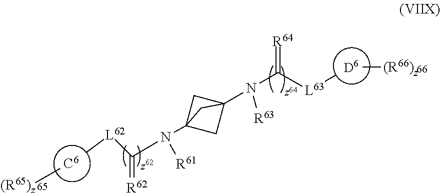

wherein:
L⁶² is selected from: a bond, —CH₂—, —NH—, CH₂—O—, —O—CH₂—, —O—CH₂—CH₂—, and —CH₂—CH₂—O—, or L⁶² is taken together with R⁶¹ to form imidazolidinyl or pyrrolidinyl;
L⁶³ is taken together with R⁶³ to form imidazolidinyl or pyrrolidinyl;
R⁶¹ is selected from: hydrogen, C₁₋₆alkyl, substituted C₁₋₆alkyl, and oxetanyl, or R⁶¹ is taken together with L⁶² to form imidazolidinyl or pyrrolidinyl;
R⁶⁵ and R⁶⁶ are independently hydrogen, methyl, or chloro;
R⁶² and R⁶⁴ are O;
C⁶ and D⁶ are independently phenyl or pyridyl;
Z⁶² and z⁶⁴ are independently 0 or 1; and
Z⁶⁵ and z⁶⁶ are independently an integer from 0 to 3;
or a salt thereof including a pharmaceutically acceptable salt thereof.

This invention also relates to pharmaceutically acceptable salts of the compounds of Formula (VIIX).

Included in the compounds of the invention and used in the methods of the invention are compounds of Formula (VIIIX):

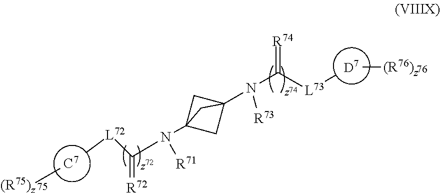

wherein:
   L⁷² is selected from: a bond, —CH₂—, —NH—, CH₂—O—, —O—CH₂—, —O—CH₂—CH₂—, and —CH₂—CH₂—O—;
   L⁷³ is taken together with R⁷³ to form imidazolidinyl or pyrrolidinyl;
   R⁷¹ is selected from: hydrogen, C₁₋₆alkyl, substituted C₁₋₆alkyl, and oxetanyl;
   R⁷⁵ and R⁷⁶ are independently hydrogen, methyl, or chloro;
   R⁷² and R⁷⁴ are O;
   C⁷ and D⁷ are independently phenyl or pyridyl;
   Z⁷² and z⁷⁴ are independently 0 or 1; and
   Z⁷⁵ and z⁷⁶ are independently an integer from 0 to 3;
or a salt thereof including a pharmaceutically acceptable salt thereof.

This invention also relates to pharmaceutically acceptable salts of the compounds of Formula (VIIIX).

Included in the compounds of the invention and used in the methods of the invention are compounds of Formula (IIIZ):

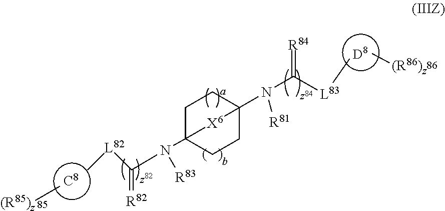

wherein:
   L⁸² is selected from: a bond, —NH—, —O—, —S—, —S(O)—, —S(O)₂—, cycloalkyl, —O-cycloalkyl, cycloalkyl-O—, —NH-cycloalkyl, cycloalkyl-NH—, azetidinyl, —O-azetidinyl, azetidinyl-O—, —N-azetidinyl, azetidinyl-N—, substituted or unsubstituted C₁₋₆alkylene and substituted or unsubstituted C₁₋₆heteroalkylene, or L⁸² is taken together with R⁸³ to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
   L⁸³ is selected from: cycloalkyl, —O-cycloalkyl, cycloalkyl-O—, —NH-cycloalkyl, cycloalkyl-NH—, azetidinyl, —O-azetidinyl, azetidinyl-O—, —N-azetidinyl, azetidinyl-N—, or L⁸³ and R⁸¹ are taken together to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
   R⁸¹ is selected from: hydrogen, C₁₋₆alkyl, substituted C₁₋₆alkyl, and heterocycloalkyl, or R⁸¹ is taken together with L⁸³ to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
   R⁸³ is selected from: hydrogen, C₁₋₆alkyl, substituted C₁₋₆alkyl, and heterocycloalkyl, or R⁸³ is taken together with L⁸² to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
   R⁸⁵ and R⁸⁶ are each independently fluoro, chloro, bromo, iodo, —OCH₃, —OCH₂Ph, —C(O)Ph, —CF₃, —CN, —S(O)CH₃, —OH, —NH₂, —COOH, —CONH₂, —NO₂, —C(O)CH₃, —C≡CH, —CH₂C≡CH, —SCH₃, —SO₃H, —SO₂NH₂, —NHC(O)NH₂, —NHC(O)H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R⁸² and R⁸⁴ are independently NR⁸⁸, O, CH₂, or S;
   R⁸⁸ is selected from: hydrogen, C₁₋₆alkyl and C₁₋₆alkyl substituted 1 to 6 times by fluoro;
   a and b are independently 0 or 1;
   C⁸ and D⁸ are independently phenyl or pyridyl;
   X⁶ is C₁₋₃alkylene or C₁₋₃alkylene substituted 1 to 3 times by fluoro;
   Z⁸² and z⁸⁴ are independently 0 or 1; and
   Z⁸⁵ and z⁸⁶ are independently an integer from 0 to 5; or a salt thereof including a pharmaceutically acceptable salt thereof.

This invention also relates to pharmaceutically acceptable salts of the compounds of Formula (IIIZ).

Included in the compounds of the invention and used in the methods of the invention are compounds of Formula (IVZ):

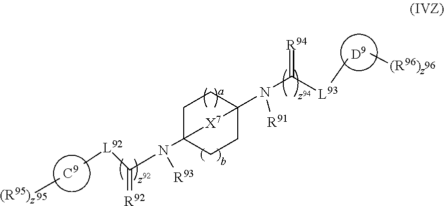

wherein:
   L⁹² is selected from: a bond, —NH—, —O—, —S—, —S(O)—, —S(O)₂—, substituted or unsubstituted C₁₋₆alkylene and substituted or unsubstituted C₁₋₆heteroalkylene;
   L⁹³ is selected from: cycloalkyl, —O-cycloalkyl, and cycloalkyl-O—, azetidinyl, —O-azetidinyl, azetidinyl-O—, or L⁹³ is taken together with R⁹¹ to form: heterocycloalkyl, heterocycloalkyl-O—, oxoheterocycloalkyl, or oxoheterocycloalkyl-O—;
   R⁹¹ is selected from: hydrogen, C₁₋₆alkyl, substituted C₁₋₆alkyl, and heterocycloalkyl, or R⁹¹ is taken together with L⁹³ to form: heterocycloalkyl, heterocycloalkyl-O—, oxoheterocycloalkyl, or oxoheterocycloalkyl-O—;
   R⁹³ is selected from: hydrogen, C₁₋₆alkyl, substituted C₁₋₆alkyl, and heterocycloalkyl;
   R⁹⁵ and R⁹⁶ are independently selected from: fluoro, chloro, bromo, iodo, —OCH₃, —OCH₂Ph, —C(O)Ph, —CF₃, —CN, —S(O)CH₃,—OH, —NH₂, —COOH, —CONH₂, —NO₂, —C(O)CH₃, —C≡CH, —CH₂C≡CH, —SCH₃, —SO₃H, —SO₂NH₂, —NHC(O)NH₂, —NHC(O)H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R⁹² and R⁹⁴ are independently NR⁹⁸, O, or S;
   R⁹⁸ is selected from: hydrogen, C₁₋₆alkyl and C₁₋₆alkyl substituted 1 to 6 times by fluoro;
   a and b are independently 0 or 1;
   C⁹ and D⁹ are independently phenyl or pyridyl;
   X⁷ is C₁₋₃alkylene or C₁₋₃alkylene substituted 1 to 3 times by fluoro;
   Z⁹² and z⁹⁴ are independently 0 or 1; and
   Z⁹⁵ and z⁹⁶ are independently an integer from 0 to 5;
or a salt thereof including a pharmaceutically acceptable salt thereof.

This invention also relates to pharmaceutically acceptable salts of the compounds of Formula (IVZ).

Included in the compounds of the invention and used in the methods of the invention are compounds of Formula (VZ):

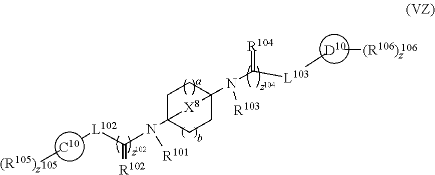

wherein:
   L¹⁰² is selected from: a bond, —CH₂—, —NH—, CH₂—O—, —O—CH₂—, cyclopropyl, —O-cyclopropyl, cyclopropyl-O—, —NH-cyclopropyl, cyclopropyl-NH—, azetidinyl, —O-azetidinyl, azetidinyl-O—, —N-azetidinyl, azetidinyl-N—, —O—CH₂—CH₂—, and —CH₂—CH₂—O—, or L¹⁰² is taken together with R¹⁰¹ to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
   L¹⁰³ is selected from: cyclopropyl, azetidinyl, —O-azetidinyl, azetidinyl-O—, —N-azetidinyl, azetidinyl-N—, or L¹⁰³ is taken together with R¹⁰³ to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
   R¹⁰¹ is selected from: hydrogen, C₁₋₆alkyl, substituted C₁₋₆alkyl, and oxetanyl, or R¹⁰¹ is taken together with L¹⁰² to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
   R¹⁰³ is selected from: hydrogen, C₁₋₆alkyl, substituted C₁₋₆alkyl, and oxetanyl, or R¹⁰³ is taken together with L¹⁰³ to form: imidazolidinyl, azetidinyl, azetidinyl-O—, azetidinyl-N—, azetidinyl-CH₂—, piperidinyl, piperidinyl-O—, piperidinyl-N-, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
   R¹⁰⁵ and R¹⁰⁶ are each independently selected from: methyl, cyclopropyl, —OCF₃, fluoro, chloro, —SCH₃, —OCH₃, —OCHF₂, and —CF₃;
   R¹⁰² and R¹⁰⁴ are O;
   a and b are independently 0 or 1;
   C¹⁰ and D¹⁰ are independently phenyl or pyridyl;
   X⁸ is selected from —CH₂— and —CH₂—CH₂—;
   Z¹⁰² and z¹⁰⁴ are independently 0 or 1; and
   Z¹⁰⁵ and z¹⁰⁶ are independently an integer from 0 to 5;
      or a salt thereof including a pharmaceutically acceptable salt thereof.

This invention also relates to pharmaceutically acceptable salts of the compounds of Formula (VZ).

Included in the compounds of the invention and used in the methods of the invention are compounds of Formula (VIZ):

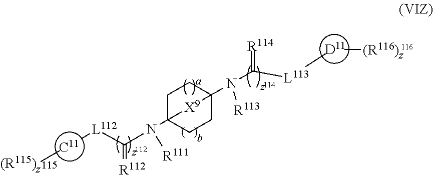

wherein:
   L¹¹² is selected from: a bond, —CH₂—, —NH—, CH₂—O—, —O—CH₂—, —O—CH₂—CH₂—, and —CH₂—CH₂—O—;
   L¹¹³ is selected from: cyclopropyl, —O-cyclopropyl, cyclopropyl-O—, azetidinyl, —O-azetidinyl, azetidinyl-O—, or L¹¹³ is taken together with R¹¹³ to form: imidazolidinyl, azetidinyl, azetidinyl-O—, piperidinyl, piperidinyl-O—, piperazinyl, piperazinyl-O—, oxopiperazinyl, oxopiperazinyl-O—, pyrrolidinyl, pyrrolidinyl-O—, oxopyrrolidinyl, or oxopyrrolidinyl-O—;
   R¹¹³ is selected from: hydrogen, C₁₋₆alkyl, substituted C₁₋₆alkyl, and oxetanyl or R¹¹³ is taken together with L¹¹³ to form: imidazolidinyl, azetidinyl, azetidinyl-O—, piperidinyl, piperidinyl-O—, piperazinyl, piperazinyl-O—, oxopiperazinyl, oxopiperazinyl-O—, pyrrolidinyl, pyrrolidinyl-O—, oxopyrrolidinyl, or oxopyrrolidinyl-O—;
   R¹¹¹ is selected from: hydrogen, C₁₋₆alkyl, substituted C₁₋₆alkyl, and oxetanyl;
   R¹¹⁵ and R¹¹⁶ are each independently selected from: methyl, cyclopropyl, —OCF₃, fluoro, chloro, —SCH₃, —OCH₃, —OCHF₂, and —CF₃;
   R¹¹² and R¹¹⁴ are O;
   a and b are independently 0 or 1;
   C¹¹ and D¹¹ are independently phenyl or pyridyl;
   X⁹ is selected from —CH₂— and —CH₂—CH₂—;
   Z¹¹² and z¹¹⁴ are independently 0 or 1; and
   Z¹¹⁵ and z¹¹⁶ are independently an integer from 0 to 5;
or a salt thereof including a pharmaceutically acceptable salt thereof.

This invention also relates to pharmaceutically acceptable salts of the compounds of Formula (VIZ).

Included in the compounds of the invention and used in the methods of the invention are compounds of Formula (VIIZ):

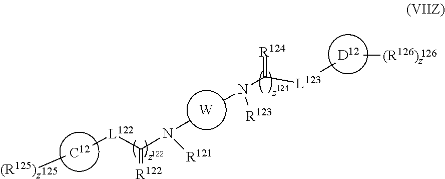

wherein:
   W is selected from bicyclopentanyl and bicyclohexanyl;
   L¹²² is selected from: a bond, —CH₂—, —NH—, CH₂—O—, —O—CH₂—, cyclopropyl, —O-cyclopropyl, cyclopropyl-O—, —NH-cyclopropyl, cyclopropyl-NH—, azetidinyl, —O-azetidinyl, azetidinyl-O—, —N-azetidinyl, azetidinyl-N—, —O—CH₂—CH₂—, and —CH₂—CH₂—O—, or L¹²² is taken together with R¹²¹ to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
   L¹²³ is selected from: cyclopropyl, —O-cyclopropyl, cyclopropyl-O—, —NH-cyclopropyl, cyclopropyl-NH—, azetidinyl, —O-azetidinyl, azetidinyl-O—, —N-azetidinyl, azetidinyl-N—, or L¹²³ is taken together with R¹²³ to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
   R¹²¹ is selected from: hydrogen, C₁₋₆alkyl, substituted C₁₋₆alkyl, and oxetanyl, or R¹²¹ is taken together with L¹²² to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—; R¹²³ is hydrogen or R¹²³ is taken together with L¹²³ to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
   R¹²⁵ and R¹²⁶ are each independently selected from: methyl, cyclopropyl, —OCF₃, fluoro, chloro, —SCH₃, —OCH₃, —OCHF₂, and —CF₃;
   R¹²² and R¹²⁴ are O;
   C¹² and D¹² are independently phenyl or pyridyl;
   Z¹²² and z¹²⁴ are independently 0 or 1; and
   Z¹²⁶ and z¹²⁶ are independently an integer from 0 to 3;
or a salt thereof including a pharmaceutically acceptable salt thereof.

This invention also relates to pharmaceutically acceptable salts of the compounds of Formula (VIIZ).

Included in the compounds of the invention and used in the methods of the invention are compounds of Formula (VIIIZ):

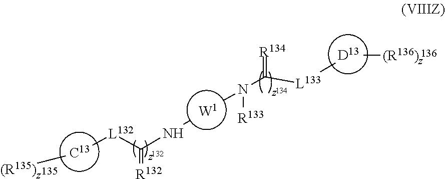

wherein:
   W¹ is selected from bicyclopentanyl and bicyclohexanyl;
   L¹³² is selected from: a bond, —CH₂—, —NH—, CH₂—O—, —O—CH₂—, —O—CH₂—CH₂—, and —CH₂—CH₂—O—;
   L¹³³ is selected from: cyclopropyl, —O-cyclopropyl, cyclopropyl-O—, azetidinyl, —O-azetidinyl, azetidinyl-O—, or L¹³³ is taken together with R¹³³ to form: imidazolidinyl, azetidinyl, azetidinyl-O—, piperidinyl, piperidinyl-O—, piperazinyl, piperazinyl-O—, oxopiperazinyl, oxopiperazinyl-O—, pyrrolidinyl, pyrrolidinyl-O—, oxopyrrolidinyl, or oxopyrrolidinyl-O—;
   R¹³³ is hydrogen or R¹³³ is taken together with L¹³³ to form: imidazolidinyl, azetidinyl, azetidinyl-O—, piperidinyl, piperidinyl-O—, piperazinyl, piperazinyl-O—, oxopiperazinyl, oxopiperazinyl-O—, pyrrolidinyl, pyrrolidinyl-O—, oxopyrrolidinyl, or oxopyrrolidinyl-O—;
   R¹³⁵ and R¹³⁶ are each independently selected from: methyl, cyclopropyl, —OCF₃, fluoro, chloro, —SCH₃, —OCH₃, —OCHF₂, and —CF₃;
   R¹³² and R¹³⁴ are O;
   C¹³ and D¹³ are each independently phenyl or pyridyl;
   Z¹³² and z¹³⁴ are each independently 0 or 1; and
   Z¹³⁵ and z¹³⁶ are each independently an integer from 0 to 3;
or a salt thereof including a pharmaceutically acceptable salt thereof.

This invention also relates to pharmaceutically acceptable salts of the compounds of Formula (VIIIZ).

Included in the compounds of the invention and used in the methods of the invention are compounds of Formula (IIIQ):

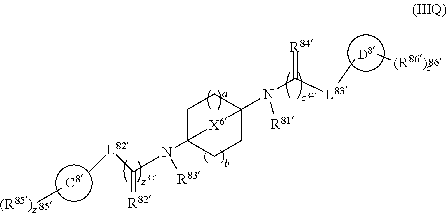

wherein:
   L^{82′} is selected from: a bond, —NH—, —O—, —S—, —S(O)—, —S(O)₂—, cycloalkyl, —O-cycloalkyl, cycloalkyl-O—, —NH-cycloalkyl, cycloalkyl-NH—, azetidinyl, —O-azetidinyl, azetidinyl-O—, —N-azetidinyl, azetidinyl-N—, substituted or unsubstituted C₁₋₆alkylene and substituted or unsubstituted C₁₋₆heteroalkylene, or L^{82′} is taken together with R^{83′} to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—, or, L^{82′} is taken together with an R^{85′} substituent adjacent to the point of attachment of L^{82′} to C^{8′} to form a cycloalkyl ring, a heterocycloalkyl ring, or heteroaryl ring fused to C^{8′};
   L^{83′} is selected from: cycloalkyl, —O-cycloalkyl, cycloalkyl-O—, —NH-cycloalkyl, cycloalkyl-NH—, azetidinyl, —O-azetidinyl, azetidinyl-O—, —N-azetidinyl, azetidinyl-N—, or L^{83′} and R^{81′} are taken together to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—, or, L^{83′} is taken together with an R^{86′} substituent adjacent to the point of attachment of L^{83′} to D^{8′} to form a cycloalkyl ring, a heterocycloalkyl ring, or heteroaryl ring fused to D^{8′};
   R^{81′} is selected from: hydrogen, C₁₋₆alkyl, substituted C₁₋₆alkyl, and heterocycloalkyl, or R^{81′} is taken together with L^{83′} to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
   R^{83′} is selected from: hydrogen, C₁₋₆alkyl, substituted C₁₋₆alkyl, and heterocycloalkyl, or R^{83′} is taken together with L^{82′} to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
   R^{85′} is selected from: fluoro, chloro, bromo, iodo, —OCH₃, —OCH₂Ph, —C(O)Ph, —CF₃, —CN, —S(O)CH₃, —OH, —NH₂, —COOH, —CONH₂, —NO₂, —C(O)CH₃, —C≡CH, —CH₂C≡CH, —SCH₃, —SO₃H, —SO₂NH₂, —NHC(O)NH₂, —NHC(O)H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or, two adjacent R^{85′} substituents can combine to form a cycloalkyl ring, a heterocycloalkyl ring, or heteroaryl ring fused to C^{8′}, or, an R^{85′} substituent adjacent to the point of attachment of L^{82′} to C^{8′} can combine with L^{82′} to form a cycloalkyl ring, a heterocycloalkyl ring, or heteroaryl ring fused to C^{8′};
   R^{86′} is selected from: fluoro, chloro, bromo, iodo, 13 OCH₃, —OCH₂Ph, —C(O)Ph, —CF₃, —CN, —S(O)CH₃, —OH, —NH₂, —COOH, —CONH₂, —NO₂, —C(O)CH₃, —C≡CH, —CH₂C≡CH, —SCH₃, —SO₃H, —SO₂NH₂, —NHC(O)NH₂, —NHC(O)H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or, two adjacent R^{86′} substituents can combine to form a cycloalkyl ring, a heterocycloalkyl ring, or heteroaryl ring fused to D^{8′}, or, an R^{86′} substituent adjacent to the point of attachment of L^{83′} to D^{8′} can combine with L^{83′} to form a cycloalkyl ring, a heterocycloalkyl ring, or heteroaryl ring fused to D^{8′};
   R^{82′} and R^{84′} are independently NR^{88′}, O, CH₂, or S;
   R^{88′} is selected from: hydrogen, C₁₋₆alkyl and C₁₋₆alkyl substituted 1 to 6 times by fluoro;
   a and b are independently 0 or 1;
   C^{8′} and D^{8′} are independently phenyl or pyridyl;
   X^{6′} is C₁₋₃alkylene or c₁₋₃alkylene substituted 1 to 3 times by fluoro;
   Z^{82′} and z^{84′} are independently 0 or 1; and
   Z^{85′} and z^{86′} are independently an integer from 0 to 5;
or a salt thereof including a pharmaceutically acceptable salt thereof.

This invention also relates to pharmaceutically acceptable salts of the compounds of Formula (IIIQ).

Included in the compounds of the invention and used in the methods of the invention are compounds of Formula (IVQ):

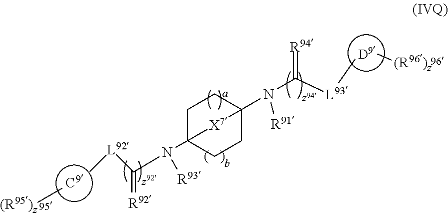

wherein:
L^{92′} is selected from: a bond, —NH—, —O—, —S—, —S(O)—, —S(O)₂—, substituted or unsubstituted C₁₋₆alkylene and substituted or unsubstituted C₁₋₆heteroalkylene;
L^{93′} is selected from: cycloalkyl, —O-cycloalkyl, and cycloalkyl-O—, azetidinyl, —O-azetidinyl, azetidinyl-O—, or L^{93′} is taken together with R^{91′} to form: heterocycloalkyl, heterocycloalkyl-O—, oxoheterocycloalkyl, or oxoheterocycloalkyl-O—, or, L^{93′} is taken together with an R^{96′} substituent adjacent to the point of attachment of L^{93′} to form a cycloalkyl ring, a heterocycloalkyl ring, or heteroaryl ring;
R^{91′} is selected from: hydrogen, C₁₋₆alkyl, substituted C₁₋₆alkyl, and heterocycloalkyl, or R^{91′} is taken together with L^{93′} to form:

heterocycloalkyl, heterocycloalkyl-O—, oxoheterocycloalkyl, or oxoheterocycloalkyl-O—;
R^{93′} is selected from: hydrogen, C₁₋₆alkyl, substituted C₁₋₆alkyl, and heterocycloalkyl;
R^{95′} is selected from: fluoro, chloro, bromo, iodo, —OCH₃, —OCH₂Ph, —C(O)Ph, —CF₃, —CN, —S(O)CH₃, —OH, —NH₂, —COOH, —CONH₂, —NO₂, —C(O)CH₃, —CH₂C≡CH, —SCH₃, —SO₃H, —SO₂NH₂, —NHC(O)NH₂, —NHC(O)H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{96′} is selected from: fluoro, chloro, bromo, iodo, —OCH₃, —OCH₂Ph, —C(O)Ph, —CF₃, —CN, —S(O)CH₃, —OH, —NH₂, —COOH, —CONH₂, —NO₂, —C(O)CH₃, —C≡CH, —CH₂C≡CH, —SCH₃, —SO₃H, —SO₂NH₂, —NHC(O)NH₂, —NHC(O)H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or, two adjacent R^{96′} substituents can combine to form a cycloalkyl ring, a heterocycloalkyl ring, or heteroaryl ring fused to D^{9′}, or, an R^{96′} substituent adjacent to the point of attachment of L^{93′} to D^{9′} can combine with L^{93′} to form a cycloalkyl ring, a heterocycloalkyl ring, or heteroaryl ring fused to D^{9′};
R^{92′} and R^{94′} are independently NR^{98′}, O, or S;
R^{98′} is selected from: hydrogen, C₁₋₆alkyl and C₁₋₆alkyl substituted 1 to 6 times by fluoro;
a and b are independently 0 or 1;
C^{9′} and D^{9′} are independently phenyl or pyridyl;
X^{7′} is C₁₋₃alkylene or C₁₋₃alkylene substituted 1 to 3 times by fluoro;
Z^{92′} and z^{94′} are independently 0 or 1; and
Z^{95′} and z^{96′} are independently an integer from 0 to 5;
or a salt thereof including a pharmaceutically acceptable salt thereof.

This invention also relates to pharmaceutically acceptable salts of the compounds of Formula (IVQ).

Included in the compounds of the invention and used in the methods of the invention are compounds of Formula (VQ):

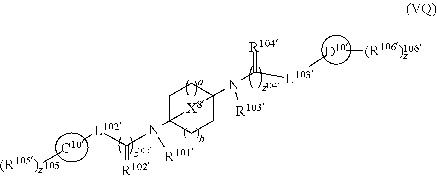

wherein:
   L^{102′} is selected from: a bond, —CH₂—, —NH—, CH₂—O—, —O—CH₂—, cyclopropyl, —O-cyclopropyl, cyclopropyl-O—, —NH-cyclopropyl, cyclopropyl-NH—, azetidinyl, —O-azetidinyl, azetidinyl-O—, —N-azetidinyl, azetidinyl-N—, —O—CH₂—CH₂-, and —CH₂—CH₂—O—, or L^{102′} is taken together with R^{101′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—, or, L^{102′} is taken together with an R^{105′} substituent adjacent to the point of attachment of L^{102′} to form a heterocycloalkyl ring;
   L^{103′} is selected from: cyclopropyl, —O-cyclopropyl, cyclopropyl-O—, —NH-cyclopropyl, cyclopropyl-NH—, azetidinyl, —O-azetidinyl, azetidinyl-O—, —N-azetidinyl, azetidinyl-N—, or L^{103′} is taken together with R^{103′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—, or, L^{103′} is taken together with an R^{106′} substituent adjacent to the point of attachment of L^{103′} to form a heterocycloalkyl ring;
   R^{101′} is selected from: hydrogen, C₁₋₆alkyl, substituted C₁₋₆alkyl, and oxetanyl, or R¹⁰¹ is taken together with L^{102′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
   R^{103′} is selected from: hydrogen, C₁₋₆alkyl, substituted C₁₋₆alkyl, and oxetanyl, or R^{103′} is taken together with L^{103′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
   R^{105′} is selected from: methyl, cyclopropyl, —OCF₃, fluoro, chloro, —SCH₃, —OCH₃, —OCHF₂, and —CF₃, or, an R^{105′} substituent adjacent to the point of attachment of L^{102′} to C^{10′} can combine with L^{102′} to form a heterocycloalkyl ring fused to C^{10′};
   R^{106′} is selected from: methyl, cyclopropyl, —OCF₃, fluoro, chloro, —SCH₃, —OCH₃, —OCHF₂, and —CF₃, or, an R^{106′} substituent adjacent to the point of attachment of L^{103′} to D^{10′} can combine with L^{103′} to form a heterocycloalkyl ring fused to D^{10′};
   R^{102′} and R^{104′} are O;
   a and b are independently 0 or 1;
   C^{10′} and D^{10′} areindependently phenyl or pyridyl;
   X^{8′} is selected from —CH₂— and —CH₂—CH₂—;
   Z^{102′} and z^{104′} are independently 0 or 1; and
   Z^{105′}and z^{106′} are independently an integer from 0 to 5;
or a salt thereof including a pharmaceutically acceptable salt thereof.

This invention also relates to pharmaceutically acceptable salts of the compounds of Formula (VQ).

Included in the compounds of the invention and used in the methods of the invention are compounds of Formula (VIQ):

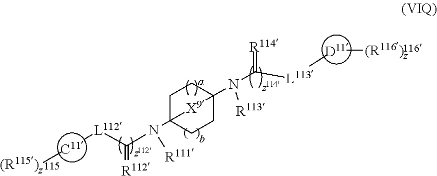

wherein:
   L^{112′} is selected from: a bond, −CH₂—, —NH—, CH₂—O—, —O—CH₂—, —O—CH₂—CH₂—, and —CH₂—CH₂—O—;
   L^{113′} is selected from: cyclopropyl, —O-cyclopropyl, cyclopropyl-O—, azetidinyl, —O-azetidinyl, azetidinyl-O—, or L^{113′} is taken together with R^{113′} to form: imidazolidinyl, azetidinyl, azetidinyl-O—, piperidinyl, piperidinyl-O—, piperazinyl, piperazinyl-O—, oxopiperazinyl, oxopiperazinyl-O—, pyrrolidinyl, pyrrolidinyl-O—, oxopyrrolidinyl, or oxopyrrolidinyl-O—, or, L^{113′} is taken together with an R^{116′} substituent adjacent to the point of attachment of L^{113′} to form a heterocycloalkyl ring;
   R^{113′} is selected from: hydrogen, C₁₋₆alkyl, substituted C₁₋₆alkyl, and oxetanyl or R^{113′} is taken together with L^{113′} to form: imidazolidinyl, azetidinyl, azetidinyl-O—, piperidinyl, piperidinyl-O—, piperazinyl, piperazinyl-O—, oxopiperazinyl, oxopiperazinyl-O—, pyrrolidinyl, pyrrolidinyl-O—, oxopyrrolidinyl, or oxopyrrolidinyl-O—;
   R^{111′} is selected from: hydrogen, C₁₋₆alkyl, substituted C₁₋₆alkyl, and oxetanyl;
   R^{115′} is selected from: methyl, cyclopropyl, —OCF₃, fluoro, chloro, —SCH₃, —OCH₃, —OCHF₂, and —CF₃;
   R^{116′} is selected from: methyl, cyclopropyl, —OCF₃, fluoro, chloro, —SCH₃, —OCH₃, —OCHF₂, and —CF₃, or, an R^{116′} substituent adjacent to the point of attachment of L^{113′} to D^{11′} can combine with L^{113′} to form a heterocycloalkyl ring fused to D^{11′};
   R^{112′} and R^{114′} are O;
   a and b are independently 0 or 1;
   C^{11′} and D^{11′} are independently phenyl or pyridyl;
   X^{9′} is selected from —CH₂— and —CH₂—CH₂—;
   Z^{112′} and z^{114′} are independently 0 or 1; and
   Z^{115′} and z^{116′} are independently an integer from 0 to 5;
or a salt thereof including a pharmaceutically acceptable salt thereof.

This invention also relates to pharmaceutically acceptable salts of the compounds of Formula (VIQ).

Included in the compounds of the invention and used in the methods of the invention are compounds of Formula (VIIQ):

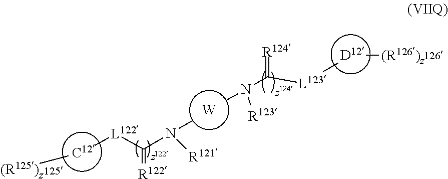

wherein:
   W is selected from bicyclopentanyl and bicyclohexanyl;
   L^{122′} is selected from: a bond, —CH₂—, —NH—, CH₂—O—, —O—CH₂—, cyclopropyl, —O-cyclopropyl, cyclopropyl-O—, —NH-cyclopropyl, cyclopropyl-NH—, azetidinyl, —O-azetidinyl, azetidinyl-O—, —N-azetidinyl, azetidinyl-N—, —O—CH₂—CH₂—, and —CH₂—CH₂—O—, or L^{122′} is taken together with R^{121′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—, or, L^{122′} is taken together with an R^{125′} substituent adjacent to the point of attachment of L^{122′} to form a cyclohexyl ring, a cyclobutyl ring, or a tetrahydro-pyran ring;
   L^{123′} is selected from: cyclopropyl, azetidinyl, —O-azetidinyl, azetidinyl-O—, —N-azetidinyl, azetidinyl-N—, or L^{123′} is taken together with R^{123′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—, or, L^{123′} is taken together with an R^{126′} substituent adjacent to the point of attachment of L^{123′} to form a cyclohexyl ring, a cyclobutyl ring, or a tetrahydro-pyran ring;
   R^{121′} is selected from: hydrogen, C₁₋₆alkyl, substituted C₁₋₆alkyl, and oxetanyl, or R^{121′} is taken together with L^{122′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
   R^{123′} is hydrogen or R^{123′} is taken together with L^{123′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
   R^{125′} is selected from: methyl, cyclopropyl, —OCF₃, fluoro, chloro, —SCH₃, —OCH₃, —OCHF₂, and —CF₃, or, an R^{125′} substituent adjacent to the point of attachment of L^{122′} to C^{12′} can combine with L^{122′} to form a cyclohexyl ring, a cyclobutyl ring, or a tetrahydro-pyran ring fused to C^{12′};
   R^{126′} is selected from: methyl, cyclopropyl, —OCF₃, fluoro, chloro, —SCH₃, —OCH₃, —OCHF₂, and —CF₃, or, an R^{126′} substituent adjacent to the point of attachment of L^{123′} to D^{12′} can combine with L^{123′} to form a cyclohexyl ring, a cyclobutyl ring, or a tetrahydro-pyran ring fused to D^{12′};
   R^{122′} and R^{124′} are O;
   C^{12′} and D^{12′} are independently phenyl or pyridyl;
   Z^{122′} and z^{124′} are independently 0 or 1; and
   Z^{125′} and z^{126′} are independently an integer from 0 to 3;
or a salt thereof including a pharmaceutically acceptable salt thereof.

This invention also relates to pharmaceutically acceptable salts of the compounds of Formula (VIIQ).

Included in the compounds of the invention and used in the methods of the invention are compounds of Formula (VIIIQ):

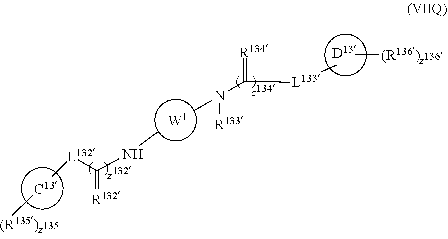

wherein:
   W¹ is selected from bicyclopentanyl and bicyclohexanyl;
   L^{132′} is selected from: a bond, —CH₂′, —NH—, CH₂—O—, —O—CH₂—, —O—CH₂—CH₂—, and —CH₂—CH₂—O—;
   L^{133′} is selected from: cyclopropyl, —O-cyclopropyl, cyclopropyl-O—, azetidinyl, —O-azetidinyl, azetidinyl-O—, or L^{133′} is taken together with R^{133′} to form: imidazolidinyl, azetidinyl, azetidinyl-O—, piperidinyl, piperidinyl-O—, piperazinyl, piperazinyl-O—, oxopiperazinyl, oxopiperazinyl-O—, pyrrolidinyl, pyrrolidinyl-O—, oxopyrrolidinyl, or oxopyrrolidinyl-O— or, L^{133′} is taken together with an R^{136′} substituent adjacent to the point of attachment of L^{133′} to form a cyclohexyl ring, a cyclobutyl ring, or a tetrahydro-pyran ring;
   R^{133′} is hydrogen or R^{133′} is taken together with L^{133′} to form: imidazolidinyl, azetidinyl, azetidinyl-O—, piperidinyl, piperidinyl-O—, piperazinyl, piperazinyl-O—, oxopiperazinyl, oxopiperazinyl-O—, pyrrolidinyl, pyrrolidinyl-O—, oxopyrrolidinyl, or oxopyrrolidinyl-O—;
   R^{135′} is selected from: methyl, cyclopropyl, —OCF₃, fluoro, chloro, —SCH₃, —OCH₃, —OCHF₂, and —CF₃;
   R^{136′} is selected from: methyl, cyclopropyl, —OCF₃, fluoro, chloro, —SCH₃, —OCH₃, —OCHF₂, and —CF₃, or, an R^{136′} substituent adjacent to the point of attachment of L^{133′} to D^{13′} can combine with L^{133′} to form a cyclohexyl ring, a cyclobutyl ring, or a tetrahydro-pyran ring fused to D^{13′};
   R^{132′} and R^{134′} are O;
   C^{13′} and D^{13′} are each independently phenyl or pyridyl;
   Z^{132′} and z^{134′} are each independently 0 or 1; and
   Z^{135′} and z^{136′} are each independently an integer from 0 to 3;
or a salt thereof including a pharmaceutically acceptable salt thereof.

This invention also relates to pharmaceutically acceptable salts of the compounds of Formula (VIIIQ).

Included in the compounds of the invention are:

N,N′-(bicyclo[2.2.2]octane-1,4-diyl)bis(2-(4-chlorophenoxy)acetamide);

2-(4-chlorophenoxy)-N-(4-(2-((6-chloropyridin-3-yl)oxy)acetamido)bicyclo[2.2.2]octan-1-yl)acetamide;

N,N′-(bicyclo[2.2.2]octane-1,4-diyl)bis(2-((6-chloropyridin-3-yl)oxy)acetamide);

N,N′-(bicyclo[1.1.1]pentane-1,3-diyl)bis(2-(4-chlorophenoxy)acetamide);

N,N′-(bicyclo[1.1.1]pentane-1,3-diyl)bis(2-phenoxyacetamide);

2-(4-chlorophenoxy)-N-(3-(2-(4-chlorophenyl)acetamido)bicyclo[1.1.1]pentan-1-yl)acetamide;

2-(4-chlorophenoxy)-N-(3-(2-(p-tolyloxy)acetamido)bicyclo[1.1.1]pentan-1-yl)acetamide;

2-(4-chlorophenoxy)-N-(3-(2-((6-chloropyridin-3-yl)oxy)acetamido)bicyclo[1.1.1]pentan-1-yl)acetamide;

2-(4-chlorophenoxy)-N-(3-(2-((6-methylpyridin-3-yl)oxy)acetamido)bicyclo[1.1.1]pentan-1-yl)acetamide;

2-(4-chlorophenoxy)-N-(3-(2-((5-chloropyridin-2-yl)oxy)acetamido)bicyclo[1.1.1]pentan-1-yl)acetamide;

2-(4-chlorophenoxy)-N-(3-(2-phenoxyacetamido)bicyclo[1.1.1]pentan-1-yl)acetamide;

4-chloro-N-(3-(2-(4-chlorophenoxy)acetamido)bicyclo[1.1.1]pentan-1-yl)benzamide;

2-((3-(2-(4-chlorophenoxy)acetamido)bicyclo[1.1.1]pentan-1-yl)(2-(4-chlorophenoxy)ethyl)amino)-N,N-dimethylacetamide;

2-(4-chlorophenoxy)-N-(3-((2-(4-chlorophenoxy)ethyl)amino)bicyclo[1.1.1]pentan-1-yl)acetamide;

N-(3-((2-(4-chlorophenoxy)ethyl)amino)bicyclo[1.1.1]pentan-1-yl)-2-((5-chloropyridin-2-yl)oxy)acetamide;

methyl N-(3-(2-(4-chlorophenoxy)acetamido)bicyclo[1.1.1]pentan-1-yl)-N-(2-(4-chlorophenoxy)ethyl)glycinate;

ethyl 4-((3-(2-(4-chlorophenoxy)acetamido)bicyclo[1.1.1]pentan-1-yl)(2-(4-chlorophenoxy)ethyl)amino)butanoate;

2-(4-chlorophenoxy)-N-(3-((2-(4-chlorophenoxy)ethyl)(methyl)amino)bicyclo[1.1.1]pentan-1-yl)acetamide;

2-(4-chlorophenoxy)-N-(3-(N-(2-(4-chlorophenoxy)ethyl)acetamido)bicyclo[1.1.1]pentan-1-yl)acetamide;

2-(4-chlorophenoxy)-N-(3-((2-(4-chlorophenoxy)ethyl)(oxetan-3-yl)amino)bicyclo[1.1.1]pentan-1-yl)acetamide;

2-(4-chlorophenoxy)-N-(3-((2-((4-chlorophenyl)amino)ethyl)amino)bicyclo[1.1.1]pentan-1-yl)acetamide;

2-(4-chlorophenoxy)-N-(3-(3-(4-chlorophenyl)-2-oxoimidazolidin-1-yl)bicyclo[1.1.1]pentan-1-yl)acetamide;

1-(3-((2-(4-chlorophenoxy)ethyl)amino)bicyclo[1.1.1]pentan-1-yl)-3-(4-chlorophenyl)imidazolidin-2-one;

2-(4-chlorophenoxy)-N-(3-(3-(4-chlorophenoxy)pyrrolidin-1-yl)bicyclo[1.1.1]pentan-1-yl)acetamide;

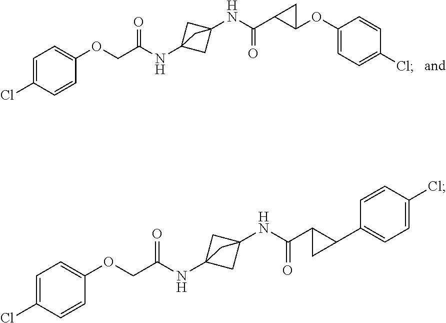 [SMILES: O=C(COc1ccc(Cl)cc1)NC12CC(NC(=O)C3CC3Oc3ccc(Cl)cc3)(C1)C2 ; O=C(COc1ccc(Cl)cc1)NC12CC(NC(=O)C3CC3c3ccc(Cl)cc3)(C1)C2]

and salts thereof including pharmaceutically acceptable salts thereof.

Included in the compounds of the invention are:

2-(4-chlorophenoxy)-N-(3-(3-(4-chlorophenyl)-2-oxoimidazolidin-1-yl)bicyclo[1.1.1]pentan-1-yl)acetamide;

2-(4-chlorophenoxy)-N-(3-(3-(4-fluorophenyl)-2-oxoimidazolidin-1-yl)bicyclo[1.1.1]pentan-1-yl)acetamide;

N-(3-(3-(4-chlorophenyl)-2-oxoimidazolidin-1-yl)bicyclo[1.1.1]pentan-1-yl)-2-(4-fluorophenoxy)acetamide;

N-(3-(3-(4-chloro-2-methylphenyl)-2-oxoimidazolidin-1-yl)bicyclo[1.1.1]pentan-1-yl)-2-(4-chlorophenoxy)acetamide;

N-(3-(3-(4-chlorophenyl)-2-oxoimidazolidin-1-yl)bicyclo[1.1.1]pentan-1-yl)-2-(4-cyclopropylphenoxy)acetamide;

2-(4-chlorophenoxy)-N-(3-(3-(5-chloropyridin-2-yl)-2-oxoimidazolidin-1-yl)bicyclo[1.1.1]pentan-1-yl)acetamide;

2-(3-chlorophenoxy)-N-(3-(3-(4-chlorophenyl)-2-oxoimidazolidin-1-yl)bicyclo[1.1.1]pentan-1-yl)acetamide;

N-(3-(3-(4-chlorophenyl)-2-oxoimidazolidin-1-yl)bicyclo[1.1.1]pentan-1-yl)-2-(4-(trifluoromethoxy)phenoxy)acetamide;

2-(4-chlorophenoxy)-N-(3-(3-(4-chlorophenyl)-2-oxoimidazolidin-1-yl)bicyclo[1.1.1]pentan-1-yl)acetamide;

2-(4-chloro-3-(trifluoromethyl)phenoxy)-N-(3-(3-(4-chlorophenyl)-2-oxoimidazolidin-1-yl)bicyclo[1.1.1]pentan-1-yl)acetamide;

2-(4-chlorophenoxy)-N-(3-(3-(3-chlorophenyl)-2-oxoimidazolidin-1-yl)bicyclo[1.1.1]pentan-1-yl)acetamide;

N-(3-(2-(4-chlorophenoxy)acetamido)bicyclo[1.1.1]pentan-1-yl)-2-(4-chlorophenyl)cyclopropane-1-carboxamide;

N-(4-(2-(4-chlorophenoxy)acetamido)bicyclo[2.1.1]hexan-1-yl)-2-(4-chlorophenyl)cyclopropane-1-carboxamide;

2-(4-chlorophenoxy)-N-(3-(2-(4-chlorophenoxy)acetamido)bicyclo[1.1.1]pentan-1-yl)cyclopropane-1-carboxamide;

2-(4-chlorophenoxy)-N-(3-((1-(4-chlorophenyl)azetidin-3-yl)amino)bicyclo[1.1.1]pentan-1-yl)acetamide;

2-(4-chlorophenoxy)-N-(3-(3-(4-chlorophenoxy)azetidin-1-yl)bicyclo[1.1.1]pentan-1-yl)acetamide;

2-(4-chlorophenoxy)-N-(3-(2-((5,6,7,8-tetrahydronaphthalen-2-yl)oxy)acetamido)bicyclo[1.1.1]pentan-1-yl)acetamide;

5-chloro-N-(3-(2-(4-chlorophenoxy)acetamido)bicyclo[1.1.1]pentan-1-yl)-2,3-dihydrobenzofuran-2-carboxamide;

2-(bicyclo[4.2.0]octa-1(6),2,4-trien-3-yloxy)-N-(3-(2-(4-chlorophenoxy)acetamido)bicyclo[1.1.1]pentan-1-yl)acetamide;

2-(4-chlorophenoxy)-N-(3-(2-(chroman-6-yloxy)acetamido)bicyclo[1.1.1]pentan-1-yl)acetamide;

2-(4-chlorophenoxy)-N-(3-(4-(4-chlorophenyl)piperidin-1-yl)bicyclo[1.1.1]pentan-1-yl)acetamide;

2-(4-chlorophenoxy)-N-(3-(4-(4-chlorophenyl)piperazin-1-yl)bicyclo[1.1.1]pentan-1-yl)acetamide;

2-(bicyclo[4.2.0]octa-1,3,5-trien-3-yloxy)-N-(4-(2-(4-chlorophenoxy)acetamido)bicyclo[2.2.1]heptan-1-yl)acetamide;

2-(4-chlorophenoxy)-N-(3-(3-(4-chlorophenoxy)pyrrolidin-1-yl)bicyclo[1.1.1]pentan-1-yl)acetamide;

(S)-2-(4-chlorophenoxy)-N-(3-(3-(4-chlorophenoxy)pyrrolidin-1-yl)bicyclo[1.1.1]pentan-1-yl)acetamide;

(R)-2-(4-chlorophenoxy)-N-(3-(3-(4-chlorophenoxy)pyrrolidin-1-yl)bicyclo[1.1.1]pentan-1-yl)acetamide;

N-(3-(3-(4-chlorophenoxy)pyrrolidin-1-yl)bicyclo[1.1.1]pentan-1-yl)-2-(4-fluorophenoxy)acetamide isomer 1;

N-(3-(3-(4-chlorophenoxy)pyrrolidin-1-yl)bicyclo[1.1.1]pentan-1-yl)-2-(4-fluorophenoxy)acetamide isomer 2;

2-(4-chlorophenoxy)-N-(3-(3-(4-fluorophenoxy)pyrrolidin-1-yl)bicyclo[1.1.1]pentan-1-yl)acetamide;

N-(3-(3-(3-chloro-4-fluorophenoxy)pyrrolidin-1-yl)bicyclo[1.1.1]pentan-1-yl)-2-(4-chlorophenoxy)acetamide isomer 1;

N-(3-(3-(3-chloro-4-fluorophenoxy)pyrrolidin-1-yl)bicyclo[1.1.1]pentan-1-yl)-2-(4-chlorophenoxy)acetamide isomer 2;

N-(3-(3-(4-chlorophenoxy)pyrrolidin-1-yl)bicyclo[1.1.1]pentan-1-yl)-2-((5-chloropyridin-2-yl)oxy)acetamide;

N-(3-(3-(4-chlorophenoxy)pyrrolidin-1-yl)bicyclo[1.1.1]pentan-1-yl)-2-(4-(trifluoromethyl)phenoxy)acetamide;

N-(3-(3-(4-chlorophenoxy)pyrrolidin-1-yl)bicyclo[1.1.1]pentan-1-yl)-2-(4-(trifluoromethoxy)phenoxy)acetamide;

2-(4-chloro-3-(trifluoromethyl)phenoxy)-N-(3-(3-(4-chlorophenoxy)pyrrolidin-1-yl)bicyclo[1.1.1]pentan-1-yl)acetamide;

2-(4-chlorophenoxy)-N-(3-(3-(4-(trifluoromethyl)phenoxy)pyrrolidin-1-yl)bicyclo[1.1.1]pentan-1-yl)acetamide;

2-(4-chloro-3-fluorophenoxy)-N-(3-(3-(4-chlorophenoxy)pyrrolidin-1-yl)bicyclo[1.1.1]pentan-1-yl)acetamide;

N-(3-(3-(4-chlorophenoxy)pyrrolidin-1-yl)bicyclo[1.1.1]pentan-1-yl)-2-(4-cyclopropylphenoxy)acetamide;

N-(3-(3-(4-chloro-3-fluorophenoxy)pyrrolidin-1-yl)bicyclo[1.1.1]pentan-1-yl)-2-(4-chlorophenoxy)acetamide isomer 1;

N-(3-(3-(4-chloro-3-fluorophenoxy)pyrrolidin-1-yl)bicyclo[1.1.1]pentan-1-yl)-2-(4-chlorophenoxy)acetamide isomer 2;

2-(4-chlorophenoxy)-N-(3-(3-(pyridin-4-yloxy)pyrrolidin-1-yl)bicyclo[1.1.1]pentan-1-yl)acetamide;

1-(3-((2-(4-chlorophenoxy)ethyl)amino)bicyclo[1.1.1]pentan-1-yl)-3-(4-chlorophenyl)imidazolidin-2-one;

2-(4-chloro-3-fluorophenoxy)-N-(3-(3-(4-chlorophenyl)-2-oxoimidazolidin-1-yl)bicyclo[1.1.1]pentan-1-yl)acetamide;

N-(3-(3-(4-chlorophenyl)-2-oxoimidazolidin-1-yl)bicyclo[1.1.1]pentan-1-yl)-2-(4-methoxyphenoxy)acetamide;

2-(3-chloro-4-fluorophenoxy)-N-(3-(3-(4-chlorophenyl)-2-oxoimidazolidin-1-yl)bicyclo[1.1.1]pentan-1-yl)acetamide;

N-(3-(3-(4-chloro-3-(trifluoromethyl)phenyl)-2-oxoimidazolidin-1-yl)bicyclo[1.1.1]pentan-1-yl)-2-(4-chlorophenoxy)acetamide;

2-(4-chlorophenoxy)-N-(3-(3-(4-fluoro-3-(trifluoromethyl)phenyl)-2-oxoimidazolidin-1-yl)bicyclo[1.1.1]pentan-1-yl)acetamide;

N-(3-(5-chloroisoindolin-2-yl)bicyclo[1.1.1]pentan-1-yl)-2-(4-chlorophenoxy)acetamide;

N-(3-(3-(4-chlorophenyl)-2-oxoimidazolidin-1-yl)bicyclo[1.1.1]pentan-1-yl)-2-((5-chloropyridin-2-yl)oxy)acetamide;

2-(4-chlorophenoxy)-N-(3-(2-oxo-3-(4-(trifluoromethyl)phenyl)imidazolidin-1-yl)bicyclo[1.1.1]pentan-1-yl)acetamide;

N-(3-(3-(4-chlorophenyl)-2-oxoimidazolidin-1-yl)bicyclo[1.1.1]pentan-1-yl)-2-(4-fluoro-3-(trifluoromethyl)phenoxy)acetamide;

N-(3-(3-(4-chlorophenoxy)pyrrolidin-1-yl)bicyclo[1.1.1]pentan-1-yl)-2-(4-fluoro-3-(trifluoromethyl)phenoxy)acetamide;

N-(3-(3-(4-chlorophenoxy)pyrrolidin-1-yl)bicyclo[1.1.1]pentan-1-yl)-2-(4-(difluoromethoxy)phenoxy)acetamide;

2-(4-chloro-3-fluorophenoxy)-N-(3-(3-(4-chlorophenoxy)pyrrolidin-1-yl)bicyclo[1.1.1]pentan-1-yl)acetamide isomer 1;

(R)-2-(4-chloro-3-fluorophenoxy)-N-(3-(3-(4-chlorophenoxy)pyrrolidin-1-yl)bicyclo[1.1.1]pentan-1-yl)acetamide isomer 2;

2-(4-chlorophenoxy)-N-(3-(3-((5-chloropyridin-2-yl)oxy)pyrrolidin-1-yl)bicyclo[1.1.1]pentan-1-yl)acetamide;

2-((5-chloropyridin-2-yl)oxy)-N-(3-(3-((5-chloropyridin-2-yl)oxy)pyrrolidin-1-yl)bicyclo[1.1.1]pentan-1-yl)acetamide;

2-(4-chlorophenoxy)-N-(3-(3-(4-methoxyphenyl)-2-oxoimidazolidin-1-yl)bicyclo[1.1.1]pentan-1-yl)acetamide;

N-(3-(3-(4-chloro-2-fluorophenyl)-2-oxoimidazolidin-1-yl)bicyclo[1.1.1]pentan-1-yl)-2-(4-chlorophenoxy)acetamide;

N-(3-(3-(bicyclo[4.2.0]octa-1,3,5-trien-3-yloxy)pyrrolidin-1-yl)bicyclo[1.1.1]pentan-1-yl)-2-(4-chlorophenoxy)acetamide;

(S)-2-(4-chlorophenoxy)-N-(3-(4-(4-chlorophenoxy)-2-oxopyrrolidin-1-yl)bicyclo[1.1.1]pentan-1-yl)acetamide;

(S)-2-(4-chlorophenoxy)-N-(3-(3-(4-chlorophenoxy)-2-oxopyrrolidin-1-yl)bicyclo[1.1.1]pentan-1-yl)acetamide;

(R)-2-(4-chlorophenoxy)-N-(3-(4-(4-chlorophenoxy)-2-oxopyrrolidin-1-yl)bicyclo[1.1.1]pentan-1-yl)acetamide;

(R)-2-(4-chlorophenoxy)-N-(3-(3-(4-chlorophenoxy)-2-oxopyrrolidin-1-yl)bicyclo[1.1.1]pentan-1-yl)acetamide;

2-(4-chlorophenoxy)-N-(3-(3-(4-(methylthio)phenyl)-2-oxoimidazolidin-1-yl)bicyclo[1.1.1]pentan-1-yl)acetamide; and

2-(4-chlorophenoxy)-N-(3-(4-(4-chlorophenoxy)piperidin-1-yl)bicyclo[1.1.1]pentan-1-yl)acetamide;
and salts thereof including pharmaceutically acceptable salts thereof.

Included in the compounds of the invention are:

2-(4-chlorophenoxy)-N-(3-(3-(4-chlorophenyl)-2-oxoimidazolidin-1-yl)bicyclo[1.1.1]pentan-1-yl)acetamide;

2-(4-chlorophenoxy)-N-(3-(3-(4-fluorophenyl)-2-oxoimidazolidin-1-yl)bicyclo[1.1.1]pentan-1-yl)acetamide;

N-(3-(3-(4-chlorophenyl)-2-oxoimidazolidin-1-yl)bicyclo[1.1.1]pentan-1-yl)-2-(4-fluorophenoxy)acetamide;

N-(3-(3-(4-chloro-2-methylphenyl)-2-oxoimidazolidin-1-yl)bicyclo[1.1.1]pentan-1-yl)-2-(4-chlorophenoxy)acetamide;

N-(3-(3-(4-chlorophenyl)-2-oxoimidazolidin-1-yl)bicyclo[1.1.1]pentan-1-yl)-2-(4-cyclopropylphenoxy)acetamide;

2-(4-chlorophenoxy)-N-(3-(3-(5-chloropyridin-2-yl)-2-oxoimidazolidin-1-yl)bicyclo[1.1.1]pentan-1-yl)acetamide;

2-(3-chlorophenoxy)-N-(3-(3-(4-chlorophenyl)-2-oxoimidazolidin-1-yl)bicyclo[1.1.1]pentan-1-yl)acetamide;

N-(3-(3-(4-chlorophenyl)-2-oxoimidazolidin-1-yl)bicyclo[1.1.1]pentan-1-yl)-2-(4-(trifluoromethoxy)phenoxy)acetamide;

2-(4-chlorophenoxy)-N-(3-(3-(4-chlorophenyl)-2-oxoimidazolidin-1-yl)bicyclo[1.1.1]pentan-1-yl)acetamide;

2-(4-chloro-3-(trifluoromethyl)phenoxy)-N-(3-(3-(4-chlorophenyl)-2-oxoimidazolidin-1-yl)bicyclo[1.1.1]pentan-1-yl)acetamide;

2-(4-chlorophenoxy)-N-(3-(3-(3-chlorophenyl)-2-oxoimidazolidin-1-yl)bicyclo[1.1.1]pentan-1-yl)acetamide;

N-(3-(2-(4-chlorophenoxy)acetamido)bicyclo[1.1.1]pentan-1-yl)-2-(4-chlorophenyl)cyclopropane-1-carboxamide;

N-(4-(2-(4-chlorophenoxy)acetamido)bicyclo[2.1.1]hexan-1-yl)-2-(4-chlorophenyl)cyclopropane-1-carboxamide;

2-(4-chlorophenoxy)-N-(3-(2-(4-chlorophenoxy)acetamido)bicyclo[1.1.1]pentan-1-yl)cyclopropane-1-carboxamide;

2-(4-chlorophenoxy)-N-(3-((1-(4-chlorophenyl)azetidin-3-yl)amino)bicyclo[1.1.1]pentan-1-yl)acetamide;

2-(4-chlorophenoxy)-N-(3-(3-(4-chlorophenoxy)azetidin-1-yl)bicyclo[1.1.1]pentan-1-yl)acetamide;

2-(4-chlorophenoxy)-N-(3-(4-(4-chlorophenyl)piperidin-1-yl)bicyclo[1.1.1]pentan-1-yl)acetamide;

2-(4-chlorophenoxy)-N-(3-(4-(4-chlorophenyl)piperazin-1-yl)bicyclo[1.1.1]pentan-1-yl)acetamide;

2-(bicyclo[4.2.0]octa-1,3,5-trien-3-yloxy)-N-(4-(2-(4-chlorophenoxy)acetamido)bicyclo[2.2.1]heptan-1-yl)acetamide;

2-(4-chlorophenoxy)-N-(3-(3-(4-chlorophenoxy)pyrrolidin-1-yl)bicyclo[1.1.1]pentan-1-yl)acetamide;

(S)-2-(4-chlorophenoxy)-N-(3-(3-(4-chlorophenoxy)pyrrolidin-1-yl)bicyclo[1.1.1]pentan-1-yl)acetamide;

(R)-2-(4-chlorophenoxy)-N-(3-(3-(4-chlorophenoxy)pyrrolidin-1-yl)bicyclo[1.1.1]pentan-1-yl)acetamide;

N-(3-(3-(4-chlorophenoxy)pyrrolidin-1-yl)bicyclo[1.1.1]pentan-1-yl)-2-(4-fluorophenoxy)acetamide isomer 1;

N-(3-(3-(4-chlorophenoxy)pyrrolidin-1-yl)bicyclo[1.1.1]pentan-1-yl)-2-(4-fluorophenoxy)acetamide isomer 2;

2-(4-chlorophenoxy)-N-(3-(3-(4-fluorophenoxy)pyrrolidin-1-yl)bicyclo[1.1.1]pentan-1-yl)acetamide;

N-(3-(3-(3-chloro-4-fluorophenoxy)pyrrolidin-1-yl)bicyclo[1.1.1]pentan-1-yl)-2-(4-chlorophenoxy)acetamide isomer 1;

N-(3-(3-(3-chloro-4-fluorophenoxy)pyrrolidin-1-yl)bicyclo[1.1.1]pentan-1-yl)-2-(4-chlorophenoxy)acetamide isomer 2;

N-(3-(3-(4-chlorophenoxy)pyrrolidin-1-yl)bicyclo[1.1.1]pentan-1-yl)-2-((5-chloropyridin-2-yl)oxy)acetamide;

N-(3-(3-(4-chlorophenoxy)pyrrolidin-1-yl)bicyclo[1.1.1]pentan-1-yl)-2-(4-(trifluoromethyl)phenoxy)acetamide;

N-(3-(3-(4-chlorophenoxy)pyrrolidin-1-yl)bicyclo[1.1.1]pentan-1-yl)-2-(4-(trifluoromethoxy)phenoxy)acetamide;

2-(4-chloro-3-(trifluoromethyl)phenoxy)-N-(3-(3-(4-chlorophenoxy)pyrrolidin-1-yl)bicyclo[1.1.1]pentan-1-yl)acetamide;

2-(4-chlorophenoxy)-N-(3-(3-(4-(trifluoromethyl)phenoxy)pyrrolidin-1-yl)bicyclo[1.1.1]pentan-1-yl)acetamide;

2-(4-chloro-3-fluorophenoxy)-N-(3-(3-(4-chlorophenoxy)pyrrolidin-1-yl)bicyclo[1.1.1]pentan-1-yl)acetamide;

N-(3-(3-(4-chlorophenoxy)pyrrolidin-1-yl)bicyclo[1.1.1]pentan-1-yl)-2-(4-cyclopropylphenoxy)acetamide;

N-(3-(3-(4-chloro-3-fluorophenoxy)pyrrolidin-1-yl)bicyclo[1.1.1]pentan-1-yl)-2-(4-chlorophenoxy)acetamide isomer 1;

N-(3-(3-(4-chloro-3-fluorophenoxy)pyrrolidin-1-yl)bicyclo[1.1.1]pentan-1-yl)-2-(4-chlorophenoxy)acetamide isomer 2;

2-(4-chlorophenoxy)-N-(3-(3-(pyridin-4-yloxy)pyrrolidin-1-yl)bicyclo[1.1.1]pentan-1-yl)acetamide;

1-(3-((2-(4-chlorophenoxy)ethyl)amino)bicyclo[1.1.1]pentan-1-yl)-3-(4-chlorophenyl)imidazolidin-2-one;

2-(4-chloro-3-fluorophenoxy)-N-(3-(3-(4-chlorophenyl)-2-oxoimidazolidin-1-yl)bicyclo[1.1.1]pentan-1-yl)acetamide;

N-(3-(3-(4-chlorophenyl)-2-oxoimidazolidin-1-yl)bicyclo[1.1.1]pentan-1-yl)-2-(4-methoxyphenoxy)acetamide;

2-(3-chloro-4-fluorophenoxy)-N-(3-(3-(4-chlorophenyl)-2-oxoimidazolidin-1-yl)bicyclo[1.1.1]pentan-1-yl)acetamide;

N-(3-(3-(4-chloro-3-(trifluoromethyl)phenyl)-2-oxoimidazolidin-1-yl)bicyclo[1.1.1]pentan-1-yl)-2-(4-chlorophenoxy)acetamide;

2-(4-chlorophenoxy)-N-(3-(3-(4-fluoro-3-(trifluoromethyl)phenyl)-2-oxoimidazolidin-1-yl)bicyclo[1.1.1]pentan-1-yl)acetamide;

N-(3-(3-(4-chlorophenyl)-2-oxoimidazolidin-1-yl)bicyclo[1.1.1]pentan-1-yl)-2-((5-chloropyridin-2-yl)oxy)acetamide;

2-(4-chlorophenoxy)-N-(3-(2-oxo-3-(4-(trifluoromethyl)phenyl)imidazolidin-1-yl)bicyclo[1.1.1]pentan-1-yl)acetamide;

N-(3-(3-(4-chlorophenyl)-2-oxoimidazolidin-1-yl)bicyclo[1.1.1]pentan-1-yl)-2-(4-fluoro-3-(trifluoromethyl)phenoxy)acetamide;

N-(3-(3-(4-chlorophenoxy)pyrrolidin-1-yl)bicyclo[1.1.1]pentan-1-yl)-2-(4-fluoro-3-(trifluoromethyl)phenoxy)acetamide;

N-(3-(3-(4-chlorophenoxy)pyrrolidin-1-yl)bicyclo[1.1.1]pentan-1-yl)-2-(4-(difluoromethoxy)phenoxy)acetamide;

2-(4-chloro-3-fluorophenoxy)-N-(3-(3-(4-chlorophenoxy)pyrrolidin-1-yl)bicyclo[1.1.1]pentan-1-yl)acetamide isomer 1;

(R)-2-(4-chloro-3-fluorophenoxy)-N-(3-(3-(4-chlorophenoxy)pyrrolidin-1-yl)bicyclo[1.1.1]pentan-1-yl)acetamide isomer 2;

2-(4-chlorophenoxy)-N-(3-(3-((5-chloropyridin-2-yl)oxy)pyrrolidin-1-yl)bicyclo[1.1.1]pentan-1-yl)acetamide;

2-((5-chloropyridin-2-yl)oxy)-N-(3-(3-((5-chloropyridin-2-yl)oxy)pyrrolidin-1-yl)bicyclo[1.1.1]pentan-1-yl)acetamide;

2-(4-chlorophenoxy)-N-(3-(3-(4-methoxyphenyl)-2-oxoimidazolidin-1-yl)bicyclo[1.1.1]pentan-1-yl)acetamide;

N-(3-(3-(4-chloro-2-fluorophenyl)-2-oxoimidazolidin-1-yl)bicyclo[1.1.1]pentan-1-yl)-2-(4-chlorophenoxy)acetamide;

(S)-2-(4-chlorophenoxy)-N-(3-(4-(4-chlorophenoxy)-2-oxopyrrolidin-1-yl)bicyclo[1.1.1]pentan-1-yl)acetamide;

(S)-2-(4-chlorophenoxy)-N-(3-(3-(4-chlorophenoxy)-2-oxopyrrolidin-1-yl)bicyclo[1.1.1]pentan-1-yl)acetamide;

(R)-2-(4-chlorophenoxy)-N-(3-(4-(4-chlorophenoxy)-2-oxopyrrolidin-1-yl)bicyclo[1.1.1]pentan-1-yl)acetamide;

(R)-2-(4-chlorophenoxy)-N-(3-(3-(4-chlorophenoxy)-2-oxopyrrolidin-1-yl)bicyclo[1.1.1]pentan-1-yl)acetamide;

2-(4-chlorophenoxy)-N-(3-(3-(4-(methylthio)phenyl)-2-oxoimidazolidin-1-yl)bicyclo[1.1.1]pentan-1-yl)acetamide; and

2-(4-chlorophenoxy)-N-(3-(4-(4-chlorophenoxy)piperidin-1-yl)bicyclo[1.1.1]pentan-1-yl)acetamide;
and salts thereof including pharmaceutically acceptable salts thereof.

Included in the compounds of the invention are:

N-(3-(5-chloroisoindolin-2-yl)bicyclo[1.1.1]pentan-1-yl)-2-(4-chlorophenoxy)acetamide; and

N-(3-(3-(bicyclo[4.2.0]octa-1,3,5-trien-3-yloxy)pyrrolidin-1-yl)bicyclo[1.1.1]pentan-1-yl)-2-(4-chlorophenoxy)acetamide;
and salts thereof including pharmaceutically acceptable salts thereof.

Included in the compounds of the invention are:

2-(4-chlorophenoxy)-N-(3-(2-((5,6,7,8-tetrahydronaphthalen-2-yl)oxy)acetamido)bicyclo[1.1.1]pentan-1-yl)acetamide;

5-chloro-N-(3-(2-(4-chlorophenoxy)acetamido)bicyclo[1.1.1]pentan-1-yl)-2,3-dihydrobenzofuran-2-carboxamide;

2-(bicyclo[4.2.0]octa-1(6),2,4-trien-3-yloxy)-N-(3-(2-(4-chlorophenoxy)acetamido)bicyclo[1.1.1]pentan-1-yl)acetamide; and

2-(4-chlorophenoxy)-N-(3-(2-(chroman-6-yloxy)acetamido)bicyclo[1.1.1]pentan-1-yl)acetamide; and salts thereof including pharmaceutically acceptable salts thereof.

To clarify the obvious intent, in any of the above Formulas, when “z” in a

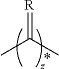

moiety is 0, and the adjacent “R*” and “L*” moieties form a ring, such as a heterocycloalkyl, for example a pyrrolidinyl, the “R*” and “L*” moieties do not have to be adjacent in the ring.

Further, in any of the above Formulas, in a

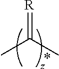

moiety, it is understood that “R*” will be absent whenever “Z*” is 0.

Further, in any of the above Formulas, in a

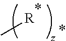

moiety, it is understood that whenever “z*” is 0, any substituent that could be an “R*” group, will be hydrogen.

Further, in the above Formulas, R^{85′} and R^{86′} are indicated by “each is independently selected from . . . ”. To clarify the obvious intent, for R^{85′} and R^{86′}, and all corresponding groups in each of the above Formulas, when two of the same groups are on the same molecule, (for example when two R^{85′} groups are on the same molecule), each ^{85′} can be a different substituent. For Example, one R^{85′} can be F and the other R^{85′} can be Cl.

In embodiments, R⁵ is independently fluoro, chloro, bromo, iodo, —OCH₃, —OCH₂Ph, —C(O)Ph, —CF₃, —CN, —S(O)CH₃, —OH, —NH₂, —COOH, —CONH₂, —NO₂, —C(O)CH₃, —C≡CH, —CH₂C≡CH, —SO₃H, —SO₂NH₂, —NHC(O)NH₂, —NHC(O)H, —NHOH, —OCH₃, —OCF₃, —OCHF₂, substituted or unsubstituted substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁵ is independently hydrogen, fluoro, chloro, bromo, iodo, —OCH₃, —OCH₂Ph, —CH₃, —OH, —CF₃, —CN, —S(O)CH₃, —NO₂, —C(O)CH₃, —C(O)Ph, —CH(CH₃)₂, or —C≡CH. In embodiments, R⁵ is —F. In embodiments, R⁵ is —Cl. In embodiments, R⁵ is —Br. In embodiments, R⁵ is —I. In embodiments, R⁵ is substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁵ is unsubstituted C₁₋₆alkyl, unsubstituted heteroalkyl, unsubstituted cycloalkyl, unsubstituted heterocycloalkyl, unsubstituted aryl, or unsubstituted heteroaryl. In embodiments, R⁵ is —OCH₃. In embodiments, R⁵ is —OCH₂Ph. In embodiments, R⁵ is —CH₃. In embodiments, R⁵ is —OH. In embodiments, R⁵ is —CF₃. In embodiments, R⁵ is —CN. In embodiments, R⁵ is —S(O)CH₃. In embodiments, R⁵ is —NO₂. In embodiments, R⁵ is —C(O)CH₃. In embodiments, R⁵ is —C(O)Ph. In embodiments, R⁵ is —CH(CH₃)₂. In embodiments, R⁵ is —C≡CH. In embodiments, R⁵ is —CH₂C≡CH. In embodiments, R⁵ is —SO₃H. In embodiments, R⁵ is —SO₂NH₂. In embodiments, R⁵ is —NHC(O)NH₂. In embodiments, R⁵ is —NHC(O)H. In embodiments, R⁵ is —NHOH. In embodiments, R⁵ is —OCH₃. In embodiments, R is —OCF₃. In embodiments, R⁵ is —OCHF₂.

In embodiments, R⁶ is independently fluoro, chloro, bromo, iodo, -OCH₃, —OCH₂Ph, —C(O)Ph, —CF₃, —CN, —S(O)CH₃, —OH, —NH₂, —COOH, —CONH₂, —NO₂, —C(O)CH₃, —C≡CH, —CH₂C≡CH, —SO₃H, —SO₂NH₂, —NHC(O)NH₂, —NHC(O)H, —NHOH, —OCH₃, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁶ is independently hydrogen, fluoro, chloro, bromo, iodo, —OCH₃, —OCH₂Ph, —CH₃, —OH, —CF₃, —CN, —S(O)CH₃, —NO₂, —C(O)CH₃, —C(O)Ph, —CH(CH₃)₂, or —C≡CH. In embodiments, R⁶ is —F. In embodiments, R⁶ is —Cl. In embodiments, R⁶ is —Br. In embodiments, R⁶ is —I. In embodiments, R⁶ is substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁶ is unsubstituted C₁₋₆alkyl, unsubstituted heteroalkyl, unsubstituted cycloalkyl, unsubstituted heterocycloalkyl, unsubstituted aryl, or unsubstituted heteroaryl. In embodiments, R⁶ is —OCH₃. In embodiments, R⁶ is —OCH₂Ph. In embodiments, R⁶ is —CH₃. In embodiments, R⁶ is —OH. In embodiments, R⁶ is —CF₃. In embodiments, R⁶ is —CN. In embodiments, R⁶ is —S(O)CH₃. In embodiments, R⁶ is —NO₂. In embodiments, R⁶ is —C(O)CH₃. In embodiments, R⁶ is —C(O)Ph. In embodiments, R⁶ is —CH(CH₃)₂. In embodiments, R⁶ is —C≡CH. In embodiments, R⁶ is —CH₂C≡CH. In embodiments, R⁶ is —SO₃H. In embodiments, R⁶ is —SO₂NH₂. In embodiments, R⁶ is —NHC(O)NH₂. In embodiments, R⁶ is —NHC(O)H. In embodiments, R⁶ is —NHOH. In embodiments, R⁶ is —OCH₃. In embodiments, R⁶ is —OCF₃. In embodiments, R⁶ is —OCHF₂.

In embodiments, R² is NR⁸. In embodiments, R² is NH. In embodiments, R² is O. In embodiments, R² is S. In embodiments, R² is CH₂. In embodiments, R⁴ is NR⁸. In embodiments, R⁴ is NH. In embodiments, R⁴ is O. In embodiments, R⁴ is S. In embodiments, R⁴ is CH₂. In embodiments, R² and R⁴ are NH. In embodiments, R² and R⁴ are O. In embodiments, R² and R⁴ are S. In embodiments, R² and R⁴ are NR⁸.

In embodiments, L² is a bond. In embodiments, L² is a substituted or unsubstituted C₁₋₆alkylene. In embodiments, L² is a substituted or unsubstituted C₁₋₆heteroalkylene. In embodiments, L² is L^{2A}-L^{2B}-L^{2C} and L^{2A} is bonded to the substituted or unsubstituted phenyl, which may be substituted with R⁵. L^{2A} is a bond, —O—, —S—, —NH—, —S(O)—, or —S(O)₂—. L^{2B} is a bond or substituted or unsubstituted C₁₋₆alkylene. L^{2C} is a bond, —O—, or —NH—. In embodiments, L^{2A} is a bond. In embodiments, L^{2A} is —O—. In embodiments, L^{2A} is —S—. In embodiments, L^{2A} is —NH—. In embodiments, L^{2A} is —S(O)—. In embodiments, L^{2A} is —S(O)₂—. In embodiments, L^{2B} is a bond. In embodiments, L^{2B} is a substituted or unsubstituted C₁₋₆alkylene. In embodiments, L^{2B} is an unsubstituted C₁₋₆alkylene. In embodiments, L^{2B} is a substituted or unsubstituted C₁-C₅ alkylene. In embodiments, L^{2B} is an unsubstituted C₁-C₅ alkylene. In embodiments, L^{2B} is a substituted or unsubstituted C₁-C₄ alkylene. In embodiments, L^{2B} is an unsubstituted C₁-C₄ alkylene. In embodiments, L^{2B} is a substituted or unsubstituted C₁-C₃ alkylene. In embodiments, L^{2B} is an unsubstituted C₁-C₃ alkylene. In embodiments, L^{2B} is a substituted C₁-C₅ alkylene. In embodiments, L^{2B} is a substituted C₁-C₆ alkylene. In embodiments, L^{2B} is a substituted C₁-C₅ alkylene. In embodiments, L^{2B} is a substituted C₁-C₄ alkylene. In embodiments, L^{2B} is a C₁-C₆ alkylene substituted with —CF₃. In embodiments, L^{2C} is a bond. In embodiments, L^{2C} is —O—. In embodiments, L^{2C} is —NH—. In embodiments, L^{2A} is a bond; L^{2B} is unsubstituted methylene; and L^{2C} is —O—.

In embodiments, L³ is a bond. In embodiments, L³ is a substituted or unsubstituted C₁₋₆₆alkylene. In embodiments, L³ is a substituted or unsubstituted C₁₋₆heteroalkylene. In embodiments, L³ is L^{3A}-L^{3B}-L^{3C} and L^{3A} is bonded to the substituted or unsubstituted phenyl, which may be substituted with R⁵. L^{3A} is a bond, —O—, —S—, —NH—, —S(O)—, or —S(O)₂—. L^{3B} is a bond or substituted or unsubstituted C₁₋₆alkylene. L^{3C} is a bond, —O—, or —NH—. In embodiments, L^{3A} is a bond. In embodiments, L^{3A} is —O—. In embodiments, L^{3A} is —S—. In embodiments, L^{3A} is —NH—. In embodiments, L^{3A} is —S(O)—. In embodiments, L^{3A} is —S(O)₂—. In embodiments, L^{3B} is a bond. In embodiments, L^{3B} is a substituted or unsubstituted C₁₋₆alkylene. In embodiments, L^{3B} is an unsubstituted C₁₋₆alkylene. In embodiments, L^{3B} is a substituted or unsubstituted C₁-C₅ alkylene. In embodiments, L^{3B} is an unsubstituted C₁-C₅ alkylene. In embodiments, L^{3B} is a substituted or unsubstituted C₁-C₄ alkylene. In embodiments, L^{3B} is an unsubstituted C₁-C₄ alkylene. In embodiments, L^{3B} is a substituted or unsubstituted C₁-C₃alkylene. In embodiments, L^{3B} is an unsubstituted C₁-C₃ alkylene. In embodiments, L^{3B} is a substituted C₁-C₅ alkylene. In embodiments, L^{3B} is a substituted C₁-C₆ alkylene. In embodiments, L^{3B} is a substituted C₁-C₅ alkylene. In embodiments, L^{3B} is a substituted C₁-C₄ alkylene. In embodiments, L^{3B} is a C₁-C₆ alkylene substituted with —CF₃. In embodiments, L^{3C} is a bond. In embodiments, L^{3C} is —O—. In embodiments, L^{3C} is —NH—. In embodiments, L^{3A} is a bond; L^{3B} is unsubstituted methylene; and L^{3C} is —O—.

In embodiments, L³ is taken together with R¹ to form heterocycloalkyl. Suitably the heterocycloalkyl is imidazolidinyl or pyrrolidinyl. Suitably the heterocycloalkyl is imidazolidinyl. Suitably the heterocycloalkyl is pyrrolidinyl.

In embodiments, L² is taken together with R³ to form heterocycloalkyl. Suitably the heterocycloalkyl is imidazolidinyl or pyrrolidinyl. Suitably the heterocycloalkyl is imidazolidinyl. Suitably the heterocycloalkyl is pyrrolidinyl.

In embodiments, L²² is taken together with R²³ to form heterocycloalkyl. Suitably the heterocycloalkyl is imidazolidinyl or pyrrolidinyl. Suitably the heterocycloalkyl is imidazolidinyl. Suitably the heterocycloalkyl is pyrrolidinyl.

In embodiments, L²³ is taken together with R²¹ to form heterocycloalkyl. Suitably the heterocycloalkyl is imidazolidinyl or pyrrolidinyl. Suitably the heterocycloalkyl is imidazolidinyl. Suitably the heterocycloalkyl is pyrrolidinyl.

In embodiments, L³³ is taken together with R³¹ to form heterocycloalkyl. Suitably the heterocycloalkyl is imidazolidinyl or pyrrolidinyl. Suitably the heterocycloalkyl is imidazolidinyl. Suitably the heterocycloalkyl is pyrrolidinyl.

In embodiments, L⁴² is taken together with R⁴¹ to form imidazolidinyl or pyrrolidinyl. Suitably L⁴² is taken together with R⁴¹ to form imidazolidinyl. Suitably L⁴² is taken together with R⁴¹ to form pyrrolidinyl.

In embodiments, L⁴³ is taken together with R⁴³ to form imidazolidinyl or pyrrolidinyl. Suitably L⁴³ is taken together with R⁴³ to form imidazolidinyl. Suitably L⁴³ is taken together with R⁴³ to form pyrrolidinyl.

In embodiments, L⁵³ is taken together with R⁵³ to form imidazolidinyl or pyrrolidinyl. Suitably L⁵³ is taken together with R⁵³ to form imidazolidinyl. Suitably L⁵³ is taken together with R⁵³ to form pyrrolidinyl.

In embodiments, L⁶² is taken together with R⁶¹ to form imidazolidinyl or pyrrolidinyl. Suitably L⁶² is taken together with R⁶¹ to form imidazolidinyl. Suitably L⁶² is taken together with R⁶¹ to form pyrrolidinyl.

In embodiments, L⁶³ is taken together with R⁶³ to form imidazolidinyl or pyrrolidinyl. Suitably L⁶³ is taken together with R⁶³ to form imidazolidinyl. Suitably L⁶³ is taken together with R⁶³ to form pyrrolidinyl.

In embodiments, L⁷³ is taken together with R⁷³ to form imidazolidinyl or pyrrolidinyl. Suitably L⁷³ is taken together with R⁷³ to form imidazolidinyl. Suitably L⁷³ is taken together with R⁷³ to form pyrrolidinyl.

In embodiments, L⁸³ is taken together with R⁸¹ to form heterocycloalkyl. In other words, the moiety comprising —NR⁸¹—(C═R⁸⁴)z⁸⁴-L⁸³- represents heterocycloalkyl. Suitably the heterocycloalkyl is imidazolidinyl or pyrrolidinyl. Suitably the heterocycloalkyl is imidazolidinyl. Suitably the heterocycloalkyl is pyrrolidinyl. In embodiments, L⁸³ is taken together with R⁸¹ to form oxoheterocycloalkyl. In other words, the moiety comprising —NR⁸¹—(C═R⁸⁴)z⁸⁴-L⁸³- represents oxoheterocycloalkyl. Suitably the oxoheterocycloalkyl is 2-oxoimidazolidinyl. Suitably the oxoheterocycloalkyl is oxopyrrolidinyl. In embodiments, L⁸³ is taken together with R⁸¹ to form heterocycloalkyl-O—. In other words, the moiety comprising —NR⁸¹—(C═R⁸⁴)z⁸⁴-L⁸³- represents heterocycloalkyl-O—, wherein the —O— is an oxygen linking atom connecting the heterocycloalkyl to D⁸. Suitably the heterocycloalkyl-O— is azetidinyl-O— or pyrrolidinyl-O—. Suitably the heterocycloalkyl-O— is pyrrolidinyl-O—.

In embodiments, L⁸² is taken together with R⁸³ to form heterocycloalkyl. Suitably the heterocycloalkyl is imidazolidinyl or pyrrolidinyl. Suitably the heterocycloalkyl is imidazolidinyl. Suitably the heterocycloalkyl is pyrrolidinyl.

In embodiments, L⁹³ is taken together with R⁹¹ to form heterocycloalkyl. Suitably the heterocycloalkyl is imidazolidinyl or pyrrolidinyl. Suitably the heterocycloalkyl is imidazolidinyl. Suitably the heterocycloalkyl is pyrrolidinyl. In embodiments, L⁹³ is taken together with R⁹¹ to form oxoheterocycloalkyl. Suitably the oxoheterocycloalkyl is 2-oxoimidazolidinyl. Suitably the oxoheterocycloalkyl is oxopyrrolidinyl. In embodiments, L⁹³ is taken together with R⁹¹ to form heterocycloalkyl-O—. Suitably the heterocycloalkyl-O— is azetidinyl-O— or pyrrolidinyl-O—. Suitably the heterocycloalkyl-O— is pyrrolidinyl-O—.

In embodiments, L¹⁰² is taken together with R¹⁰¹ to form imidazolidinyl or pyrrolidinyl. Suitably L¹⁰² is taken together with R¹⁰¹ to form imidazolidinyl. Suitably L¹⁰² is taken together with R¹⁰¹ to form pyrrolidinyl. In embodiments, L¹⁰² is taken together with R¹⁰¹ to form oxoheterocycloalkyl. Suitably the oxoheterocycloalkyl is 2-oxoimidazolidinyl. Suitably the oxoheterocycloalkyl is oxopyrrolidinyl. In embodiments, L¹⁰² is taken together with R¹⁰¹ to form heterocycloalkyl-O—. Suitably the heterocycloalkyl-O— is azetidinyl-O— or pyrrolidinyl-O—. Suitably the heterocycloalkyl-O— is pyrrolidinyl-O—.

In embodiments, L¹⁰³ is taken together with R¹⁰³ to form imidazolidinyl or pyrrolidinyl. Suitably L¹⁰³ is taken together with R¹⁰³ to form imidazolidinyl. Suitably L¹⁰³ is taken together with R¹⁰³ to form pyrrolidinyl. In embodiments, L¹⁰³ is taken together with R¹⁰³ to form oxoheterocycloalkyl. Suitably the oxoheterocycloalkyl is 2-oxoimidazolidinyl. Suitably the oxoheterocycloalkyl is oxopyrrolidinyl. In embodiments, L¹⁰³ is taken together with R¹⁰³ to form heterocycloalkyl-O—. Suitably the heterocycloalkyl-O— is azetidinyl-O— or pyrrolidinyl-O—. Suitably the heterocycloalkyl-O— is pyrrolidinyl-O—.

In embodiments, L¹¹³ is taken together with R¹¹³ to form imidazolidinyl, pyrrolidinyl or cyclopropyl. In embodiments, L¹¹³ is taken together with R¹¹³ to form imidazolidinyl. In embodiments, L¹¹³ is taken together with R¹¹³ to form pyrrolidinyl. In embodiments, L¹¹³ is taken together with R¹¹³ to form oxoheterocycloalkyl. Suitably the oxoheterocycloalkyl is 2-oxoimidazolidinyl. Suitably the oxoheterocycloalkyl is oxopyrrolidinyl. In embodiments, L¹¹³ is taken together with R¹¹³ to form heterocycloalkyl-O—. Suitably the heterocycloalkyl-O— is azetidinyl-O— or pyrrolidinyl-O—. Suitably the heterocycloalkyl-O— is pyrrolidinyl-O—.

In embodiments, L¹²² is taken together with R¹²¹ to form imidazolidinyl or pyrrolidinyl. Suitably L¹²² is taken together with R¹²¹ to form imidazolidinyl. Suitably L¹²² is taken together with R¹²¹ to form pyrrolidinyl. In embodiments, L¹²² is taken together with R¹²¹ to form oxoheterocycloalkyl. Suitably the oxoheterocycloalkyl is 2-oxoimidazolidinyl. Suitably the oxoheterocycloalkyl is oxopyrrolidinyl. In embodiments, L¹²² is taken together with R¹²¹ to form heterocycloalkyl-O—. Suitably the heterocycloalkyl-O— is azetidinyl-O— or pyrrolidinyl-O—. Suitably the heterocycloalkyl-O— is pyrrolidinyl-O—.

In embodiments, L¹²³ is taken together with R¹²³ to form imidazolidinyl or pyrrolidinyl. Suitably L¹²³ is taken together with R¹²³ to form imidazolidinyl. Suitably L¹²³ is taken together with R¹²³ to form pyrrolidinyl. In embodiments, L¹²³ is taken together with R¹²³ to form oxoheterocycloalkyl. Suitably the oxoheterocycloalkyl is 2-oxoimidazolidinyl. Suitably the oxoheterocycloalkyl is oxopyrrolidinyl. In embodiments, L¹²³ is taken together with R¹²³ to form heterocycloalkyl-O—. Suitably the heterocycloalkyl-O— is azetidinyl-O— or pyrrolidinyl-O—. Suitably the heterocycloalkyl-O— is pyrrolidinyl-O—.

In embodiments, L¹³³ is taken together with R¹³³ to form imidazolidinyl or pyrrolidinyl. Suitably L¹³³ is taken together with R¹³³ to form imidazolidinyl. Suitably L¹³³ is taken together with R¹³³ to form pyrrolidinyl. In embodiments, L¹³³ is taken together with R¹³³ to form oxoheterocycloalkyl. Suitably the oxoheterocycloalkyl is 2-oxoimidazolidinyl. Suitably the oxoheterocycloalkyl is oxopyrrolidinyl. In embodiments, L¹³³ is taken together with R¹³³ to form heterocycloalkyl-O—. Suitably the heterocycloalkyl-O— is azetidinyl-O— or pyrrolidinyl-O—. Suitably the heterocycloalkyl-O— is pyrrolidinyl-O—.

In embodiments, the symbol z² is 0. In embodiments, the symbol z² is 1. In embodiments, the symbol z⁴ is 0. In embodiments, the symbol z⁴ is 1. In embodiments, the symbols z² and z⁴ are 0. In embodiments, the symbols z² and z⁴ are 1. In embodiments, the symbol z⁵ is 0. In embodiments, the symbol z⁵ is 1. In embodiments, the symbol z⁵ is 2. In embodiments, the symbol z⁵ is 3. In embodiments, the symbol z⁵ is 4. In embodiments, the symbol z⁶ is 0. In embodiments, the symbol z⁶ is 1. In embodiments, the symbol z⁶ is 2. In embodiments, the symbol z⁶ is 3. In embodiments, the symbol z⁶ is 4.

The skilled artisan will appreciate that salts, including pharmaceutically acceptable salts, of the compounds according to Formula (IIIQ) may be prepared. Indeed, in certain embodiments of the invention, salts including pharmaceutically-acceptable salts of the compounds according to Formula (IIIQ) may be preferred over the respective free or unsalted compound. Accordingly, the invention is further directed to salts, including pharmaceutically-acceptable salts, of the compounds according to Formula (IIIQ).

The salts, including pharmaceutically acceptable salts, of the compounds of the invention are readily prepared by those of skill in the art.

Typically, the salts of the present invention are pharmaceutically acceptable salts. Salts encompassed within the term “pharmaceutically acceptable salts” refer to non-toxic salts of the compounds of this invention.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulf ate, mucate, naphthalene-1 ,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminum, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

The compounds according to Formula (IIIQ) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may be present in a substituent such as an alkyl group. Where the stereochemistry of a chiral center present in a compound of Formula (IIIQ), or in any chemical structure illustrated herein, if not specified the structure is intended to encompass all individual stereoisomers and all mixtures thereof. Thus, compounds according to Formula (IIIQ) containing one or more chiral centers may be used as racemic mixtures, enantiomerically or diastereomerically enriched mixtures, or as enantiomerically or diastereomerically pure individual stereoisomers.

The compounds according to Formula (IIIQ) and pharmaceutically acceptable salts thereof may contain isotopically-labelled compounds, which are identical to those recited in Formula (IIIQ) and following, but for the fact that one or more atoms are replaced by an atom having an atomic mass or mass number different from the atomic mass or mass number usually found in nature. Examples of such isotopes include isotopes of hydrogen, carbon, nitrogen, oxygen, phosphorous, sulphur, fluorine, iodine, and chlorine, such as 2H, 3H, 11C, 13C, 14C, 15N, 17O, 18O, 31P, 32P, 35S, 18F, 36Cl, 123I and 125I.

Isotopically-labelled compounds, for example those into which radioactive isotopes such as 3H or 14C are incorporated, are useful in drug and/or substrate tissue distribution assays. Tritiated, i.e., 3H, and carbon-14, i.e., 14C, isotopes are particularly preferred for their ease of preparation and detectability. 11C and 18F isotopes are particularly useful in PET (positron emission tomography), and 125I isotopes are particularly useful in SPECT (single photon emission computerized tomography), both are useful in brain imaging. Further, substitution with heavier isotopes such as deuterium, i.e., 2H, can afford certain therapeutic advantages resulting from greater metabolic stability, for example increased in vivo half-life or reduced dosage requirements and, hence, may be preferred in some circumstances. Isotopically labelled compounds can generally be prepared by substituting a readily available isotopically labelled reagent for a non-isotopically labelled reagent.

The compounds according to Formula (IIIQ) may also contain double bonds or other centers of geometric asymmetry. Where the stereochemistry of a center of geometric asymmetry present in Formula (IIIQ), or in any chemical structure illustrated herein, is not specified, the structure is intended to encompass the trans (E) geometric isomer, the cis (Z) geometric isomer, and all mixtures thereof. Likewise, all tautomeric forms are also included in Formula (IIIQ) whether such tautomers exist in equilibrium or predominately in one form.

The compounds of Formula (IIIQ) or salts, including pharmaceutically acceptable salts, thereof may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically acceptable solvates may be formed wherein solvent molecules are incorporated into the crystalline lattice during crystallization. Solvates wherein water is the solvent that is incorporated into the crystalline lattice are typically referred to as “hydrates.” Hydrates include stoichiometric hydrates as well as compositions containing vaiable amounts of water.

The skilled artisan will further appreciate that certain compounds of Formula (IIIQ) or salts, including pharmaceutically acceptable salts thereof that exist in crystalline form, including the various solvates thereof, may exhibit polymorphism (i.e. the capacity to occur in different crystalline structures). These different crystalline forms are typically known as “polymorphs.” Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

While aspects for each variable have generally been listed above separately for each variable this invention includes those compounds in which several or each aspect in Formula (IIIQ) is selected from each of the aspects listed above. Therefore, this invention is intended to include all combinations of aspects for each variable.

### Definitions

“Alkyl” and “alkylene”, and derivatives thereof, refer to a hydrocarbon chain having the specified number of “member atoms”. Alkyl being monovalent and alkylene being bivalent. For example, C₁-C₆ alkyl refers to an alkyl group having from 1 to 6 member atoms. Alkyl and alkylene groups may be saturated, unsaturated, straight or branched. Representative branched alkyl groups have one, two, or three branches. Alkyl and alkylene include: methyl, ethyl, ethylene, propyl (n-propyl and isopropyl), butene, butyl (n-butyl, isobutyl, and t-butyl), pentyl and hexyl.

“Alkoxy” refers to an —O-alkyl group wherein “alkyl” is as defined herein. For example, C₁-C₄alkoxy refers to an alkoxy group having from 1 to 4 member atoms. Representative branched alkoxy groups have one, two, or three branches. Examples of such groups include methoxy, ethoxy, propoxy, and butoxy.

“Aryl” refers to an aromatic hydrocarbon ring. Aryl groups are monocyclic, bicyclic, and tricyclic ring systems having a total of five to fourteen ring member atoms, wherein at least one ring system is aromatic and wherein each ring in the system contains 3 to 7 member atoms, such as phenyl, naphthalene, tetrahydronaphthalene and biphenyl. Suitably aryl is phenyl.

“Cycloalkyl”, unless otherwise defined, refers to a saturated or unsaturated non aromatic hydrocarbon ring having from three to seven carbon atoms. Cycloalkyl groups are monocyclic ring systems. For example, C₃-C₇ cycloalkyl refers to a cycloalkyl group having from 3 to 7 member atoms. Examples of cycloalkyl as used herein include: cyclopropyl, cyclobutyl, cyclopentyl, cyclohexyl, cyclobutenyl, cyclopentenyl, cyclohexenyl and cycloheptyl. Suitably cycolalkyl is selected from: cyclopropyl, cyclobutyl and cyclohexyl. Suitably cycolalkyl is cyclopropyl.

“Halo” refers to fluoro, chloro, bromo, and iodo.

“Heteroaryl” refers to a monocyclic aromatic 4 to 8 member ring containing 1 to 7 carbon atoms and containing 1 to 4 heteroatoms, provided that when the number of carbon atoms is 3, the aromatic ring contains at least two heteroatoms, or to such aromatic ring is fused one or more rings, such as heteroaryl rings, aryl rings, heterocyclic rings, or cycloalkyl rings. Heteroaryl groups containing more than one heteroatom may contain different heteroatoms. Heteroaryl includes but is not limited to: benzoimidazolyl, benzothiazolyl, benzothiophenyl, benzopyrazinyl, benzotriazolyl, benzotriazinyl, benzo[1,4]dioxanyl, benzofuranyl, 9H-a-carbolinyl, cinnolinyl, furanyl, pyrazolyl, imidazolyl, indolizinyl, naphthyridinyl, oxazolyl, oxothiadiazolyl, oxadiazolyl, phthalazinyl, pyridyl, pyrrolyl, purinyl, pteridinyl, phenazinyl, pyrazolopyrimidinyl, pyrazolopyridinyl, pyrrolizinyl, pyrimidyl, isothiazolyl, furazanyl, pyrimidinyl, tetrazinyl, isoxazolyl, quinoxalinyl, quinazolinyl, quinolinyl, quinolizinyl, thienyl, thiophenyl, triazolyl, triazinyl, tetrazolopyrimidinyl, triazolopyrimidinyl, tetrazolyl, thiazolyl and thiazolidinyl. Suitably heteroaryl is selected from: pyrazolyl, imidazolyl, oxazolyl and thienyl. Suitably heteroaryl is a pyridyl group or an imidazolyl group. Suitably heteroaryl is a pyridyl.

“Heterocycloalkyl” refers to a saturated or unsaturated non-aromatic ring containing 4 to 12 member atoms, of which 1 to 11 are carbon atoms and from 1 to 6 are heteroatoms. Heterocycloalkyl groups containing more than one heteroatom may contain different heteroatoms. Heterocycloalkyl groups are monocyclic ring systems or a monocyclic ring fused with an aryl ring or to a heteroaryl ring having from 3 to 6 member atoms. Heterocycloalkyl includes: pyrrolidinyl, tetrahydrofuranyl, dihydrofuranyl, pyranyl, tetrahydropyranyl, dihydropyranyl, tetrahydrothienyl, pyrazolidinyl, oxazolidinyl, imidazolidinyl, oxetanyl, thiazolidinyl, piperidinyl, homopiperidinyl, piperazinyl, morpholinyl, thiamorpholinyl, 1,3-dioxolanyl, 1,3-dioxanyl, 1,4-dioxanyl, 1,3-oxathiolanyl, 1,3-oxathianyl, 1,3-dithianyl, 1,3oxazolidin-2-one, hexahydro-1H-azepin, 4,5,6,7,tetrahydro-1H-benzimidazol, piperidinyl, 1,2,3,6-tetrahydro-pyridinyl and azetidinyl. Suitably, “heterocycloalkyl” includes: piperidinyl, tetrahydrofuranyl, tetrahydropyranyl, imidazolidinyl, oxetanyl, and pyrrolidinyl. Suitably, “heterocycloalkyl” is selected from: imidazolidinyl, tetrahydropyranyl and pyrrolidinyl.

“Heteroatom” refers to a nitrogen, sulfur or oxygen atom.

“Heteroalkyl” and “heteroalkylene” by itself or in combination with another term, means, unless otherwise stated, a non-cyclic stable straight or branched chain, or combinations thereof, including at least one carbon atom (and up to the number specified) and at least one heteroatom selected from the group consisting of O, N, P, Si, and S, and wherein the nitrogen and sulfur atoms may optionally be oxidized, and the nitrogen heteroatom may optionally be quaternized. For example, C₁₋₆heteroalkyl(ene) contains at least one and up to 6 carbon atoms, in addition to at least one heteroatom. Heteroalkyl being monovalent and heteroalkylene being bivalent. The heteroalkyl and heteroalkylene groups may be taken together with another substituent to form a heterocycloalkyl group. The heteroatom(s) O, N, P, S, and Si may be placed at any interior position of the heteroalkyl or heteroalkylene group or at the position at which the alkyl group is attached to the remainder of the molecule. Heteroalkyl examples include, but are not limited to:

—CH₂—CH₂—O—CH₃, —CH₂—CH₂—NH—CH₃, —CH₂—CH₂—N(CH₃)₂, —CH₂—S—CH₂—CH₃, —S(O)—CH₃, —CH₂—CH₂—S(O)₂—CH₃, —CH═CH—O—CH₃, —Si(CH₃)₃, —CH₂—CH═N—OCH₃, —CH═CHN(CH₃)₂, —O—CH₃, —O—CH₂—CH₃, —CN. Heteroalkylene examples include, but are not limited to: —CH₂—CH₂—O—CH₂—, —CH₂—CH₂—NH—CH₂—, —CH₂—CH₂—N(CH₃)CH₂—, —CH₂—S—CH₂—CH₂—, —S(O)—CH₂—, —CH₂—CH₂—S(O)₂—CH₂—, —CH═CH—O—CH₂—, —Si(CH₃)₂CH₂—, —N(CH₃)CH₂—, —O—CH₂—CH₂—CH₂—, —CH₂—CH═N—OCH₂—, —CH═CHN(CH₃)CH₂—, —O—CH₂—, and —O—CH₂—CH₂—. Up to two or three heteroatoms may be consecutive, such as, for example, —CH₂—NH—OCH₃ and —CH₂—O—Si(CH₃)₃.

To clarify the obvious intent, “2-oxoimidazolidinyl” as used herein, is meant the monovalent substituent

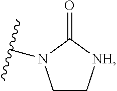 [SMILES: *N1CCNC1=O]

or the bivalent substituent

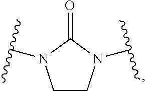 [SMILES: *N1CCN(*)C1=O]

depending on it's linkage to the rest of the molecule. Similarly, all other ring substituents used herein may be monovalent, bivalent, etc, depending on it's linkage to the rest of the molecule.

“Substituted” as used herein, unless otherwise defined, is meant that the subject chemical moiety has from one to nine substituents, suitably from one to five substituents, selected from the group consisting of:
fluoro,
chloro,
bromo,
iodo,
C₁₋₆alkyl,
C₁₋₆alkyl substituted with from 1 to 6 substituents independently selected from: fluoro, oxo, —OH, —COOH, —NH₂, and —CN,
—OC₁₋₆alkyl,
—OC₁₋₆alkyl substituted with from 1 to 6 substituents independently selected from: fluoro, oxo, —OH, —COOH, —NH₂, and —CN,
mercapto,
—SR^{x}, where R^{x} is selected from C₁₋₆alkyl, and C₁₋₆alkyl substituted with from 1 to 6 substituents independently selected from: fluoro, oxo, —OH, —COOH, —NH₂, and —CN,
—S(O)R^{x}, where R^{x} is selected from C₁₋₆alkyl, and C₁₋₆alkyl substituted with from 1 to 6 substituents independently selected from: fluoro, oxo, —OH, —COOH, —NH₂, and —CN,
—S(O)₂H,
—S(O)₂R^{x}, where R^{x} is selected from C₁₋₆alkyl, and C₁₋₆alkyl substituted with from 1 to 6 substituents independently selected from: fluoro, oxo, —OH, —COOH, —NH₂, and —CN,
oxo,
hydroxy,
amino,
—NHR^{x}, where R^{x} is selected from C₁₋₆alkyl, and C₁₋₆alkyl substituted with from 1 to 6 substituents independently selected from: fluoro, oxo, —OH, —COOH, —NH₂, and —CN,
—NR^{x1}R^{x2}, where R^{x1} and R^{x2} are each independently selected from C₁₋₆alkyl, and C₁₋₆alkyl substituted with from 1 to 6 substituents independently selected from: fluoro, oxo, —OH, —COOH, —NH₂, and —CN,
guanidino,
—C(O)OH,
—C(O)OR^{x}, where R^{x} is selected from C₁₋₆alkyl, and C₁₋₆alkyl substituted with from 1 to 6 substituents independently selected from: fluoro, oxo, —OH, —COOH, —NH₂, and —CN,
—C(O)NH₂,
—C(O)NHR^{x}, where R^{x} is selected from C₁₋₆alkyl, and C₁₋₆alkyl substituted with from 1 to 6 substituents independently selected from: fluoro, oxo, —OH, —COOH, —NH₂, and —CN,
C(O)NR^{x1}R^{x2}, where R^{x1} and R^{x2} are each independently selected from C₁₋₆alkyl, and C₁₋₆alkyl substituted with from 1 to 6 substituents independently selected from: fluoro, oxo, —OH, —COOH, —NH₂, and —CN,
—S(O)₂NH₂,
—S(O)₂NHR^{x}, where R^{x} is selected from C₁₋₆alkyl, and C₁₋₆alkyl substituted with from 1 to 6 substituents independently selected from: fluoro, oxo, —OH, —COOH, —NH₂, and —CN,
—S(O)₂NR^{x1}R^{x2}, where R^{x1} and R^{x2} are each independently selected from C₁₋₆alkyl, and C₁₋₆alkyl substituted with from 1 to 6 substituents independently selected from: fluoro, oxo, —OH, —COOH, —NH₂, and —CN,
—NHS(O)₂H,
—NHS(O)₂R^{x}, where R^{x} is selected from C₁₋₆alkyl, and C₁₋₆alkyl substituted with from 1 to 6 substituents independently selected from: fluoro, oxo, —OH, —COOH, —NH₂, and —CN,
—NHC(O)H,
—NHC(O)R^{x}, where R^{x} is selected from C₁₋₆alkyl, and C₁₋₆alkyl substituted with from 1 to 6 substituents independently selected from: fluoro, oxo, —OH, —COOH, —NH₂, and —CN,
—NHC(O)NH₂,
—NHC(O)NHR^{x}, where R^{x} is selected from C₁₋₆alkyl, and C₁₋₆alkyl substituted with from 1 to 6 substituents independently selected from: fluoro, oxo, —OH, —COOH, —NH₂, and —CN,
—NHC(O)NR^{x1}R^{x2}, where R^{x1} and R^{x2} are each independently selected from C₁₋₆alkyl, and C₁₋₆alkyl substituted with from 1 to 6 Substituents independently selected from: fluoro, oxo, —OH, —COOH, —NH₂, and —CN,
nitro, and
cyano.

Suitably “substituted” means the subject chemical moiety has from one to four substituents selected from the group consisting of:
fluoro,
chloro,
bromo,
iodo,
C₁₋₄alkyl,
C₁₋₄alkyl substituted with from 1 to 4 substituents independently selected from: fluoro, oxo, —OH, —COOH, —NH₂, and —CN,
—OC₁₋₄alkyl,
—OC₁₋₄alkyl substituted with from 1 to 4 substituents independently selected from: fluoro, oxo, —OH, —COOH, —NH₂, and —CN,
—SH
—S(O)₂H,
oxo
hydroxy,
amino,
—NHR^{x}, where R^{x} is selected from C₁₋₄alkyl, and C₁₋₆alkyl substituted one to 4 times by fluoro,
—NR^{x1}R^{x2}, where R^{x1} and R^{x2} are each independently selected from C₁₋₄alkyl, and C₁₋₄alkyl substituted one to four times by fluoro,
guanidino,
—C(O)OH,
—C(O)OR^{x}, where R^{x} is selected from C₁₋₄alkyl, and C₁₋₄alkyl substituted one to four times by fluoro,
—C(O)NH₂,
—C(O)NHR^{x}, where R^{x} is selected from C₁₋₄alkyl, and C₁₋₄alkyl substituted one to four times by fluoro,
—C(O)NR^{x1} R^{x2}, where R^{x1} and R^{x2} are each independently selected from C₁₋₄alkyl, and C₁₋₄alkyl substituted one to four times by fluoro,
—S(O)₂NH₂,
—NHS(O)₂H,
—NHC(O)H,
—NHC(O)NH₂,
nitro, and
cyano.

Suitably “substituted” means the subject chemical moiety has from one to four substituents selected from the group consisting of:
fluoro,
chloro,
bromo,
iodo,
C₁₋₄alkyl,
C₁₋₄alkyl substituted with from 1 to 4 substituents independently selected from: fluoro, oxo, —OH, —COOH, —NH₂, —NHC₁₋₃alkyl, —N(C₁₋₃alkyl)₂, —OC₁₋₄alkyl and —CN,
—OC₁₋₄alkyl,
—OC₁₋₄alkyl substituted with from 1 to 4 substituents independently selected from: fluoro, oxo, —OH, —COOH, —NH₂, —NHC₁₋₃alkyl, —N(C₁₋₃alkyl)₂, and —CN,
—SH,
—S(O)₂H,
oxo,
hydroxy,
amino,
—NHR^{x}, where R^{x} is selected from C₁₋₄alkyl, and C₁₋₄alkyl substituted one to 4 times by fluoro,
—NR^{x1}R^{x2}, where R^{x1} and R^{x2} are each independently selected from C₁₋₄alkyl, and C₁₋₄alkyl substituted one to four times by fluoro,
guanidino,
—C(O)OH,
—C(O)OR^{x}, where R^{x} is selected from C₁₋₄alkyl, and C₁₋₄alkyl substituted one to four times by fluoro,
—C(O)NH₂,
—C(O)NHR^{x}, where R^{x} is selected from C₁₋₄alkyl, and C₁₋₄alkyl substituted one to four times by fluoro,
—C(O)NR¹ R^{x2}, where R^{x1} and R^{x2} are each independently selected from C₁₋₄alkyl, and C₁₋₄alkyl substituted one to four times by fluoro,
—S(O)₂NH₂,
—NHS(O)₂H,
—NHC(O)H,
—NHC(O)NH₂,
nitro, and
cyano.

Suitably “substituted” means the subject chemical moiety has from one to four substituents selected from the group consisting of:
fluoro,
chloro,
bromo,
iodo,
C₁₋₄alkyl,
C₁₋₄alkyl substituted with from 1 to 4 substituents independently selected from: fluoro, oxo, —OH, —COOH, —NH₂, —NHCH₃, —N(CH₃)₂, —OCH₃, —OCH₂CH₃, and —CN,
—OC₁₋₄alkyl,
—OC₁₋₄alkyl substituted with from 1 to 4 substituents independently selected from: fluoro, oxo, —OH, —COOH, —NH₂, —NHCH₃, —N(CH₃)₂, and —CN,
—SH
—S(O)₂H,
oxo
hydroxy,
amino,
—NHR^{x}, where R^{x} is selected from C₁₋₄alkyl, and C₁₋₆alkyl substituted one to 4 times by fluoro,
—NR^{x1}R^{x2}, where R^{x1} and R^{x2} are each independently selected from C₁₋₄alkyl, and C₁₋₄alkyl substituted one to four times by fluoro,
guanidino,
—C(O)OH,
—C(O)OR^{x}, where R^{x} is selected from C₁₋₄alkyl, and C₁₋₄alkyl substituted one to four times by fluoro,
—C(O)NH₂,
—C(O)NHR^{x}, where R^{x} is selected from C₁₋₄alkyl, and C₁₋₄alkyl substituted one to four times by fluoro,
—C(O)NR^{x1}R^{x2}, where R^{x1} and R^{x2} are each independently selected from C₁₋₄alkyl, and C₁₋₄alkyl substituted one to four times by fluoro,
—S(O)₂NH₂,
—NHS(O)₂H,
—NHC(O)H,
—NHC(O)NH₂,
nitro, and
cyano.

Suitably “substituted” means the subject chemical moiety has from one to three substituents selected from the group consisting of:
fluoro,
chloro,
bromo,
C₁₋₄alkyl,
—OC₁₋₄alkyl,
oxo,
hydroxy,
amino,
—C(O)OH,
—C(O)NH₂,
nitro, and cyano.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
Ac (acetyl);
ACN (acetonitrile);
BH₃.Me₂S (borane dimethylsulfide compex);
Bn (benzyl);
Boc (tert-Butoxycarbonyl);
CAN (cerric ammonium nitrate);
C18 (refers to 18-carbon alkyl groups on silicon in HPLC stationary phase);
CH₃CN (acetonitrile);
DCM (dichloromethane);
DIAD (diisopropyl azodicarboxylate);
Dioxane (1,4-dioxane);
DMF (N,N-dimethylformamide);
DMSO (dimethylsulfoxide);
Et₃N (triethylamine);
EtOAc (ethyl acetate);
Et₂O (diethyl ether);
HCl (hydrochloric acid);
HEPES (4-(2-hydroxyethyl)-1-piperazine ethane sulfonic acid);
HPLC (high pressure liquid chromatography);
IPA (isopropyl alcohol);
K₂CO₃ (potassium carbonate);
LiOH.H₂O (lithium hydroxide monohydrate);
MeOH (methanol);
NaCNBH₃ (sodium cyanoborohydride);
NaHCO₃ (sodium bicarbonate);
NaOH (sodium hydroxide);
Na₂SO₄ (sodium sulfate);
NH₄Cl (ammonium chloride);
rt (room temperature);
TLC (thin layer chromatography);
TEA (triethylamine);
TFA (trifluoroacetic acid);
THF (tetrahydrofuran); and
T3P®® (2,4,6-tripropyl-1,3,5,2,4,6-trioxatriphosphorinane-2,4,6-trioxide).
All references to ether are to diethyl ether and brine refers to a saturated aqueous solution of NaCl.

### Methods of Use

The compounds according to Formula (IIIQ) and pharmaceutically acceptable salts thereof are inhibitors of the ATF4 pathway. Compounds which are inhibitors of the ATF4 pathway are readily identified by exhibiting activity in the ATF4 Cell Based Assay below. These compounds are potentially useful in the treatment of conditions wherein the underlying pathology is attributable to (but not limited to) modulation of the eIF2alpha pathway, for example, neurodegenerative disorders, cancer, cardiovascular and metabolic diseases. Accordingly, in another aspect the invention is directed to methods of treating such conditions.

The Integrated Stress Response (ISR) is a collection of cellular stress response pathways that converge in phosphorylation of the translation initiation factor eIF2α resulting in a reduction in overall translation in cells. Mammalian cells have four eIF2α kinases that phosphorylate this initiation factor in the same residue (serine 51); PERK is activated by the accumulation of unfolded proteins in the endoplasmic reticulum (ER), GCN2 is activated by amino acid starvation, PKR by viral infection and HRI by heme deficiency. Activation of these kinases decreases bulk protein synthesis but it also culminates in increased expression of specific mRNAs that contain uORFs. Two examples of these mRNAs are the transcription factor ATF4 and the pro-apoptotic gene CHOP. Phosphorylation of eIF2α upon stress and the concomitant reduction in protein translation has been shown to both have cytoprotective and cytotoxic effects depending on the cellular context and duration and severity of the stress. An integrated stress response-associated disease is a disease characterized by increased activity in the integrated stress response (e.g. increased phosphorylation of eIF2α by an eIF2α kinase compared to a control such as a subject without the disease). A disease associated with phosphorylation of eIF2α is disease characterized by an increase in phosphorylation of eIF2α relative to a control, such as a subject without the disease.

Activation of PERK occurs upon ER stress and hypoxic conditions and its activation and effect on translation has been shown to be cytoprotective for tumor cells [17]. Adaptation to hypoxia in the tumor microenvironment is critical for survival and metastatic potential. PERK has also been shown to promote cancer proliferation by limiting oxidative DNA damage and death [18, 19]. Moreover, a newly identified PERK inhibitor has been shown to have antitumor activity in a human pancreatic tumor xenograft model [20]. Compounds disclosed herein decrease the viability of cells that are subjected to ER-stress. Thus, pharmacological and acute inhibition of the PERK branch with the compounds disclosed herein results in reduced cellular fitness. During tumor growth, compounds disclosed herein, that block the cytoprotective effects of eIF2α phosphorylation upon stress may prove to be potent anti-proliferative agents.

It is known that under certain stress conditions several eIF2α kinases can be simultaneously activated. For example, during tumor growth, the lack of nutrients and hypoxic conditions are known to both activate GCN2 and PERK. Like PERK, GCN2 and their common target, ATF4, have been proposed to play a cytoprotective role [21]. By blocking signaling by both kinases, compounds disclosed herein may bypass the ability of the ISR to protect cancer cells against the effects of low nutrients and oxygen levels encountered during the growth of the tumor.

Prolonged ER stress leads to the accumulation of CHOP, a pro-apoptotic molecule. In a prion mouse model, overexpression of the phosphatase of eIF2α increased survival of prion-infected mice whereas sustained eIF2α phosphorylation decreased survival [22]. The restoration of protein translation rates during prion disease was shown to rescue synaptic deficits and neuronal loss. The compounds disclosed herein that make cells insensitive to eIF2α phosphorylation sustain protein translation. Compounds disclosed herein could prove potent inhibitors of neuronal cell death in prion disease by blocking the deleterious effects of prolonged eIF2α phosphorylation. Given the prevalence of protein misfolding and activation on the UPR in several neurodegenerative diseases (e.g. Alzheimer's (AD) and Parkinson's (PD)), manipulation of the PERK-eIF2α branch could prevent synaptic failure and neuronal death across the spectrum of these disorders.

Another example of tissue-specific pathology that is linked to heightened eIF2α phosphorylation is the fatal brain disorder, vanishing white matter disease (VWM) or childhood ataxia with CNS hypomyelination (CACH). This disease has been linked to mutation in eIF2B, the GTP exchange factor that is necessary for eIF2 function in translation [23]. eIF2α phosphorylation inhibits the activity of eIF2B and mutations in this exchange factor that reduce its exchange activity exacerbate the effects of eIF2α phosphorylation. The severe consequences of the CACH mutations point to the dangers of UPR hyper-activation, especially as it pertains to the myelin-producing oligodendrocyte. Small molecules, such as compounds disclosed herein, that block signaling through eIF2α phosphorylation may reduce the deleterious effects of its hyper-activation in VWM.

In another aspect is provided a method of improving long-term memory in a patient, which comprises administering a therapeutically effective amount of a compound of Formula (IIIQ) to the patient. In embodiments, the patient is human. In embodiments, the patient is a mammal.

The compounds of this invention inhibit the integrated stress response which is implicated in the pathogenesis of neurological disorders. Suitably the present invention relates to a method for treating or lessening the severity of neurological disorders. Suitably, the disorders treatable with the compounds of the invention include: Alcoholism, Anxiety, Depression, Schizophrenia, Bipolar Disorder, Obsessive Compulsive Disorder, Panic Disorder, Chronic Pain, Obesity, Senile Dementia, Migraine, Bulimia, Anorexia, Social Phobia, Pre-Menstrual Syndrome (PMS), Adolescent Depression, Trichotillomania, Dysthymia and Substance Abuse.

In embodiments, the neurological disorder is treated in a human patient.

The compounds of this invention inhibit the integrated stress response which is implicated in the pathogenesis of pain. Visceral pain is pain associated with the viscera, which encompass the internal organs of the body. These organs include, e.g., the heart, lungs, reproductive organs, bladder, ureters, the digestive organs, liver, pancreas, spleen, and kidneys. There are a variety of conditions in which visceral pain may exist. such as, for example, pancreatitis, labor, abdominal surgery associated with ileus, cystitis, menstrual period, or dysmenorrhea. Likewise, kidney pain, epigastric pain, pleural pain, and painful biliary colic, appendicitis pain may all be considered to be visceral pain. Substernal pain or pressure from early myocardial infarction is also visceral. Diseases of the stomach, dudenum or colon can cause visceral pain. Commonly encountered gastrointestinal (GI) disorders that cause visceral pain include functional bowel disorder (FBD) and inflammatory bowel disease (IBD). These GI disorders include a wide range of disease states that are currently only moderately controlled, including, with respect to FBD, gastro-esophageal reflux, dyspepsia, irritable bowel syndrome (IBS) and functional abdominal pain syndrome (FAPS), and, with respect to IBD, Crohn's disease, ileitis and ulcerative colitis, all of which regularly produce visceral pain.

Suitably the present invention relates to a method for treating or lessening the severity of pain. The invention can alleviate pain from many causes, including but not limited to shock; limb amputation; severe chemical or thermal burn injury; sprains, ligament tears, fractures, wounds and other tissue injuries; dental surgery, procedures and maladies; labor and delivery; migraine; during physical therapy; post operative pain; radiation poisoning; cancer; acquired immunodeficiency syndrome (AIDS); epidural (or peridural) fibrosis; failed back surgery and failed laminectomy; sciatica; painful sickle cell crisis; arthritis; autoimmune disease; intractable bladder pain; and the like. The present invention is directed to the treatment of intractable pain, whatever its cause.

In embodiments, pain is treated in a human patient.

The compounds of this invention inhibit the unfolded protein response which is implicated in the pathogenesis of inter vertebral disc degeneration. Suitably the present invention relates to a method for treating or lessening the severity of vertebral disc degeneration.

In embodiments, the compounds set forth herein are provided as pharmaceutical compositions comprising the compound and a pharmaceutically acceptable excipient. In embodiments of the method, the compound, or a pharmaceutically acceptable salt thereof, is co-adminstered with a second agent (e.g. therapeutic agent). In embodiments of the method, the compound, or a pharmaceutically acceptable salt thereof, is co-adminstered with a second agent (e.g. therapeutic agent), which is administered in a therapeutically effective amount. In embodiments, the second agent is an agent for improving memory.

Induction of long-term memory (LTM) has been shown to be facilitated by decreased and impaired by increased eIF2α phosphorylation. The data strongly support the notion that under physiological conditions, a decrease in eIF2α phosphorylation constitutes a critical step for the long term synaptic changes required for memory formation and ATF4 has been shown to be an important regulator of these processes [24] [25] [26]. It is not known what the contributions of the different eIF2α kinases to learning is or whether each play a differential role in the different parts of the brain. Regardless of the eIF2α kinase/s responsible for phosphorylation of eIF2α in the brain, compounds disclosed herein that block translation and ATF4 production make them ideal molecules to block the effects of this phosphorylation event on memory. Pharmacological treatment with compounds disclosed herein increase spatial memory and enhance auditory and contextual fear conditioning.

Regulators of translation, such as the compounds of Formula (IIIQ), could serve as therapeutic agents that improve memory in human disorders associated with memory loss such as Alzheimer's disease and in other neurological disorders that activate the UPR in neurons and thus could have negative effects on memory consolidation such as Parkinson's disease, Amyotrophic lateral sclerosis and prion diseases. In addition, a mutation in eIF2γ, that disrupts complex integrity linked intellectual disability (intellectual disability syndrome or ID) to impaired translation initiation in humans [27]. Hence, two diseases with impaired eIF2 function, ID and VWM, display distinct phenotypes but both affect mainly the brain and impair learning.

The compounds of Formula (IIIQ) are also useful in applications where increasing protein production output is desirable, such as in vitro cell free systems for protein production. In vitro systems have basal levels of eIF2α phosphorylation that reduce translational output [28, 29]. Similarly, production of antibodies by hybridomas may also be improved by addition of compounds disclosed herein.

In another aspect is provided a method of increasing protein expression of a cell or in vitro expression system, which comprises administering an effective amount of a compound of Formula (IIIQ) to the cell or expression system. In embodiments, the method is a method of increasing protein expression by a cell and includes administering an effective amount of a compound of Formula (IIIQ) to the cell. In embodiments, the method is a method of increasing protein expression by an in vitro protein expression system and includes administering an effective amount of a compound of Formula (IIIQ) to the in vitro (e.g. cell free) protein expression system.

In embodiments, the compounds set forth herein are provided as pharmaceutical compositions comprising the compound and a pharmaceutically acceptable excipient. In embodiments of the method, the compound, or a pharmaceutically acceptable salt thereof, is co-adminstered with a second agent. In embodiments of the method, the compound, or a pharmaceutically acceptable salt thereof, is co-adminstered with a second agent, which is administered in a therapeutically effective amount. In embodiments, the second agent is an agent for improving protein expression.

Suitably, the present invention relates to a method for treating or lessening the severity of breast cancer, including inflammatory breast cancer, ductal carcinoma, and lobular carcinoma.

Suitably the present invention relates to a method for treating or lessening the severity of colon cancer.

Suitably the present invention relates to a method for treating or lessening the severity of pancreatic cancer, including insulinomas, adenocarcinoma, ductal adenocarcinoma, adenosquamous carcinoma, acinar cell carcinoma, and glucagonoma.

Suitably the present invention relates to a method for treating or lessening the severity of skin cancer, including melanoma, including metastatic melanoma.

Suitably the present invention relates to a method for treating or lessening the severity of lung cancer including small cell lung cancer, non-small cell lung cancer, squamous cell carcinoma, adenocarcinoma, and large cell carcinoma.

Suitably the present invention relates to a method for treating or lessening the severity of cancers selected from the group consisting of brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, head and neck, kidney, liver, melanoma, ovarian, pancreatic, adenocarcinoma, ductal adenocarcinoma, adenosquamous carcinoma, acinar cell carcinoma, glucagonoma, insulinoma, prostate, sarcoma, osteosarcoma, giant cell tumor of bone, thyroid, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), neuroendocrine cancers and testicular cancer.

Suitably the present invention relates to a method for treating or lessening the severity of pre-cancerous syndromes in a mammal, including a human, wherein the pre-cancerous syndrome is selected from: cervical intraepithelial neoplasia, monoclonal gammapathy of unknown significance (MGUS), myelodysplastic syndrome, aplastic anemia, cervical lesions, skin nevi (pre-melanoma), prostatic intraepithleial (intraductal) neoplasia (PIN), Ductal Carcinoma in situ (DCIS), colon polyps and severe hepatitis or cirrhosis.

Suitably the present invention relates to a method for treating or lessening the severity of neurodegenerative diseases/injury, such as Alzheimer's disease, spinal cord injury, traumatic brain injury, ischemic stroke, stroke, diabetes, Parkinson disease, Huntington's disease, Creutzfeldt-Jakob Disease, and related prion diseases, progressive supranuclear palsy, amyotrophic lateral sclerosis, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementia, cognitive impairment, atherosclerosis, ocular diseases, arrhythmias, in organ transplantation and in the transportation of organs for transplantation.

Suitably the present invention relates to a method for preventing organ damage during and after organ transplantation and in the transportation of organs for transplantation. The method of preventing organ damage during and after organ transplantation comprises the in vivo administration of a compound of Formula (IIIQ). The method of preventing organ damage during the transportation of organs for transplantation comprises adding a compound of Formula (IIIQ) to the solution housing the organ during transportation.

Suitably, the present invention relates to a method for treating or lessening the severity of neurodegernative ocular diseases, wherein the disease is retinitis pigmentosa. Suitably, the present invention relates to a method for treating or lessening the severity of ocular diseases, wherein the disease is selected from retinal dystrophies and corneal dystrophies, such as Fuch's corneal dystrophy.

Suitably the present invention relates to a method for treating or lessening the severity of ocular diseases/angiogenesis. The method of treating or lessening the severity of ocular diseases/angiogenesis comprises the in vivo administration of a compound of Formula (III). In embodiments of methods according to the invention, the disorder of ocular diseases, including vascular leakage can be: edema or neovascularization for any occlusive or inflammatory retinal vascular disease, such as rubeosis irides, neovascular glaucoma, pterygium, vascularized glaucoma filtering blebs, conjunctival papilloma; choroidal neovascularization, such as neovascular age-related macular degeneration (AMD), myopia, prior uveitis, trauma, or idiopathic; macular edema, such as post surgical macular edema, macular edema secondary to uveitis including retinal and/or choroidal inflammation, macular edema secondary to diabetes, and macular edema secondary to retinovascular occlusive disease (i.e. branch and central retinal vein occlusion); retinal neovascularization due to diabetes, such as retinal vein occlusion, uveitis, ocular ischemic syndrome from carotid artery disease, ophthalmic or retinal artery occlusion, sickle cell retinopathy, other ischemic or occlusive neovascular retinopathies, retinopathy of prematurity, or Eale's Disease; and genetic disorders, such as VonHippel-Lindau syndrome.

In some embodiments, the neovascular age-related macular degeneration is wet age-related macular degeneration. In other embodiments, the neovascular age-related macular degeneration is dry age-related macular degeneration and the patient is characterized as being at increased risk of developing wet age-related macular degeneration.

In embodiments, the ocular disease is treated in a human patient.

The methods of treatment of the invention comprise administering an effective amount of a compound according to Formula (IIIQ) or a pharmaceutically acceptable salt, thereof to a patient in need thereof.

The invention also provides a compound according to Formula (IIIQ) or a pharmaceutically-acceptable salt thereof for use in medical therapy, and particularly in therapy for: cancer, pre-cancerous syndromes, Alzheimer's disease, spinal cord injury, traumatic brain injury, ischemic stroke, stroke, diabetes, Parkinson disease, Huntington's disease, Creutzfeldt-Jakob Disease, and related prion diseases, progressive supranuclear palsy, amyotrophic lateral sclerosis, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementia, cognitive impairment, atherosclerosis, ocular diseases, in organ transplantation and arrhythmias. The invention also provides a compound according to Formula (IIIQ) or a pharmaceutically-acceptable salt thereof for use in preventing organ damage during the transportation of organs for transplantation. Thus, in further aspect, the invention is directed to the use of a compound according to Formula (IIIQ) or a pharmaceutically acceptable salt thereof in the manfacture of a medicament for the treatment of a disorder characterized by activation of the UPR, such as cancer.

The methods of treatment of the invention comprise administering a safe and effective amount of a compound of Formula (IIIQ), or a pharmaceutically acceptable salt thereof to a mammal, suitably a human, in need thereof.

As used herein, “treating”, and derivatives thereof, in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition.

The term “treating” and derivatives thereof refers to therapeutic therapy. Therapeutic therapy is appropriate to alleviate symptions or to treat at early signs of disease or its progression. Prophylactic therapy is appropriate when a subject has, for example, a strong family history of neurodegenerative diseases. Prophylactic therapy is appropriate when a subject has, for example, a strong family history of cancer or is otherwise considered at high risk for developing cancer, or when a subject has been exposed to a carcinogen.

The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” in reference to a compound of Formula (IIIQ), or a pharmaceutically acceptable salt thereof, means an amount of the compound sufficient to treat the patient's condition but low enough to avoid serious side effects (at a reasonable benefit/risk ratio) within the scope of sound medical judgment. A safe and effective amount of the compound will vary with the particular route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient”, and derivatives thereof refers to a human or other mammal, suitably a human.

The compounds of Formula (IIIQ) or pharmaceutically acceptable salts thereof may be administered by any suitable route of administration, including systemic administration. Systemic administration includes oral administration, and parenteral administration. Parenteral administration refers to routes of administration other than enteral, transdermal, or by inhalation, and is typically by injection or infusion. Parenteral administration includes intravenous, intramuscular, and subcutaneous injection or infusion.

The compounds of Formula (IIIQ) or pharmaceutically acceptable salts thereof may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Additionally, the compounds of Formula (IIIQ) or pharmaceutically-acceptable salts thereof may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of the compound in vivo; (b) modify the duration of action of the compound in vivo; (c) modify the transportation or distribution of the compound in vivo; (d) modify the solubility of the compound in vivo; and (e) overcome a side effect or other difficulty encountered with the compound. Where a —COOH or —OH group is present, pharmaceutically acceptable esters can be employed, for example methyl, ethyl, and the like for —COOH, and acetate, maleate, and the like for —OH, and those esters known in the art for modifying solubility or hydrolysis characteristics.

The compounds of Formula (IIIQ) and pharmaceutically acceptable salts thereof may be co-administered with at least one other active agent known to be useful in the treatment of cancer or pre-cancerous syndromes.

By the term “co-administration” as used herein is meant either simultaneous administration or any manner of separate sequential administration of an ATF4 pathway inhibiting compound, as described herein, and a further active agent or agents, known to be useful in the treatment of cancer, including chemotherapy and radiation treatment. The term further active agent or agents, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered by injection and another compound may be administered orally.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; cell cycle signaling inhibitors; proteasome inhibitors; and inhibitors of cancer metabolism.

Examples of a further active ingredient or ingredients (anti-neoplastic agent) for use in combination or co-administered with the presently invented ATF4 pathway inhibiting compounds are chemotherapeutic agents.

Suitably, the pharmaceutically active compounds of the invention are used in combination with a VEGFR inhibitor, suitably 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt thereof, which is disclosed and claimed in in International Application No. PCT/US01/49367, having an International filing date of Dec. 19, 2001, International Publication Number WO02/059110 and an International Publication date of Aug. 1, 2002, the entire disclosure of which is hereby incorporated by reference, and which is the compound of Example 69. 5-[[4-[(2,3-dimethyl-2H-indazol-6-yl)methylamino]-2-pyrimidinyl]amino]-2-methylbenzenesulfonamide can be prepared as described in International Application No. PCT/US01/49367.

In one embodiment, the cancer treatment method of the claimed invention includes the co-administration a compound of Formula (IIIQ) and/or a pharmaceutically acceptable salt thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, cell cycle signaling inhibitors; proteasome inhibitors; and inhibitors of cancer metabolism.

“Chemotherapeutic” or “chemotherapeutic agent” is used in accordance with its plain ordinary meaning and refers to a chemical composition or compound having antineoplastic properties or the ability to inhibit the growth or proliferation of cells.

Additionally, the compounds described herein can be co-administered with conventional immunotherapeutic agents including, but not limited to, immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, ⁹⁰Y, or ¹³¹I, etc.).

In a further embodiment, the compounds described herein can be co-administered with conventional radiotherapeutic agents including, but not limited to, radionuclides such as ⁴⁷SC, ⁶⁴C ⁶⁷C, ⁸⁹Sr, ⁸⁶Y, ⁸⁷Y, and ²¹²Bi, optionally conjugated to antibodies directed against tumor antigens.

Additional examples of a further active ingredient or ingredients (anti-neoplastic agent) for use in combination or co-administered with the presently invented ATF4 pathway inhibiting compounds are anti-PD-L1 agents.

Anti-PD-L1 antibodies and methods of making the same are known in the art.

Such antibodies to PD-L1 may be polyclonal or monoclonal, and/or recombinant, and/or humanized.

Exemplary PD-L1 antibodies are disclosed in:
U.S. Pat. No. 8,217,149; Ser. No. 12/633,339;
U.S. Pat. No. 8,383,796; Ser. No. 13/091,936;
U.S. Pat. No 8,552,154; Ser. No. 13/120,406;
US patent publication No. 20110280877; Ser. No. 13/068,337;
US Patent Publication No. 20130309250; Ser. No. 13/892,671;
WO2013019906;
WO2013079174;
U.S. application Ser. No. 13/511,538 (filed Aug. 7, 2012), which is the US National Phase of International Application No. PCT/US10/58007 (filed 2010);
and
U.S. application Ser. No. 13/478,511 (filed May 23, 2012).

Additional exemplary antibodies to PD-L1 (also referred to as CD274 or B7-H1) and methods for use are disclosed in U.S. Pat. No. 7,943,743; US20130034559, WO2014055897, U.S. Pat. No. 8,168,179; and U.S. Pat. No. 7,595,048. PD-L1 antibodies are in development as immuno-modulatory agents for the treatment of cancer.

In one embodiment, the antibody to PD-L1 is an antibody disclosed in U.S. Pat. No. 8,217,149. In another embodiment, the anti-PD-L1 antibody comprises the CDRs of an antibody disclosed in U.S. Pat. No. 8,217,149.

In another embodiment, the antibody to PD-L1 is an antibody disclosed in U.S. application Ser. No. 13/511,538. In another embodiment, the anti-PD-L1 antibody comprises the CDRs of an antibody disclosed in U.S. application Ser. No. 13/511,538.

In another embodiment, the antibody to PD-L1 is an antibody disclosed in application Ser. No. 13/478,511. In another embodiment, the anti-PD-L1 antibody comprises the CDRs of an antibody disclosed in U.S. application Ser. No. 13/478,511.

In one embodiment, the anti-PD-L1 antibody is BMS-936559 (MDX-1105). In another embodiment, the anti-PD-L1 antibody is MPDL3280A (RG7446). In another embodiment, the anti-PD-L1 antibody is MED14736. In another embodiment, the anti-PD-L1 antibody is atezolizumab. In another embodiment, the anti-PD-L1 antibody is avelumab. In another embodiment, the anti-PD-L1 antibody is durvalumab.

Additional examples of a further active ingredient or ingredients (anti-neoplastic agent) for use in combination or co-administered with the presently invented ATF4 pathway inhibiting compounds are PD-1 antagonist.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any embodiments of the aspects or embodiments of the present invention in which a human individual is to be treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in the any of the aspects of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

Examples of mAbs that bind to human PD-1, and useful in the various aspects and embodiments of the present invention, are described in U.S. Pat. Nos. 7,488,802, 7521051, 8,008,449, 8,354,509, 8,168,757, WO2004/004771, WO2004/072286, WO2004/056875, and US2011/0271358.

Specific anti-human PD-1 mAbs useful as the PD-1 antagonist in any of the aspects and embodiments of the present invention include: MK-3475, a humanized IgG4 mAb with the structure described in WHO Drug Information, Vol. 27, No. 2, pages 161-162 (2013) and which comprises the heavy and light chain amino acid sequences shown in FIG. 6; nivolumab, a human IgG4 mAb with the structure described in WHO Drug Information, Vol. 27, No. 1, pages 68-69 (2013) and which comprises the heavy and light chain amino acid sequences shown in FIG. 7; the humanized antibodies h409A11, h409A16 and h409A17, which are described in WO2008/156712, and AMP-514, which is being developed by Medimmune.

Other PD-1 antagonists useful in the any of the aspects and embodiments of the present invention include an immunoadhesin that specifically binds to PD-1, and preferably specifically binds to human PD-1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

Other examples of mAbs that bind to human PD-L1, and useful in the treatment method, medicaments and uses of the present invention, are described in WO2013/019906, WO2010/077634 A1 and U.S. Pat. No. 8,383,796. Specific anti-human PD-L1 mAbs useful as the PD-1 antagonist in the treatment method, medicaments and uses of the present invention include MPDL3280A, BMS-936559, MED14736, MSB0010718C.

KEYTRUDA®/pembrolizumab is an anti-PD-1 antibody marketed for the treatment of lung cancer by Merck. The amino acid sequence of pembrolizumab and methods of using are disclosed in U.S. Pat. No. 8,168,757.

Opdivo®/nivolumab is a fully human monoclonal antibody marketed by Bristol Myers Squibb directed against the negative immunoregulatory human cell surface receptor PD-1 (programmed death-1 or programmed cell death-1/PCD-1) with immunopotentiation activity. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of P13k/Akt pathway activation. Other names for nivolumab include: BMS-936558, MDX-1106, and ONO-4538. The amino acid sequence for nivolumab and methods of using and making are disclosed in U.S. Pat. No. 8,008,449.

Additional examples of a further active ingredient or ingredients (anti-neoplastic agent) for use in combination or co-administered with the presently invented ATF4 pathway inhibiting compounds are immuno-modulators.

As used herein “immuno-modulators” refer to any substance including monoclonal antibodies that affects the immune system. The ICOS binding proteins of the present invention can be considered immune-modulators. Immuno-modulators can be used as anti-neoplastic agents for the treatment of cancer. For example, immune-modulators include, but are not limited to, anti-CTLA-4 antibodies such as ipilimumab (YERVOY®) and anti-PD-1 antibodies (Opdivo®/nivolumab and Keytruda®/pembrolizumab). Other immuno-modulators include, but are not limited to, OX-40 antibodies, PD-L1 antibodies, LAG3 antibodies, TIM-3 antibodies, 41BB antibodies and GITR antibodies.

Yervoy® (ipilimumab) is a fully human CTLA-4 antibody marketed by Bristol Myers Squibb. The protein structure of ipilimumab and methods are using are described in U.S. Pat. Nos. 6,984,720 and 7,605,238.

Suitably, the compounds of the invention are combined with an inhibitor of the activity of the protein kinase R (PKR)-like ER kinase, PERK.

Suitably, the compounds of the invention are combined with an inhibitor of the activity of the eIF2a kinases protein kinase R, (PKR), Heme-regulated eIF2a kinase (HRI), or general control non-derepressible 2 (GCN2).

Suitably, the compounds of Formula (IIIQ) and pharmaceutically acceptable salts thereof may be co-administered with at least one other active agent known to be useful in the treatment of neurodegenerative diseases/injury.

Suitably, the compounds of Formula (IIIQ) and pharmaceutically acceptable salts thereof may be co-administered with at least one other active agent known to be useful in the treatment of diabetes.

Suitably, the compounds of Formula (IIIQ) and pharmaceutically acceptable salts thereof may be co-administered with at least one other active agent known to be useful in the treatment of cardiovascular disease.

Suitably, the compounds of Formula (IIIQ) and pharmaceutically acceptable salts thereof may be co-administered with at least one other active agent known to be useful in the treatment of ocular diseases.

The compounds described herein can be used in combination with one another, with other active agents known to be useful in treating cancer (e.g. pancreatic cancer, breast cancer, multiple myeloma, or cancers of secretory cells), neurodegenerative diseases, vanishing white matter disease, childhood ataxia with CNS hypomyelination, and/or intellectual disability syndromes (e.g. associated with impaired function of eIF2 or components in a signal transduction pathway including eIF2), or with adjunctive agents that may not be effective alone, but may contribute to the efficacy of the active agent.

In embodiments, the compounds set forth herein are provided as pharmaceutical compositions comprising the compound and a pharmaceutically acceptable excipient. In embodiments of the method, the compound, or a pharmaceutically acceptable salt thereof, is co-adminstered with a second agent (e.g. therapeutic agent). In embodiments of the method, the compound, or a pharmaceutically acceptable salt thereof, is co-adminstered with a second agent (e.g. therapeutic agent), which is administered in a therapeutically effective amount. In embodiments of the method, the second agent is an agent for treating cancer (e.g. pancreatic cancer, breast cancer, multiple myeloma, or cancers of secretory cells), neurodegenerative diseases, vanishing white matter disease, childhood ataxia with CNS hypomyelination, and/or intellectual disability syndromes (e.g. associated with impaired function of eIF2 or components in a signal transduction pathway including eIF2), or an inflammatory disease (e.g. POCD or TBI). In embodiments, the second agent is an anti-cancer agent. In embodiments, the second agent is a chemotherapeutic. In embodiments, the second agent is an agent for improving memory. In embodiments, the second agent is an agent for treating a neurodegenerative disease. In embodiments, the second agent is an agent for treating vanishing white matter disease. In embodiments, the second agent is an agent for treating childhood ataxia with CNS hypo-myelination. In embodiments, the second agent is an agent for treating an intellectual disability syndrome. In embodiments, the second agent is an agent for treating pancreatic cancer. In embodiments, the second agent is an agent for treating breast cancer. In embodiments, the second agent is an agent for treating multiple myeloma. In embodiments, the second agent is an agent for treating myeloma. In embodiments, the second agent is an agent for treating a cancer of a secretory cell. In embodiments, the second agent is an agent for reducing eIF2a phosphorylation. In embodiments, the second agent is an agent for inhibiting a pathway activated by eIF2α phosphorylation. In embodiments, the second agent is an agent for inhibiting the integrated stress response. In embodiments, the second agent is an anti-inflammatory agent.

The term “eIF2alpha” or “eIF2α” refers to the protein “Eukaryotic translation initiation factor 2A”. In embodiments, “eIF2alpha” or “eIF2α” refers to the human protein. Included in the term “eIF2alpha” or “eIF2α” are the wildtype and mutant forms of the protein. In embodiments, “eIF2alpha” or “eIF2α” refers to the protein associated with Entrez Gene 83939, OMIM 609234, UniProt Q9BY44, and/or RefSeq (protein) NP 114414.

Suitably, the present invention relates to a method for treating an integrated stress response associated disease in a patient in need of such treatment, the method including administering a therapeutically effective amount of a compound of Formula (IIIQ), or a pharmaceutically acceptable salt thereof, to the patient.

Suitably, the integrated stress response-associated disease is cancer. Suitably, the integrated stress response-associated disease is a neurodegenerative disease. Suitably, the integrated stress response-associated disease is vanishing white matter disease. Suitably, the integrated stress response-associated disease is childhood ataxia with CNS hypomyelination. Suitably, the integrated stress response-associated disease is an intellectual disability syndrome.

Suitably, the present invention relates to a method for treating a disease associated with phosphorylation of eIF2α in a patient in need of such treatment, which comprises administering a therapeutically effective amount of a compound of Formula (IIIZ), or a pharmaceutically acceptable salt thereof, to the patient.

Suitably, the disease associated with phosphorylation of eIF2α is cancer. Suitably, the disease associated with phosphorylation of eIF2α is a neurodegenerative disease. Suitably, the disease associated with phosphorylation of eIF2α is vanishing white matter disease. Suitably, the disease associated with phosphorylation of eIF2α is childhood ataxia with CNS hypomyelination. Suitably, the disease associated with phosphorylation of eIF2α is an intellectual disability syndrome.

Suitably, the present invention relates to a method for treating a disease selected from the group consisting of cancer, a neurodegenerative disease, vanishing white matter disease, childhood ataxia with CNS hypomyelination, and an intellectual disability syndrome.

Suitably, the present invention relates to a method for treating an inflammatory disease in a patient in need of such treatment, which comprises administering a therapeutically effective amount of a compound of Formula (IIIQ), or a pharmaceutically acceptable salt thereof, to the patient.

Suitably, the inflammatory disease is associated with neurological inflammation. Suitably, the inflammatory disease is postoperative cognitive dysfunction. Suitably, the inflammatory disease is traumatic brain injury or chronic traumatic encephalopathy (CTE).

In embodiments of the method of treating a disease, the disease is selected from the group consisting of cancer, a neurodegenerative disease, vanishing white matter disease, childhood ataxia with CNS hypomyelination, and an intellectual disability syndrome. In embodiments of the method of treating a disease, the disease is cancer. In embodiments of the method of treating a disease, the disease is a neurodegenerative disease. In embodiments of the method of treating a disease, the disease is vanishing white matter disease. In embodiments of the method of treating a disease, the disease is childhood ataxia with CNS hypomyelination. In embodiments of the method of treating a disease, the disease is an intellectual disability syndrome. In embodiments of the method of treating a disease, the disease is associated with phosphorylation of eIF2α. In embodiments of the method of treating a disease, the disease is associated with an eIF2α signaling pathway. In embodiments of the method of treating a disease, the disease is a cancer of a secretory cell type. In embodiments of the method of treating a disease, the disease is pancreatic cancer. In embodiments of the method of treating a disease, the disease is breast cancer. In embodiments of the method of treating a disease, the disease is multiple myeloma. In embodiments of the method of treating a disease, the disease is lymphoma. In embodiments of the method of treating a disease, the disease is leukemia. In embodiments of the method of treating a disease, the disease is a hematopoietic cell cancer.

In embodiments of the method of treating a disease, the disease is Alzheimer's disease. In embodiments of the method of treating a disease, the disease is Amyotrophic lateral sclerosis. In embodiments of the method of treating a disease, the disease is Creutzfeldt-Jakob disease. In embodiments of the method of treating a disease, the disease is frontotemporal dementia. In embodiments of the method of treating a disease, the disease is Gerstmann-Straussler-Scheinker syndrome. In embodiments of the method of treating a disease, the disease is Huntington's disease. In embodiments of the method of treating a disease, the disease is HIV-associated dementia. In embodiments of the method of treating a disease, the disease is kuru. In embodiments of the method of treating a disease, the disease is Lewy body dementia. In embodiments of the method of treating a disease, the disease is Multiple sclerosis. In embodiments of the method of treating a disease, the disease is Parkinson's disease. In embodiments of the method of treating a disease, the disease is a Prion disease.

In embodiments of the method of treating a disease, the disease is an inflammatory disease. In embodiments, the inflammatory disease is postoperative cognitive dysfunction. In embodiments, the inflammatory disease is traumatic brain injury. In embodiments, the inflammatory disease is arthritis. In embodiments, the inflammatory disease is rheumatoid arthritis. In embodiments, the inflammatory disease is psoriatic arthritis. In embodiments, the inflammatory disease is juvenile idiopathic arthritis. In embodiments, the inflammatory disease is multiple sclerosis. In embodiments, the inflammatory disease is systemic lupus erythematosus (SLE). In embodiments, the inflammatory disease is myasthenia gravis. In embodiments, the inflammatory disease is juvenile onset diabetes. In embodiments, the inflammatory disease is diabetes mellitus type 1. In embodiments, the inflammatory disease is Guillain-Barre syndrome. In embodiments, the inflammatory disease is Hashimoto's encephalitis. In embodiments, the inflammatory disease is Hashimoto's thyroiditis. In embodiments, the inflammatory disease is ankylosing spondylitis. In embodiments, the inflammatory disease is psoriasis. In embodiments, the inflammatory disease is Sjogren's syndrome. In embodiments, the inflammatory disease is vasculitis. In embodiments, the inflammatory disease is glomerulonephritis. In embodiments, the inflammatory disease is auto-immune thyroiditis. In embodiments, the inflammatory disease is Behcet's disease. In embodiments, the inflammatory disease is Crohn's disease. In embodiments, the inflammatory disease is ulcerative colitis. In embodiments, the inflammatory disease is bullous pemphigoid. In embodiments, the inflammatory disease is sarcoidosis. In embodiments, the inflammatory disease is ichthyosis. In embodiments, the inflammatory disease is Graves ophthalmopathy. In embodiments, the inflammatory disease is inflammatory bowel disease. In embodiments, the inflammatory disease is Addison's disease. In embodiments, the inflammatory disease is Vitiligo. In embodiments, the inflammatory disease is asthma. In embodiments, the inflammatory disease is allergic asthma. In embodiments, the inflammatory disease is acne vulgaris. In embodiments, the inflammatory disease is celiac disease. In embodiments, the inflammatory disease is chronic prostatitis. In embodiments, the inflammatory disease is inflammatory bowel disease. In embodiments, the inflammatory disease is pelvic inflammatory disease. In embodiments, the inflammatory disease is reperfusion injury. In embodiments, the inflammatory disease is sarcoidosis. In embodiments, the inflammatory disease is transplant rejection. In embodiments, the inflammatory disease is interstitial cystitis. In embodiments, the inflammatory disease is atherosclerosis. In embodiments, the inflammatory disease is atopic dermatitis.

In embodiments, the method of treatment is a method of prevention. For example, a method of treating postsurgical cognitive dysfunction may include preventing postsurgical cognitive dysfunction or a symptom of postsurgical cognitive dysfunction or reducing the severity of a symptom of postsurgical cognitive dysfunction by administering a compound described herein prior to surgery.

In an embodiment, this invention provides a compound of Formula (IIIQ), or a pharmaceutically acceptable salt thereof, for use in the treatment of a disease selected from the group consisting of cancer, a neurodegenerative disease, vanishing white matter disease, childhood ataxia with CNS hypomyelination, and an intellectual disability syndrome.

In an embodiment, this invention provides a compound of Formula (IIIQ), or a pharmaceutically acceptable salt thereof, for use in the treatment of an integrated stress response associated disease.

In an embodiment, this invention provides a compound of Formula (IIIQ), or a pharmaceutically acceptable salt thereof, for use in the treatment of a disease associated with phosphorylation of el F2α.

In an embodiment, this invention provides for the use of a compound of Formula (IIIQ), or a pharmaceutically acceptable salt thereof, in the manufacture of a medicament for the treatment of a disease selected from the group consisting of cancer, a neurodegenerative disease, vanishing white matter disease, childhood ataxia with CNS hypomyelination, and an intellectual disability syndrome.

In an embodiment, this invention provides for the use of a compound of Formula (IIIQ), or a pharmaceutically acceptable salt thereof, in the manufacture of a medicament for the treatment an integrated stress response associated disease.

In an embodiment, this invention provides for the use of a compound of Formula (IIIQ), or a pharmaceutically acceptable salt thereof, in the manufacture of a medicament for the treatment of a disease associated with phosphorylation of eIF2α.

### Compositions

The pharmaceutically active compounds within the scope of this invention are useful as ATF4 pathway inhibitors in mammals, particularly humans, in need thereof.

The present invention therefore provides a method of treating cancer, neurodegeneration and other conditions requiring ATF4 pathway inhibition, which comprises administering an effective amount of a compound of Formula (IIIQ) or a pharmaceutically acceptable salt thereof. The compounds of Formula (IIIQ) also provide for a method of treating the above indicated disease states because of their demonstrated ability to act as ATF4 pathway inhibitors. The drug may be administered to a patient in need thereof by any conventional route of administration, including, but not limited to, intravenous, intramuscular, oral, topical, subcutaneous, intradermal, intraocular and parenteral. Suitably, a ATF4 pathway inhibitor may be delivered directly to the brain by intrathecal or intraventricular route, or implanted at an appropriate anatomical location within a device or pump that continuously releases the ATF4 pathway inhibiting drug.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical compositions are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of a ATF4 pathway inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages, is preferred. Parenteral administration, at high dosages, however, also can be used when safe and convenient for the patient.

Optimal dosages to be administered may be readily determined by those skilled in the art, and will vary with the particular ATF4 pathway inhibitor in use, the strength of the preparation, the mode of administration, and the advancement of the disease condition. Additional factors depending on the particular patient being treated will result in a need to adjust dosages, including patient age, weight, diet, and time of administration.

When administered to prevent organ damage in the transportation of organs for transplantation, a compound of Formula (IIIQ) is added to the solution housing the organ during transportation, suitably in a buffered solution.

The method of this invention of inducing ATF4 pathway inhibitory activity in mammals, including humans, comprises administering to a subject in need of such activity an effective ATF4 pathway inhibiting amount of a pharmaceutically active compound of the present invention.

The invention also provides for the use of a compound of Formula (IIIQ) or a pharmaceutically acceptable salt thereof in the manufacture of a medicament for inhibiting the ATF4 pathway.

The invention also provides for the use of a compound of Formula (IIIQ) or a pharmaceutically acceptable salt thereof in the manufacture of a medicament for use in treating cancer, pre-cancerous syndromes, Alzheimer's disease, spinal cord injury, traumatic brain injury, ischemic stroke, stroke, diabetes, Parkinson disease, Huntington's disease, Creutzfeldt-Jakob Disease, and related prion diseases, progressive supranuclear palsy, amyotrophic lateral sclerosis, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementia, cognitive impairment, atherosclerosis, ocular diseases, arrhythmias, in organ transplantation and in the transportation of organs for transplantation.

The invention also provides for the use of a compound of Formula (IIIQ) or a pharmaceutically acceptable salt thereof in the manufacture of a medicament for use in preventing organ damage during the transportation of organs for transplantation.

The invention also provides for a pharmaceutical composition for use as a ATF4 pathway inhibitor which comprises a compound of Formula (IIIQ) or a pharmaceutically acceptable salt thereof and a pharmaceutically acceptable carrier.

The invention also provides for a pharmaceutical composition for use in the treatment of cancer which comprises a compound of Formula (IIIQ) or a pharmaceutically acceptable salt thereof and a pharmaceutically acceptable carrier.

In addition, the pharmaceutically active compounds of the present invention can be co-administered with further active ingredients, such as other compounds known to treat cancer, or compounds known to have utility when used in combination with a ATF4 pathway inhibitor.

The invention also provides novel processes and novel intermedites useful in preparing the presently invented compounds.

The invention also provides a pharmaceutical composition comprising from 0.5 to 1,000 mg of a compound of Formula (IIIQ) or pharmaceutically acceptable salt thereof and from 0.5 to 1,000 mg of a pharmaceutically acceptable excipient.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

### Example I

### N,N′-(bicyclo[2.2.2]octane-1,4-diyl)bis(2-(4-chlorophenoxy)acetamide)

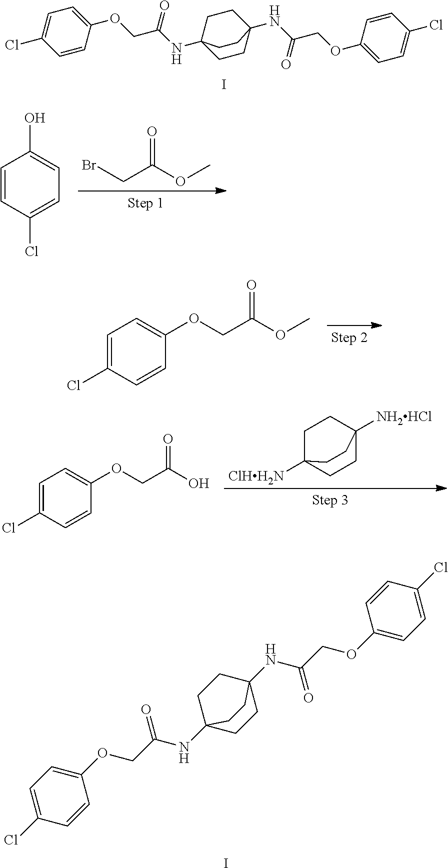

Step 1: To a solution of 4-chlorophenol (15 g, 116.67 mmol, 1 equiv) in DMF (100 mL) at room temperature was added anhydrous potassium carbonate (24.15 g, 175.01 mmol, 1.5 equiv) portionwise. After stirring for 2 minutes, methyl-2-bromoacetate (13.3 mL, 140.01 mmol, 1.2 equiv) was added. The reaction mixture was heated at 80° C. for 4 h. After consumption of the starting material (TLC, 5% EtOAc in hexane), the reaction mixture was cooled to room temperature, diluted with water (100 mL) and extracted with EtOAc (2×100 mL). The combined organic layer was washed with brine solution (50 mL), dried over anhydrous sodium sulfate, filtered and concentrated en vacuo to give the crude product. The crude product was purified by flash column chromatography (Combiflash®®) using a silica gel column and the product was eluted at 15% ethyl acetate in hexane. Fractions containing product were concentrated to give methyl 2-(4-chlorophenoxy)acetate (22.5 g, 96.5% yield) as pale yellow liquid. LCMS (ES) m/z=200.0 [M+H]⁺. ¹H NMR (400 MHz, CDCl₃): δ ppm 3.67 (s, 3H), 4.78 (s, 2H), 6.91-6.95 (m, 2H), 7.28-7.32 (m, 2H).

Step 2: To a solution of methyl 2-(4-chlorophenoxy)acetate (22.5 g, 112.15 mmol, 1 equiv) in ethanol (100 mL) at 0° C. was added a solution of sodium hydroxide (5.38 g, 134.58 mmol, 1.2 equiv) in water (100 mL). After stirring for 5 minutes at 0° C., the reaction mixture was allowed to warm to room temperature and then refluxed for 2.5 h during which the starting material was completely consumed. Heating was removed and the reaction mixture was allowed to cool down to room temperature. Ethanol was removed en vacuo and the reaction mixture was diluted with water (50 mL) followed by extraction with Et₂O (50 mL). The aqueous layer was acidified with 1 N HCl upto pH 3 and the precipitated product was filtered through a cintered funnel, washed with ice-cold water (10 mL) and dried under high vacuum to give 2-(4-chlorophenoxy)acetic acid (20 g, 95.6% yield) as white solid. LCMS (ES) m/z=186.1 [M+H]⁺. ¹H NMR (400 MHz, DMSO-d₆) δ ppm 4.65 (s, 2H), 6.91 (d, J=9.2 Hz, 2H), 7.29 (d, J=8.8 Hz, 2H), 12.98 (bs, 1H).

Step 3: To bicyclo[2.2.2]octane-1,4-diamine dihydrochloride (0.200 g, 0.938 mmol, 1 equiv) taken in DCM (10 mL) at 0° C. was added triethylamine (0.660 mL, 4.69 mmol, 5 equiv) and 2-(4-chlorophenoxy)acetic acid (0.437 g, 2.34 mmol, 2.5 equiv). After stirring for 5 minutes at 0° C., T3P®® (50 wt. % in ethyl acetate) (1.79 mL, 2.81 mmol, 3 equiv) was added and the reaction mixture was stirred at room temperature for 16 h at which time the staring materials were completely consumed. The reaction mixture was diluted with water (5 mL) and extracted with DCM (2×10 mL). The combined organic extract was washed with saturated aqueous NaHCO₃ solution (8 mL) and water (10 mL). The organic phase was dried over anhydrous sodium sulfate, filtered and concentrated under reduced pressure to give the crude product. The crude material was purified by flash column chromatography using a silica gel column where the product was eluted at 4-5% methanol in DCM. Fractions containing product were concentrated under reduced pressure to give N,N′-(bicyclo[2.2.2]octane-1,4-diyl)bis(2-(4-chlorophenoxy)acetamide) (0.040 g, 8.94% yield) as white solid. LCMS (ES) m/z=477.1 [M+H]⁺. ¹H NMR (400 MHz, DMSO-d₆) δ ppm 1.87 (s, 12H), 4.35 (s, 4H), 6.9 (d, J=9.2 Hz, 4H), 7.30 (d, J=8.8 Hz, 4H), 7.37 (s, 2H).

### Example II

### N,N′-(bicyclo[1.1.1]pentane-1,3-diyl)bis(2-(4-chlorophenoxy)acetamide)

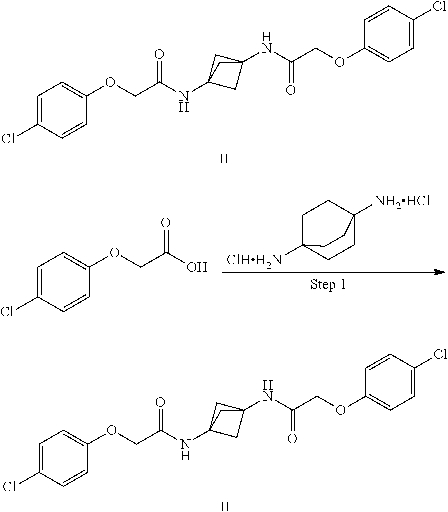

Step 1: To bicyclo[1.1.1]pentane-1,3-diamine dihydrochloride (0.200 g, 1.169 mmol, 1 equiv) taken in DCM (10 mL) at 0° C. was added triethylamine (0.820 mL, 5.845 mmol, 5 equiv) and 2-(4-chlorophenoxy)acetic acid (0.479 g, 2.572 mmol, 2.2 equiv). After stirring for 5 minutes at 0° C., T3P®® (50 wt. % in ethyl acetate) (2.22 mL, 3.507 mmol, 3 equiv) was added and the reaction mixture was stirred at room temperature for 16 h at which time starting materials were completely consumed. The reaction mixture was diluted with water (5 mL) and extracted with DCM (2×15 mL). The combined organic extract was washed with saturated aqueous NaHCO₃ solution (8 mL) and water (5 mL). The organic phase was dried over anhydrous sodium sulfate, filtered and concentrated under reduced pressure to give the crude product. The crude material was purified by flash column chromatography using a silica gel column where the product was eluted at 1-3% methanol in DCM as eluant. Fractions containing product were concentrated under reduced pressure to give N,N′-(bicyclo[1.1.1]pentane-1,3-diyl)bis(2-(4-chlorophenoxy)acetamide) (0.260 g, 51.38 yield) as white solid. LCMS (ES) m/z=435 [M+H]⁺. ¹H NMR (400 MHz, DMSO-d₆) δ ppm 2.23 (s, 6H), 4.40 (s, 4H), 6.94 (d, J=8.8 Hz, 4H), 7.31 (d, J=8.8 Hz, 4H), 8.65 (s, 2H).

### Examples III and IV

The Compounds of Examples III and IV were prepared generally according to the procedures described above for Examples I and II.

TABLE I

| Cmpd | | | LCMS m/z | ¹H-NMR (400 MHz, |
|---|---|---|---|---|
| # | Structure | Name | [M + H]⁺ | DMSO-d₆) |
| III | 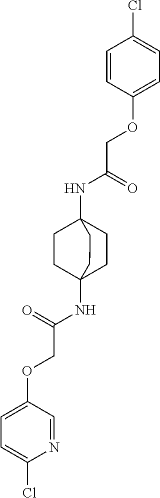 [SMILES: O=C(COc1ccc(Cl)cc1)NC12CCC(NC(=O)COc3ccc(Cl)nc3)(CC1)CC2] | 2-(4-chlorophenoxy)-N-(4- (2-((6-chloropyridin-3- yl)oxy)acetamido)bicyclo [2.2.2]octan-1-yl)acetamide | 478.1 | 1.87 (s, 12H), 4.36 (s, 2H), 4.47 (s, 2H), 6.90 (d, J = 9.2 Hz, 2H), 7.30 (d, J = 8.4 Hz, 2H), 7.37-7.40 (m, 3H), 7.48 (s, 1H), 8.06 (s, 1H). |
| IV | 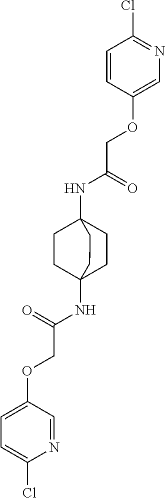 [SMILES: O=C(COc1ccc(Cl)nc1)NC12CCC(NC(=O)COc3ccc(Cl)nc3)(CC1)CC2] | N,N′- (bicyclo[2.2.2]octane-1,4- diyl)bis(2-((6- chloropyridin-3- yl)oxy)acetamide | 479.1 | 1.87 (s, 12H), 4.47 (s, 4H), 7.40 (s, 4H), 7.48 (s, 2H), 8.06 (s, 2H). |

### Example V

### N,N′-(bicyclo[1.1.1]pentane-1,3-diyl)bis(2-phenoxyacetamide)

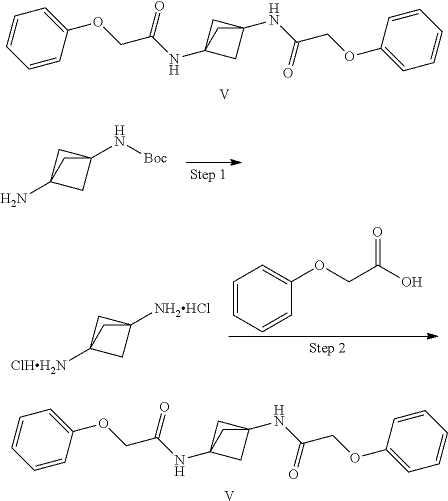

Step 1: To a stirred solution of tert-butyl (3-aminobicyclo[1.1.1]pentan-1-yl)carbamate (0.1 g, 0.50 mmol, 1.0 equiv.) in DCM (3.0 mL) was added 4M HCl in dioxane (1.0 mL) dropwise at 0° C. and the reaction mixture was stirred at room temperaturefor 3 h. After the starting material was consumed (TLC, 5% MeOH in DCM), the reaction mixture was concentrated under reduced pressure and the resultant solid was washed with n-pentane (2×10 mL and dried under high vaccum to give bicyclo[1.1.1]pentane-1,3-diamine dihydrochloride (0.08 g, 93% yield) as off white solid. ¹H NMR (400 MHz, DMSO-d₆) δ ppm 2.19 (s, 6H), 9.04 (s, 6H).

Step 2: To a solution of bicyclo[1.1.1]pentane-1,3-diamine dihydrochloride (0.08 g, 0.46 mmol, 1.0 equiv) in DCM (8.0 mL) at 0° C. was added triethylamine (0.33 mL, 2.35 mmol, 5.0 equiv). The reaction was stirred for 10 minutes and then 2 2-phenoxyacetic acid (0.17 g, 1.17 mmol, 2.5 equiv.) and T3P®® (50 wt. % in ethyl acetate) (0.56 mL, 0.94 mmol, 2.0 equiv.) were added to the reaction mixture. The reaction mixture was allowed to stir at room temperature (25° C.) for 3 h. After consumption of the starting material (TLC, 5 MeOH in DCM), the solvent was concentrated under reduced pressure and to the crude material was added saturated sodium bicarbonate solution (20 mL). The mixture was stirred for 20 mins and the solid was filtered, washed with water (10 mL) and n-pentane (20 mL), and dried under high vaccum to give N,N′-(bicyclo[1.1.1]pentane-1,3-diyl)bis(2-phenoxyacetamide) (0.135 g, 78.9% yield) as off white solid. LCMS (ES) m/z=367.2 [M+H]⁺. ¹H NMR (400 MHz, DMSO-d₆) δ ppm 2.25 (s, 6H), 4.39 (s, 4H), 6.94 (t, J=8.0 Hz, 6H), 7.27 (t, J=8.0 Hz, 4H), 8.63 (s, 2H).

TABLE II

| Cmpd | | | LCMS m/z | ¹H-NMR (400 MHz, |
|---|---|---|---|---|
| # | Structure | Name | [M + H]⁺ | DMSO-d₆) |
| V | 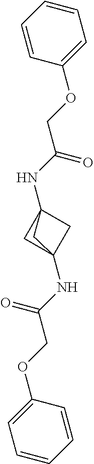 [SMILES: O=C(COc1ccccc1)NC12CC(NC(=O)COc3ccccc3)(C1)C2] | N,N′-(bicyclo[1.1.1]pentane-1,3-diyl) bis(2-phenoxyacetamide) | 367.2 | 2.25 (s, 6 H), 4.39 (s, 4 H), 6.94 (t, J = 8.0 Hz, 6 H), 7.27 (t, J = 8.0 Hz, 4 H), 8.63 (s, 2 H). |

### Example VI

### 2-(4-chlorophenoxy)-N-(3-(2-(4-chlorophenyl)acetamido)bicyclo[1.1.1]pentan-1-yl)acetamide

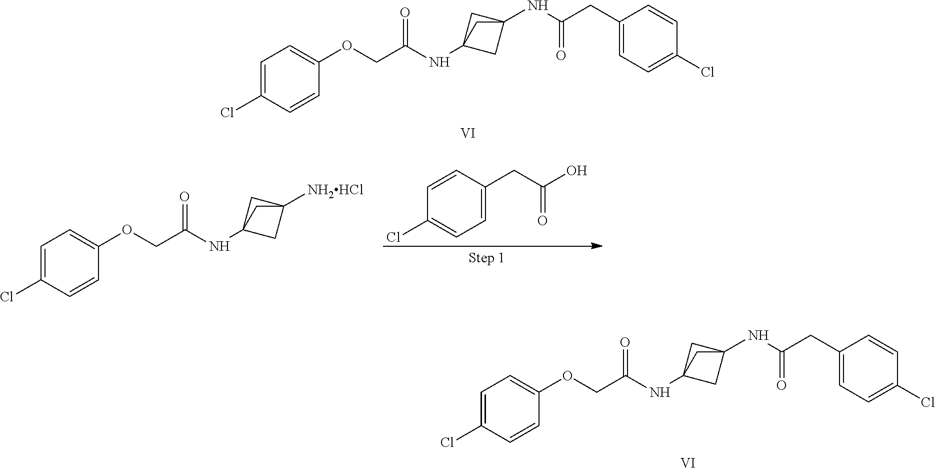

Step 1: To a solution of N-(3-aminobicyclo[1.1.1]pentan-1-yl)-2-(4-chlorophenoxy)acetamide hydrochloride (0.08 g, 0.26 mmol, 1 equiv) in DCM (15.0 mL) at 0° C. was added triethylamine (0.11 mL, 0.79 mmol, 3 equiv). The reaction was stirred for 10 minutes and then 2-(4-chlorophenyl)acetic acid (0.049 g, 0.31 mmol, 1.2 equiv) and T3P® (50 wt. % in ethyl acetate) (0.33 mL, 0.52 mmol, 2 equiv) were added to the reaction mixture. The reaction mixture was then allowed to stir at room temperature (25° C.) for 16 h. After consumption of the starting material, the reaction mixture was diluted with water (5 mL) and extracted with DCM (2×10 mL). The combined organic extract was washed with a saturated aqueous NaHCO₃ solution (8 mL) and then water (10 mL). The organic phase was dried over anhydrous sodium sulfate, filtered and concentrated under reduced pressure to give the crude product. The crude material was purified by flash column chromatography using a silica gel column where the product eluted at 2-3% methanol in DCM. Fractions containing the product were concentrated under reduced pressure to give 2-(4-chlorophenoxy)-N-(3-(2-(4-chlorophenyl)acetamido)bicyclo[1.1.1]pentan-1-yl)acetamide (0.053 g, 48% yield) as an off-white solid. LCMS (ES) m/z=419.1 [M+H]⁺. ¹H NMR (400 MHz, DMSO-d₆): δ ppm 2.18 (s, 6H), 3.34 (s, 2H), 4.39 (s, 2H), 6.93-6.95 (m. 2H), 7.21-7.23 (m, 2H), 7.30-7.33 (m, 4H), 8.62-8.63 (m, 2H).

The Compounds of Examples VII to XII were prepared generally according to the procedures described above for Example VI.

TABLE III

| Cmpd | | | LCMS m/z | ¹H-NMR (400 MHz, |
|---|---|---|---|---|
| # | Structure | Name | [M + H]⁺ | DMSO-d₆) |
| VI | 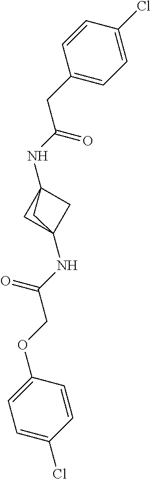 [SMILES: O=C(COc1ccc(Cl)cc1)NC12CC(NC(=O)Cc3ccc(Cl)cc3)(C1)C2] | 2-(4- chlorophenoxy)-N- (3-(2-(4- chlorophenyl) acetamido)bicyclo [1.1.1]pentan-1- yl)acetamide | 419.1 | 2.18 (s, 6 H), 3.34 (s, 2 H), 4.39 (s, 2 H), 6.93- 6.95 (m. 2 H), 7.21- 7.23 (m, 2 H), 7.30- 7.33 (m, 4 H), 8.62- 8.63 (m, 2 H). |
| VII | 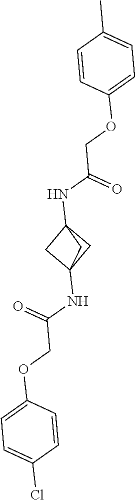 [SMILES: Cc1ccc(OCC(=O)NC23CC(NC(=O)COc4ccc(Cl)cc4)(C2)C3)cc1] | 2-(4- chlorophenoxy)-N- (3-(2-(p-tolyloxy) acetamido)bicyclo [1.1.1]pentan-1- yl)acetamide | 415.1 | 2.21 (s, 3 H), 2.24 (s, 6 H), 4.34 (s, 2 H), 4.41 (s, 2 H), 6.81 (d, J = 8.4 Hz, 2 H), 6.95 (d, J = 8.8 Hz, 2 H), 7.07 (d, J = 8.4 Hz, 2 H), 7.32 (d, J = 8.8 Hz, 2 H), 8.59 (s, 1 H), 8.65 (s, 1 H). |
| VIII | 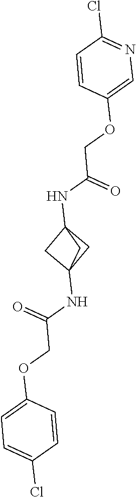 [SMILES: O=C(COc1ccc(Cl)cc1)NC12CC(NC(=O)COc3ccc(Cl)nc3)(C1)C2] | 2-(4- chlorophenoxy)-N- (3-(2-((6- chloropyridin-3- yl)oxy)acetamido) bicyclo[1.1.1] pentan-1-yl) acetamide | 436.1 | 2.24 (s, 6 H), 4.41 (s, 2 H), 4.53 (s, 2 H), 6.95 (d, J = 8.8 Hz, 2 H), 7.32 (d, J = 8.8 Hz, 2 H), 7.41- 7.46 (m, 2 H), 8.10 (s, 1 H), 8.66 (s, 1 H), 8.71 (s, 1 H). |
| IX | 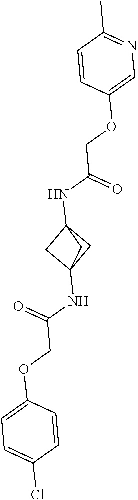 [SMILES: Cc1ccc(OCC(=O)NC23CC(NC(=O)COc4ccc(Cl)cc4)(C2)C3)cn1] | 2-(4- chlorophenoxy)-N- (3-(2-((6- methylpyridin-3- yl)oxy)acetamido) bicyclo[1.1.1]pentan- 1-yl)acetamide | 416.1 | 2.24 (s, 6 H), 2.37 (s, 3 H), 4.41 (s, 2 H), 4.44 (s, 2 H), 6.95 (d, J = 8.8 Hz, 2 H), 7.15 (d, J = 8.8 Hz, 1 H), 7.22-7.25 (m, 1 H), 7.32 (d, J = 8.8 Hz, 2 H), 8.13-8.14 (m, 1 H), 8.66 (d, J = 6.0 Hz, 2 H). |
| X | 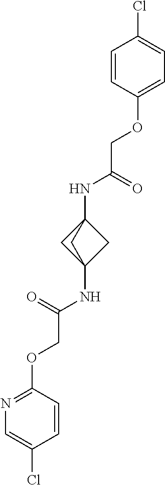 [SMILES: O=C(COc1ccc(Cl)cc1)NC12CC(NC(=O)COc3ccc(Cl)cn3)(C1)C2] | 2-(4- chlorophenoxy)-N- (3-(2-((5- chloropyridin-2- yl)oxy)acetamido) bicyclo[1.1.1]pentan- 1-yl)acetamide | 436.2 | 2.21 (s, 6 H), 4.40 (s, 2 H), 4.64 (s, 2 H), 6.91- 6.95 (m, 3 H), 7.32 (d, J = 8.8 Hz, 2 H), 7.80- 7.83 (m, 1 H), 8.15 (s, 1 H), 8.60-8.64 (m, 2 H). |
| XI | 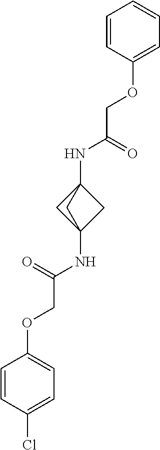 [SMILES: O=C(COc1ccccc1)NC12CC(NC(=O)COc3ccc(Cl)cc3)(C1)C2] | 2-(4- chlorophenoxy)-N- (3-(2- phenoxyacetamido) bicyclo[1.1.1]pentan- 1-yl)acetamide | 401.1 | 2.24 (s, 6 H), 4.40 (d, J = 6.8 Hz, 4 H), 6.93 (t, J = 8.0 Hz, 5 H), 7.33-7.26 (m, 4 H), 8.64 (d, J = 8.8 Hz, 2 H). |
| XII | 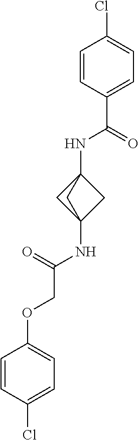 [SMILES: O=C(COc1ccc(Cl)cc1)NC12CC(NC(=O)c3ccc(Cl)cc3)(C1)C2] | 4-chloro-N-(3-(2- (4- chlorophenoxy) acetamido)bicyclo [1.1.1]pentan-1- yl)benzamide | 405.3 | 2.31 (s, 6 H), 4.42 (s, 2 H), 6.96 (d, J = 8.0 Hz, 2 H), 7.32 (d, J = 8.8 Hz, 2 H), 7.50 (d, J = 8.0 Hz, 2 H), 7.83 (d, J = 8.0 Hz, 2 H), 8.68 (bs, 1 H), 9.05 (bs, 1 H). |

### Example XIII

### 2-((3-(2-(4-chlorophenoxy)acetamido)bicyclo[1.1.1]pentan-1-yl)(2-(4-chlorophenoxy)ethynamino)-N,N-dimethylacetamide

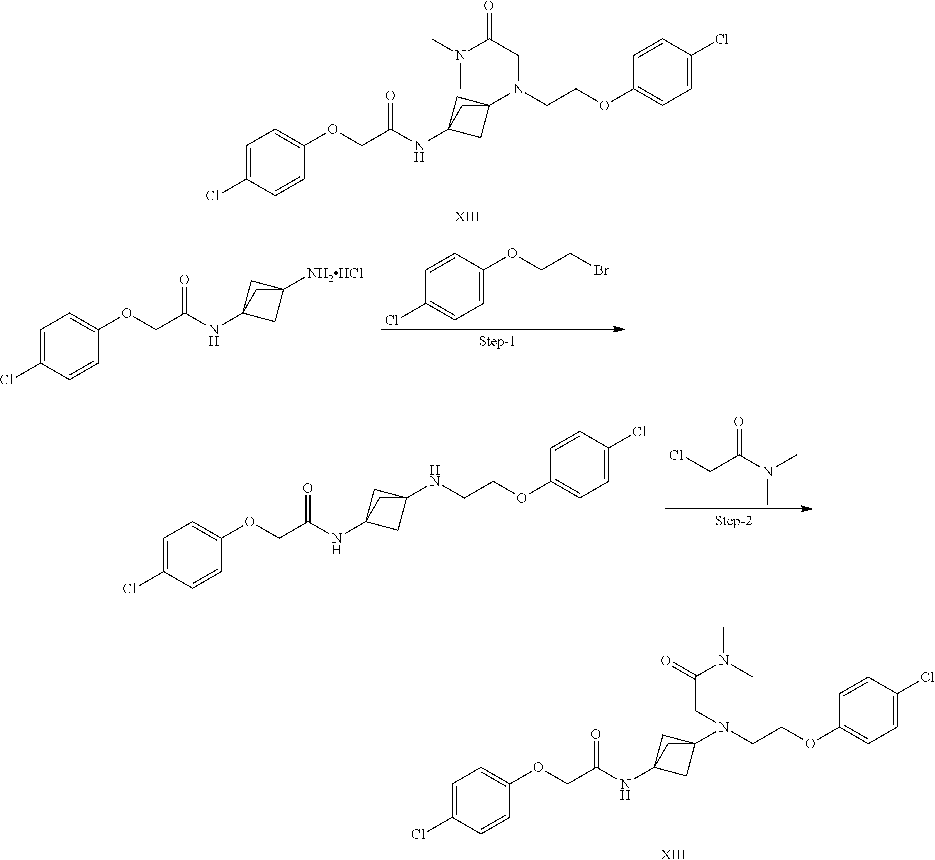

Step 1: To a solution of N-(3-aminobicyclo[1.1.1]pentan-1-yl)-2-(4-chlorophenoxy)acetamide hydrochloride (0.3 g, 0.99 mmol, 1 equiv) in TEA (0.55 mL, 3.96 mmol, 4 equiv) was added 1-(2-bromoethoxy)-4-chlorobenzene (0.27 g , 1.18 mmol, 1.2 equiv) and the reaction mixture was stirred at room temperature for 10 mins. The reaction mixture was then heated at 100° C. for 1 h. After the starting material was consumed (TLC, 5% DCM in methanol), the reaction mixture was cooled to room temperature, diluted with water (10 mL) and extracted with EtOAc (2×10 mL). The combined organic layer was washed with a brine solution (50 mL), dried over anhydrous sodium sulfate, filtered and concentrated under reduced pressure to give the crude product. The crude product was purified by flash column chromatography (Combiflash®®) using a silica gel column and the product eluted at 2% methanol in DCM. Fractions containing product were concentrated to give 2-(4-chlorophenoxy)-N-(3-((2-(4-chlorophenoxy)ethyl)amino)bicyclo[1.1.1]pentan-1-yl)acetamide (0.15 g, 36% yield) as pale brownish liquid. LCMS (ES) m/z=421.1 [M+H]⁺. ¹H NMR (400 MHz, DMSO-d₆) δ ppm 1.95 (bs, 6H), 2.81 (bs, 2H), 3.92-3.95 (m, 2H), 4.39 (s, 2H), 6.92-6.95 (m, 4H), 7.30 (t, J=8.0 Hz, 4H), 8.55 (bs, 1H).

Step 2: To a solution of 2-(4-chlorophenoxy)-N-(3-((4chlorophenoxy)ethyl)amino)bicyclo[1.1.1]pentan-1-yl)acetamide (0.04 g, 0.09 mmol 1 equiv) in TEA (0.05 mL, 0.36 mmol, 4 equiv) was added 2-chloro-N,N-dimethylacetamide (0.043 g, 0.36 mmol, 4 equiv) and the reaction mixture was heated at 80° C. for 1 h. After the starting material was consumed (TLC, 5% methanol in DCM), the reaction mixture was cooled to room temperature, diluted with water(10 mL) and extracted with EtOAc (2×10 mL). The combined organic layer was washed with a brine solution (20 mL), dried over anhydrous sodium sulfate, filtered and concentrated under reduced pressure. The crude material was purified by flash column chromatography (Combiflash®®) using a silica gel column and the product eluted at 5% ethyl methanol in DCM. Fractions containing product were concentrated to give 2-((3-(2-(4-chlorophenoxy)acetamido)bicyclo[1.1.1]pentan-1-yl)(2-(4-chlorophenoxy)ethyl)amino)-N,N-dimethylacetamide (0.02 g, 41% yield) as off white color solid. LCMS (ES) m/z=506.2 [M+H]⁺. ¹H NMR (400 MHz, DMSO-d6) δ ppm 1.99 (s, 6H), 2.74 (s, 3H), 2.90-2.91 (m, 2H), 2.93-2.95 (m, 3H), 3.37 (s, 2H), 3.93-3.96 (m, 2H), 4.39 (s, 2H), 6.89-6.95 (m, 4H), 7.31 (t, J=8.0 Hz, 4H), 8.57 (s,1H).

The Compounds of Examples XIV and XV were prepared generally according to the procedures described above for Example XIII.

TABLE XIV

| Cmpd | | | LCMS m/z | ¹H-NMR (400 MHz, |
|---|---|---|---|---|
| # | Structure | Name | [M + H]⁺ | DMSO-d₆) |
| XIII | 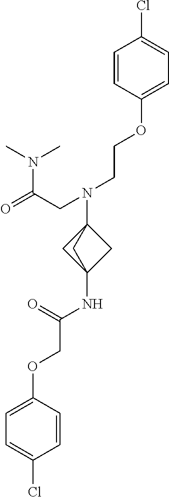 [SMILES: CN(C)C(=O)CN(CCOc1ccc(Cl)cc1)C12CC(NC(=O)COc3ccc(Cl)cc3)(C1)C2] | 2-((3-(2-(4- chlorophenoxy) acetamido)bicyclo [1.1.1]pentan-1-yl)(2- (4- chlorophenoxy)ethoxy) amino)-N,N- dimethylacetamide | 506.2 | 1.99 (s, 6 H), 2.74 (s, 3 H), 2.90-2.91 (m, 2 H), 2.93-2.95 (m, 3 H), 3.37 (s, 2 H), 3.93-3.96 (m, 2 H), 4.39 (s, 2 H), 6.89-6.95 (m, 4 H), 7.31 (t, J = 8.0 Hz, 4 H), 8.57 (s, 1 H). |
| XIV | 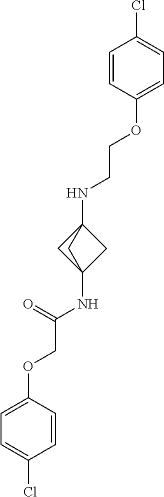 [SMILES: O=C(COc1ccc(Cl)cc1)NC12CC(NCCOc3ccc(Cl)cc3)(C1)C2] | 2-(4- chlorophenoxy)-N- (3-((2-(4- chlorophenoxy)ethyl) amino)bicyclo[1.1.1] pentan-1-yl) acetamide | 421.1 | 2.19 (s, 6 H), 2.98 (t, J = 5.6 Hz, 2 H), 4.02 (t, J = 5.2 Hz, 2 H), 4.39 (s, 2 H), 6.81-6.86 (m, 5 H), 7.21-7.28 (m, 4 H). |
| XV | 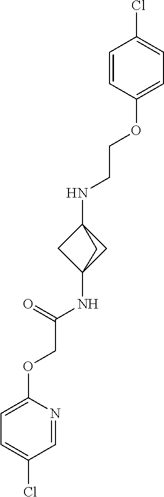 [SMILES: O=C(COc1ccc(Cl)cn1)NC12CC(NCCOc3ccc(Cl)cc3)(C1)C2] | N-(3-((2-(4- chlorophenoxy)ethyl) amino)bicyclo[1.1.1] pentan-1-yl)-2- ((5-chloropyridin-2- yl)oxy)acetamide | 422.0 | 1.92 (s, 6 H), 2.80 (t, J = 5.6 Hz, 2 H), 3.93 (t, J = 5.6 Hz, 2 H), 4.63 (s, 2 H), 6.92 (d, J = 8.8 Hz, 3 H), 7.29 (d, J = 8.8 Hz, 2 H), 7.81 (dd, J = 6.4 Hz, J = 2.4 Hz, 1 H), 8.15 (d, J = 2.4 Hz, 1 H), 8.50 (s, 1 H). |

### Example XVI

### Methyl N-(3-(2-(4-chlorophenoxy)acetamido)bicyclo[1.1.1]pentan-1-yl)-N-(2-(4-chlorophenoxy)ethyl)glycinate

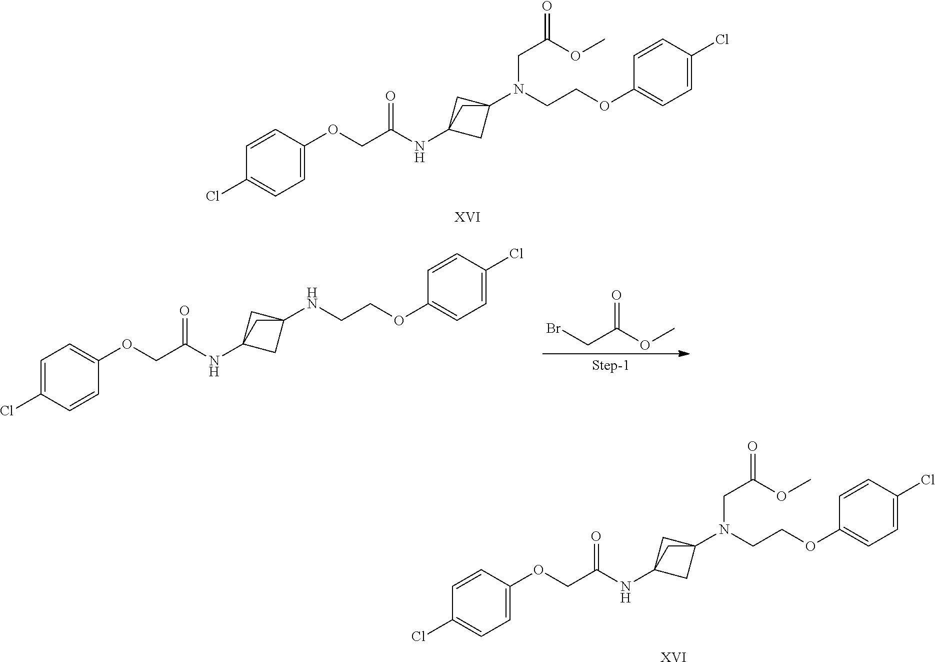

Step 1: To a solution of 2-(4-chlorophenoxy)-N-(3-((2-(4-chlorophenoxy)ethyl)amino)bicyclo[1.1.1]pentan-1-yl)acetamide (0.06 g, 0.14 mmol, 1 equiv) in DMF (15 mL) at room temperature was added potassium carbonate (0.029 g, 0.21 mmol, 1.5 equiv) portionwise. After stirring for 2 minutes, methyl 2-bromoacetate (0.016 g, 0.16 mmol, 1.2 equiv) was added. The reaction mixture was heated at 80° C. for 6 h. After the starting material was consumed (TLC, 15% EtOAc in hexane), the reaction mixture was cooled to room temperature, diluted with water (10 mL) and extracted with EtOAc (2×10 mL). The combined organic layer was washed with a brine solution (20 mL), dried over anhydrous sodium sulfate, filtered and concentrated under reduced pressure to give the crude product. The crude product was purified by flash column chromatography using a silica gel column and the product eluted at 40% ethyl acetate in hexane. Fractions containing product were concentrated to give methyl N-(3-(2-(4-chlorophenoxy)acetamido)bicyclo[1.1.1]pentan-1-yl)-N-(2-(4-chlorophenoxy)ethyl)glycinate (0.006 g, 8% yield) as colorless gum. LCMS (ES) m/z=493.1 [M+H]⁺. ¹H NMR (400 MHz, DMSO-d₆) δ ppm 1.98 (s, 6H), 2.99-3.02 (m, 2H), 3.47 (s, 2H), 3.56 (s, 3H), 3.95-3.98 (m, 2H), 4.39 (s, 2H), 6.95-6.89 (m, 4H), 7.31 (t, J=8.0 Hz, 4H), 8.58 (s, 1H).

TABLE V

| Cmpd | | | LCMS m/z | ¹H-NMR (400 MHz, |
|---|---|---|---|---|
| # | Structure | Name | [M + H]⁺ | DMSO-d₆) |
| XVI | 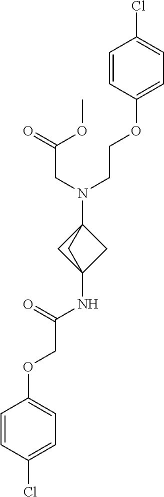 [SMILES: COC(=O)CN(CCOc1ccc(Cl)cc1)C12CC(NC(=O)COc3ccc(Cl)cc3)(C1)C2] | methyl N-(3-(2-(4- chlorophenoxy) acetamido)bicyclo [1.1.1]pentan-1-yl)-N- (2-(4- chlorophenoxy)ethyl) glycinate | 493.1 | 1.98 (s, 6 H), 2.99-3.02 (m, 2 H), 3.47 (s, 2 H), 3.56 (s, 3 H), 3.95-3.98 (m, 2 H), 4.39 (s, 2 H), 6.95-6.89 (m, 4 H), 7.31 (t, J = 8.0 Hz, 4 H), 8.58 (s, 1 H). |

### Example XVII

### ethyl 4-((3-(2-(4-chlorophenoxy)acetamido)bicyclo[1.1.1]pentan-1-yl)(2-(4-chlorophenoxy)ethyl)amino)butanoate

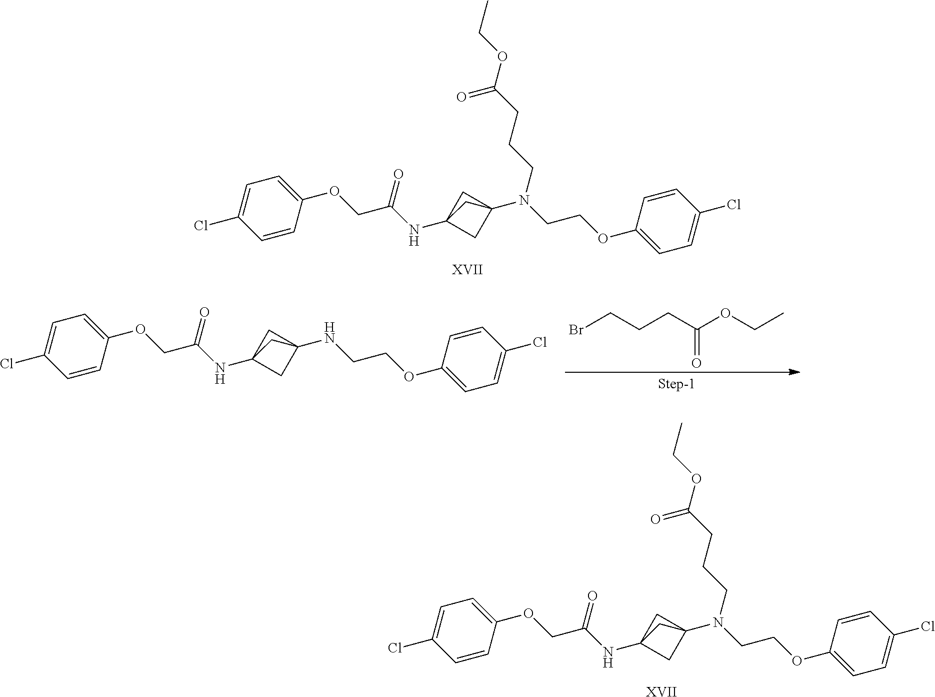

Step 1: To a solution of 2-(4-chlorophenoxy)-N-(3-((4chlorophenoxy)ethyl)amino)bicyclo[1.1.1]pentan-1-yl)acetamide (0.09 g, 0.21 mmol, 1 equiv) in TEA (0.086 mL, 0.84 mmol, 4 equiv) was added ethyl 4-bromobutanoate (0.053 mL, 0.31 mmol, 1.5 equiv) and the reaction mixture was heated at 100° C. for 1 h. After the starting material was consumed (TLC, 5% methanol in DCM), the reaction mixture was cooled to room temperature, diluted with water (10 mL) and extracted with EtOAc (2×10 mL). The combined organic layer was washed with a brine solution (20 mL), dried over anhydrous sodium sulfate, filtered and concentrated under reduced pressure. The crude material was purified by flash column chromatography using a silica gel column and the product eluted at 2.5% methanol in DCM. Fractions containing product were concentrated to provide ethyl 4-((3-(2-(4-chlorophenoxy)acetamido)bicyclo[1.1.1]pentan-1-yl)(2-(4-chlorophenoxy)ethyl)amino)butanoate (0.04 g, 35% yield) as pale yellow gum. LCMS (ES) m/z=535.2 [M+H]⁺. ¹H NMR (400 MHz, DMSO-d₆ δ ppm 1.12-1.22 (m, 3H), 1.59-1.63 (m, 2H), 1.98 (s, 6H), 2.26-2.30 (m, 2H), 2.48 (bs, 2H), 2.80-2.83 (m, 2H), 3.90-3.93 (m, 2H), 3.98-4.03 (m, 2H), 4.40 (s, 2H), 6.90-6.95 (m, 4H), 7.30 (t, J=8.0 Hz, 4H), 8.58 (s, 1H).

TABLE VI

| Cmpd | | | LCMS m/z | ¹H-NMR (400 MHz, |
|---|---|---|---|---|
| # | Structure | Name | [M + H]⁺ | DMSO-d₆) |
| XVII | 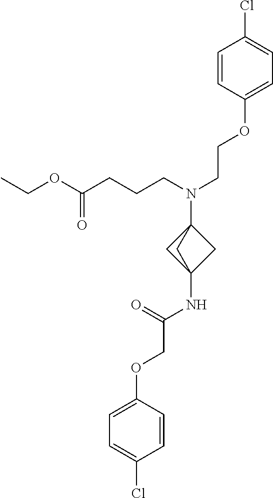 [SMILES: CCOC(=O)CCCN(CCOc1ccc(Cl)cc1)C12CC(NC(=O)COc3ccc(Cl)cc3)(C1)C2] | ethyl 4-((3-(2-(4- chlorophenoxy) acetamido)bicyclo [1.1.1]pentan-1- yl)(2-(4- chlorophenoxy) ethyl)amino) butanoate | 535.2 | 1.12-1.22 (m, 3 H), 1.59- 1.63 (m, 2 H), 1.98 (s, 6 H), 2.26-2.30 (m, 2 H), 2.48 (bs, 2 H), 2.80-2.83 (m, 2 H), 3.90- 3.93 (m, 2 H), 3.98-4.03 (m, 2 H), 4.40 (s, 2 H), 6.90-6.95 (m, 4 H), 7.30 (t, J = 8.0 Hz, 4 H), 8.58 (s, 1 H). |

### Example XVIII

### 2-(4-chlorophenoxy)-N-(3-((2-(4-chlorophenoxy)ethyl)(methyl)amino)bicyclo[1.1.1]pentan-1-yl)acetamide

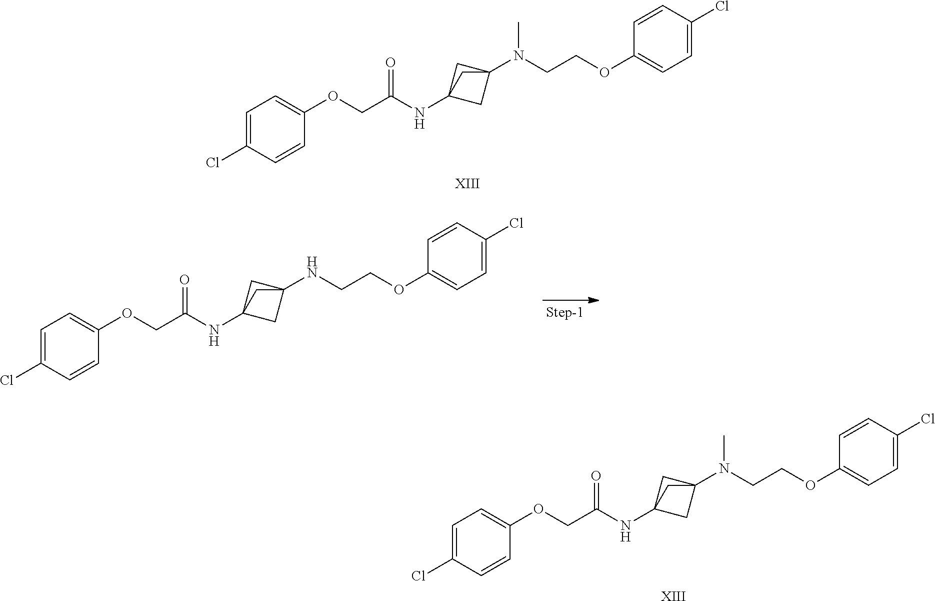

Step 1: To a solution of 2-(4-chlorophenoxy)-N-(3-((2-(4-chlorophenoxy)ethyl)amino)bicyclo[1.1.1]pentan-1-yl)acetamide (0.050 g, 0.118 mmol, 1 equiv) in tetrahydrofuran (5 mL) at 0° C. were added aqueous formaldehyde (0.2 mL) and acetic acid (0.050 mL). After stirring for 1 h at room temperature sodiumtriacetoxy borohydrate (0.037 g, 0.178 mmol, 1.5 equiv) was added at 0° C. After stirring for 5 minutes, the reaction mixture was allowed to stir at room temperature for 14 h. The reaction mixture was then quenched with a saturated sodium bicarbonate (1 mL) and extracted with ethyl acetate (2×15 mL). The combined organic layer was washed with water (2.0 mL), brine (3 mL) and then dried over anhydrous sodium sulfate. The organic layer was filtered and concentrated under reduced pressure to give the crude product which was purified by preparative TLC using 3% methanol in DCM as mobile phase. The product was concentrated under reduced pressure and dried under high vacuum to provide 2-(4-chlorophenoxy)-N-(3-((2-(4-chlorophenoxy)ethyl)(methyl)amino)bicyclo[1.1.1]pentan-1-yl)acetamide (0.027 g, 52.94% yield) white solid. LCMS (ES) m/z=435.1 [M+H]+. 1H NMR (400 MHz, DMSO-d6): δ ppm 1.96 (s, 6H), 2.20 (s, 3H), 2.70 (t, J=5.6 Hz, 2H), 3.99 (t, J=5.6 Hz, 2H), 4.40 (s, 2H), 6.92-6.95 (m, 4H), 7.28-7.33 (m, 4H), 8.60 (s, 1H).

TABLE VII

| | | | LCMS | |
|---|---|---|---|---|
| | | | m/z | |
| Cmpd # | Structure | Name | [M + H]⁺ | ¹H-NMR (400 MHz, DMSO-d₆) |
| XVIII | 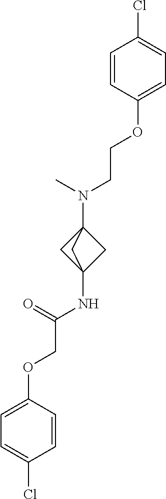 [SMILES: CN(CCOc1ccc(Cl)cc1)C12CC(NC(=O)COc3ccc(Cl)cc3)(C1)C2] | 2-(4- chlorophenoxy)- N-(3-((2-(4- chlorophenoxy)eth- yl)(methyl)amino) bicyclo[1.1.1]pentan- 1-yl)acetamide | 435.1 | 1.96 (s, 6 H), 2.20 (s, 3 H), 2.70 (t, J = 5.6 Hz, 2 H), 3.99 (t, J = 5.6 Hz, 2 H), 4.40 (s, 2 H), 6.92- 6.95 (m, 4 H), 7.28-7.33 (m, 4 H), 8.60 (s, 1 H). |

### Example XIX

### 2-(4-chlorophenoxv)-N-(3-(N-(2-(4-chlorophenoxy)ethyl)acetamido)bicyclo[1.1.1]pentan-1-yl)acetamide

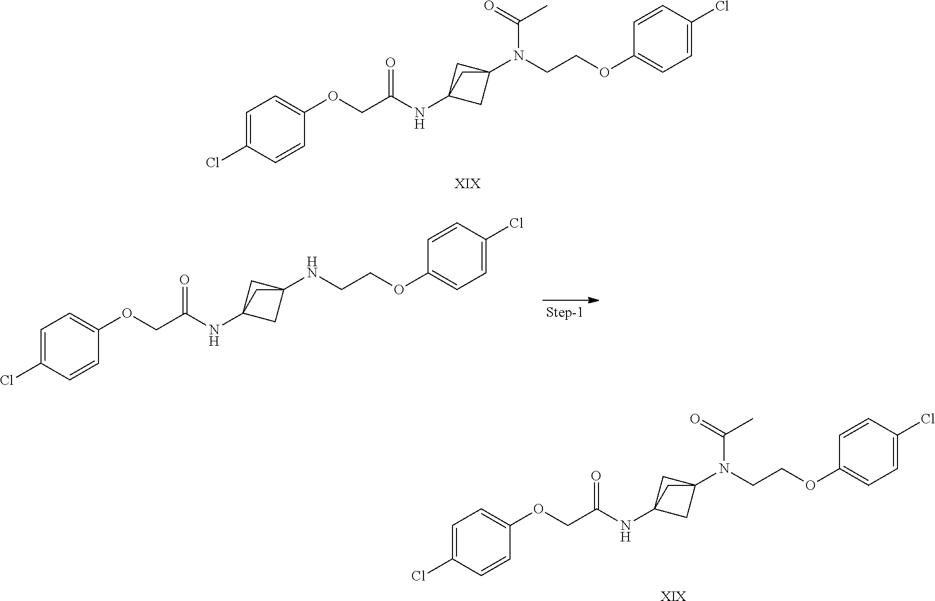

Step 1: To a solution of 2-(4-chlorophenoxy)-N-(3-((2-(4-chlorophenoxy)ethyl)amino)bicyclo[1.1.1]pentan-1-yl)acetamide (0.050 g, 0.118 mmol, 1 equiv) in DMF (5 mL) at 0° C. were added triethylamine (0.066 mL, 0.47 mmol, 4 equiv) and acetic acid (0.009 mL, 0.166 mmol, 1.4 equiv). After stirring for 5 minutes, 1-ethyl-3-(3-dimethylaminopropyl)carbodiimide (0.034 g, 0.178 mmol, 1.5 equiv) and hydroxybenzotriazole (0.027 g, 0.178 mmol, 1.5 equiv) were added. Then reaction mixture was allowed to stir at room temperature for 14 h. The reaction mixture was diluted with water (5 mL) and extracted with ethyl acetate (2×10 mL). The combined organic extract was washed with a saturated solution of aqueous NaHCO3 (3 mL), water (10 mL) and dried over anhydrous sodium sulfate. The organic layer was filtered and concentrated under reduced pressure to give the crude product which was purified by following preparative HPLC method.

Column: intersil ODS 3V (250 mm×4.6 mm×5 mic)

Mobile phase (A): 0.1% Ammonia in water

Mobile phase (B): ACN

Flow rate: 1.0 mL/min

T/% B: 0/20, 10/80, 25/90, 27/20, 30/20

Yield: 0.029 g, white solid, 53.70%

LCMS (ES) m/z=463.1 [M+H]+. 1H NMR (400 MHz, DMSO-d6): δ ppm 2.05 (s, 3H), 2.30 (s, 6H), 3.62 (t, J=5.6 Hz, 2H), 4.03 (t, J=5.2 Hz, 2H), 4.41 (s, 2H), 6.93-6.97 (m, 4 H), 7.27-7.31 (m, 4H), 8.41 (s, 1H).

TABLE VIII

| | | | LCMS | |
|---|---|---|---|---|
| | | | m/z | |
| Cmpd # | Structure | Name | [M + H]⁺ | ¹H-NMR (400 MHz, DMSO-d₆) |
| XIX | 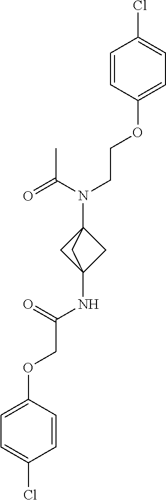 [SMILES: CC(=O)N(CCOc1ccc(Cl)cc1)C12CC(NC(=O)COc3ccc(Cl)cc3)(C1)C2] | 2-(4- chlorophenoxy)-N- (3-(N-(2-(4- chlorophenoxy)ethyl) acetamido)bi- cyclo[1.1.1]pentan-1- yl)acetamide | 463.1 | 2.05 (s, 3 H), 2.30 (s, 6 H), 3.62 (t, J = 5.6 Hz, 2 H), 4.03 (t, J = 5.2 Hz, 2 H), 4.41 (s, 2 H), 6.93- 6.97 (m, 4 H), 7.27-7.31 (m, 4 H), 8.41 (s, 1 H). |

### Example XX

### 2-(4-chlorophenoxy)-N-(3-((2-(4-chlorophenoxy)ethyl)(oxetan-3-yl)amino)bicyclo[1.1.1]pentan-1-yl)acetamide

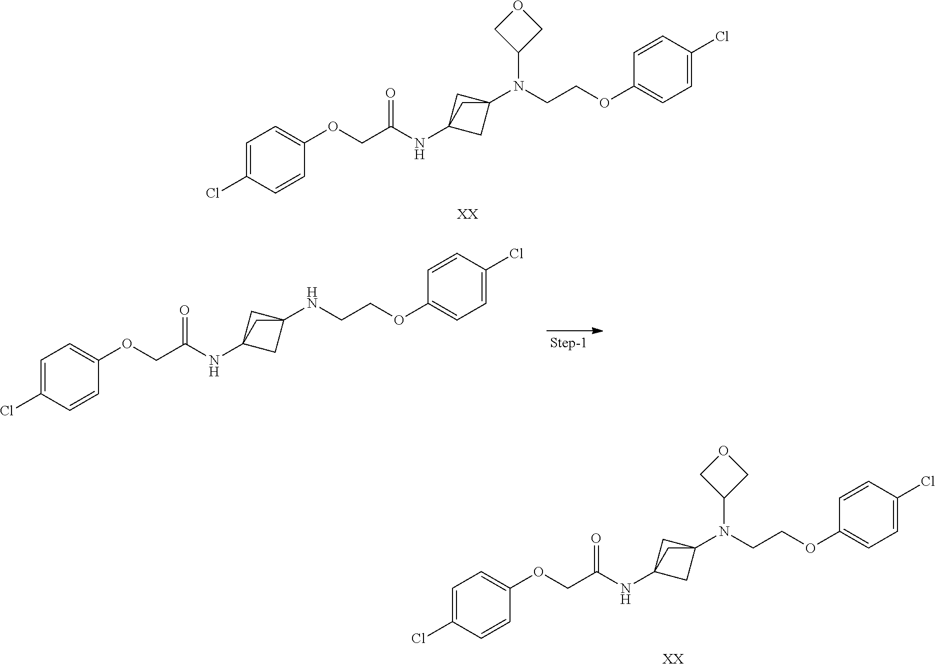

Step 1: To a solution of 2-(4-chlorophenoxy)-N-(3-((2-(4-chlorophenoxy)ethyl)amino)bicyclo[1.1.1]pentan-1-yl)acetamide (0.060 g, 0.142 mmol, 1 equiv) in methanol (5 mL) was added oxetan-3-one (0.012 mL, 0.213 mmol, 1.5 equiv) and ZnCl₂ (0.5 M in THF, 1.13 mL, 0.569 mmol, 4 equiv). Then reaction mixture was then cooled with an ice bath and sodium cyanoborohydride (0.026 g, 0.427 mmol, 3 equiv) was added at 0° C. The reaction mixture was then stirred at 50° C. for 5 h then cooled to room temperature and concentrated under reduced pressure. The residue was diluted with water (5 mL) and then extracted with ethyl acetate (2×10 mL). The combined organic layer was washed with water (5 mL), brine (5 mL), dried over anhydrous sodium sulfate, filtered and concentrated under reduced pressure to give the crude product which was purified by preparative TLC using 3% methanol in DCM as mobile phase. The fractions containing product were concentrated under reduced pressure and dried under high vacuum to provide 2-(4-chlorophenoxy)-N-(3-((2-(4-chlorophenoxy)ethyl)(oxetan-3-yl)amino)bicyclo[1.1.1]pentan-1-yl)acetamide (0.024 g, 35.82% yield) as a white solid. LCMS (ES) m/z=477.1 [M+H]+. 1H NMR (400 MHz, DMSO-d6): δ ppm 1.96 (s, 6H), 2.82-2.84 (m, 2H), 3.89-3.96 (m, 3H), 4.39-4.43 (m, 4H), 4.52-4.55 (m, 2H), 6.93 (d, J=8.8 Hz, 4H), 7.29-7.32 (m, 4H), 8.60 (s, 1H).

TABLE IX

| | | | LCMS m/z | |
|---|---|---|---|---|
| Cmpd # | Structure | Name | [M + H]⁺ | ¹H-NMR (400 MHz, DMSO-d₆) |
| XX | 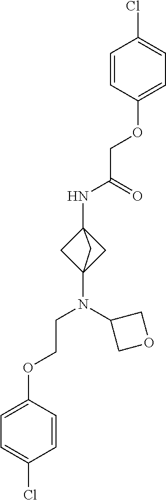 [SMILES: O=C(COc1ccc(Cl)cc1)NC12CC(N(CCOc3ccc(Cl)cc3)C3COC3)(C1)C2] | 2-(4- chlorophenoxy)- N-(3-((2-(4- chlorophenoxy)eth- yl)(oxetan-3- yl)amino)bi- cyclo[1.1.1]pentan- 1-yl)acetamide | 477.1 | 1.96 (s, 6 H), 2.82-2.84 (m, 2 H), 3.89-3.96 (m, 3 H), 4.39- 4.43 (m, 4 H), 4.52-4.55 (m, 2 H), 6.93 (d, J = 8.8 Hz, 4 H), 7.29-7.32 (m, 4 H), 8.60 (s, 1 H). |

### Example XXI

### 2-(4-chlorophenoxy)-N-(3-((2-((4-chlorophenyl)amino)ethyl)amino)bicyclo[1.1.1]pentan-1-yl)acetamide

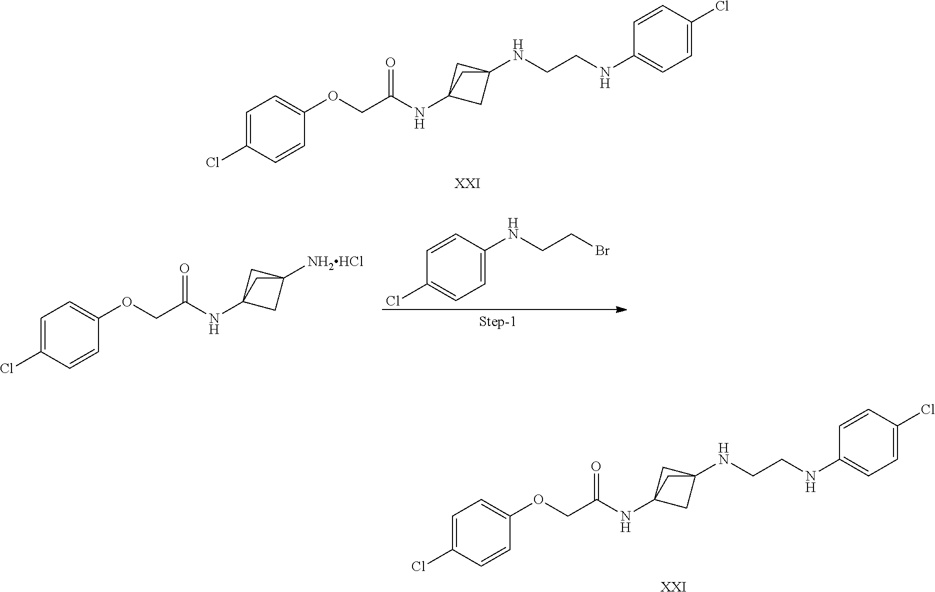

Step 1: N-(3-aminobicyclo[1.1.1]pentan-1-yl)-2-(4-chlorophenoxy)acetamide hydrochloride (0.300 g, 0.989 mmol, 1 equiv), triethylamine (0.556 mL, 3.95 mmol, 4 equiv) and N-(2-bromoethyl)-4-chloroaniline (0.278 g, 1.18 mmol, 1.2 equiv) were added to a sealed tube. The reaction mixture was then sealed and heated at 100° C. for 1 h. at which time it was cooled to room temperature, diluted with water (5 mL) and extracted with EtOAc (2×20 mL). The combined organic extract was washed with water (10 mL) followed by a saturated brine solution (8 mL), dried over anhydrous sodium sulfate, filtered and concentrated under reduced pressure. The crude material was purified by flash column chromatography using a silica gel column and a mixture of methanol in DCM as eluent and the product eluted at 4-5% methanol in DCM. Fractions containing product were concentrated to give 2-(4-chlorophenoxy)-N-(3-((2-((4-chlorophenyl)amino)ethyl)amino)bicyclo[1.1.1]pentan-1-yl)acetamide (0.130 g, 31.32% yield) as off white solid. LCMS (ES) m/z=420.1 [M+H]+, 1H NMR (400 MHz, DMSO-d6) δ ppm 1.93 (s, 6H), 2.61-2.64 (m, 2H), 2.99-3.03 (m, 2H), 4.39 (s, 2H), 5.63-5.66 (m, 1H), 6.53 (d, J=8.8 Hz, 2H), 6.94 (d, J=8.8 Hz, 2H), 7.05 (d, J=8.8 Hz, 2H), 7.31 (d, J=8.8 Hz, 2H), 8.54 (s, 1H).

TABLE X

| | | | LCMS m/z | |
|---|---|---|---|---|
| Cmpd # | Structure | Name | [M + H]⁺ | ¹H-NMR (400 MHz, DMSO-d₆) |
| XXI | 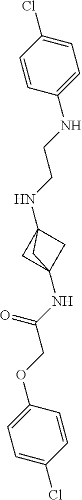 [SMILES: O=C(COc1ccc(Cl)cc1)NC12CC(NCCNc3ccc(Cl)cc3)(C1)C2] | 2-(4-chlorophenoxy)- N-(3-((2-((4- chlorophenyl)amino)eth- yl)amino)bicyclo[1.1.1] pentan-1-yl)acetamide | 420.1 | 1.93 (s, 6 H), 2.61-2.64 (m, 2 H), 2.99-3.03 (m, 2 H), 4.39 (s, 2 H), 5.63-5.66 (m, 1 H), 6.53 (d, J = 8.8 Hz, 2 H), 6.94 (d, J = 8.8 Hz, 2 H), 7.05 (d, J = 8.8 Hz, 2 H), 7.31 (d, J = 8.8 Hz, 2 H), 8.54 (s, 1 H). |

### Example XXII

### 2-(4-chlorophenoxy)-N-(3-(3-(4-chlorophenyl)-2-oxoimidazolidin-1-yl)bicyclo[1.1.1]pentan-1-yl)acetamide

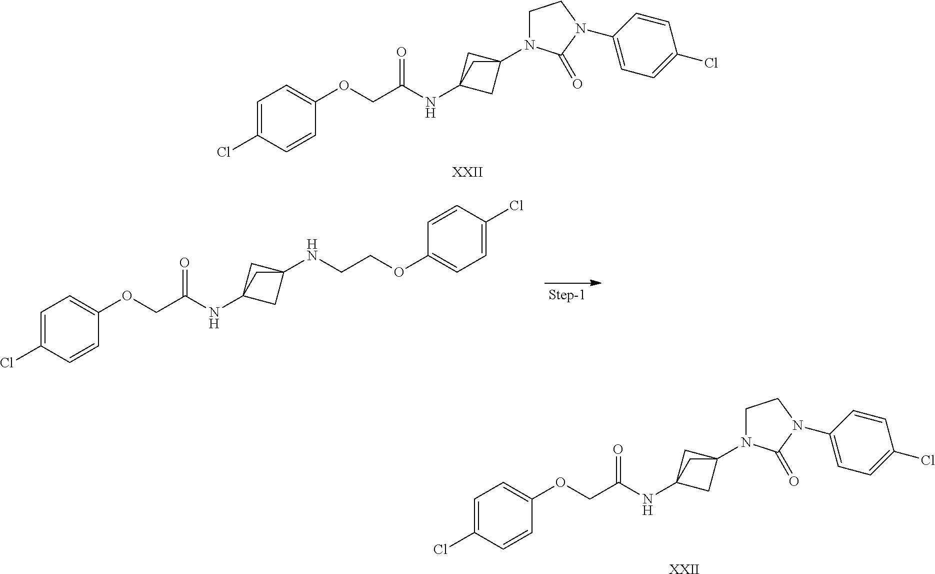

Step 1: To a solution of 2-(4-chlorophenoxy)-N-(3-((2-((4-chlorophenyl)amino)ethyl)amino)bicyclo[1.1.1]pentan-1-yl)acetamide (0.090 g, 0.21 mmol, 1 equiv) in DCM (5 mL) at 0° C., triethylamine (0.150 mL, 1.07 mmol, 5 equiv) and triphosgene (0.038 g, 0.128 mmol, 0.6 equiv) were added. After stirring for 5 minutes, the reaction mixture was allowed to stir at room temperature for 3 h. Then reaction mixture was quenched with a saturated sodium bicarbonate solution, extracted with DCM (2×10 mL). The combined organic extract was washed with cold water (8 mL) followed by a saturated brine solution (6 mL), dried over anhydrous sodium sulfate, filtered and concentrated under reduced pressure to give the crude product. The crude product was purified by flash column chromatography using a silica gel column and a mixture ethyl acetate in hexane as eluent and the product eluted at 35-40% ethyl acetate in hexane to provide 2-(4-chlorophenoxy)-N-(3-(3-(4-chlorophenyl)-2-oxoimidazolidin-1-yl)bicyclo[1.1.1]pentan-1-yl)acetamide (0.044 g, 46.31% yield) as white solid. LCMS (ES) m/z=446.1 [M+H]⁺. ¹H NMR (400 MHz, DMSO-d₆) δ ppm 2.29 (s, 6H), 3.42 (t, J=8.4 Hz, 2H), 3.75 (t, J=6.8 Hz, 2H), 4.42 (s, 2H), 6.96 (d, J=9.2 Hz, 2H), 7.32 (d, J=8.8 Hz, 4H), 7.55 (d, J=8.8 Hz, 2H), 8.71 (s, 1H).

TABLE XI

| | | | LCMS m/z | |
|---|---|---|---|---|
| Cmpd # | Structure | Name | [M + H]⁺ | ¹H-NMR (400 MHz, DMSO-d₆) |
| XXII | 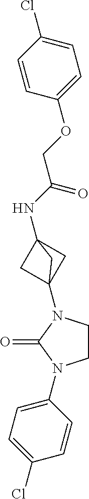 [SMILES: O=C(COc1ccc(Cl)cc1)NC12CC(N3CCN(c4ccc(Cl)cc4)C3=O)(C1)C2] | 2-(4- chlorophenoxy)-N- (3-(3-(4- chlorophenyl)-2- oxoimidazolidin-1- yl)bicyclo[1.1.1] pentan-1- yl)acetamide | 446.1 | 2.29 (s, 6 H), 3.42 (t, J = 8.4 Hz, 2 H), 3.75 (t, J = 6.8 Hz, 2 H), 4.42 (s, 2 H), 6.96 (d, J = 9.2 Hz, 2 H), 7.32 (d, J = 8.8 Hz, 4 H), 7.55 (d, J = 8.8 Hz, 2 H), 8.71 (s, 1 H). |

### Example XXIII

### 1-(3-((2-(4-chlorophenoxy)ethyl)amino)bicyclo[1.1.1]pentan-1-yl)-3-(4-chlorophenyl)imidazolidin-2-one

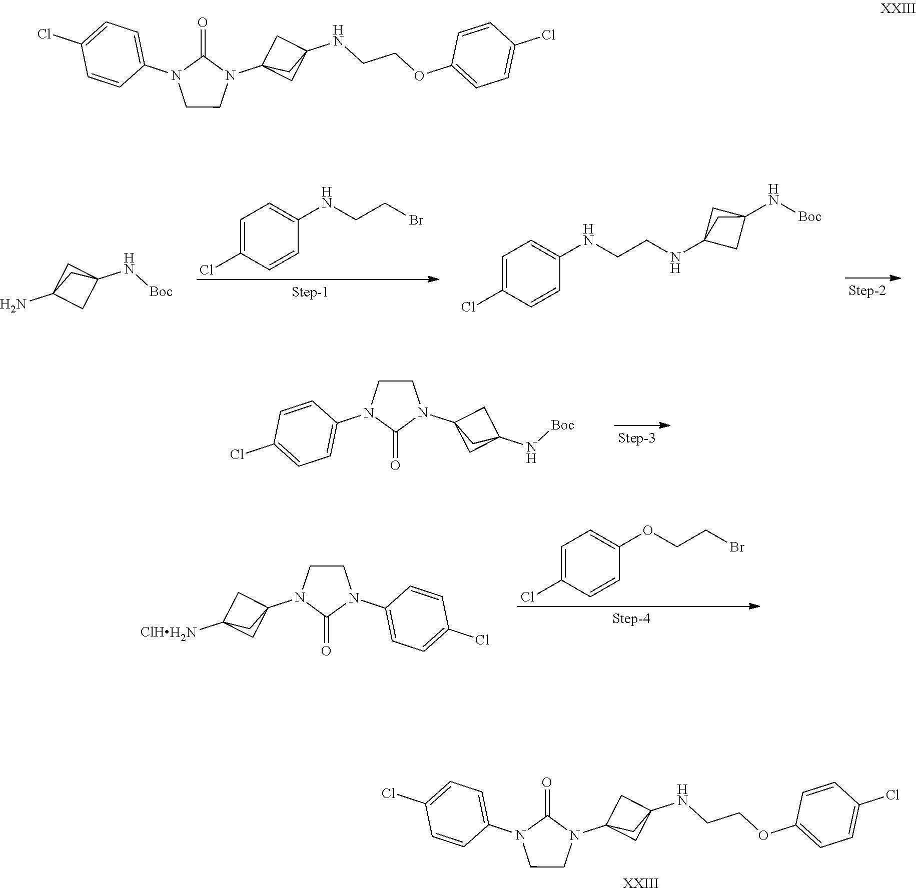

Step 1: In a sealed tube tert-butyl(3-aminobicyclo[1.1.1]pentan-1-yl)carbamate (0.400 g, 2.01 mmol, 1 equiv), triethylamine (0.709 mL, 5.04 mmol, 2.5 equiv) and N-(2-bromoethyl)-4-chloroaniline (0.567 g, 2.42 mmol, 1.2 equiv) were added. The reaction mixture was sealed and heated at 100° C. for 1 h. The reaction mixture was cooled to room temperature, diluted with water (5 mL) and extracted with EtOAc (2×15 mL). The combined organic extract was washed with cold water (5 mL) followed by a saturated brine solution (5 mL), dried over anhydrous sodium sulfate, filtered and concentrated under reduced pressure to give the crude product. The crude product was purified by flash column chromatography using a silica gel column and a mixture of methanol in DCM as eluent and the product eluted at 4-5% methanol in DCM. Fractions containing product were concentrated to give tert-butyl (3-((2-((4-chlorophenyl)amino)ethyl)amino)bicyclo[1.1.1]pentan-1-yl)carbamate (0.305 g, 43.01% yield) as light yellow gum. LCMS (ES) m/z=352.3[M+H]+, 1H NMR (400 MHz, DMSO-d6) δ ppm 1.34 (s, 9H), 1.80 (s, 6H), 2.58-2.60 (m, 2H), 2.98-3.02 (m, 2H), 5.64 (bs, 1H), 6.53 (d, J=8.8 Hz, 2H), 7.04 (d, J=8.8 Hz, 2H), 7.35 (bs, 1H).

Step 2: To a solution of tert-butyl (3-((2-((4-chlorophenyl)amino)ethyl)amino)bicyclo[1.1.1]pentan-1-yl)carbamate (0.305 g, 0.86 mmol, 1 equiv) in DCM (10 mL) at 0° C., triethylamine (0.609 mL, 4.33 mmol, 5 equiv) and triphosgene (0.154 g, 0.520 mmol, 0.6 equiv) were added. After stirring 5 minutes, the reaction mixture was allowed to stir at room temperature for 2.5 h. Then reaction mixture was quenched with a saturated sodium bicarbonate solution, extracted with DCM (2×10 mL). The combined organic extract was washed with water (10 mL) followed by a saturated brine solution (5 mL), dried over anhydrous sodium sulfate, filtered and concentrated under reduced pressure to give the crude product. The crude product was purified by flash column chromatography using a silica gel column and a mixture ethyl acetate in hexane as eluent and the product eluted at 80-90% ethyl acetate in hexane. Fractions containing product were concentrated to give tert-butyl (3-(3-(4-chlorophenyl)-2-oxoimidazolidin-1-yl)bicyclo[1.1.1]pentan-1-yl)carbamate (0.240 g, 73.39% yield) as white solid. LCMS (ES) m/z=378.1 [M+H]+. 1H NMR (400 MHz, DMSO-d6) δ ppm 1.36 (s, 9H), 2.16 (s, 6H), 3.40 (t, J=8.0 Hz, 2H), 3.74 (t, J=6.8 Hz, 2H), 7.32 (d, J=8.8 Hz, 2H), 7.53-7.55 (m, 3H).

Step 3: To a solution of tert-butyl (3-(3-(4-chlorophenyl)-2-oxoimidazolidin-1-yl)bicyclo[1.1.1]pentan-1-yl)carbamate (0.240 g, 0.635 mmol, 1 equiv) in DCM (8 mL) at 0° C. was added 4 M HCl in 1,4-dioxane (5 mL). Then reaction mixture was allowed to stir at room temperature (25° C.) for 2.5 h. Then solvent was evaporated under reduced pressure. The resultant crude material was washed with n-pentane (10 mL). The pentane was then decanted and the solid dried to give crude product of 1-(3-aminobicyclo[1.1.1]pentan-1-yl)-3-(4-chlorophenyl)imidazolidin-2-one hydrochloride (0.185 g, white solid, Crude). LCMS (ES) m/z=278.1 [M+H]+. 1H NMR (400 MHz, DMSO-d6) δ ppm 2.27 (s, 6H), 3.43 (t, J=8.0 Hz, 2H), 3.77 (t, J=7.6 Hz, 2H), 7.34 (d, J=8.8 Hz, 2H), 7.54 (d, J=9.2 Hz, 2H), 8.74 (s, 3H).

Step 4: 1-(3-aminobicyclo[1.1.1]pentan-1-yl)-3-(4-chlorophenyl)imidazolidin-2-one hydrochloride (0.120 g, 0.381 mmol, 1 equiv), triethylamine (0.214 mL, 1.52 mmol, 4 equiv) and 1-(2-bromoethoxy)-4-chlorobenzene (0.107 g, 0.458 mmol, 1.2 equiv) were added to a sealed tube. The reaction mixture was sealed and heated at 100° C. for 1 h. Reaction mixture was cooled to room temperature, diluted with water (5 mL) and extracted with EtOAc (2×15 mL). The combined organic extract was washed with cold water (5 mL) followed by a saturated brine solution (5 mL), dried over anhydrous sodium sulfate, filtered and concentrated under reduced pressure to give the crude product, which was purified by preparative TLC using 2.5% methanol in DCM as mobile phase. The mixture was concentrated under reduced pressure and dried under high vacuum to provide 1-(3-((2-(4-chlorophenoxy)ethyl)amino)bicyclo[1.1.1]pentan-1-yl)-3-(4-chlorophenyl)imidazolidin-2-one (0.028 g, 16.96% yield) off-white solid. LCMS (ES) m/z=432.1 [M+H]+. 1H NMR (400 MHz, DMSO-d6): δ ppm 2.00 (s, 6H), 2.60 (bs, 1H), 2.82-2.84 (m, 2H), 3.40 (t, J=8.4 Hz, 2H), 3.74 (t, J=7.2 Hz, 2H), 3.96 (t, J=5.6 Hz, 2H), 6.94 (d, J=8.0 Hz, 2H), 7.28-7.33 (m, 4H), 7.54 (d, J=9.2 Hz, 2H).

TABLE XII

| | | | LCMS m/z | |
|---|---|---|---|---|
| Cmpd # | Structure | Name | [M + H]⁺ | ¹H-NMR (400 MHz, DMSO-d₆) |
| XXIII | 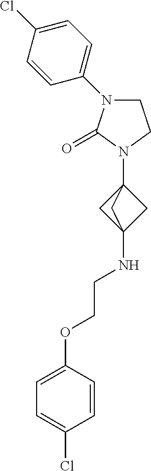 [SMILES: O=C1N(c2ccc(Cl)cc2)CCN1C12CC(NCCOc3ccc(Cl)cc3)(C1)C2] | 1-(3-((2-(4- chlorophenoxy)eth- yl)amino)bi- cyclo[1.1.1]pentan- 1-yl)-3-(4- chlorophenyl)imida- zolidin-2-one | 432.1 | 2.00 (s, 6 H), 2.60 (bs, 1 H), 2.82-2.84 (m, 2 H), 3.40 (t, J = 8.4 Hz, 2 H), 3.74 (t, J = 7.2 Hz, 2 H), 3.96 (t, J = 5.6 Hz, 2 H), 6.94 (d, J = 8.0 Hz, 2H), 7.28- 7.33 (m, 4 H), 7.54 (d, J = 9.2 Hz, 2 H). |

### Example XXIV and Example 6a

### 2-(4-chlorophenoxy)-N-(3-(3-(4-chlorophenoxy)pyrrolidin-1-yl)bicyclo[1.1.1]pentan-1-yl)acetamide

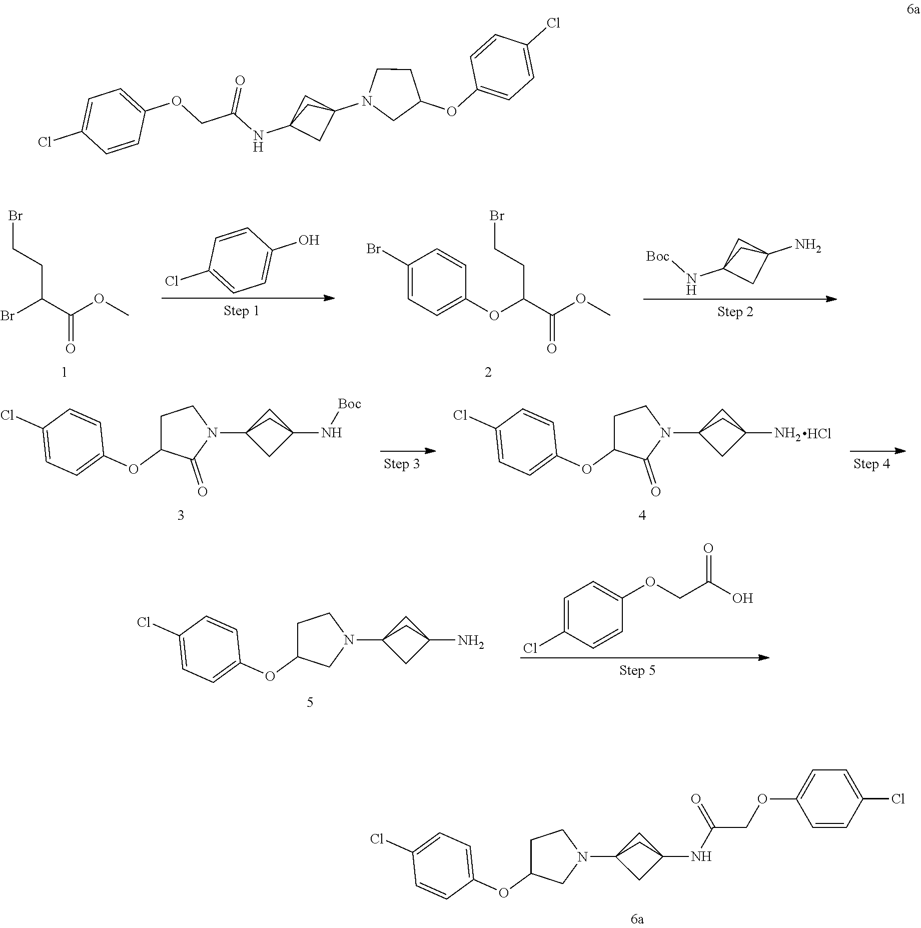

Step 1: To a solution of methyl 2,4-dibromobutanoate (1.2 g, 1 equiv) in DMF (15 mL) was added 4-chlorophenol (0.59 g, 1 equiv) at rt followed by K₂CO₃ (0.636 g, 1 equiv) and the reaction was stirred at 60 ⁰0 for 3 h. The reaction mixture was then allowed to come to rt. Water (5 mL) was added and the mixture was extracted with EtOAc (3×50 mL). The combined EtOAc extracts were dried over anhydrous Na₂SO₄, filtered and distilled under vacuum. The crude material was purified by flash column chromatography eluting the product at 10% EtOAc in hexane. The fractions containing the product were combined and the solvent was evaporated to provide methyl 4-bromo-2-(4-chlorophenoxy)butanoate as gum (1 g). ¹H NMR (400 MHz, CDCl₃) δ ppm 2.3-2.5 (m, 2H), 3.55-3.64 (m, 2H), 3.76 (s, 3H), 4.83-4.85 (m, 1H), 6.86 (d, J=3.2 Hz, 2H), 7.23-7.25 (m, 2H).

Step 2: Methyl 4-bromo-2-(4-chlorophenoxy)butanoate (0.3 g, 1.01 mmol, 1 equiv) and tert-butyl (3-aminobicyclo[1.1.1]pentan-1-yl)carbamate (0.2 g, 1.01 mmol, 1 equiv) were charged to a sealed tube and Et₃N (0.6 mL) was added. The mixture was then heated at 100 ⁰0 using an oil bath for 1 h. The reaction mixture was diluted with water (50 mL) and extracted with EtOAc (2×50 mL). The combined EtOAc extracts were dried over anhydrous Na₂SO₄, filtered and evaporated under vacuum. The crude material was purified by column chromatography using an eluent of 45% EtOAc in hexane to obtain the desired product tert-butyl (3-(3-(4-chlorophenoxy)-2-oxopyrrolidin-1-yl)bicyclo[1.1.1]pentan-1-yl)carbamate as off-white solid (0.1 g, 25.6%). LCMS (ES) m/z=337.1 [M+H]⁺ (loss of tert-butyl group)

¹H NMR (400 MHz, DMSO-d₆) δ ppm 1.44 (s, 9H), 2.04-2.18 (m, 1H), 2.39 (s, 6H), 2.46-2.54 (m, 1H), 3.29-3.35 (m, 1H), 3.42-3.47 (m, 1H), 4.77 (t, J=7.2 Hz, 1H), 4.94 (bs, 1H), 6.98 (d, J=9.2 Hz, 2H), 7.21 (d, J=8.8 Hz, 2H).

Step 3: To a solution of tert-butyl (3-(3-(4-chlorophenoxy)-2-oxopyrrolidin-1-yl)bicyclo[1.1.1]pentan-1-yl)carbamate (0.1 g, 0.25 mmol) in DCM (5 mL) at 0° C. was added 2 mL of 4 M HCl in 1,4-dioxane and the mixture was stirred at rt for 16 h. The reaction mixture was concentrated to give 1-(3-aminobicyclo[1.1.1]pentan-1-yl)-3-(4-chlorophenoxy)pyrrolidin-2-one, hydrochloride (crude yield 0.08 g, 96.3%), which was taken to the next step without further purification. LCMS (ES) m/z=293 [M+H]⁺. ¹H NMR (400 MHz, DMSO-d₆) δ ppm 1.89-1.90 (m, 1H), 2.18-2.19 (m, 1H), 2.28-2.33 (m, 6H), 4.98 (t, J=7.2 Hz, 1H), 7.02 (d, J=8.8 Hz, 2H), 7.3 (d, J=9.6 Hz, 1H), 8.79 (s, 3H).

Step 4: To a solution of 1-(3-aminobicyclo[1.1.1]pentan-1-yl)-3-(4-chlorophenoxy)pyrrolidin-2-one hydrochloride (0.08 g, 0.24 mmol, 1 equiv) in THF (5 mL) was added BH₃.Me₂S (0.06 mL, 0.61 mmol, 2.5 equiv) at 0° C. The reaction mixture was then allowed to stir at rt for 16 h. Then the reaction mixture quenched with MeOH (1 mL) at 0° C. and stirred for 30 min, and concentrated under reduced pressure at rotavapor to obtain the crude product. This crude material was then dissolved in DCM (50 mL) and washed with saturated Na₂HCO₃ solution. The organic phase was dried over anhydrous Na₂SO₄, filtered and evaporated under vacuum to yield 3-(3-(4-chlorophenoxy)pyrrolidin-1-yl)bicyclo[1.1.1]pentan-1-amine (0.06 g, crude yield), which was used for the next step without further purification. LCMS (ES) m/z=279 [M+H]⁺.

Step 5: To a solution of 2-(4-chlorophenoxy)acetic acid (0.06 g, 0.23 mmol, 1.5 equiv) in DCM (5 mL) at 0° C. was added triethylamine (0.15 mL, 1.07 mmol, 5 equiv) followed by T3P®® (50 wt % in EtOAc) (0.25 mL, 0.43 mmol, 2 equiv). The mixture was stirred at 0° C. for 10 min, at which time, T3Pe (50 wt. % in ethyl acetate) (1.12 g, 1.768 mmol, 2 equiv) in dichloromethane (10 mL) was added at 0° C. and stirred for 10 min. To this reaction mixture was added 3-(3-(4-chlorophenoxy)pyrrolidin-1-yl)bicyclo[1.1.1]pentan-1-amine (0.06 g) in DCM (1 mL) slowly at 0° C. and the reaction was stirred at rt for 16 h. The reaction mixture was diluted with water (50 mL) and extracted with DCM (2×50 mL). The combined organic layer was washed with saturated aqueous NaHCO₃ solution (50 mL) and then dried over anhydrous Na₂SO₄, filtered and evaporated under vacuum under reduced pressure. The crude product was purified by preparative TLC using 40% EtOAc in hexanes. After final drying, the desired product 2-(4-chlorophenoxy)-N-(3-(3-(4-chlorophenoxy)pyrrolidin-1-yl)bicyclo[1.1.1]pentan-1-yl)acetamide was obtained as white solid (0.0108 g, 11.2%). LCMS (ES) m/z=447.3 [M+H]⁺. ¹H NMR (400 MHz, DMSO-d₆) δ ppm 1.76-1.77 (m, 1.5H), 1.95 (s, 6H), 2.2-2.27 (m, 1.5H), 2.58-2.61 (m, 1H), 2.65-2.69 (m, 1H), 2.84 (m, 1H), 4.4 (s, 2H), 4.85 (bs, 1H), 6.88 (d, J=8.8 Hz, 2H), 6.94 (d, J=8.8 Hz, 2H), 7.28 (d, J=8.4 Hz, 2H), 7.32 (d, J=8.8 Hz, 2H), 8.61 (s, 1H).

TABLE XIII

| | | | LCMS | |
|---|---|---|---|---|
| | | | m/z | |
| Cmpd # | Structure | Name | [M + H]⁺ | ¹H-NMR (400 MHz, DMSO-d₆) |
| XXIV | 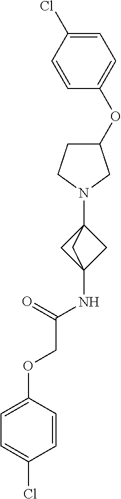 [SMILES: O=C(COc1ccc(Cl)cc1)NC12CC(N3CCC(Oc4ccc(Cl)cc4)C3)(C1)C2] | 2-(4- chlorophenoxy)- N-(3-(3-(4- chlorophenoxy) pyrrolidin-1- yl)bicyclo[1.1.1] pentan-1- yl)acetamide | 447.3 | 1.76-1.77 (m, 1.5 H), 1.95 (s, 6 H), 2.2-2.27 (m, 1.5 H), 2.58- 2.61 (m, 1 H), 2.65-2.69 (m, 1 H), 2.84 (m, 1 H), 4.4 (s, 2 H), 4.85 (bs, 1 H), 6.88 (d, J = 8.8 Hz, 2 H), 6.94 (d, J = 8.8 Hz, 2 H), 7.28 (d, J = 8.4 Hz, 2 H), 7.32 (d, J = 8.8 Hz, 2 H), 8.61 (s, 1 H). |

### Example XXIV A and B

The compounds of the following two structures are readily prepared by those of skill in the art generally according to the above Examples.

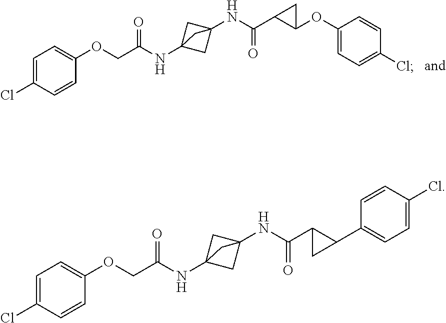 [SMILES: O=C(COc1ccc(Cl)cc1)NC12CC(NC(=O)C3CC3Oc3ccc(Cl)cc3)(C1)C2 ; O=C(COc1ccc(Cl)cc1)NC12CC(NC(=O)C3CC3c3ccc(Cl)cc3)(C1)C2]

### Example 1a and XXII

### 2-(4-chlorophenoxy-N-(3-(3-(4-chlorophenyl)-2-oxoimidazolidin-1-yl)bicyclo[1.1.1]pentan-1-yl)acetamide

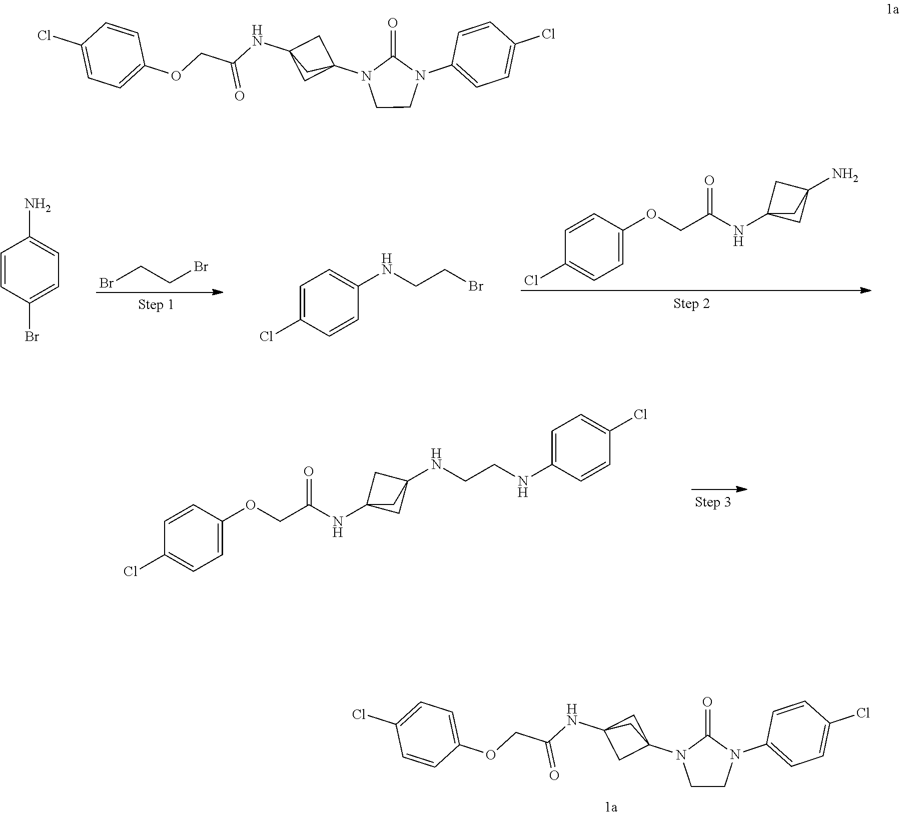

Step 1: To a solution of 4-chloroaniline (5 g, 39.37 mmol, 1 equiv) in acetonitrile (30 mL) was added 1,2-dibromoethane (6.7 mL, 78.74 mmol, 2 equiv) at rt. After stirring at 85° C. for 16 h, the reaction mixture was allowed to come to rt and concentrated under vacuum. The crude material was purified by flash column chromatography (6% EtOAc in hexanes). The fractions containing the product were combined and concentrated to provide N-(2-bromoethyl)-4-chloroaniline as a brown liquid (1 g, 11%). ¹H NMR (400 MHz, DMSO-d₆) δ ppm 3.29-3.43 (m, 2H), 3.52-3.55 (m, 2H), 6.05 (bs, 1H), 6.59 (d, J=8.8 Hz, 2H), 7.08 (d, J=8.8 Hz, 2H).

Step 2: [Note:—This reaction was performed in two batches, each batch with 0.25 g, ie 0.25×2=0.5 g]

In a sealed tube was added N-(3-aminobicyclo[1.1.1]pentan-1-yl)-2-(4-chlorophenoxy)acetamide (0.25 g, 0.93 mmol, 1 equiv), followed by triethylamine (0.6 mL, 4.68 mmol) and N-(2-bromoethyl)-4-chloroaniline (0.26 g, 1.12 mmol, 1.2 equiv). The reaction mixture was sealed and heated at 100° C. using an oil bath for 1 h. The reaction mixture was cooled to rt, diluted with water (50 mL), and extracted with EtOAc (2×15 mL). The combined EtOAc extracts were dried over anhydrous Na₂SO₄, filtered and evaporated under vacuum. The crude material obtained was purified by flash column chromatography (5% MeOH in DCM) to obtain the desired product 2-(4-chlorophenoxy)-N-(3-((2-((4-chlorophenyl)amino)ethyl)amino)bicyclo[1.1.1]pentan-1-yl)acetamide (0.4 g, 78%). LCMS (ES) m/z=420.1 [M+H]⁺

Step 3: To a solution of 2-(4-chlorophenoxy)-N-(3-((2-((4-chlorophenyl)amino)ethyl)amino)bicyclo[1.1.1]pentan-1-yl)acetamide (0.3 g, 0.71 mmol, 1 equiv) in DCM (15 mL) at 0° C. was added triethylamine (0.5 mL, 3.57 mmol, 5 equiv) followed by addition of triphosgene (0.21 g, 0.71 mmol, 1 equiv). After the reaction stirred at rt for 12 h, the reaction mixture was quenched with saturated aqueous NaHCO₃ solution (5 mL) at 0° C. and extracted with DCM (2×15 mL). The combined organic phase was dried over anhydrous Na₂SO₄, filtered and concentrated under reduced pressure to obtain the crude product (0.08 g) which was purified by preparative HPLC to afford the product 2-(4-chlorophenoxy)-N-(3-(3-(4-chlorophenyl)-2-oxoimidazolidin-1-yl)bicyclo[1.1.1]pentan-1-yl)acetamide (0.04 g) as an off-white solid. LCMS (ES) m/z=446.3 [M+H]⁺. ¹H NMR (400 MHz, DMSOd₆) δ ppm 2.29 (s, 6H), 3.43 (t, J=8 Hz, 2H), 3.75 (t, J=8 Hz, 2H), 4.43 (s, 2H), 6.96 (d, J=8.4 Hz, 2H), 7.33 (d, J=8.4 Hz, 4H), 7.55 (d, J=8.4 Hz, 2H), 8.73 (s, 1H).

The Compounds of Examples 1b to 1c were prepared generally according to the procedure described above for Example 1a.

TABLE 1

| | | | LCMS | |
|---|---|---|---|---|
| | | | m/z | ¹H-NMR (400 MHz, DMSO- |
| Cmpd # | Structure | Name | [M + H]⁺ | d₆) |
| 1a | 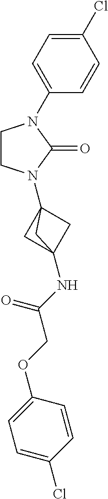 [SMILES: O=C(COc1ccc(Cl)cc1)NC12CC(N3CCN(c4ccc(Cl)cc4)C3=O)(C1)C2] | 2-(4-chlorophenoxy)- N-(3-(3-(4- chlorophenyl)-2- oxoimidazolidin-1- yl)bicyclo[1.1.1]pentan- 1-yl)acetamide | 446.3 | 2.29 (s, 6 H), 3.43 (t, J = 8 Hz, 2 H), 3.75 (t, J = 8 Hz, 2 H), 4.43 (s, 2 H), 6.96 (d, J = 8.4 Hz, 2 H), 7.33 (d, J = 8.4 Hz, 4 H), 7.55 (d, J = 8.4 Hz, 2 H), 8.73 (s, 1 H). |
| 1b | 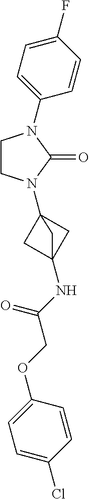 [SMILES: O=C(COc1ccc(Cl)cc1)NC12CC(N3CCN(c4ccc(F)cc4)C3=O)(C1)C2] | 2-(4-chlorophenoxy)- N-(3-(3-(4- fluorophenyl)-2- oxoimidazolidin-1- yl)bicyclo[1.1.1]pentan- 1-yl)acetamide | 430.3 | 2.29 (s, 6 H), 3.42 (t, J = 8 Hz, 2 H), 3.75 (t, J = 7.6 Hz, 2 H), 4.43 (s, 2 H), 6.97 (s, 2 H), 7.13 (t, J = 8.8 Hz, 2 H), 7.33 (d, J = 8.4 Hz, 2 H), 7.53 (t, J = 8.4 Hz, 2 H), 8.73 (s, 1 H). |
| 1c | 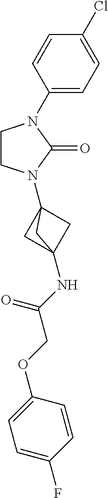 [SMILES: O=C(COc1ccc(F)cc1)NC12CC(N3CCN(c4ccc(Cl)cc4)C3=O)(C1)C2] | N-(3-(3-(4- chlorophenyl)-2- oxoimidazolidin-1- yl)bicyclo[1.1.1]pentan- 1-yl)-2-(4- fluorophenoxy)acetamide | 430.3 | 2.29 (s, 6 H), 3.42 (t, J = 8.0 Hz, 2 H), 3.75 (t, J = 7.6 Hz, 2 H), 4.40 (s, 2 H), 6.94-6.97 (m, 2 H), 7.09- 7.13 (m, 2 H), 7.32 (d, J = 8.8 Hz, 2 H), 7.55 (d, J = 8.8 Hz, 2 H), 8.70 (s, 1 H). |

### Example 1d

### N-(3-(3-(4-chloro-2-methylphenyl)-2-oxoimidazolidin-1-yl)bicyclo[1.1.1]pentan-1-yl)-2-(4-chlorophenoxy)acetamide

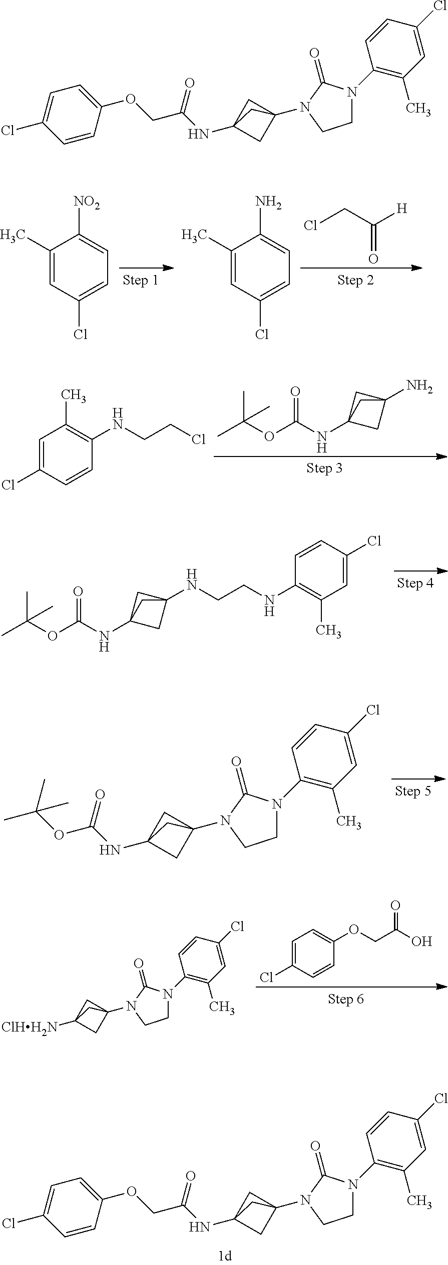

Step 1: To a solution of 4-chloro-2-methyl-1-nitrobenzene (5.0 g, 29.14 mmol, 1.0 equiv) in methanol (250 mL) at 0° C. was added ammonium chloride (8.57 g, 160.27 mmol, 5.5 equiv), stirred for 5 mins and then zinc dust (28.58 g, 437.1 mmol, 15.0 equiv) was added and the reaction mixture was stirred at room temperature for 3 h. After the consumption of the starting material (TLC, 10% ethyl acetate in hexane), the reaction mixture was filtered through a Celite®® bed and concentrated under reduced pressure. The crude material was purified by column chromatography (8-10% EtOAc in hexane) to obtain the desired product 4-chloro-2-methylaniline as a brown liquid (3.0 g, 73.2% yield). LCMS (ES) m/z=142.1 [M+H]⁺. ¹H NMR (400 MHz, DMSO-d₆) δ ppm 2.01 (s, 3H), 4.94 (s, 2H), 6.56 (d, J=8.0 Hz, 1H), 6.87 (d, J=8.4 Hz, 1H), 6.92 (s, 1H).

Step 2: To a solution of 4-chloro-2-methylaniline (0.5 g, 3.53 mmol, 1.0 equiv.) in methanol (20 mL) was added 2-chloroacetaldehyde (55% aqueous solution) (0.76 mL, 5.29 mmol, 1.5 equiv.) at room temperature followed by a catalytic amount of acetic acid (5-6 drops with syringe). After the reaction mixture stirred for 30 min, it was cooled to 0° C. and sodium cyanoborohydride (0.44 g, 7.06 mmol, 2.0 equiv.) was added. The reaction mixture was stirred at room temperature for 16 h. After the consumption of the starting material (TLC, 10% ethyl acetate in hexane), the reaction mixture was concentrated under reduced pressure. The crude was dissolved in ethyl acetate (200 mL) and washed with water (2×50 mL). The combined organic layer was dried over anhydrous Na₂SO₄, filtered and concentrated under reduced pressure. The crude material was purified by column chromatography (5% EtOAc in hexane) to obtain the desired product 4-chloro-N-(2-chloroethyl)-2-methylaniline as a yellow liquid (0.34 g, 47.3% yield). LCMS (ES) m/z=204.0 [M+H]⁺. ¹H NMR (400 MHz, DMSO-d₆) δ ppm 2.05 (s, 3H), 3.42 (d, J=6.0 Hz, 2H), 3.68-3.71 (m, 2H), 5.19 (s, 1H), 6.51-6.56 (m, 1H), 7.01 (s, 2H).

Step 3: To a solution of tert-butyl (3-aminobicyclo[1.1.1]pentan-1-yl)carbamate (0.3 g, 1.51 mmol, 1.0 equiv.) in triethylamine (0.85 mL, 6.04 mmol, 4.0 equiv.) was added 4-chloro-N-(2-chloroethyl)-2-methylaniline (0.37 g, 1.81 mmol, 1.2 equiv.) at room temperature. After the reaction mixture stirred at 100° C. for 16 h and consumption of the starting material (TLC, 50% ethyl acetate in hexane), the reaction mixture was diluted with ethyl acetate (100 mL) and washed with water (2×10 mL). The combined organic layer was dried over anhydrous Na₂SO₄, filtered and evaporated under reduced pressure. The crude material was purified by column chromatography (50-60% EtOAc in hexane) to obtain the desired product tert-butyl (3-((2-((4-chloro-2-methylphenyl)amino)ethyl)amino)bicyclo[1.1.1]pentan-1-yl)carbamate as pale yellow liquid (0.28 g, 50.9% yield). LCMS (ES) m/z=366.1 [M+H]⁺

Step 4: To a solution of tert-butyl (3-((2-((4-chloro-2-methylphenyl)amino)ethyl)amino)bicyclo[1.1.1]pentan-1-yl)carbamate (0.26 g, 0.71 mmol, 1.0 equiv.) in DCM (20 mL) was added triethylamine (0.5 mL, 3.55 mmol, 5.0 equiv.) at 0° C., stirred for 10 mins and then triphosgene (0.21 g, 0.71 mmol, 1.0 equiv.) was added at 0° C. and the reaction mixture was stirred at room temperature for 3 h. After the consumption of the starting material (TLC, 50% ethyl acetate in hexane), the reaction mixture was quenched with sodium bicarbonate solution at 0° C. and extracted with ethyl acetate (2×70 mL). The combined organic layer was dried over anhydrous Na₂SO₄, filtered and evaporated under reduced pressure. The crude material was purified by column chromatography (50-60% EtOAc in hexane) to obtain the desired product tert-butyl (3-(3-(4-chloro-2-methylphenyl)-2-oxoimidazolidin-1-yl)bicyclo[1.1.1]pentan-1-yl)carbamate as a pale yellow liquid (0.09 g, 32.1% yield). LCMS (ES) m/z=392.1 [M+H]⁺. ¹H NMR (400 MHz, DMSO-d₆) δ ppm 1.36 (s, 9H), 2.13-2.15 (m, 9H), 3.39 (t, J=7.6 Hz, 2H), 3.60 (t, J=7.8 Hz, 2H), 7.19-7.21 (m, 2H), 7.32 (s, 1H), 7.52 (bs, 1H).

Step 5: To a solution of tert-butyl (3-(3-(4-chloro-2-methylphenyl)-2-oxoimidazolidin-1-yl)bicyclo[1.1.1]pentan-1-yl)carbamate (0.09 g, 0.23 mmol, 1.0 equiv.) in DCM (5.0 mL) at 0° C. was added 2.0 mL of 4 M HCl in 1,4-dioxane and the mixture was stirred at room temperature for 16 h. After the consumption of the starting material (TLC, 5% MeOH in DCM), the reaction mixture was concentrated and the crude was triturated with n-pentane (2×5 mL) and dried under high vacuum to afford 1-(3-aminobicyclo[1.1.1]pentan-1-yl)-3-(4-chloro-2-methylphenyl)imidazolidin-2-one hydrochloride (crude yield 0.07 g, 93.4% yield), which was taken to the next step without further purification. LCMS (ES) m/z=292.1[M+H]⁺. ¹H NMR (400 MHz, DMSO-d₆) δ ppm 2.15 (s, 3H), 2.25 (s, 6H), 3.43-3.45 (m, 2H), 3.61-3.63 (m, 2H), 7.19-7.23 (m, 2H), 7.33 (s, 1H), 8.89 (bs, 3H).

Step 6: To a solution of 2-(4-chlorophenoxy)acetic acid (0.05 g, 0.26 mmol, 1.2 equiv.) in DCM (8 mL) at 0° C. was added triethylamine (0.12 mL, 0.84 mmol, 4.0 equiv), stirred for 10 mins and T3P®® (50 wt % in EtOAc) (0.25 mL, 0.42 mmol, 2.0 equiv.) was added. After the reaction mixture was stirred at 0° C. for 10 min, 1-(3-aminobicyclo[1.1.1]pentan-1-yl)-3-(4-chloro-2-methylphenyl)imidazolidin-2-one hydrochloride ((0.07 g, 0.21 mmol, 1.0 equiv.) which was neutralized with 0.2 mL of triethylamine in DCM) was added at 0° C. and the reaction stirred at room temperature for 16 h. After the consumption of the starting material (TLC, 70% EtOAc in hexane), the reaction mixture was diluted with DCM (100 mL) and was washed with saturated aqueous NaHCO₃ solution (2×10 mL) and water (2×10 mL). The combined organic layer was dried over anhydrous Na₂SO₄, filtered and concentrated under reduced pressure. The crude product was purified by silica gel column chromatography (50-60% EtOAc in hexane). It was again purified by Prep HPLC (Analytical conditions: lnertsil ODS 3V (250 mm×4.6 mm×5 mic); Mobile phase (A): 0.1% ammonia in water; Mobile phase (B): Acetonitrile; Flow rate: 1.0 mL/min) to afford the desired product N-(3-(3-(4-chloro-2-methylphenyl)-2-oxoimidazolidin-1-yl)bicyclo[1.1.1]pentan-1-yl)-2-(4-chlorophenoxy)acetamide as an off-white solid (0.025 g, 25.5% yield). LCMS (ES) m/z=460.3 [M+H]⁺. ¹H NMR (400 MHz, DMSO-d₆) δ ppm 2.16 (s, 3H), 2.26 (s, 6H), 3.42 (t, J=7.4 Hz, 2H), 3.62 (t, J=7.4 Hz, 2H), 4.42 (s, 2H), 6.96 (d, J=8.4 Hz, 2H), 7.22 (s, 2H), 7.32 (s, 3H), 8.70 (s, 1H).

The Compounds of Examples 1e to 1k were prepared generally according to the procedure described above for Example 1d.

TABLE 2

| | | | LCMS | |
|---|---|---|---|---|
| | | | m/z | ¹H-NMR (400 MHz, |
| Cmpd # | Structure | Name | [M + H]⁺ | DMSO-d₆) |
| 1d | 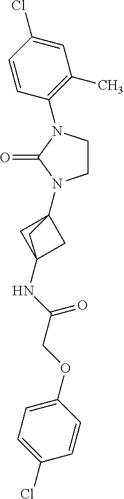 [SMILES: Cc1cc(Cl)ccc1N1CCN(C23CC(NC(=O)COc4ccc(Cl)cc4)(C2)C3)C1=O] | N-(3-(3-(4-chloro-2-methylphenyl)- 2-oxoimidazolidin-1- yl)bicyclo[1.1.1]pentan-1-yl)-2-(4- chlorophenoxy)acetamide | 460.3 | 2.16 (s, 3 H), 2.26 (s, 6 H), 3.42 (t, J = 7.4 Hz, 2 H), 3.62 (t, J = 7.4 Hz, 2 H), 4.42 (s, 2 H), 6.96 (d, J = 8.4 Hz, 2 H), 7.22 (s, 2 H), 7.32 (s, 3 H), 8.70 (s, 1 H). |
| 1e | 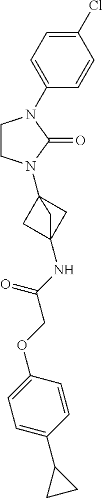 [SMILES: O=C(COc1ccc(C2CC2)cc1)NC12CC(N3CCN(c4ccc(Cl)cc4)C3=O)(C1)C2] | N-(3-(3-(4-chlorophenyl)-2- oxoimidazolidin-1- yl)bicyclo[1.1.1]pentan-1-yl)-2-(4- cyclopropylphenoxy)acetamide | 452.4 | 0.55-0.56 (m, 2 H), 0.84-0.86 (m, 2 H), 1.82-1.87 (m, 1 H), 2.29 (s, 6 H), 3.42 (t, J = 7.6 Hz, 2 H), 3.75 (t, J = 7.8 Hz, 2 H), 4.36 (s, 2 H), 6.82 (d, J = 8.4 Hz, 2 H), 6.99 (d, J = 8.4 Hz, 2 H), 7.33 (d, J = 8.8 Hz, 2 H), 7.55 (d, J = 8.8 Hz, 2 H), 8.66 (s, 1 H). |
| 1f | 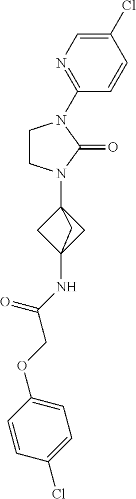 [SMILES: O=C(COc1ccc(Cl)cc1)NC12CC(N3CCN(c4ccc(Cl)cn4)C3=O)(C1)C2] | 2-(4-chlorophenoxy)-N-(3-(3-(5- chloropyridin-2-yl)-2- oxoimidazolidin-1- yl)bicyclo[1.1.1]pentan-1- yl)acetamide | 447.3 | 2.36 (s, 6 H), 3.44 (t, J = 8.0 Hz, 2 H), 3.88 (t, J = 8.0 Hz, 2 H), 4.43 (s, 2 H), 6.96 (d, J = 8.8 Hz, 2 H), 7.33 (d, J = 8.8 Hz, 2 H), 7.80 (d, J = 9.2 Hz, 1 H), 7.16 (d, J = 9.2 Hz, 1 H), 8.30 (bs, 1 H), 8.73 (s, 1 H). |
| 1g | 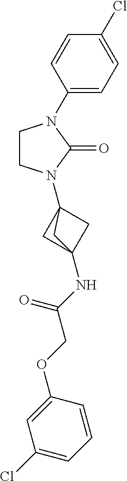 [SMILES: O=C(COc1cccc(Cl)c1)NC12CC(N3CCN(c4ccc(Cl)cc4)C3=O)(C1)C2] | 2-(3-chlorophenoxy)-N-(3-(3-(4- chlorophenyl)-2-oxoimidazolidin-1- yl)bicyclo[1.1.1]pentan-1- yl)acetamide | 446.3 | 2.29 (s, 6 H), 3.42 (t, J = 7.2 Hz, 2 H), 3.74 (t, J = 8.0 Hz, 2 H), 4.46 (s, 2 H), 6.92 (d, J = 7.6 Hz, 1 H), 7.00- 7.04 (m, 2 H), 7.29- 7.34 (m, 3 H), 7.55 (d, J = 8.8 Hz, 2 H), 8.75 (s, 1 H). |
| 1h | 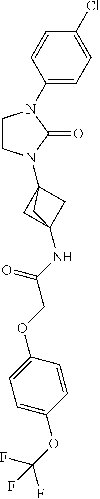 [SMILES: O=C(COc1ccc(OC(F)(F)F)cc1)NC12CC(N3CCN(c4ccc(Cl)cc4)C3=O)(C1)C2] | N-(3-(3-(4-chlorophenyl)-2- oxoimidazolidin-1- yl)bicyclo[1.1.1]pentan-1-yl)-2-(4- (trifluoromethoxy)phenoxy)acetamide | 496.3 | 2.29 (s, 6 H), 3.45- 3.41 (m, 2 H), 3.77- 3.73 (m, 2 H), 4.46 (s, 2 H), 7.04-7.02 (m, 2 H), 7.34-7.72 (m, 4 H), 7.56-7.54 (m, 2 H), 8.73 (s, 1 H). |
| 1i and XXIII | 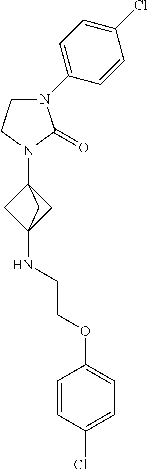 [SMILES: O=C1N(c2ccc(Cl)cc2)CCN1C12CC(NCCOc3ccc(Cl)cc3)(C1)C2] | 1-(3-((2-(4- chlorophenoxy)ethyl)amino)bi- cyclo[1.1.1]pentan-1-yl)-3-(4- chlorophenyl)imidazolidin-2-one | 432.1 | 2.00 (s, 6 H), 2.60 (s, 1 H), 2.82-2.84 (m, 2 H), 3.40 (t, J = 8.4 Hz, 2 H), 3.74 (t, J = 7.2 Hz, 2 H), 3.96 (t, J = 5.6 Hz, 2 H), 6.94 (d, J = 8.0 Hz, 2 H), 7.28- 7.33 (m, 4 H), 7.54 (d, J = 9.2 Hz, 2 H). |
| 1j | 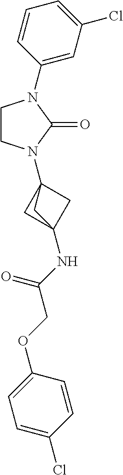 [SMILES: O=C(COc1ccc(Cl)cc1)NC12CC(N3CCN(c4cccc(Cl)c4)C3=O)(C1)C2] | 2-(4-chlorophenoxy)-N-(3-(3-(3- chlorophenyl)-2-oxoimidazolidin-1- yl)bicyclo[1.1.1]pentan-1- yl)acetamide | 446.3 | 2.30 (s, 6 H), 3.45- 3.43 (m, 2 H), 3.87- 3.75 (m, 2 H), 4.43 (s, 2 H), 7.03-7.95 (m, 3 H), 7.36-7.32 (m, 4 H), 7.74 (s, 1 H), 8.72 (s, 1 H). |
| 1k | 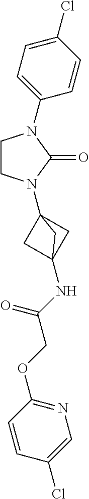 [SMILES: O=C(COc1ccc(Cl)cn1)NC12CC(N3CCN(c4ccc(Cl)cc4)C3=O)(C1)C2] | N-(3-(3-(4-chlorophenyl)-2- oxoimidazolidin-1- yl)bicyclo[1.1.1]pentan-1-yl)-2-((5- chloropyridin-2-yl)oxy)acetamide | 447.32 | 2.25 (s, 6 H), 3.41 (t, J = 8.0 Hz, 2 H), 3.74 (t, J = 7.6 Hz, 2 H), 4.65 (s, 2 H), 6.93 (d, J = 9.2 Hz, 1 H), 7.32 (d, J = 8.8 Hz, 2 H), 7.54 (d, J = 8.8 Hz, 2 H), 7.82 (d, J = 8.8 Hz, 1 H), 8.16 (s, 1 H), 8.67 (s, 1 H). |

### Example 1l

### 2-(4-chloro-3-(trifluoromethyl)phenoxy)-N-(3-(3-(4-chlorophenyl)-2-oxoimidazolidin-1-yl)bicyclo[1.1.1]pentan-1-yl)acetamide

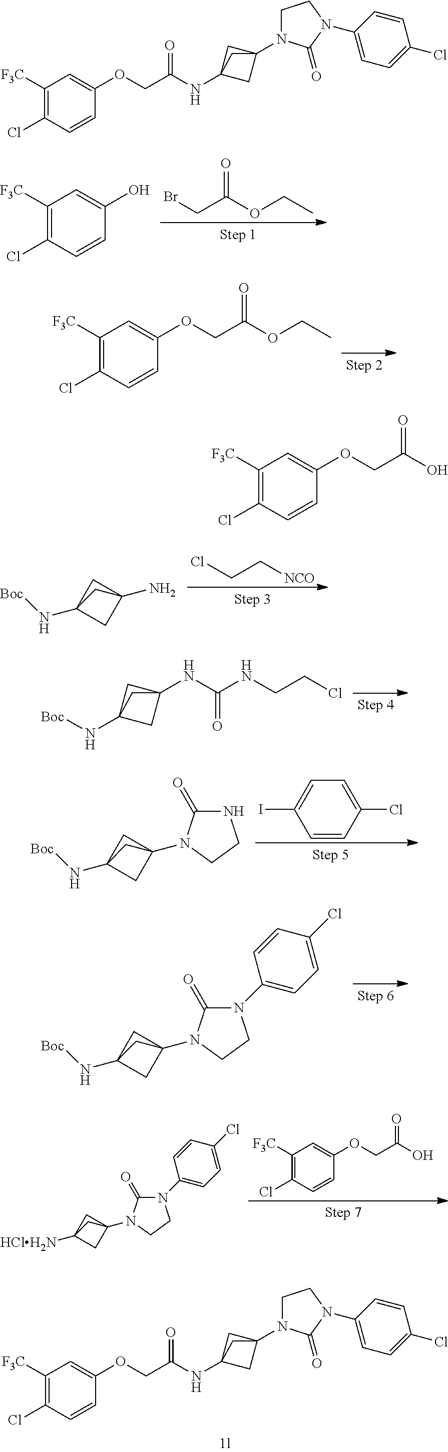

Step 1: To a solution of 4-chloro-3-(trifluoromethyl)phenol (1.5 g, 7.63 mmol, 1 equiv.) in acetone (30 mL) was added K₂CO₃ (3.15 g, 22.82 mmol, 3 equiv.) followed by the addition of ethyl 2-bromoacetate (1.52 g, 9.10 mmol, 1.2 equiv) dropwise at 0° C. The reaction mixture stirred at 60° C. for 4 h. After consumption of the starting material (TLC, 5% EtOAc in Hexane), the reaction mixture was filtered through a Buchner funnel, and concentrated under reduced pressure. The crude product was purified by flash column chromatography (Combiflash®) using a silica gel column and the product was eluted at 5% ethyl acetate in hexane as eluent to obtain the title compound ethyl 2-(4-chloro-3-(trifluoromethyl)phenoxy)acetate (1.3 g) as a colourless liquid. LCMS (ES) m/z=282.0 [M+H]⁺. ¹H NMR (400 MHz, DMSO-d₆) δ ppm 1.18 (t, J=7.2 Hz, 3H), 4.13-4.18 (m, 2H), 4.91 (s, 2H), 7.25-7.28 (m, 1H), 7.34 (d, J=2.8 Hz, 1H), 7.61 (d, J=9.2 Hz, 1H).

Step 2: To a solution of ethyl 2-(4-chloro-3-(trifluoromethyl)phenoxy)acetate (1.3 g, 4.59 mmol, 1 equiv.) in a mixture of THF (20 mL) and water (20 mL) was added LiOH.H₂O (0.27 g, 11.45 mmol, 2.5 equiv.) at 0° C. and the resulting mixture stirred at room temperature for 3 h. After consumption of the starting material (TLC, 5% Methanol in DCM), THF was removed under reduced pressure. The residue was diluted with water (20 mL), and washed with Et₂O (2×15 mL) to remove unreacted ethyl 2-bromoacetate. The aqueous layer was acidified with 1 N HCl up to pH ˜2 at 0° C. and extracted with EtOAc (2×30 mL). The combined organic layers were dried over anhydrous sodium sulfate, filtered and concentrated under reduced pressure to obtain title compound 2-(4-chloro-3-(trifluoromethyl)phenoxy)acetic acid (1.05 g, 90% yield) as a white solid. LCMS (ES) m/z=253.0 [M−H]⁻. ¹H NMR (400 MHz, DMSO-d₆) δ ppm 4.81 (s, 2H), 7.24 (d, J=8.8 Hz, 1H), 7.32 (d, J=2.4 Hz, 1H), 7.61 (d, J=8.8 Hz, 1H), 13.10 (s, 1H).

Step 3: To a solution of tert-butyl (3-aminobicyclo[1.1.1]pentan-1-yl)carbamate (5 g, 25.21 mmol, 1.0 equiv.) in CHCl₃ (50 mL) was added 1-chloro-2-isocyanatoethane (3.22 mL, 37.82 mmol, 1.5 equiv.) at 0° C. and heated to 60° C. for 1 h. After the completion of the reaction, (TLC in 5% Methanol in DCM) the reaction mixture was cooled to room temperature and concentrated under reduced pressure to yield crude product. To the above crude, n-pentane (150 mL) was added and stirred for 0.5 h at room temperature. The solid formed and was filtered and washed with n-pentane to afford tert-butyl (3-(3-(2-chloroethyl)ureido)bicyclo[1.1.1]pentan-1-yl)carbamate as a white solid (7 g, 91% yield). LCMS (ES) m/z=304.1 [M+H]⁺. ¹H NMR (400 MHz, DMSO-d₆) δ ppm 1.34 (s, 9H), 2.00 (s, 6H), 3.24-3.25 (m, 2H), 3.52 (t, J=6.0 Hz, 2H), 5.93 (s, 1H), 6.66 (s, 1H), 7.41 (bs, 1H).

Step 4: To a solution of tert-butyl (3-(3-(2-chloroethyl)ureido)bicyclo[1.1.1]pentan-1-yl)carbamate (7 g, 23.04 mmol, 1.0 equiv.) in acetonitrile (70 mL) was added cesium carbonate (15 g, 46.08 mmol, 2.0 equiv.) at room temperature. The reaction was gradually allowed to heat to 100° C. and stirred for 12 h. After the completion of the reaction, (TLC in 5% Methanol in DCM) reaction mixture was allowed to cool to room temperature, diluted with water (150 mL) and extracted with EtOAc (3×100 mL). The combined EtOAc extracts were dried over anhydrous Na₂SO₄, filtered and concentrated under vacuum to give crude product. To the above crude, diethyl ether (100 mL) was added and stirred for 0.5 h at room temperature. Solid formed and was filtered and washed with diethyl ether to afford tert-butyl (3-(2-oxoimidazolidin-1-yl)bicyclo[1.1.1]pentan-1-yl)carbamate as a white solid (4 g, 65% yield). LCMS (ES) m/z=268.1 [M+H]⁺. ¹H NMR (400 MHz, DMSO-d₆) δ ppm 1.35 (s, 9H), 2.06 (s, 6H), 3.15-3.17 (m, 2H), 3.21-3.23 (m, 2H), 6.31 (s, 1H), 7.47 (bs, 1H).

Step 5: To a solution of tert-butyl (3-(2-oxoimidazolidin-1-yl)bicyclo[1.1.1]pentan-1-yl)carbamate (2.0 g, 7.49 mmol, 1.0 equiv.), in DMSO (20 mL) was added K₃PO₄ (3.18 g, 14.98 mmol, 2.0 equiv.), DMEDA (0.16 mL, 1.498 mmol, 0.2 equiv.) and Cul (0.142 g, 0.749 mmol, 0.1 equiv.) and stirred at room temperature for 10-15 minutes. To the above reaction mixture 1-chloro-4-iodobenzene (2.14 g, 8.98 mmol, 1.2 equiv.) was added drop wise and allowed to stir at room temperature for 12 h. After the completion of the reaction (TLC in 40% ethyl acetate in hexane) the reaction mixture was diluted with water (30 mL) and extracted with EtOAc (3×30 mL). The combined EtOAc extracts were dried over anhydrous Na₂SO₄, filtered and concentrated under vacuum to give crude product. The crude product was purified by silica gel column chromatography (60% Ethyl acetate in Hexane) to afford tert-butyl (3-(3-(4-chlorophenyl)-2-oxoimidazolidin-1-yl)bicyclo[1.1.1]pentan-1-yl)carbamate (0.8 g, 30% yield) as a brown solid. LCMS (ES) m/z=378.1 [M+H]⁺. ¹H NMR (400 MHz, CDCl3) δ ppm 1.45 (s, 9H), 2.36 (s, 6H), 3.46 (t, J=8.4 Hz, 2H), 5.00 (bs, 1H), 7.25-7.27 (m, 2H), 7.47 (d, J=8.8 Hz, 2H).

Step 6: To a solution of tert-butyl (3-(3-(4-chlorophenyl)-2-oxoimidazolidin-1-yl)bicyclo[1.1.1]pentan-1-yl)carbamate (0.8 g, 2.11 mmol, 1 equiv) in DCM (8 mL) at 0° C. was added 4M HCl in 1,4-dioxane (8 mL) and the reaction mixture was allowed to stir at room temperature for 6 h. After the completion of the reaction, the reaction mixture was evaporated under vacuum to afford crude 1-(3-aminobicyclo[1.1.1]pentan-1-yl)-3-(4-chlorophenyl)imidazolidin-2-one hydrochloride (0.587 g, 100%) as an off-white solid. LCMS (ES) m/z=278.1 [M+H]+. ¹H NMR (400 MHz, DMSO-d₆) δ ppm 2.27 (s, 6H), 3.43-3.45 (m, 2H), 3.75-3.77 (m, 2H), 7.34 (d, J=8.4 Hz, 2H), 7.54 (d, J=8.4 Hz, 2H), 8.80 (bs, 3H).

Step 7: To a solution of 2-(4-chloro-3-(trifluoromethyl)phenoxy)acetic acid (0.078 g, 0.30 mmol, 1.2 equiv) in dichloromethane (2 mL) was added triethylamine (0.05 g, 0.5 mmol, 2.0 equiv) at room temperature. After stirring for 5 min, T3P® (50 wt. % in ethyl acetate) (0.12 g, 0.37 mmol, 1.5 equiv) was added to the reaction mass at room temperature and stirred for 10 min before adding 1-(3-aminobicyclo[1.1.1]pentan-1-yl)-3-(4-chlorophenyl)imidazolidin-2-one hydrochloride (0.08 g, 0.25 mmol, 1.0 equiv) along with triethylamine (0.05 g, 0.5 mmol, 2.0 equiv) in DCM (2 mL). The reaction mixture stirred at room temperature for 16 h. After completion of the reaction, the reaction mixture was diluted with water (10 mL) and extracted with DCM (2×25 mL). The combined organic extract was washed with saturated aqueous NaHCO₃ solution (10 mL), brine solution (7 mL), dried over anhydrous sodium sulfate, filtered and concentrated under reduced pressure to give the crude product. The crude material was purified by flash column chromatography using a silica gel column where the product along with a very close impurity was eluted at 3% methanol in DCM. Finally, this crude product was purified by preparative HPLC to afford 2-(4-chloro-3-(trifluoromethyl)phenoxy)-N-(3-(3-(4-chlorophenyl)-2-oxoimidazolidin-1-yl)bicyclo[1.1.1]pentan-1-yl)acetamide (0.04 g, 17.5% yield) as a white solid. LCMS (ES) m/z=514.4 [M+H]⁺. ¹H NMR (400 MHz, DMSO-d₆) δ ppm 2.29 (s, 6H), 3.43 (t, J=8.0 Hz, 2H), 3.75 (t, J=8.0 Hz, 2H), 4.55 (s, 2H), 7.26 (d, J=8.4 Hz, 1H), 7.33 (d, J=8.8 Hz, 2H), 7.40 (d, J=2.4 Hz, 1H), 7.55 (d, J=9.2 Hz, 2H), 7.63 (d, J=8.8 Hz, 1H), 8.77 (s, 1H).

### Example 1m

### 2-(4-chloro-3-fluorophenoxy)-N-(3-(3-(4-chlorophenyl)-2-oxoimidazolidin-1-yl)bicyclo[1.1.1]pentan-1-yl)acetamide

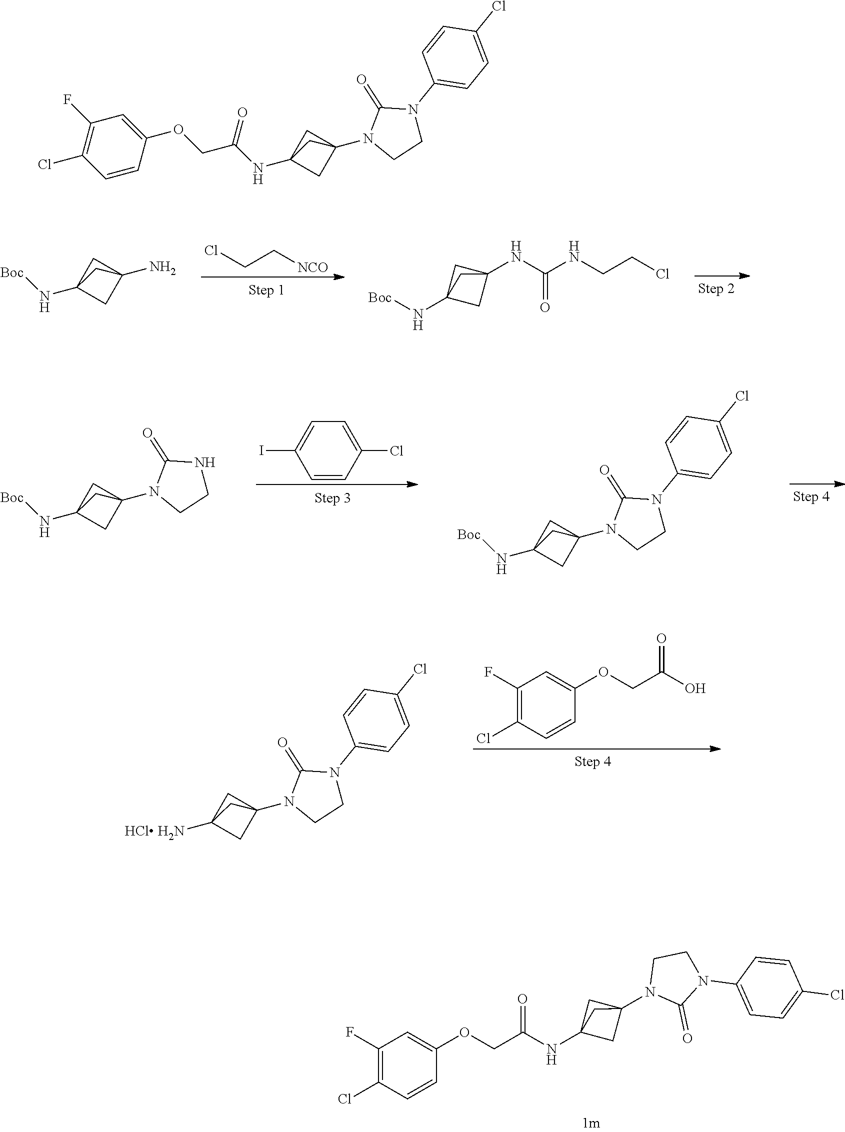

Step 1: To a stirred solution of tert-butyl (3-aminobicyclo[1.1.1]pentan-1-yl)carbamate (5.0 g, 25.21 mmol, 1.0 equiv.) in CHCl₃ (50 mL) was added 1-chloro-2-isocyanatoethane (3.22 mL, 37.82 mmol, 1.5 equiv.) at 0° C. and the reaction mixture was stirred at 60° C. for 1 h. After consumption of the starting material (TLC, 5% MeOH in DCM), the reaction mixture was cooled to RT and concentrated under reduced pressure to give crude product. To this was added n-pentane (150 mL), stirred for 0.5 h at RT, and then filtered. The compound was washed with n-pentane to give tert-butyl (3-(3-(2-chloroethyl)ureido)bicyclo[1.1.1]pentan-1-yl)carbamate (7.0 g, 91% yield) as a white solid. LCMS (ES) m/z=304.1 [M+H]⁺. ¹H NMR (400 MHz, DMSO-d₆) δ ppm 1.34 (s, 9H),2.00 (s, 6H), 3.24-3.25 (m, 2H), 3.52(t, J=6.0 Hz, 2H), 5.93 (s, 1H), 6.66 (s, 1H), 7.41 (bs, 1H).

Step 2: To a stirred solution of tert-butyl (3-(3-(2-chloroethyl)ureido)bicyclo[1.1.1]pentan-1-yl)carbamate (7.0 g, 23.04 mmol, 1.0 equiv.) in CH₃CN (70 mL) at RT was added CS₂CO₃ (15 g, 46.08 mmol, 2.0 equiv). The reaction was then heated to 100° C. for 12 h. After consumption of the starting material (TLC, 5% MeOH in DCM), the reaction mixture was diluted with H₂O (200 mL) and extracted with EtOAc (3×100 mL).The combined organics were dried over anhydrous Na₂SO₄, filtered and concentrated to give the crude compound. To the crude was added diethyl ether (100 mL), stirred for 0.5 h at RT and then filtered. The product was washed with diethyl ether to give tert-butyl (3-(2-oxoimidazolidin-1-yl)bicyclo[1.1.1]pentan-1-yl)carbamate (4.0 g, 65% yield) as a white solid. LCMS (ES) m/z=268.1 [M+H]⁺. ¹H NMR (400 MHz, DMSO-d₆) δ ppm 1.35 (s, 9H), 2.06 (s, 6H), 3.15-3.17 (m, 2H), 3.21-3.23(m, 2H), 6.31 (s, 1H), 7.47 (bs, 1H).

Step 3: To a sealed tube, tert-butyl (3-(2-oxoimidazolidin-1-yl)bicyclo[1.1.1]pentan-1-yl)carbamate (2.0 g, 7.49 mmol, 1.0 equiv.), 1-chloro-4-iodobenzene (2.14 g, 8.98 mmol, 1.2 equiv.), copper iodide(0.142 g, 0.749 mmol, 0.1 equiv.), DMEDA (0.16 mL, 1.498 mmol, 0.2 equiv.), K₃PO₄ (3.18 g, 14.98 mmol, 2.0 equiv.) and DMSO (20 mL) were added at RT. The resulting reaction mixture stirred for 12 h at RT. After consumption of the starting material (TLC, 40% EtOAc in hexane), the reaction mixture was filtered through a Celite® bed and washed with EtOAc (50 mL). The filtered organics were diluted with H_{d 2}O (100 mL) and extracted into EtOAc (3×100 mL). The combined organics were washed with water (50 mL), brine (50 mL), dried over anhydrous sodium sulfate, filtered and concentrated under vacuum to afford the crude product. The crude product was purified by flash column chromatography (Combiflash®) using a silica gel column (60% ethyl acetate in hexane) to obtain tert-butyl (3-(3-(4-chlorophenyl)-2-oxoimidazolidin-1-yl)bicyclo[1.1.1]pentan-1-yl)carbamate (0.8 g, 30% yield) as a brown solid. LCMS (ES) m/z=378.1 [M+H]⁺. ¹H NMR (400 MHz, CDCl3) δ ppm 1.45 (s, 9H), 2.36 (s, 6H), 3.46 (t, J=8.4 Hz, 2H), 5.00(bs, 1H), 7.25-7.27 (m, 2H), 7.47 (d, J=8.8 Hz, 2H).

Step 4: 4M HCl in dioxane (8 mL) was added to the stirred solution of tert-butyl (3-(3-(4-chlorophenyl)-2-oxoimidazolidin-1-yl)bicyclo[1.1.1]pentan-1-yl)carbamate (0.8 g, 2.11 mmol, 1 equiv) in DCM (8 mL) at 0° C. The resulting mixture was allowed to warm to 27° C. and stirred for 6 h. The progress of the reaction was monitored by TLC. After completion of reaction, the mixture was concentrated under reduced pressure to obtain 1-(3-aminobicyclo[1.1.1]pentan-1-yl)-3-(4-chlorophenyl)imidazolidin-2-one hydrochloride (0.587 g, 100%) as an off-white solid. LCMS (ES) m/z=278.1 [M+H]+. ¹H NMR (400 MHz, DMSO-d6) δ ppm 2.27 (s, 6H), 3.43-3.45 (m, 2H), 3.75-3.77 (m, 2H), 7.34 (d, J=8.4 Hz, 2H), 7.54 (d, J=8.4 Hz, 2H), 8.80 (bs, 3H).

Step 5: To a mixture of 1-(3-aminobicyclo[1.1.1]pentan-1-yl)-3-(4-chlorophenyl)imidazolidin-2-one hydrochloride (0.075 g, 0.238 mmol, 1 equiv), 2-(4-chloro-3-fluorophenoxy)acetic acid (0.058 g, 0.285 mmol, 1.2 equiv) and triethylamine (0.166 mL, 1.19 mmol, 5.0 equiv) in dichloromethane (5 mL) was added T3P® (50 wt. % in ethyl acetate) (0.3 g, 0.476 mmol, 2.0 equiv) at 0° C. The reaction mixture was allowed to warm to 27° C., and stirred for 16 h. The progress of the reaction was monitored by TLC. After completion of reaction, the reaction mixture was diluted with dichloromethane (10 mL), washed with 10% sodium bicarbonate solution (10 mL), water (5 mL), brine (5 mL), dried over anhydrous sodium sulfate, filtered and concentrated under reduced pressure to give the crude product. The crude product was purified by flash column chromatography (Combiflash®) using a silica gel column (2% methanol in DCM) to obtain the title compound as 99% pure, but contained a small impurity. Again purified by preparative HPLC [Analytical conditions: Column: lnertsil ODS 3V (250 mm×4.6 mm×5 um), Mobile phase (A): 0.1% Ammonia in water, Mobile phase (B): ACN; Flow rate: 1.0 mL/min, Time % B: 0/10, 10/80, 25/90, 27/10, 30/10]. Pure fractions were concentrated under reduced pressure and lyophilized to give 2-(4-chloro-3-fluorophenoxy)-N-(3-(3-(4-chlorophenyl)-2-oxoimidazolidin-1-yl)bicyclo[1.1.1]pentan-1-yl)acetamide (0.03 g, 27%) as white solid. LCMS (ES) m/z=464.3 [M+H]+. ¹H NMR (400 MHz, DMSO-d6) δ ppm 2.29 (s, 6H), 3.42 (t, J=8.0 Hz, 2H), 3.75 (t, J=8.4 Hz, 2H), 4.47(s, 2H), 6.82-6.85 (m, 1H), 7.04-7.07 (m, 1H), 7.33(d, J=8.8 Hz, 2H), 7.48(t, J=8.8 Hz, 1H), 7.55(d, J=9.2 Hz, 2H), 8.73 (s, 1H).

The compounds of Examples 1n-1u were prepared generally according to the procedure described above for Examples 1l and 1 m.

The Compounds of Examples 1n to 1u were prepared generally according to the procedure described above for Examples 1l and 1m.

TABLE 2b

| | | | LCMS | |
|---|---|---|---|---|
| Cmpd | | | m/z | ¹H-NMR (400 MHz, |
| # | Structure | Name | [M + H]⁺ | DMSO-d₆) |
| 1l | 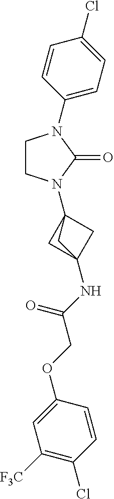 [SMILES: O=C(COc1ccc(Cl)c(C(F)(F)F)c1)NC12CC(N3CCN(c4ccc(Cl)cc4)C3=O)(C1)C2] | 2-(4-chloro-3- (trifluoromethyl)phenoxy)- N-(3-(3-(4- chlorophenyl)-2- oxoimidazolidin-1- yl)bicyclo[1.1.1]pentan- 1-yl)acetamide | 514.4 | 2.29 (s, 6 H), 3.43 (t, J = 8.0 Hz, 2 H), 3.75 (t, J = 8.0 Hz, 2 H), 4.55 (s, 2 H), 7.26 (d, J = 8.4 Hz, 1 H), 7.33 (d, J = 8.8 Hz, 2 H), 7.40 (d, J = 2.4 Hz, 1 H), 7.55 (d, J = 9.2 Hz, 2 H), 7.63 (d, J = 8.8 Hz, 1 H), 8.77 (s, 1 H). |
| 1m | 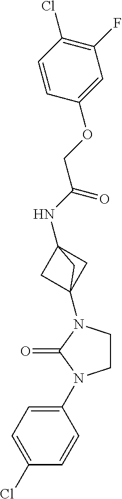 [SMILES: O=C(COc1ccc(Cl)c(F)c1)NC12CC(N3CCN(c4ccc(Cl)cc4)C3=O)(C1)C2] | 2-(4-chloro-3- fluorophenoxy)-N-(3- (3-(4-chlorophenyl)-2- oxoimidazolidin-1- yl)bicyclo[1.1.1]pentan- 1-yl)acetamide | 464.3 | 2.29 (s, 6 H), 3.42 (t, J = 8.0 Hz, 2 H), 3.75 (t, J = 8.4 Hz, 2 H), 4.47 (s, 2 H), 6.82-6.85 (m, 1 H), 7.04- 7.07 (m, 1 H), 7.33 (d, J = 8.8 Hz, 2 H), 7.48 (t, J = 8.8 Hz, 1 H), 7.55 (d, J = 9.2 Hz, 2 H), 8.73 (s, 1 H). |
| 1n | 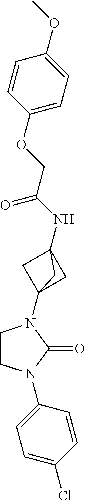 [SMILES: COc1ccc(OCC(=O)NC23CC(N4CCN(c5ccc(Cl)cc5)C4=O)(C2)C3)cc1] | N-(3-(3-(4- chlorophenyl)-2- oxoimidazolidin-1- yl)bicyclo[1.1.1]pentan- 1-yl)-2-(4- methoxyphenoxy)aceta- mide | 442.4 | 2.29 (s, 6 H), 3.42 (t, J = 8.0 Hz, 2 H), 3.68 (s, 3 H), 3.73-3.77 (m, 2 H), 4.34 (s, 2 H), 6.83-6.90 (m, 4 H), 7.33 (d, J = 9.2 Hz, 2 H), 7.55 (d, J = 9.2 Hz, 2 H), 8.65 (s, 1 H). |
| 1o | 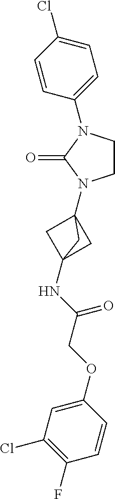 [SMILES: O=C(COc1ccc(F)c(Cl)c1)NC12CC(N3CCN(c4ccc(Cl)cc4)C3=O)(C1)C2] | 2-(3-chloro-4- fluorophenoxy)-N-(3- (3-(4-chlorophenyl)- 2-oxoimidazolidin-1- yl)bicyclo[1.1.1]pentan- 1-yl)acetamide | 464.0 | 2.29 (s, 6 H), 3.42 (t, J = 8.0 Hz, 2 H), 3.75 (t, J = 7.2 Hz, 2 H), 4.45 (s, 2 H), 6.97-6.95 (m, 1 H), 7.36- 7.18 (m, 4 H), 7.55 (d, J = 8.8 Hz, 2 H), 8.71 (s, 1 H). |
| 1p | 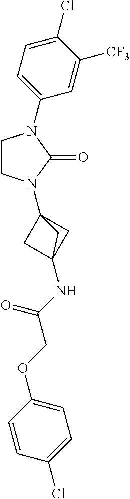 [SMILES: O=C(COc1ccc(Cl)cc1)NC12CC(N3CCN(c4ccc(Cl)c(C(F)(F)F)c4)C3=O)(C1)C2] | N-(3-(3-(4-chloro-3- (trifluoromethyl)phenyl)- 2-oxoimidazolidin-1- yl)bicyclo[1.1.1]pentan- 1-yl)-2-(4- chlorophenoxy)aceta- mide | 514.33 | 2.30 (s, 6 H), 3.46 (t, J = 7.6 Hz, 2 H), 3.82 (t, J = 8 Hz, 2 H), 4.43 (s, 2 H), 6.96 (d, J = 8.8 Hz, 2 H), 7.33 (d, J = 8.8 Hz, 2 H), 7.62 (s, 2 H), 8.22 (s, 1 H), 8.74 (s, 1 H). |
| 1q | 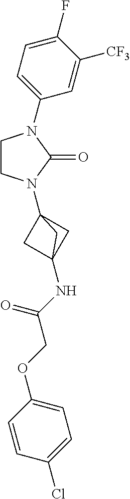 [SMILES: O=C(COc1ccc(Cl)cc1)NC12CC(N3CCN(c4ccc(F)c(C(F)(F)F)c4)C3=O)(C1)C2] | 2-(4-chlorophenoxy)- N-(3-(3-(4-fluoro-3- (trifluoromethyl)phenyl)- 2-oxoimidazolidin-1- yl)bicyclo[1.1.1]pentan- 1-yl)acetamide | 497.87 | 2.30 (s, 6 H), 3.44 (t, J = 7.8 Hz, 2 H), 3.82 (t, J = 8 Hz, 2 H), 4.43 (s, 2 H), 6.96 (d, J = 8.8 Hz, 2 H), 7.33 (d, J = 8.8 Hz, 2 H), 7.42-7.47 (m, 1 H), 7.65- 7.68 (m, 1 H), 8.07- 8.08 (m, 1 H), 8.73 (s, 1 H). |
| 1r | 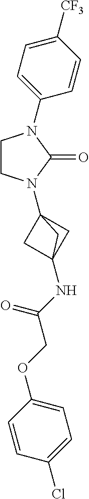 [SMILES: O=C(COc1ccc(Cl)cc1)NC12CC(N3CCN(c4ccc(C(F)(F)F)cc4)C3=O)(C1)C2] | 2-(4-chlorophenoxy)- N-(3-(2-oxo-3-(4- (trifluoromethyl)phenyl) imidazolidin-1- yl)bicyclo[1.1.1]pentan- 1-yl)acetamide | 479.88 | 2.48 (s, 6 H), 3.46 (t, J = 8.4 Hz, 2 H), 3.85 (t, J = 7.2 Hz, 2 H), 4.43 (s, 2 H), 6.96 (d, J = 8.8 Hz, 2 H), 7.33 (d, J = 8.8 Hz, 2 H), 7.74-7.62 (m, 4 H), 8.73 (s, 1 H). |
| 1s | 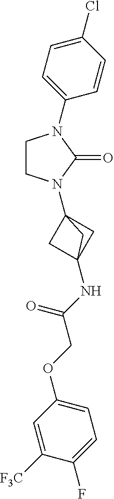 [SMILES: O=C(COc1ccc(F)c(C(F)(F)F)c1)NC12CC(N3CCN(c4ccc(Cl)cc4)C3=O)(C1)C2] | N-(3-(3-(4- chlorophenyl)-2- oxoimidazolidin-1- yl)bicyclo[1.1.1]pentan- 1-yl)-2-(4-fluoro-3- (trifluoromethyl)phe- noxy)acetamide | 498.0 | 2.29 (s, 6 H), 3.43 (t, J = 8.0 Hz, 2 H), 3.75 (t, J = 7.8 Hz, 2 H), 4.52 (s, 2 H), 7.30-7.34 (m, 4 H), 7.44 (t, J = 10.0 Hz, 1 H), 7.55 (d, J = 8.8 Hz, 2 H), 8.75 (s, 1 H). |
| 1t | 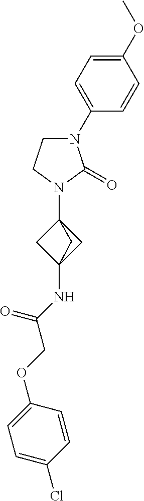 [SMILES: COc1ccc(N2CCN(C34CC(NC(=O)COc5ccc(Cl)cc5)(C3)C4)C2=O)cc1] | 2-(4-chlorophenoxy)- N-(3-(3-(4- methoxyphenyl)-2- oxoimidazolidin-1- yl)bicyclo[1.1.]pentan- 1-yl)acetamide | 442.1 | 2.27 (s, 6 H), 3.36-3.40 (m, 2 H), 3.70-3.73 (m, 5 H), 4.42 (s, 2 H), 6.86 (d, J = 9.2 Hz, 2 H), 6.96 (d, J = 8.8 Hz, 2 H), 7.32 (d, J = 8.8 Hz, 2 H), 7.40 (d, J = 9.2 Hz, 2 H), 8.70 (s, 1 H). |
| 1u | 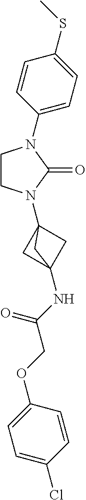 [SMILES: CSc1ccc(N2CCN(C34CC(NC(=O)COc5ccc(Cl)cc5)(C3)C4)C2=O)cc1] | 2-(4-chlorophenoxy)- N-(3-(3-(4- (methylthio)phenyl)-2- oxoimidazolidin-1- yl)bicyclo[1.1.1]pentan- 1-yl)acetamide | 458.0 | 2.28 (s, 6 H), 2.41 (s, 3 H), 3.41 (t, J = 8.0 Hz, 2 H), 3.73 (t, J = 8.4 Hz, 2 H), 4.42 (s, 2 H), 6.96 (d, J = 8.8 Hz, 2 H), 7.21 (d, J = 8.8 Hz, 2 H), 7.32 (d, J = 8.8 Hz, 2 H), 7.48 (d, J = 8.8 Hz, 2 H), 8.71 (s, 1 H). |

### Example 1v

### N-(3-(3-(4-chloro-2-fluorophenyl)-2-oxoimidazolidin-1-yl)bicyclo[1.1.1] pentan-1-yl)-2-(4-chlorophenoxy)acetamide

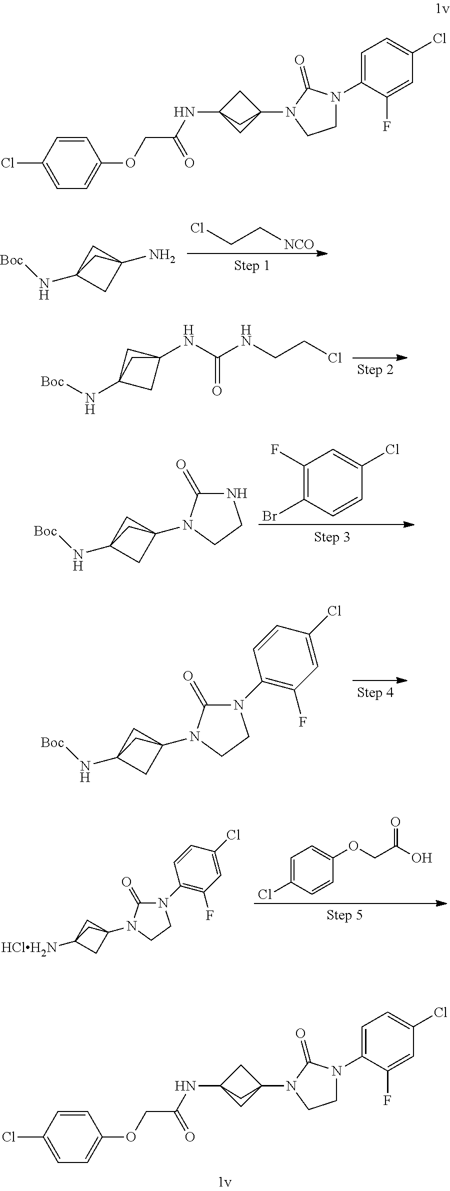

Step 1: To a stirred solution of tert-butyl (3-aminobicyclo[1.1.1]pentan-1-yl)carbamate (2 g, 10.08 mmol, 1.0 equiv) in chloroform (20 mL) was added 1-chloro-2-isocyanatoethane (1.29 mL, 15.13 mmol, 1.5 equiv) at 0° C. and the mixture was heated to 60° C. for 1 h. After the completion of the reaction (TLC in 5% Methanol in DCM), the reaction mixture was cooled to room temperature and concentrated under reduced pressure to yield crude product. The obtained crude was triturated with n-pentane (50 mL) and stirred for 0.5 h at room temperature. The solid compound was filtered and washed with n-pentane to afford tert-butyl (3-(3-(2-chloroethyl)ureido)bicyclo[1.1.1]pentan-1-yl)carbamate (2.5 g, 83% yield) as a white solid. LCMS (ES) m/z=304.1 [M+H]⁺; ¹H NMR (400 MHz, DMSO-d₆) δ ppm 1.35 (s, 9H), 2.01 (s, 6H), 3.25-3.24 (m, 2H), 3.52-3.53 (m, 2H), 5.93 (s, 1H), 6.66 (s, 1H), 7.41 (bs, 1H).

Step 2: To a stirred solution of tert-butyl (3-(3-(2-chloroethyl)ureido)bicyclo[1.1.1]pentan-1-yl)carbamate (2.5 g, 8.22 mmol, 1.0 equiv) in acetonitrile (25 mL) was added cesium carbonate (4.64 g, 14.24 mmol, 2.0 equiv) at room temperature and the mixture was heated to 80° C. and stirred for 12 h. After the completion of the reaction (TLC in 5% Methanol in DCM), the reaction mixture was allowed to cool to room temperature, diluted with water (100 mL) and extracted with ethyl acetate (2×50 mL). The combined ethyl acetate extract was dried over anhydrous Na₂SO₄, filtered and concentrated under reduced pressure to afford the crude product which was purified by silica gel column chromatography (Combiflash®) using 10% MeOH in dichloromethane as eluent to afford tert-butyl (3-(2-oxoimidazolidin-1-yl)bicyclo[1.1.1]pentan-1-yl)carbamate (0.4 g, 18% yield) as a white solid. ¹H NMR (400 MHz, DMSO-d₆) δ ppm 1.35 (s, 9H), 2.06 (s, 6H), 3.15-3.17 (m, 2H), 3.22-3.24 (m, 2H), 6.31 (s, 1H), 7.47 (bs, 1H).

Step 3: In a sealed tube, to a stirred solution of tert-butyl (3-(2-oxoimidazolidin-1-yl)bicyclo[1.1.1]pentan-1-yl)carbamate (0.25 g, 0.935 mmol, 1.0 equiv.) in 1,4-dioxane (5 mL) was added 1-bromo-4-chloro-2-fluorobenzene (0.19 g, 0.935 mmol, 1.0 equiv). The mixture was degassed by purging with argon for 5 minutes. Then Pd₂(dba)₃ (0.085 g, 0.093 mmol, 0.1 equiv), xanthphos (0.1 g, 0.187 mmol, 0.2 equiv) and CS₂CO₃ (1.21 g, 3.74 mmol, 4.0 equiv) were added to the reaction mixture under argon atmosphere, and the sealed tube was capped and the mixture was stirred at 80° C. for 16 h. The reaction mixture was cooled to room temperature, diluted with ethyl acetate (20 mL), filtered through a Celite® bed, and the Celite® bed washed with excess ethyl acetate. The filtrate was concentrated under reduced pressure to afford the crude product, which was purified by silica gel column chromatography (Combiflash®) using 50% Ethyl acetate in hexane as eluent to obtain tert-butyl 3-(3-(4-chloro-2-fluorophenyl)-2-oxoimidazolidin-1-yl)bicyclo[1.1.1]pentan-1-yl)carbamate (0.17 g, 65% yield) as an off-white solid. LCMS (ES) m/z=396.1 [M+H]⁺.

Step 4: To a stirred solution of tert-butyl (3-(3-(4-chloro-2-fluorophenyl)-2-oxoimidazolidin-1-yl)bicyclo[1.1.1]pentan-1-yl)carbamate (0.17 g, 0.42 mmol, 1.0 equiv.) in dichloromethane (2 mL) was added 4 M HCl solution in 1,4-dioxane (1.5 mL) at 0° C. The resulting mixture was allowed to warm to room temperature and stirred for 3 h. The progress of the reaction was monitored by TLC (30% ethyl acetate in hexane). After completion of reaction, the reaction mixture was concentrated under reduced pressure, and the obtained residue was triturated with n-pentane. The product was dried under high vaccum to obtain 1-(3-aminobicyclo[1.1.1]pentan-1-yl)-3-(4-chloro-2-fluorophenyl)imidazolidin-2-one hydrochloride (0.09 g, crude) as a pale brown solid. Used without further purification. LCMS (ES) m/z=296.1[M+H]⁺.

Step 5: To a stirred solution of 2-(4-chlorophenoxy)acetic acid (0.05 g, 0.27 mmol, 1 equiv.) and triethylamine (0.076 mL, 0.54 mmol, 2 equiv) in dichloromethane (10 mL) was added T3P® (50% wt. in ethyl acetate) at 0° C. The resulting mixture was allowed to warm to room temperature and stirred for 20 min. After 20 min, the reaction mixture was cooled to 0° C. and a solution of 1-(3-aminobicyclo[1.1.1]pentan-1-yl)-3-(4-chloro-2-fluorophenyl)imidazolidin-2-one hydrochloride (0.09 g crude, 0.27 mmol, 1.0 equiv) and triethylamine (0.11 mL, 0.81 mmol, 3 equiv) in dichloromethane (10 mL) was added at 0° C. The resulting mixture was allowed to warm to room temperature and stirred for 3 h. The progress of the reaction was monitored by TLC (5% methanol in DCM). After completion of the reaction, the reaction mixture was diluted with dichloromethane (100 mL), washed with saturated aqueous sodium bicarbonate solution (50 mL), water (30 mL) and brine (30 mL), and finally dried over anhydrous sodium sulfate, filtered and concentrated under reduced pressure. The crude product was purified by silica gel column chromatography (Combiflash®) using 4% methanol in dichloromethane as eluent to obtain the title compound N-(3-(3-(4-chloro-2-fluorophenyl)-2-oxoimidazolidin-1-yl)bicyclo[1.1.1]pentan-1-yl)-2-(4-chlorophenoxy)acetamide (0.08 g) as a white solid. LCMS (ES) m/z=464.0 [M+H]⁺; ¹H NMR (400 MHz, DMSO-d₆) δ ppm 2.27 (s, 6H), 3.44 (t, J=7.6 Hz, 2H), 3.73 (t, J=7.2 Hz, 2H), 4.42 (s, 2H), 6.96 (d, J=8.8 Hz, 2H), 7.25 (d, J=7.2 Hz, 1H), 7.32 (d, J=8.8 Hz, 2H), 7.45-7.52 (m, 2H), 8.70 (s, 1H).

TABLE 3

| | | | LCMS | |
|---|---|---|---|---|
| | | | m/z | ¹H-NMR (400 MHz, |
| Cmpd # | Structure | Name | [M + H]⁺ | DMSO-d₆) |
| 1v | 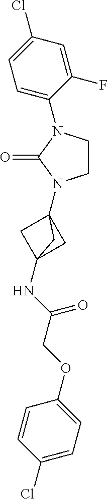 [SMILES: O=C(COc1ccc(Cl)cc1)NC12CC(N3CCN(c4ccc(Cl)cc4F)C3=O)(C1)C2] | N-(3-(3-(4-chloro-2- fluorophenyl)-2- oxoimidazolidin-1- yl)bicyclo[1.1.1]pentan-1- yl)-2-(4- chlorophenoxy)acetamide | 464.0 | 2.27 (s, 6 H), 3.44 (t, J = 7.6 Hz, 2 H), 3.73 (t, J = 7.2 Hz, 2 H), 4.42 (s, 2 H), 6.96 (d, J = 8.8 Hz, 2 H), 7.25 (d, J = 7.2 Hz, 1 H), 7.32 (d, J = 8.8 Hz, 2 H), 7.45-7.52 (m, 2 H), 8.70 (s, 1 H). |

### Example 2a

### N-(4-(2-(4-chlorophenoxy)acetamido)bicyclo[2.1.1]hexan-1-yl)-2-(4-chlorophenyl)cyclopropane-1-carboxamide

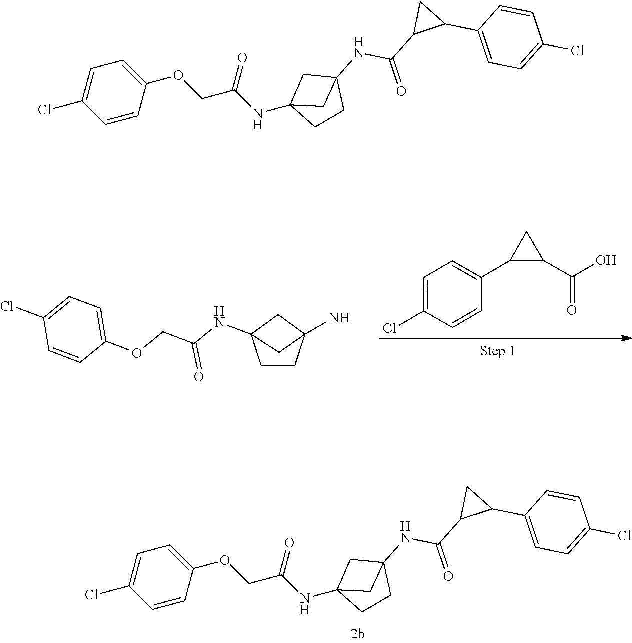 [SMILES: O=C(COc1ccc(Cl)cc1)NC12CCC(NC(=O)C3CC3c3ccc(Cl)cc3)(C1)C2 ; NC12CCC(NC(=O)COc3ccc(Cl)cc3)(C1)C2 ; O=C(O)C1CC1c1ccc(Cl)cc1 ; O=C(COc1ccc(Cl)cc1)NC12CCC(NC(=O)C3CC3c3ccc(Cl)cc3)(C1)C2]

Step 1: To a solution of 2-(4-chlorophenyl)cyclopropane-1-carboxylic acid (0.06 g, 0.29 mmol, 1.2 equiv.) in DCM (5 mL) at 0° C. was added triethylamine (0.14 mL, 1.00 mmol, 4.0 equiv), and stirred for 10 min followed by addition of T3P®® (50 wt % in EtOAc) (0.3 mL, 0.50 mmol, 2.0 equiv.). The reaction mixture was stirred at 0° C. for 10 min and then N-(4-aminobicyclo[2.1.1]hexan-1-yl)-2-(4-chlorophenoxy)acetamide (0.07 g, 0.25 mmol, 1.0 equiv.) was added at 0° C. and the reaction was stirred at rt for 16 h. After the consumption of the starting material (TLC, 5% MeOH in DCM), the reaction mixture was diluted with DCM (50 mL) and was washed with saturated aqueous NaHCO₃ solution (2×10 mL) and water (2×10 mL). The combined organic layer was dried over anhydrous Na₂SO₄, filtered and evaporated under reduced pressure to get the crude. The crude product was purified by silica gel column chromatography using 1-2% MeOH in DCM as eluent to afford the title compound which was then triturated with n-pentane (2×5 mL). The solid was dried under high vacuum to afford the desired product N-(4-(2-(4-chlorophenoxy)acetamido)bicyclo[2.1.1]hexan-1-yl)-2-(4-chlorophenyl)cyclopropane-1-carboxamide as an off-white solid (0.07 g, 61.4%). LCMS (ES) m/z=459.3 [M+H]⁺. ¹H NMR (400 MHz, DMSO-d₆) δ ppm 1.11-1.15 (m, 1H), 1.28-1.33 (m, 1H), 1.72-1.75 (m, 6H), 1.79-1.83 (m, 1H), 2.06 (s, 2H), 2.19-2.23 (m, 1H), 4.40 (s, 2H), 6.95 (d, J=8.8 Hz, 2H), 7.13 (d, J=8.0 Hz, 2H), 7.31 (t, J=9.2 Hz, 4H), 8.40 (s, 1H), 8.48 (s, 1H).

The compound of Example 2b was prepared generally according to the procedure described above for Example 2a.

TABLE 4

| | | | LCMS | |
|---|---|---|---|---|
| | | | m/z | ¹H-NMR (400 MHz, |
| Cmpd # | Structure | Name | [M + H]⁺ | DMSO-d₆) |
| 2a | 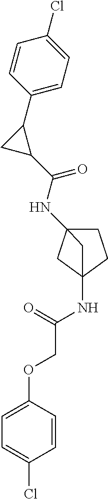 [SMILES: O=C(COc1ccc(Cl)cc1)NC12CCC(NC(=O)C3CC3c3ccc(Cl)cc3)(C1)C2] | N-(4-(2-(4- chlorophenoxy)acetamido) bicyclo[2.1.1]hexan-1-yl)-2- (4- chlorophenyl)cyclopropane- 1-carboxamide | 459.3 | 1.11-1.15 (m, 1H), 1.28-1.33 (m, 1 H), 1.72-1.75 (m, 6 H), 1.79-1.83 (m, 1 H), 2.06 (s, 2 H), 2.19- 2.23 (m, 1 H), 4.40 (s, 2 H), 6.95 (d, J = 8.8 Hz, 2 H), 7.13 (d, J = 8.0 Hz, 2 H), 7.31 (t, J = 9.2 Hz, 4 H), 8.40 (s, 1 H), 8.48 (s, 1 H). |
| 2b and XXIVB | 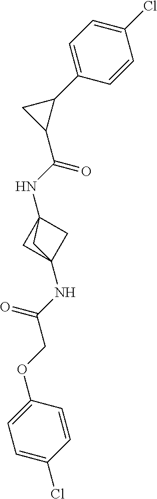 [SMILES: O=C(COc1ccc(Cl)cc1)NC12CC(NC(=O)C3CC3c3ccc(Cl)cc3)(C1)C2] | N-(3-(2-(4- chlorophenoxy)acetamido) bicyclo[1.1.1]pentan-1-yl)- 2-(4- chlorophenyl)cyclopropane- 1-carboxamide | 445.3 | 1.16-1.20 (m, 1 H), 1.30-1.32 (m, 1 H), 1.74-1.76 (m, 1 H), 2.30 (s, 7 H), 4.40 (s, 2 H), 6.95 (d, J = 8.8 Hz, 2 H), 7.12 (d, J = 8.4 Hz, 2 H), 7.28- 7.33 (m, 4 H), 8.64 (bs, 1 H), 8.69 (bs, 1 H). |

### Example 2c and XXIVA

### 2-(4-chlorophenoxy)-N-(3-(2-(4-chlorophenoxy)acetamido)bicyclo[1.1.1]pentan-1-yl)cyclopropane-1-carboxamide

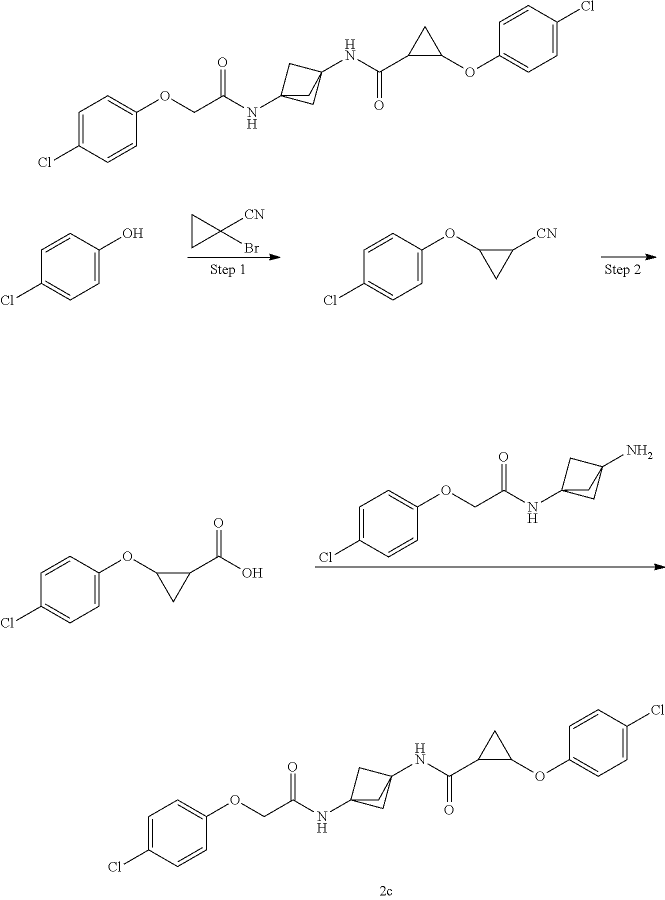

Step 1: To a stirred solution of 4-chlorophenol (0.5 g, 3.90 mmol, 1 equiv) in DMF (15 mL) at room temperature was added cesium carbonate (1.98 g, 5.85 mmol, 1.5 equiv.) and 1-bromocyclopropane-1-carbonitrile (0.57 g, 3.90 mmol, 1 equiv). The resulting mixture was heated to 90° C. and stirred for 16 h. The progress of the reaction was monitored by TLC. After consumption of the starting material, the reaction mixture was cooled to room temperature, diluted with water (50 mL) and extracted with EtOAc (2×100 mL). The combined organic layer was washed with brine (50 mL), dried over anhydrous sodium sulfate, filtered and concentrated under reduced pressure to afford the crude product. The crude product was purified by flash column chromatography using a silica gel column (10% ethyl acetate in hexane) to afford 2-(4-chlorophenoxy)cyclopropane-1-carbonitrile (0.15 g, 20% yield) as an off-white solid. ¹H NMR (400 MHz, DMSO-d₆): δ ppm 1.47-1.49 (m, 1H), 1.54-1.59 (m, 1H), 2.13-2.16 (m, 1H), 4.40-4.49 (s, 1H), 7.04 (d, J=8.8 Hz, 2H), 7.4 (d, J=8.8 Hz, 2H). ¹³C NMR (100 MHz, DMSO-d₆): δ ppm 4.47, 14.40, 55.86, 117.19, 120.17, 126.29, 129.93, 156.63.

Step 2: To a stirred solution of 2-(4-chlorophenoxy)cyclopropane-1-carbonitrile (0.15 g, 0.773 mmol, 1 equiv) in water (5 mL) was added a 10% aqueous solution of NaOH (5 mL) and the resulting mixture was heated to 70° C. for 12 h. The progress of the reaction was monitored by TLC. After consumption of the starting material the reaction mixture was cooled to room temperature and acidified with 1 N HCl to pH ˜2. The product was extracted with EtOAc (2×50 mL). The combined organic layer was washed with brine solution (50 mL), dried over anhydrous sodium sulfate, filtered and concentrated under reduced pressure to afford 2-(4-chlorophenoxy)cyclopropane-1-carboxylic acid (0.085 g, 50% yield) as sticky solid. LCMS (ES) m/z=212.2 [M−H]⁺. ¹H NMR (400 MHz, DMSO-d₆): δ ppm 1.32-1.36 (m, 1H), 1.36-1.41 (m, 1H), 1.92-1.97 (m, 1H), 4.09-4.11(m, 1H), 7.03 (d, J=8.4 Hz, 2H), 7.32 (d, J=8.4 Hz, 2H), 12.03 (bs, 1H).

Step 3: To a stirred solution of 2-(4-chlorophenoxy)cyclopropane-1-carboxylic acid (0.083 g, 0.393 mmol, 1.5 equiv) and triethylamine (0.15 mL, 1.04 mmol, 4.0 equiv) in dichloromethane (5 mL) was added T3P® (50 wt. % in ethyl acetate) (0.25 mL, 0.393 mmol, 1.5 equiv) at 0° C. and the mixture was stirred for 10 min. Then N-(3-aminobicyclo[1.1.1]pentan-1-yl)-2-(4-chlorophenoxy)acetamide (0.07 g, 0.262 mmol, 1 equiv) was added to the above reaction mixture. The resulting mixture was stirred for 16 h during which it warmed up to room temperature. The progress of the reaction was monitored by TLC. After completion of the reaction, the mixture was diluted with dichloromethane (50 mL), washed with water (2×30 mL), brine (30 mL), dried over anhydrous sodium sulfate, filtered and concentrated under reduced pressure to give the crude product. The crude product was purified by silica gel flash column chromatography using 4% methanol in dichloromethane as eluent. The obtained product was re-purified by preparative HPLC [Analytical condition: Column: ZORBAX (150 mm×4.6 mm×5 mic), mobile phase(A): 0.1% Ammonia in water, mobile phase(B): CH₃CN, flow rate: 1.0 mL/min, gradient: 0/10,10/60, 25/90, 27/10, 30/10] to afford the title compound 2-(4-chlorophenoxy)-N-(3-(2-(4-chlorophenoxy)acetamido)bicyclo[1.1.1]pentan-1-yl)cyclopropane-1-carboxamide (0.03 g, 25% yield) as a white solid. LCMS (ES) m/z=461.3 [M+H]⁺. ¹H NMR (400 MHz, DMSO-d₆): δ ppm 1.10-1.13 (m, 1H), 1.39-1.40 9m, 1H), 1.84-1.88 (m, 1H), 1.94-2.00 (m, 6H), 3.98-3.99 (m, 1H), 4.37 (s, 2H), 6.92-7.00 (m, 4H), 7.25-7.32 (m, 4H), 8.45 (s, 1H), 8.56 (s, 1H). ¹³C NMR (100 MHz, DMSO-d₆): δ ppm 10.88, 22.47, 44.59, 44.96, 54.74, 55.61, 67.47, 116.95, 117.26, 125.11, 125.27, 129.24, 129.62, 157.08, 157.49, 167.27, 168.07. HPLC Purity 99.79% at 225 nm.

TABLE 5

| | | | LCMS | |
|---|---|---|---|---|
| | | | m/z | |
| Cmpd # | Structure | Name | [M + H]⁺ | ¹H-NMR (400 MHz, DMSO-d₆) |
| 2c | 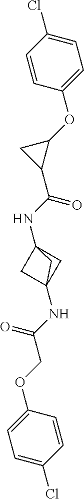 [SMILES: O=C(COc1ccc(Cl)cc1)NC12CC(NC(=O)C3CC3Oc3ccc(Cl)cc3)(C1)C2] | 2-(4-chlorophenoxy)- N-(3-(2-(4- chlorophenoxy)aceta- mido)bicyclo[1.1.1] pentan-1- yl)cyclopropane-1- carboxamide | 461.3 | 1.10-1.13 (m, 1 H), 1.39- 1.40 (m, 1 H), 1.84-1.88 (m, 1 H), 1.94-2.00 (m, 6 H), 3.98- 3.99 (m, 1 H), 4.37 (s, 2 H), 6.92-7.0 (m, 4 H), 7.25-7.32 (m, 4 H), 8.45 (s, 1 H), 8.56 (s, 1 H). |

### Example 3a

### 2-(4-chlorophenoxy)-N-(3-((1-(4-chlorophenyl)azetidin-3-yl)amino)bicyclo[1.1.1]pentan-1-yl)acetamide

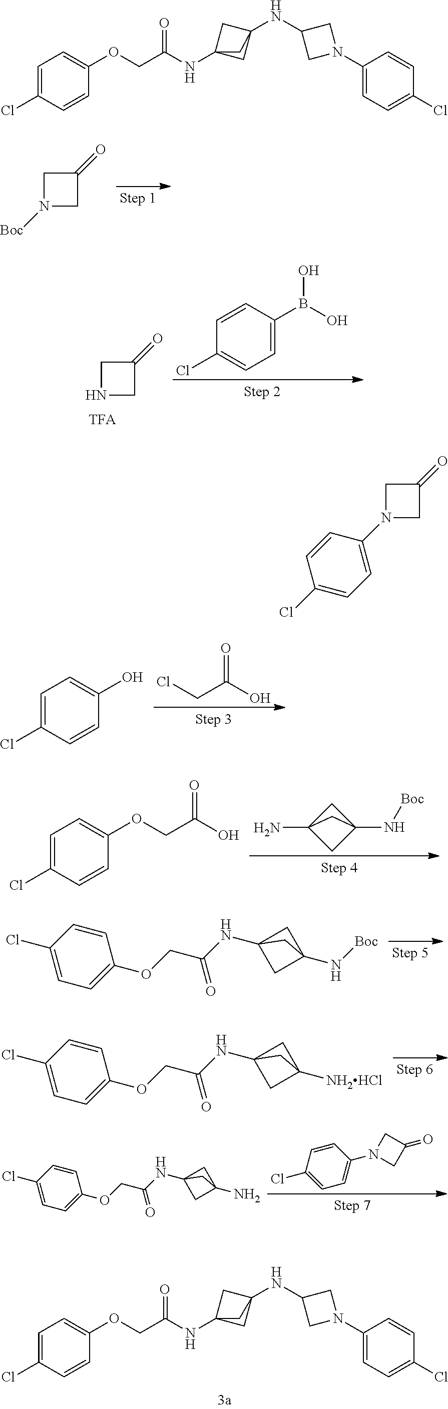

Step 1: Tert-butyl 3-oxoazetidine-1-carboxylate (4.0 g, 23.364 mmol, 1 equiv) at 0° C. was treated with TFA (8 mL). After the reaction mixture was stirred at 0° C. for 4 h, the reaction mixture was concentrated to give crude azetidin-3-one 2,2,2-trifluoroacetate (5.1 g, crude) as a light yellow gum. LCMS (ES) m/z=72.0 [M+H]⁺. ¹H NMR (400 MHz, DMSO-d6): δ ppm 5.01 (s, 4H), 9.49 (s, 2H).

Step 2: To a stirred solution of azetidin-3-one 2,2,2-trifluoroacetate (3.0 g, 16.189 mmol, 1 equiv, this 3 g was performed as 5×0.600 g batches) in DCM (30 mL), was added triethylamine (9.10 mL, 64.75 mmol, 4.0 equiv) followed by copper(II)acetate (5.88 g 32.37 mmol, 2 equiv), and purged with air for 45 minutes. Then (4-chlorophenyl)boronic acid was added and again purged with air for 10 min. The reaction mixture stirred at room temperature for 5 h. After completion of the reaction, the reaction mixture was filtered through a Celite® bed which was washed with DCM. The filtrate was concentrated under reduced pressure to give the crude product, which was purified by silica gel column chromatography (20% ethylacetate in n-Hexane) to afford the title compound 1-(4-chlorophenyl)azetidin-3-one (0.042 g, 1.43% yield) as an off-white solid. LCMS (ES) m/z=181.9 [M+H]⁺.¹H NMR (400 MHz, CDCl3): δ ppm 4.65 (s, 4H), 6.51 (d, J=8.4 Hz, 2H), 7.23-7.25 (m, 2H).

Step 3: To a solution of 4-chlorophenol (117 g, 914.06 mmol, 1 equiv) in water (1200 mL) at 0° C. was added a solution of sodium hydroxide (146.25 g, 3656.25 mmol, 4 equiv), and stirred at 0° C. for 15 min. Then 4-chloroacetic acid (120.9 g, 1279.68 mmol, 1.4 equiv) was added portion-wise at 0° C. and stirred for 10 min at the same temperature. Then the reaction mixture was heated at 100° C. for 12 h. After consumption of the starting material (TLC, 5% methanol in DCM), the reaction mixture was allowed to cool to room temperature. The reaction mixture was diluted with water (200 mL), and the aqueous layer was washed with ethyl acetate (2×150 mL). The aqueous layer was acidified with conc. HCl up to pH=1 and the precipitated product was filtered through a sintered funnel, washed with ice-cold water (100 mL), n-hexane (300 mL), and dried under high vacuum to afford 2-(4-chlorophenoxy)acetic acid (68.0 g, 40% yield) as a white solid. LCMS (ES) m/z=185 [M+H]⁺. ¹H NMR (400 MHz, DMSO-d₆) δ ppm 4.65 (s, 2H), 6.91 (d, J=8.8 Hz, 2 H), 7.30 (d, J=8.8 Hz, 2H), 12.99 (bs, 1H).

Step 4: To a solution of 2-(4-chlorophenoxy)acetic acid (33.87 g, 181.57 mmol, 1.2 equiv) in DCM (300 mL) at 0° C. was added triethylamine (63.35 mL, 453.93 mmol, 3 equiv) and was stirred for 5 minutes at 0° C. T3P® (50 wt. % in ethyl acetate) (135.1 mL, 226.96 mmol, 1.5 equiv) was added and the reaction mixture was stirred at 0° C. for 10 mins. Then tert-butyl (3-aminobicyclo[1.1.1]pentan-1-yl)carbamate (30.0 g, 151.31 mmol, 1 equiv) was added to the reaction mixture, and the reaction mixture was stirred at room temperature for 16 hours. After completion of the reaction, the reaction mixture was diluted with water (200 mL) and extracted with DCM (2×300 mL). The combined organic layer was washed with saturated sodium bicarbonate solution (200 mL), filtered and concentrated under reduced pressure to afford the product. Following the same procedure another 30 g batch reaction of tert-butyl (3-aminobicyclo[1.1.1]pentan-1-yl)carbamate was performed to give the final combined yield of 108 g of tert-butyl (3-(2-(4-chlorophenoxy)acetamido)bicyclo[1.1.1]pentan-1-yl)carbamate (97.24% yield) as an off-white solid. LCMS (ES) m/z=311.1 [M+H]⁺ (t-butyl cleavage mass was observed). ¹H NMR (400 MHz, DMSO-d₆) δ ppm 1.35 (s, 9H), 2.11 (s, 6H), 4.39 (s, 2H), 6.94 (d, J=8.8 Hz, 2H), 7.31 (d, J=8.8 Hz, 2H), 7.46 (bs, 1H), 8.60 (s, 1H).

Step 5: To a solution of tert-butyl (3-(2-(4-chlorophenoxy)acetamido)bicyclo[1.1.1]pentan-1-yl)carbamate (27 g, 73.57 mmol, 1 equiv) in DCM (400 mL) was added 4M HCl in 1,4-dioxane (90 mL) at 0° C. and the reaction mixture stirred at room temperature for 12 h. After consumption of the starting material (TLC, 5% methanol in DCM), DCM was evaporated under reduced pressure, and the obtained solid was triturated with diethyl ether (300 mL) and dried under high vacuum to afford N-(3-aminobicyclo[1.1.1]pentan-1-yl)-2-(4-chlorophenoxy)acetamide hydrochloride. Following the same procedure another 3 batches were performed to give a total of 84 g (94.52% yield) of N-(3-aminobicyclo[1.1.1]pentan-1-yl)-2-(4-chlorophenoxy)acetamide hydrochloride as an off-white solid. From this, 29.7 g was blended with 105.6 g, prepared by following a similar procedure, by dissolving in 500 mL of DCM and finally concentrated under reduced pressure to afford the product as an off-white solid (135.3 g crude). LCMS (ES) m/z=not ionised. ¹H NMR (400 MHz, DMSO-d₆) δ ppm 2.22 (s, 6H), 4.44 (s, 2H), 6.95 (d, J=8.8 Hz, 2H), 7.32 (d, J=9.2 Hz, 2H), 8.87 (s, 1H), 9.0 (bs, 3H).

Step 6: To a stirred solution of N-(3-aminobicyclo[1.1.1]pentan-1-yl)-2-(4-chlorophenoxy)acetamide hydrochloride (15 .0 g, 49.66 mmol, 1 equiv) in ethyl acetate (200 mL) at room temperature was added saturated sodium bicarbonate (300 mL). After 30 min at room temperature, the reaction mixture was extracted with ethyl acetate (2×250 mL). The combined organic extract was washed with water (100 mL) and brine (50 mL), dried over anhydrous sodium sulfate, filtered and concentrated under reduced pressure to afford the crude N-(3-aminobicyclo[1.1.1]pentan-1-yl)-2-(4-chlorophenoxy)acetamide (13 g, crude) as a brown gum. 5 g of the crude material was further purified by using reversed phase HPLC purification [Column: C18, Mobile phase (A): 0.1% ammonia in water, Mobile phase (B): Acetonitrile]. The obtained material was stirred in n-pentane (40 mL) at room temperature for 1 h. Then the solid was filtered and dried to give N-(3-aminobicyclo[1.1.1]pentan-1-yl)-2-(4-chlorophenoxy)acetamide (3.0 g, 60% yield) as a white solid. LCMS (ES) m/z=267.1 [M+H]⁺. ¹H NMR (400 MHz, DMSO-d₆) δ ppm 1.90 (s, 6H), 2.11 (s, 2H), 4.37 (s, 2H), 6.93 (d, J=8.8 Hz, 2H), 7.30-7.32 (m, 2H), 8.47 (s, 1H).

Step 7: To a stirred solution of N-(3-aminobicyclo[1.1.1]pentan-1-yl)-2-(4-chlorophenoxy)acetamide (0.070 g, 0.262 mmol, 1 equiv) in methanol (3 mL) at room temperature, 1-(4-chlorophenyl)azetidin-3-one (0.052 g, 0.288 mmol, 1.1 equiv) and acetic acid (0.05 mL) were added. After stirring for 45 minutes at room temperature, the reaction mixture was cooled to 0° C. and sodium cyanoborohydride (0.032 g, 0.524 mmol, 2 equiv) was added. After the reaction stirred at room temperature for 4 h, the solvent was evaporated under reduced pressure. Obtained crude was diluted with water (5 mL) and extracted with ethyl acetate (2×10 mL). The combined organic extract was washed with water (10 mL) and brine (5 mL). The organic phase was dried over anhydrous sodium sulfate, filtered, and concentrated under reduced pressure to afford the crude product. The crude material was purified by flash column chromatography using a silica gel column where the product was eluted along with a polar impurity at 2-3% methanol in DCM. This mixture was further purified by using preparative TLC (2.5% methanol in DCM) to afford 2-(4-chlorophenoxy)-N-(3-((1-(4-chlorophenyl)azetidin-3-yl)amino)bicyclo[1.1.1]pentan-1-yl)acetamide (0.028 g, 24.77% yield) as an off-white solid. LCMS (ES) m/z=432.3 [M+H]⁺. ¹H NMR (400 MHz, DMSO-d₆) δ ppm 1.93 (s, 6H), 3.12 (d, J=10.0 Hz, 1H), 3.37 (t, J=6.8 Hz, 2H), 3.62-3.68 (m, 1H), 4.01 (t, J=6.8 Hz, 2H), 4.44 (s, 2H), 6.38 (d, J=8.8 Hz, 2H), 6.94 (d, J=8.8 Hz, 2H), 7.14 (d, J=8.4 Hz, 2H), 7.32 (d, J=8.8 Hz, 2H), 8.56 (s, 1H).

TABLE 6

| | | | LCMS | |
|---|---|---|---|---|
| | | | m/z | ¹H-NMR (400 MHz, DMSO- |
| Cmpd # | Structure | Name | [M + H]⁺ | d₆) |
| 3a | 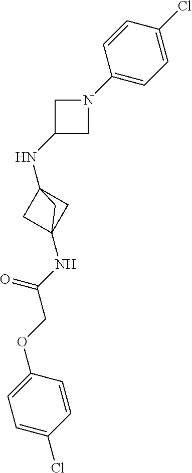 [SMILES: O=C(COc1ccc(Cl)cc1)NC12CC(NC3CN(c4ccc(Cl)cc4)C3)(C1)C2] | 2-(4-chlorophenoxy)-N-(3- ((1-(4-chlorophenyl) azetidin-3-yl)amino)bi- cyclo[1.1.1]pentan- 1-yl)acetamide | 432.3 | 1.93 (s, 6 H), 3.12 (d, J = 10.0 Hz, 1 H), 3.37 (t, J = 6.8 Hz, 2 H), 3.62-3.68 (m, 1 H), 4.01 (t, J = 6.8 Hz, 2 H), 4.44 (s, 2 H), 6.38 (d, J = 8.8 Hz, 2 H), 6.94 (d, J = 8.8 Hz, 2 H), 7.14 (d, J = 8.4 Hz, 2 H), 7.32 (d, J = 8.8 Hz, 2 H), 8.56 (s, 1 H). |

### Example 3b

### 2-(4-chlorophenoxy)-N-(3-(3-(4-chlorophenoxy)azetidin-1-yl)bicyclo[1.1.1]pentan-1-yl)acetamide

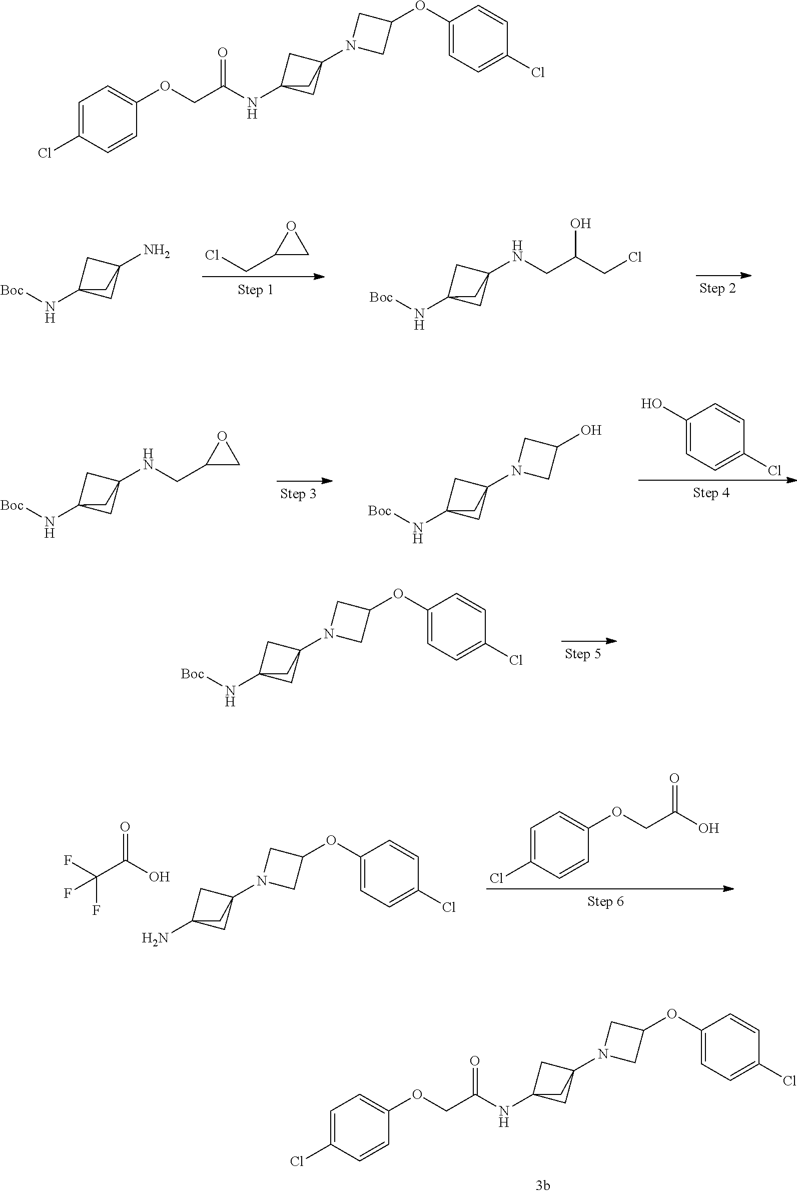

Step 1: To a solution of tert-butyl (3-aminobicyclo[1.1.1]pentan-1-yl)carbamate (3.0 g, 15.15 mmol, 1 equiv) in IPA (30 mL) was added 2-(chloromethyl)oxirane (1.4 g, 15.15 mmol, 1 equiv) at 0° C. and the reaction mixture was stirred at room temperature for 48 h. After this time, solvent was evaparated under reduced pressure to give tert-butyl (3-((3-chloro-2-hydroxypropyl)amino)bicyclo[1.1.1]pentan-1-yl)carbamate (crude yield 4.4 g, 100%), which was taken to the next step without further purification. LCMS (ES) m/z=291 [M+H]⁺.

Step 2: To a solution of tert-butyl (3-((3-chloro-2-hydroxypropyl)amino)bicyclo[1.1.1]pentan-1-yl)carbamate (4.4 g, 15.20 mmol, 1 equiv) in diethyl ether (45 mL) was added potassium hydroxide (1.7 g, 30.40 mmol, 2 equiv) at 0° C. After the reaction mixture was stirred at room temperature for 16 h, solvent was evaporated under reduced pressure to afford the crude product. The obtained crude product was purified by Combiflash® using a 40 g silica gel cartridge with gradient elution of 0% MeOH in DCM to 5% MeOH/DCM over a 30 min period to afford tert-butyl (3-((oxiran-2-ylmethyl)amino)bicyclo[1.1.1]pentan-1-yl)carbamate (colorless syrup, 1.27 g, 33%). LCMS (ES) m/z=255 [M+H]⁺. ¹H NMR (400 MHz, CDCl₃) δ ppm 1.43 (s, 9H), 2.02 (s, 6H), 2.58-2.63 (m, 2H), 2.76-2.78 (m, 1H), 2.89-2.93 (m, 1H), 3.05-3.08 (m, 1H), 4.88 (bs, 1H).

Step 3: To a solution of tert-butyl (3-((oxiran-2-ylmethyl)amino)bicyclo[1.1.1]pentan-1-yl)carbamate (4.4 g, 15.20 mmol, 1 equiv) in 1,4-dioxane (45 mL) was added magnesium bromide (1.7 g, 30.40 mmol, 2 equiv) at room temperature. After the reaction mixture was stirred at 90° C. for 16 h, the solvent was evaporated under reduced pressure to afford the crude product. The obtained crude product was purified by Combiflash® using a 24 g silica gel cartridge with gradient elution of 0% MeOH in DCM to 5% MeOH/DCM over a 30 min period to afford tert-butyl (3-(3-hydroxyazetidin-1-yl)bicyclo[1.1.1]pentan-1-yl)carbamate (colorless syrup, 0.42 g, 32%). LCMS (ES) m/z=255 [M+H]⁺, ¹H NMR (400 MHz, CDCl₃) δ ppm 1.43 (s, 9H), 1.95 (s, 6H), 2.99-3.02 (m, 2H), 3.54-3.57 (m, 2H), 4.40-4.54 (m, 1H), 4.89 (bs, 1H).

Step 4: To a solution of tert-butyl (3-(3-hydroxyazetidin-1-yl)bicyclo[1.1.1]pentan-1-yl)carbamate (0.4 g, 1.57 mmol, 1 equiv) and 4-chlorophenol (0.22 g, 1.73 mmol, 1.1 equiv) in DCM (10 mL) was added triphenylphosphine (0.61 g, 2.35 mmol, 1.5 equiv) followed by DIAD (0.47 g, 2.35 mmol, 1.5 equiv) at 0° C. After the reaction mixture was stirred at room temperature for 16 h, the solvent was evaporated under reduced pressure to afford the crude product. The obtained crude product was purified by Combiflash® using a 24 g silica gel cartridge with gradient elution of 0% MeOH in DCM to 5% MeOH/DCM over a 30 min period to afford tert-butyl (3-(3-(4-chlorophenoxy)azetidin-1-yl)bicyclo[1.1.1]pentan-1-yl)carbamate (0.4 g). LCMS (ES) m/z=365 [M+H]⁺. ¹H NMR (400 MHz, DMSO-d₆) δ ppm 1.34 (s, 9H), 1.78 (s, 6H), 3.01-3.04 (m, 2H), 3.58-3.63 (m, 2H), 4.72-4.75 (m, 1H), 6.82 (d, J=8.8 Hz, 2H), 7.28 (d, J=8.4 Hz, 2H).

Step 5: To a solution of tert-butyl (3-(3-(4-chlorophenoxy)azetidin-1-yl)bicyclo[1.1.1]pentan-1-yl)carbamate (0.4 g, 1.09 mmol, 1 equiv) in DCM (4 mL) was added 2,2,2-trifluoroacetic acid (2 mL) at 0° C. After the reaction mixture was stirred at room temperature for 16 h, the solvent was evaporated under reduced pressure to afford the crude product. The obtained crude product was triturated with diethyl ether (3×25 mL) to give 3-(3-(4-chlorophenoxy)azetidin-1-yl)bicyclo[1.1.1]pentan-1-amine 2,2,2-trifluoroacetate (0.21 g). LCMS (ES) m/z=265.1 [M+H]⁺. ¹H NMR (400 MHz, DMSO-d₆) δ ppm 2.05 (s, 6H), 3.49 (bs, 2H), 3.99 (bs, 2H), 4.87 (bs, 1H), 6.85 (d, J=8.4 Hz, 2H), 7.32 (d, J=8.8 Hz, 2H), 8.69 (bs, 3H).

Step 6: To a solution of 2-(4-chlorophenoxy)acetic acid (0.12 g, 0.63 mmol, 1.2 equiv) in dichloromethane (4 mL) was added triethylamine (0.11 g, 1.04 mmol, 2.0 equiv) at room temperature. After stirring for 5 min, T3P® (50 wt. % in ethyl acetate) (0.24 g, 0.78 mmol, 1.5 equiv) was added to the reaction mixture at room temperature and stirred for 10 min followed by 3-(3-(4-chlorophenoxy)azetidin-1-yl)bicyclo[1.1.1]pentan-1-amine 2,2,2-trifluoroacetate (0.2 g, 0.52 mmol, 1.0 equiv) and triethylamine (0.1 g, 1.04 mmol, 2.0 equiv) in DCM (3 mL). After the reaction mixture stirred at room temperature for 16 h, the reaction mixture was diluted with water (10 mL) and extracted with DCM (2×25 mL). The combined organic extract was washed with saturated aqueous NaHCO₃ solution (10 mL), brine solution (7 mL), dried over anhydrous sodium sulfate, filtered and concentrated under reduced pressure to give the crude product. The crude material was purified by flash column chromatography using a silica gel column where the product along with a very close impurity was eluted at 2-3% methanol in DCM. Finally, this crude product was purified by preparative HPLC to afford 2-(4-chlorophenoxy)-N-(3-(3-(4-chlorophenoxy)azetidin-1-yl)bicyclo[1.1.1]pentan-1-yl)acetamide (0.04 g, 17.5% yield) as a white solid. LCMS (ES) m/z=433.3 [M+H]⁺. ¹H NMR (400 MHz, DMSO-d₆) δ ppm 1.91 (s, 6H), 3.04-3.08 (m, 2H), 3.62-3.63 (m, 2H), 4.40 (s, 2H), 4.74-4.76 (m, 1H), 6.83 (d, J=8.4 Hz, 2H), 6.94 (d, J=8.4 Hz, 2H), 7.28-7.32 (m, 4H), 8.64 (s, 1H).

TABLE 7

| | | | LCMS | |
|---|---|---|---|---|
| | | | m/z | |
| Cmpd # | Structure | Name | [M + H]⁺ | ¹H-NMR (400 MHz, DMSO-d₆) |
| 3b | 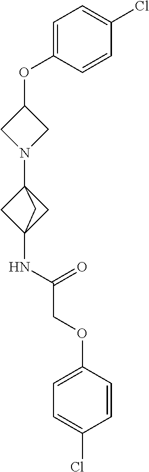 [SMILES: O=C(COc1ccc(Cl)cc1)NC12CC(N3CC(Oc4ccc(Cl)cc4)C3)(C1)C2] | 2-(4- chlorophenoxy)- N-(3-(3-(4- chlorophenoxy)azeti- din-1-yl)bi- cyclo[1.1.1]pentan- 1-yl)acetamide | 433.3 | 1.91 (s, 6 H), 3.04-3.08 (m, 2 H), 3.62-3.63 (m, 2 H), 4.40 (s, 2 H), 4.74-4.76 (m, 1 H), 6.83 (d, J = 8.4 Hz, 2 H), 6.94 (d, J = 8.4 Hz, 2 H), 7.28-7.32 (m, 4 H), 8.64 (s, 1 H). |

### Example 4a

### 2-(4-chlorophenoxy)-N-(3-(2-((5,6,7,8-tetrahydronaphthalen-2-yl)oxy)acetamido)bicyclo[1.1.1]pentan-1-yl)acetamide

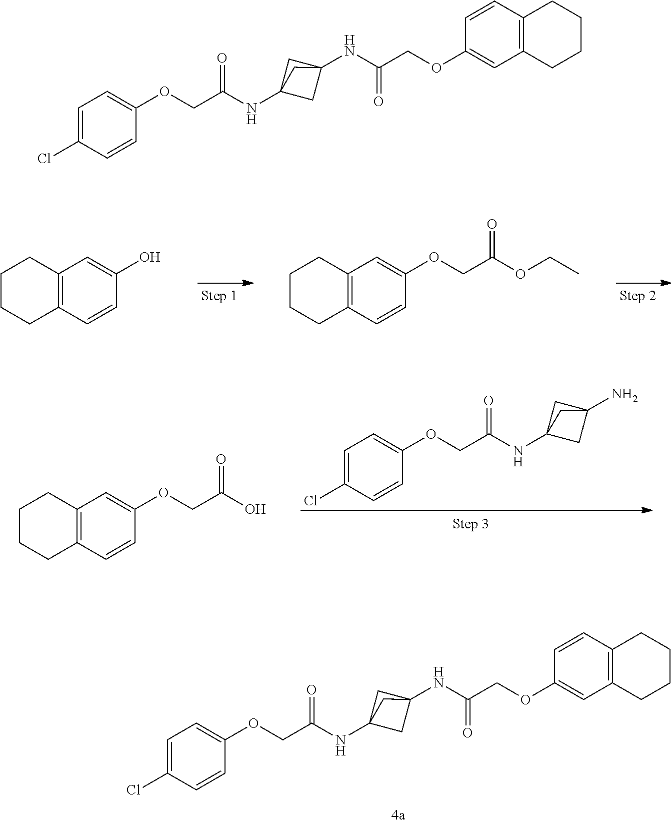 [SMILES: O=C(COc1ccc(Cl)cc1)NC12CC(NC(=O)COc3ccc4c(c3)CCCC4)(C1)C2 ; Oc1ccc2c(c1)CCCC2 ; CCOC(=O)COc1ccc2c(c1)CCCC2 ; O=C(O)COc1ccc2c(c1)CCCC2 ; NC12CC(NC(=O)COc3ccc(Cl)cc3)(C1)C2 ; O=C(COc1ccc(Cl)cc1)NC12CC(NC(=O)COc3ccc4c(c3)CCCC4)(C1)C2]

Step 1: To a solution 5,6,7,8-tetrahydronaphthalen-2-ol (0.5 g, 3.373 mmol 1 equiv) in DMF (8 mL) was added K₂CO₃ (0.69 g, 5.059 mmol, 1.5 equiv) followed by addition of ethyl bromoacetate (0.44 mL, 4.048 mmol, 1.2 equiv) dropwise at 0° C. The reaction mixture was allowed to stir at 80° C. for 4 h. After consumption of the starting material (TLC, 5% EtOAc in Hexane), the reaction mixture was allowed to cool to room temperature, diluted with water (20 mL) and extracted with EtOAc (2×25 mL). The combined organic layer was washed with water (2×10 mL), brine (20 mL), dried over anhydrous sodium sulfate, filtered and concentrated under reduced pressure to get the crude product. The crude product was purified by flash column chromatography using a silica gel column (9.8% ethyl acetate in hexane) to obtain the title compound ethyl 2-((5,6,7,8-tetrahydronaphthalen-2-yl)oxy)acetate (0.35 g, 44% yield) as a gum. ¹H NMR (400 MHz, CDCl₃) δ ppm 1.31-1.26 (m, 3H), 1.76 (s, 4H), 2.70 (d, J=13.6 Hz, 4H), 4.26-4.24 (m, 2H), 4.57 (s, 2H), 6.61 (s, 1H). 6.67 (d, J=8.4 Hz, 1H), 6.96 (d, J=8 Hz, 1H).

Step 2: To a solution of ethyl 2-((5,6,7,8-tetrahydronaphthalen-2-yl)oxy)acetate (0.35 g 1.495 mmol, 1 equiv) in a mixture of THF (4 mL) and water (1 mL) was added LiOH.H₂O (0.154 g, 3.739 mmol 2.5 equiv) at 0° C. The resulting mixture was stirred at room temperature for 1 h. THF was removed under reduced pressure and the residue was diluted with water (10 mL), and washed with Et₂O (20 mL). The aqueous layer was acidified with 1 N HCl up to pH ˜2 at 0° C. and then extracted with EtOAc (2×15 mL). The combined organic layer was dried over anhydrous sodium sulfate, filtered and concentrated under reduced pressure to obtain the title compound 2-((5,6,7,8-tetrahydronaphthalen-2-yl)oxy)acetic acid (0.237 g, 79% yield) as a white solid. LCMS (ES) m/z=205.1 [M−H]⁺. ¹H NMR (400 MHz, DMSO-d₆) δ ppm 1.67 (s, 4H), 2.64-2.60 (m, 4H), 4.56 (s, 2H), 6.55 (s, 1H), 6.60 (d, J=8 Hz 1H), 6.91 (s, J=8.4 Hz, 1H), 12.85 (s, 1H). This compound was directly taken to the next step without further purification.

Step 3: To a stirred solution of 2-((5,6,7,8-tetrahydronaphthalen-2-yl)oxy)acetic acid (0.11 g, 0.563 mmol, 1.5 equiv) and triethylamine (0.20 mL, 1.5 mmol, 4.0 equiv) in dichloromethane (4 mL) was added propylphosphonic anhydride solution (T3P®, 50 wt. % in ethyl acetate) (0.477 mL, 0.75 mmol, 2 equiv) at 0° C. and stirred for 10 min. Then a solution of N-(3-aminobicyclo[1.1.1]pentan-1-yl)-2-(4-chlorophenoxy)acetamide (0.1 g, 0.375 mmol, 1.0 equiv) in dichloromethane (5 mL) was added to the reaction mixture at 0° C. The reaction mixture was then stirred at room temperature for 16 h. After consumption of the starting material (TLC, 5% MeOH in DCM), the reaction mixture was diluted with DCM (100 mL), washed with saturated aqueous sodium bicarbonate solution (2×20 mL) and water (2×20 mL), brine (20 mL), dried over anhydrous sodium sulfate, filtered and concentrated under reduced pressure to afford the crude product. The obtained crude was purified by flash column chromatography (2-3% of methanol in dichloromethane) to afford the title compound 2-(4-chlorophenoxy)-N-(3-(2-((5,6,7,8-tetrahydronaphthalen-2-yl)oxy)acetamido)bicyclo[1.1.1]pentan-1-yl)acetamide (0.032 g, 18% yield) as an off-white solid. LCMS (ES) m/z=455.4 [M+H]⁺. ¹H NMR (400 MHz, DMSO-d₆) δ ppm 1.68 (s, 4H), 2.24 (s, 6H), 2.65-2.61 (m, 4H), 4.32 (s, 2H), 4.41 (s, 2H), 6.66-6.62 (m, 2H), 6.96-6.92 (m, 3H), 7.32 (d, J=8.8 Hz, 2H), 8.58 (s, 1H), 8.65 (s, 1H).

The Compounds of Examples 4b to 4d were prepared generally according to the procedure described above for Example 4a.

TABLE 8

| | | | LCMS | |
|---|---|---|---|---|
| | | | m/z | ¹H-NMR (400 MHz, |
| Cmpd # | Structure | Name | [M + H]⁺ | DMSO-d₆) |
| 4a | 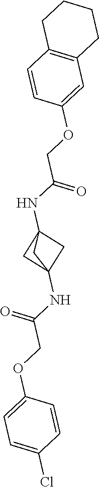 [SMILES: O=C(COc1ccc(Cl)cc1)NC12CC(NC(=O)COc3ccc4c(c3)CCCC4)(C1)C2] | 2-(4-chlorophenoxy)- N-(3-(2-((5,6,7,8- tetrahydronaphthalen- 2-yl)oxy)acetamido) bicyclo[1.1.1]pentan-1- yl)acetamide | 455.4 | 1.68 (s, 4 H), 2.24 (s, 6 H), 2.65-2.61 (m, 4 H), 4.32 (s, 2 H), 4.41 (s, 2 H), 6.66-6.62 (m, 2 H), 6.96-6.92 (m, 3 H), 7.32 (d, J = 8.8 Hz, 2 H), 8.58 (s, 1 H), 8.65 (s, 1 H). |
| 4b | 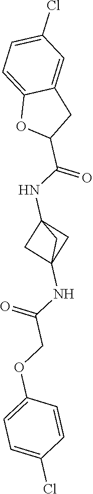 [SMILES: O=C(COc1ccc(Cl)cc1)NC12CC(NC(=O)C3Cc4cc(Cl)ccc4O3)(C1)C2] | 5-chloro-N-(3-(2-(4- chlorophenoxy) acetamido) bicyclo[1.1.1] pentan-1-yl)-2,3- dihydrobenzofuran- 2-carboxamide | 447.3 | 2.22 (s, 6 H), 3.39-3.46 (m, 2 H), 4.40 (s, 2 H), 5.06-5.11 (m, 1 H), 6.81 (d, J = 8.4 Hz, 1 H), 6.95 (d, J = 8.8 Hz, 2 H), 7.13 (d, J = 8.4 Hz, 1 H), 7.24 (s, 1 H), 7.32 (d, J = 8.4 Hz, 2 H), 8.64 (s, 1 H), 8.74 (s, 1 H). |
| 4c | 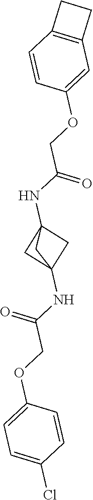 [SMILES: O=C(COc1ccc(Cl)cc1)NC12CC(NC(=O)COc3ccc4c(c3)CC4)(C1)C2] | 2-(bicyclo[4.2.0]octa- 1(6),2,4-trien-3- yloxy)-N-(3-(2-(4- chlorophenoxy) acetamido)bicyclo[1.1.1] pentan-1- yl)acetamide | 427.3 | 2.24 (s, 6 H), 3.03 (d, J = 3.2 Hz, 4 H), 4.33 (s, 2 H), 4.41 (s, 2 H), 6.70-6.76 (m, 2 H), 6.95 (d, J = 8.8 Hz, 3 H), 7.32 (d, J = 8.8 Hz, 2 H), 8.59 (s, 1 H), 8.66 (s, 1 H). |
| 4d | 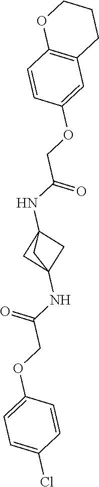 [SMILES: O=C(COc1ccc(Cl)cc1)NC12CC(NC(=O)COc3ccc4c(c3)CCCO4)(C1)C2] | 2-(4-chlorophenoxy)- N-(3-(2-(chroman-6- yloxy)acetamido) bicyclo[1.1.1]pentan- 1-yl)acetamide | 457.3 | 1.84-1.87 (m, 2 H), 2.24 (s, 6 H), 2.66-2.69 (m, 2 H), 4.02- 4.05 (m, 2 H), 4.28 (s, 2 H), 4.41 (s, 2 H), 6.61-6.67 (m, 3 H), 6.95 (d, J = 8.8 Hz, 2 H), 7.32 (d, J = 8.4 Hz, 2 H), 8.56 (s, 1 H), 8.61 (s, 1 H). |

### Example 4e

### N-(3-(5-chloroisoindolin-2-yl)bicyclo[1.1.1]pentan-1-yl)-2-(4-chlorophenoxy)acetamide

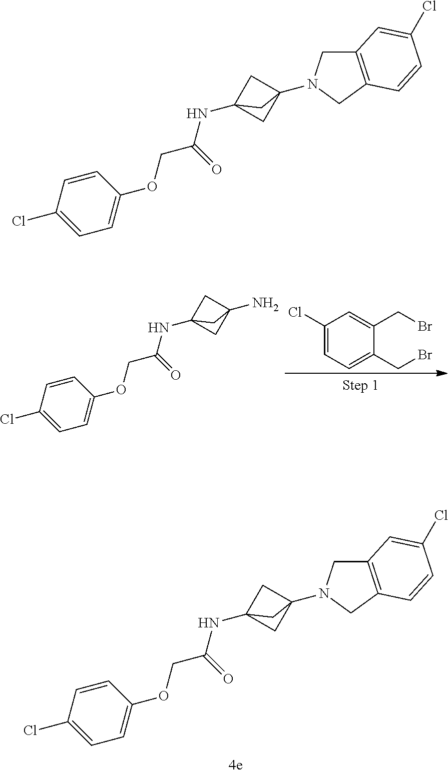

Step 1: To a stirred solution of N-(3-aminobicyclo[1.1.1]pentan-1-yl)-2-(4-chlorophenoxy)acetamide (0.15 g, 0.56 mmol, 1.0 equivalent) in acetonitrile (3 mL), was added triethylamine (0.31 mL) and 1,2-bis(bromomethyl)-4-chlorobenzene (0.167 g, 0.56 mmol) in a sealed tube at room temperature. The reaction mixture was heated at 100° C. for 1 h. The reaction mixture was evaporated by using high vacuum to afford the crude product. The crude product was purified by flash column chromatography (Combiflash®) using a silica gel column (2% methanol in DCM) to afford the product as a white solid. The solid was washed with MeOH (2 mL) and submitted for LCMS. From LCMS data the desired product was 98% pure with a 2% deschloro product observed. To remove the impurity the crude product was purified by preparative HPLC [Column: X-Bridge C18 (100 mm×4.6 mm×3.5 mic), Mobile phase (A): 0.1% Ammonia in water, Mobile phase (B): ACN, Flow rate: 1.0 mL/min, T/% B: 0/20, 7/60, 13/20, 15/20]. Fractions containing product were concentrated under reduced pressure to afford the title compound N-(3-(5-chloroisoindolin-2-yl)bicyclo[1.1.1]pentan-1-yl)-2-(4-chlorophenoxy)acetamide (0.08 g, 35% yield) as a white solid. LCMS (ES) m/z=403.3 [M+H]⁺. ¹H NMR (400 MHz, DMSO-d₆) δ ppm 2.04 (s, 6H), 3.85-3.87 (m, 4H), 4.41 (s, 2H), 6.95 (d, J=8.8 Hz, 2H), 7.22 (s, 2H), 7.32 (d, J=8.8 Hz, 3H), 8.64 (s, 1H).

TABLE 8A

| | | | LCMS | |
|---|---|---|---|---|
| | | | m/z | |
| Cmpd # | Structure | Name | [M + H]⁺ | ¹H-NMR (400 MHz, DMSO-d₆) |
| 4e | 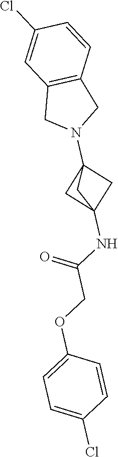 [SMILES: O=C(COc1ccc(Cl)cc1)NC12CC(N3Cc4ccc(Cl)cc4C3)(C1)C2] | N-(3-(5- chloroisoindolin-2- yl)bicyclo[1.1.1] pentan-1-yl)-2-(4- chlorophenoxy) acetamide | 403.3 | 2.04 (s, 6 H), 3.85-3.87 (m, 4 H), 4.41 (s, 2 H), 6.95 (d, J = 8.8 Hz, 2 H), 7.22 (s, 2 H), 7.32 (d, J = 8.8 Hz, 3 H), 8.64 (s, 1 H). |

### Example 5a

### 2-(4-chlorophenoxy)-N-(3-(4-(4-chlorophenyl)piperidin-1-yl)bicyclo[1.1.1]pentan-1-yl)acetamide

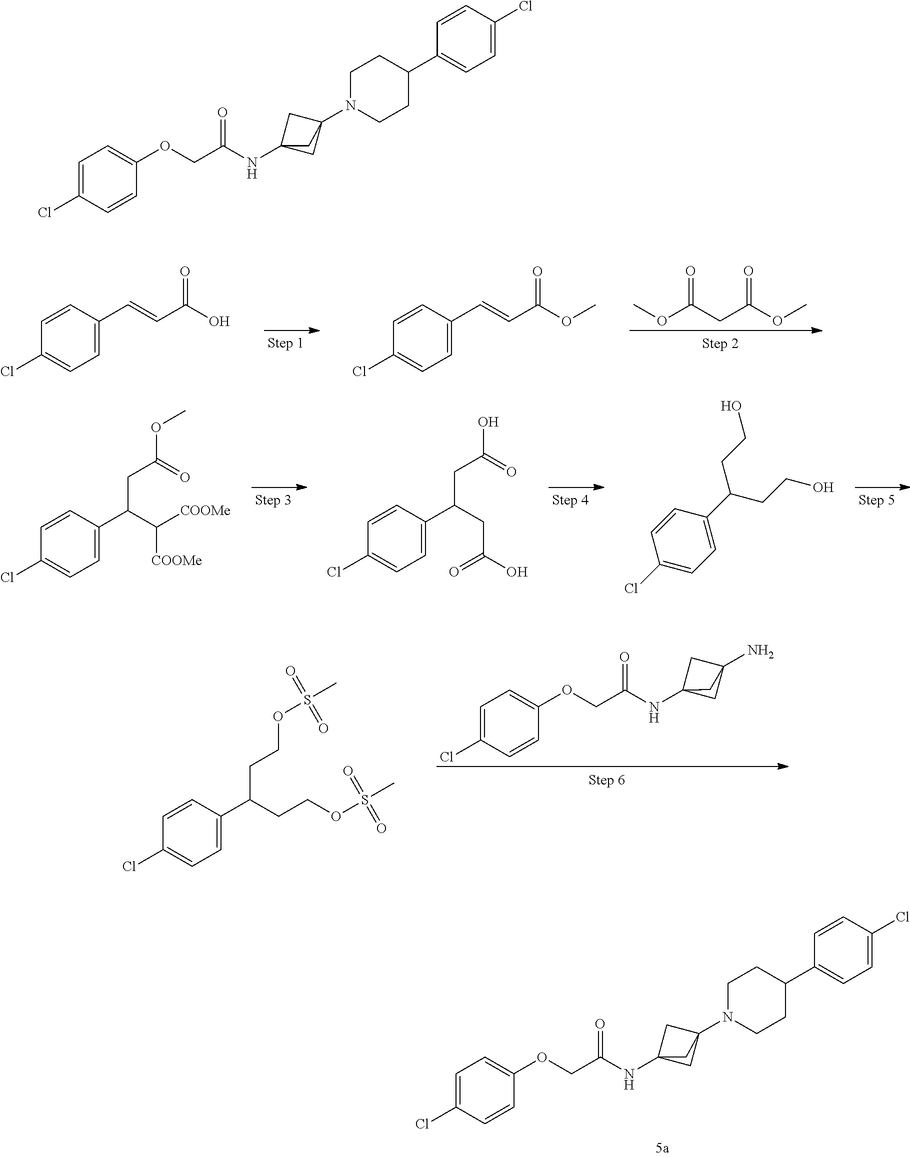

Step 1: To a solution of (E)-3-(4-chlorophenyl)acrylic acid (2.0 g, 1 equiv) in MeOH (20 mL) was added thionyl chloride (3.16 mL, 4 equiv) at rt. The resulting solution stirred at rt for 18 h, and then was evaporated to dryness. The crude compound was diluted with EtOAc (50 mL), washed with saturated sodium bicarbonate solution (25 mL) and brine (15 mL). The organics were dried over anhydrous Na₂SO₄, filtered and distilled under vacuum to provide methyl (E)-3-(4-chlorophenyl) acrylate (2.0 g, 93%) as a white solid. ¹H NMR (400 MHz, CDCl₃) δ ppm 3.80 (s, 3H), 6.41 (d, J=16.0 Hz, 1H), 7.35 (d, J=8.0 Hz, 2H), 7.45 (d, J=8.4 Hz, 2H), 7.63 (d, J=15.6 Hz, 1H).

Step 2: To a solution of sodium methoxide in MeOH (prepared by dissolving sodium metal (0.31 g, 13.41 mmol, 1.2 equiv) in 15 mL of anhydrous MeOH at 0° C.) under dry atmosphere at 0° C. was added a solution of dimethyl malonate (1.54 mL, 13.41 mmol, 1.2 equiv) in MeOH (1.0 mL) and stirred for 0.5 h at 0° C. Finally methyl (E)-3-(4-chlorophenyl) acrylate (2.2 g, 11.18 mmol, 1 equiv) was added to the reaction mixture, and the reaction was gradually warmed to rt and subsequently heated at reflux (80° C.). The reaction mixture was evaporated and the residue was dissolved into EtOAc (50 mL) and washed with water (25 mL) and brine (15 mL). The organic phase was dried over anhydrous Na₂SO₄, filtered and evaporated under reduced pressure. The crude material was purified by flash column chromatography (20% EtOAc in hexane) to obtain the desired product trimethyl 2-(4-chlorophenyl)propane-1,1,3-tricarboxylate as a semi-solid (2.6 g, 70%). LCMS (ES) m/z=329.0 [M+H]⁺. ¹H NMR (400 MHz, CDCl₃) δ ppm 2.58-2.75 (m, 2H), 2.81-2.86 (m, 1H), 3.51-3.54 (m, 6H), 3.75 (s, 3H), 3.89-3.93 (m, 1H), 7.18 (d, J=8.8 Hz, 2H), 7.25 (d, J=8.4 Hz, 2H).

Step 3: A suspension of trimethyl 2-(4-chlorophenyl)propane-1,1,3-tricarboxylate (3.0 g, 9.12 mmol) in 2 N NaOH (8 mL) was gently reluxed for 12 h at 90° C. The reaction mixture was cooled to rt and acidified with concentrated HCl to pH ˜0-1 and then heated to 100° C. for 12 h. The aqueous solution was distilled to remove most of the water and then extracted with EtOAc (2×30 mL), dried over anhydrous Na₂SO₄, filtered and evaporated under reduced pressure to afford 3-(4-chlorophenyl)pentanedioic acid as an off-white solid (2 g, 90%). LCMS (ES) m/z=240.9 [M−H]⁺. ¹H NMR (400 MHz, DMSO-d₆) δ ppm 2.68-2.92 (m, 4H), 3.58-3.77 (m, 1H), 7.17 (d, J=8.0 Hz, 2H), 7.31 (d, J=8.4 Hz, 1H), 10.39 (s, 2H).

Step 4: To a solution of 3-(4-chlorophenyl)pentanedioic acid (1.0 g, 4.12 mmol, 1 equiv) in THF (10 mL) was added BH₃.Me₂S (1.17 mL, 12.36 mmol, 3.0 equiv) at 0° C. The reaction mixture was then allowed to stir at rt for 12 h. Then the reaction mixture was quenched with MeOH (1 mL) at 0° C. and stirred for 30 min, and concentrated under reduced pressure to obtain the crude product. The crude material was purified by column chromatography (50% EtOAc in hexane) to afford 3-(4-chlorophenyl)pentane-1,5-diol (0.7 g, 80%) as an oily compound. LCMS (ES) m/z=215.1 [M+H]⁺. ¹H NMR (400 MHz, CDCl3) δ ppm 1.68-1.83 (m, 2H), 1.92-2.08 (m, 2H), 2.88-2.97 (m, 1H), 3.42-3.48 (m, 2H), 3.53-3.79 (m, 2H), 7.13 (d, J=8.0 Hz, 2H), 7.25-7.28(m, 2H).

Step 5: To a stirred solution of 3-(4-chlorophenyl)pentane-1,5-diol (0.2 g, 0.93 mmol, 1.0 equiv.) in DCM (10 mL) was added methanesulfonyl chloride (0.22 mL, 3.0 equiv.) at 0° C. followed by dropwise addition of Et₃N (0.51 mL, 3.72 mmol, 4.00 equiv.). After stirring at 0° C. for 0.5 h, the reaction mixture was slowly brought to rt and stirred at rt for 2 h. To the reaction mixture was added aq. NH₄Cl (5 mL) and the aqueous phase was extracted with EtOAc (2×25 mL). The combined organic phase was dried over anhydrous Na₂SO₄, filtered and evaporated under reduced pressure to afford 3-(4-chlorophenyl)pentane-1,5-diyl-dimethanesulfonate (0.25 g, crude) as a semi-solid which was directly used for the next step without further purification. ¹H NMR (400 MHz, CDCl3) δ ppm 1.91-2.00 (m, 2H), 2.15-2.34 (m, 2H), 2.93 (s, 6 h), 3.13-3.22 (m, 1H), 3.84-4.06 (m, 2H), 4.10-4.28 (m, 2H), 7.13 (d, J=8.0 Hz, 2H), 7.32 (d, J=8.0 Hz, 2H).

Step 6: 3-(4-Chlorophenyl)pentane-1,5-diyl-dimethanesulfonate (0.2 g, 0.539 mmol, 1 equiv) and N-(3-aminobicyclo[1.1.1]pentan-1-yl)-2-(4-chlorophenoxy)acetamide (0.14 g, 0.539 mmol, 1 equiv) were charged to a sealed tube and Et₃N (0.37 mL, 2.69 mmol, 5 equiv)) was added. The mixture was then heated at 90° C. using an oil bath for 1 h. The reaction mixture was evaporated under reduced pressure to obtain crude compound. The crude material was purified by column chromatography using an eluent of 50% EtOAc in hexane. The product was further re-purified by preparative TLC using 50% EtOAc in hexane as the mobile phase. After prep TLC purification, the compound was dissolved in 0.5 mL CH₃CN. To this was added 5 mL n-pentane, stirred for 0.5 h and then filtered to afford 2-(4-chlorophenoxy)-N-(3-(4-(4-chlorophenyl)piperidin-1-yl)bicyclo[1.1.1]pentan-1-yl)acetamide (0.003 g, 1.2%) as an off-white solid. LCMS (ES) m/z=445.4 [M+H]⁺. ¹H NMR (400 MHz, DMSO-d₆) δ ppm 1.55-1.58 (m, 2H), 1.69 (s, 2H), 1.95-2.03 (m, 9H), 2.86-2.88 (m, 2H), 4.40 (m, 2H), 6.95 (d, J=7.6 Hz, 2H), 7.25-7.30 (m, 6H), 8.61 (s, 1H).

TABLE 9

| | | | LCMS | |
|---|---|---|---|---|
| | | | m/z | |
| Cmpd # | Structure | Name | [M + H]⁺ | ¹H-NMR (400 MHz, DMSO-d₆) |
| 5a | 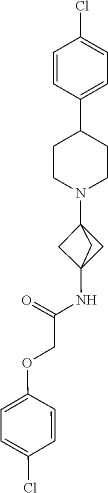 [SMILES: O=C(COc1ccc(Cl)cc1)NC12CC(N3CCC(c4ccc(Cl)cc4)CC3)(C1)C2] | 2-(4-chlorophenoxy)-N-(3- (4-(4- chlorophenyl)piperidin-1- yl)bicyclo[1.1.1]pentan-1- yl)acetamide | 445.4 | 1.55-1.58 (m, 2 H), 1.69 (s, 2 H), 1.95-2.03 (m, 9 H), 2.86-2.88 (m, 2 H), 4.40 (m, 2 H), 6.95 (d, J = 7.6 Hz, 2 H), 7.25-7.30 (m, 6 H), 8.61 (s, 1 H). |

### Example 5b

### 2-(4-chlorophenoxy)-N-(3-(4-(4-chlorophenylpiperazin-1-yl)bicyclo[1.1.1]pentan-1-yl)acetamide

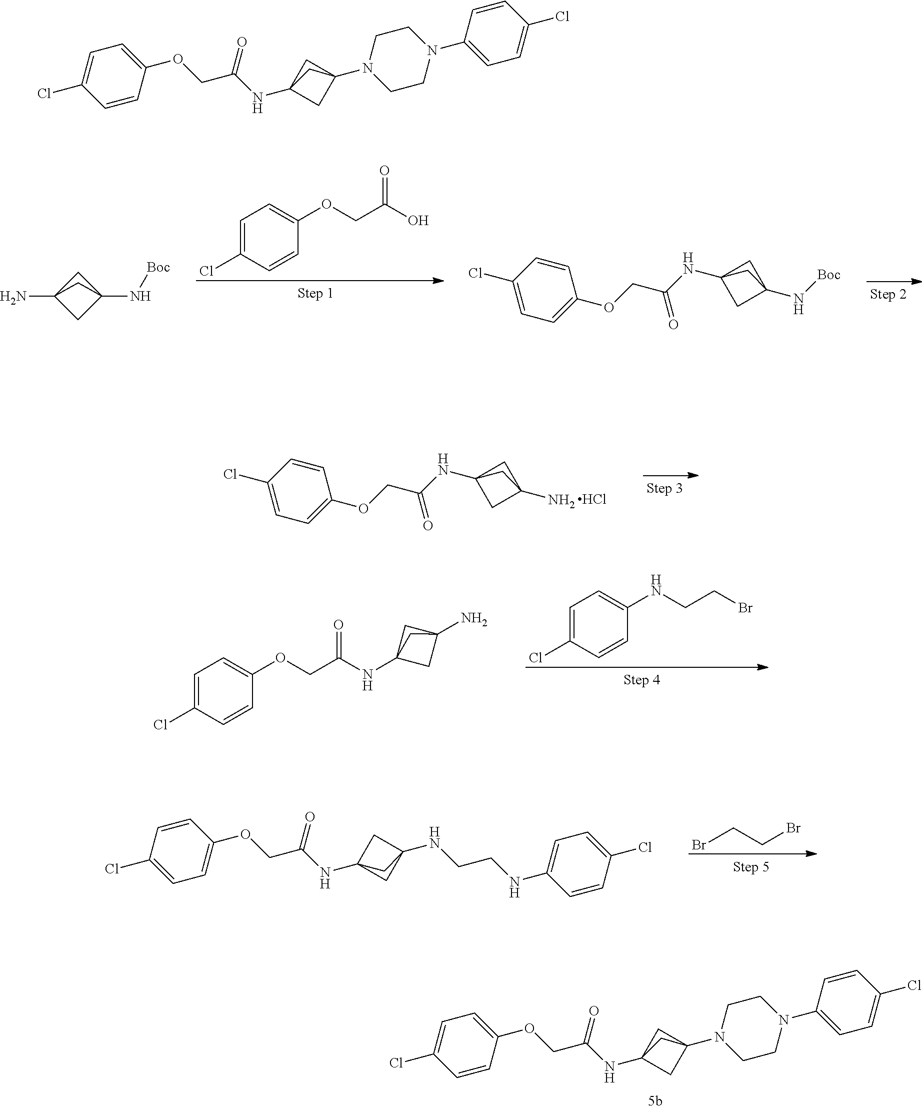

Step 1: To a solution of 2-(4-chlorophenoxy)acetic acid (25.32 g, 136.17 mmol, 1.2 equiv) in DCM (250 mL) at 0° C. was added triethylamine (63 mL, 453.92 mmol, 4 equiv) and was stirred for 5 minutes at 0° C. T3P® (50 wt. % in ethyl acetate) (108.4 mL, 170.22 mmol, 1.5 equiv) was added and the reaction mixture was stirred at 0° for 10 mins. Then, tert-butyl (3-aminobicyclo[1.1.1]pentan-1-yl)carbamate (22.5 g, 113.48 mmol, 1 equiv) was added to the reaction mixture, and the reaction mixture was stirred at room temperature for 12 hours. After completion of the reaction, the reaction mixture was concentrated under reduced pressure to obtain the crude product, which was triturated by adding saturated aqueous NaHCO₃ solution (50 mL) and water (50 mL). The obtained light brown solid was filtered through a sintered funnel and dried. The obtained solid was dissolved in DCM and washed with water. The organic layer was dried over anhydrous Na₂SO₄, filtered and concentrated under reduced pressure to afford tert-butyl (3-(2-(4-chlorophenoxy)acetamido)bicyclo[1.1.1]pentan-1-yl)carbamate (39 g, 93% yield) as a light brown solid. LCMS (ES) m/z=311.1 [M+H]⁺ (t-butyl cleavage mass was observed). ¹H NMR (400 MHz, DMSO-d₆) δ ppm 1.35 (s, 9H), 2.11 (s, 6H), 4.39 (s, 2H), 6.94 (d, J=8.8 Hz, 2H), 7.31 (d, J=8.8 Hz, 2H), 7.46 (bs, 1H), 8.60 (s, 1H).

Step 2: To a solution of tert-butyl (3-(2-(4-chlorophenoxy)acetamido)bicyclo[1.1.1]pentan-1-yl)carbamate (20 g, 54.49 mmol, 1 equiv) in DCM (225 mL) was added 4M HCl in 1,4-dioxane (60 mL) at 0° C. The reaction mixture stirred at room temperature for 12 h. After consumption of the starting material (TLC, 5% methanol in DCM), DCM was evaporated under reduced pressure, and the obtained solid was triturated with n-pentane (100 mL) and diethyl ether (100 mL) and dried under high vacuum to afford N-(3-aminobicyclo[1.1.1]pentan-1-yl)-2-(4-chlorophenoxy)acetamide hydrochloride (14 g, 84%) as a light brown solid. LCMS (ES) m/z=267.1 [M+H]⁺. (Free amine mass was observed). ¹H NMR (400 MHz, DMSO-d₆) δ ppm 2.22 (s, 6H), 4.43 (s, 2H), 6.95 (d, J=9.2 Hz, 2H), 7.32 (d, J=8.8 Hz, 2H), 8.65 (s, 3H), 8.81 (s, 1H).

Step 3: To a stirred solution of N-(3-aminobicyclo[1.1.1]pentan-1-yl)-2-(4-chlorophenoxy)acetamide hydrochloride (15.0 g, 49.66 mmol, 1 equiv) in ethyl acetate (200 mL) at room temperature was added saturated sodium bicarbonate. After stirring at room temperature for 30 minutes, it was extracted with ethyl acetate (2×250 mL). The combined organic extract was washed with water (100 mL) and brine (50 mL). The organic phase was dried over anhydrous sodium sulfate, filtered and concentrated under reduced pressure to give N-(3-aminobicyclo[1.1.1]pentan-1-yl)-2-(4-chlorophenoxy)acetamide (13 g, crude) as a brown gum. The crude material was purified by reverse phase HPLC: [Column: C18, Mobile phase (A): 0.1% ammonia in water, Mobile phase (B): Acetonitrile]. Fractions containing product were concentrated under reduced pressure and the obtained material was stirred in n-pentane (40 mL) at room temperature for 1 h. Then the solid was filtered and dried to afford N-(3-aminobicyclo[1.1.1]pentan-1-yl)-2-(4-chlorophenoxy)acetamide (6.6 g, 50.7% yield) as a white solid. LCMS (ES) m/z=267.1 [M+H]⁺. ¹H NMR (400 MHz, DMSO-d₆) δ ppm 1.90 (s, 6H), 2.11 (s, 2H), 4.37 (s, 2H), 6.93 (d, J=8.8 Hz, 2H), 7.30-7.32 (m, 2H), 8.47 (s, 1H).

Step 4: To a solution of N-(3-aminobicyclo[1.1.1]pentan-1-yl)-2-(4-chlorophenoxy)acetamide (0.2 g, 0.74 mmol, 1.0 equiv) in triethylamine (0.52 mL, 3.7 mmol, 5.0 equiv) was added N-(2-bromoethyl)-4-chloroaniline (0.21 g, 0.89 mmol, 1.2 equiv) at room temperature in a sealed tube. The reaction mixture was maintained at 100° C. for 2 h. The reaction mixture was diluted with DCM (400 mL), and the combined organic layers were washed with cold water (2×50 mL), dried over anhydrous sodium sulfate, filtered and concentrated under reduced pressure to give the crude product. The crude material was purified by flash chromatography using 0.1% to 10% methanol in DCM as an eluent to obtain 2-(4-chlorophenoxy)-N-(3-((2-((4-chlorophenyl)amino)ethyl)amino)bicyclo[1.1.1]pentan-1-yl)acetamide (0.5 g, 41.66% yield, 0.2 g scale reactions with 4 batches (0.8 g)) as an off-white solid. LCMS (ES) m/z=420.0 [M+H]⁺. ¹H NMR (400 MHz, DMSO-d₆) δ ppm 1.94 (s, 6H), 2.64 (d, J=8.0 Hz, 2H), 2.99-3.04 (m, 2H), 4.39 (s, 2H), 5.66 (bs, 1H), 6.54 (d, J=8.8 Hz, 2H),6.94 (d, J=8.8 Hz, 2H), 7.05 (d, J=8.4 Hz, 2H), 7.32 (d, J=8.4 Hz, 2H), 8.56 (s, 1H). NH proton was not observed.

Step 5: To a solution of 2-(4-chlorophenoxy)-N-(3-((2-((4-chlorophenyl)amino)ethyl)amino)bicyclo[1.1.1]pentan-1-yl)acetamide (0.2 g, 0.47 mmol, 1.0 equiv) in DMF (5 mL) was added 1,2-dibromoethane (0.041 mL, 0.47 mmol, 1.0 equiv) and K₂CO₃ (0.32 g, 2.3 mmol, 5.0 equiv) at room temperature. After the reaction mixture was maintained at 100° C. for 2 h, it was cooled to room temperature and quenched with crushed ice (25 mL) and extracted with DCM (2×50 mL). The combined organic layers were washed with cold water (2×25 mL), dried over anhydrous sodium sulfate, filtered and concentrated under reduced pressure to give the crude product. The crude material was purified by flash chromatography using 0.5% to 70% ethyl acetate in n-hexane as an eluent and repurified by preparative HPLC [Analytical conditions: Column: lnertsil ODS 3V (250 mm×4.6 mm×5 μm). Mobile phase (A): 0.1% Ammonia in water, Mobile phase (B): Acetonitrile, Flow rate: 1.0 mL/min, compound RT: 20.99 minutes] to obtain 2-(4-chlorophenoxy)-N-(3-(4-(4-chlorophenyl)piperazin-1-yl)bicyclo[1.1.1]pentan-1-yl)acetamide (0.045 g, 21.5% yield) as a white solid. LCMS (ES) m/z=446.3 [M+H]⁺. ¹H NMR (400 MHz, DMSO-d₆) δ ppm 1.97 (s, 6H), 2.48 (s, 4H), 3.41 (s, 4H), 4.41 (s, 2H), 6.91-6.96 (m, 4H), 7.20 (d, J=8.0 Hz, 2H), 7.32 (d, J=8.4 Hz, 2H), 8.63 (s, 1H).

TABLE 10

| | | | LCMS | |
|---|---|---|---|---|
| | | | m/z | ¹H-NMR (400 MHz, |
| Cmpd # | Structure | Name | [M + H]⁺ | DMSO-d₆) |
| 5b | 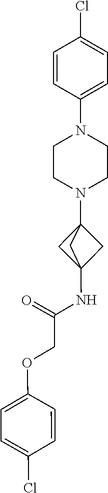 [SMILES: O=C(COc1ccc(Cl)cc1)NC12CC(N3CCN(c4ccc(Cl)cc4)CC3)(C1)C2] | 2-(4-chlorophenoxy)-N-(3-(4- (4-chlorophenyl)piperazin-1- yl)bicyclo[1.1.1]pentan-1- yl)acetamide | 446.3 | 1.97 (s, 6 H), 2.48 (s, 4 H), 3.41 (s, 4 H), 4.41 (s, 2 H), 6.91- 6.96 (m, 4 H), 7.20 (d, J = 8.0 Hz, 2 H), 7.32 (d, J = 8.4 Hz, 2 H), 8.63 (s, 1 H). |

### Example 5c

### 2-(bicyclo[4.2.0]octa-1,3,5-trien-3-yloxy)-N-(4-(2-(4-chlorophenoxy)acetamido)bicyclo[2.2.1]heptan-1-yl)acetamide

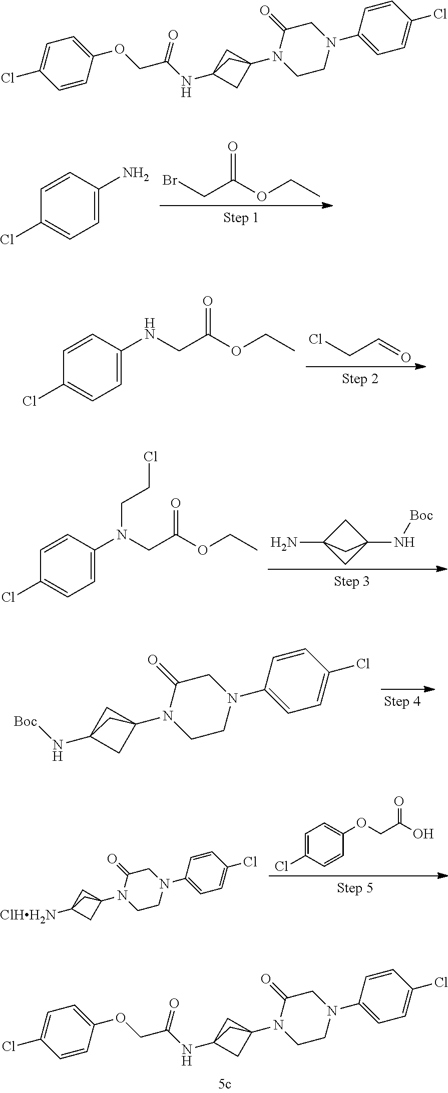

Step 1: To a stirred solution of 4-chloroaniline (2.0 g, 15.75 mmol, 1.0 equivalent) in acetone (50 mL) was added K₂CO₃ (3.04 g, 22.05 mmol, 1.4 equiv) followed by ethyl 2-bromoacetate (1.91 mL, 17.32 mmol, 1.1 equiv) and the reaction mixture was heated to reflux for 16 h. After completion of the reaction, the reaction mixture was filtered through a Buchner funnel with a Celite® bed. The Celite® bed was washed with ethyl acetate (100 mL). The filtrate was concentrated under reduced pressure to give the crude product. The crude material was purified by silica gel column chromatography using 15% ethyl acetate in n-Hexane as an eluent to afford ethyl (4-chlorophenyl)glycinate (1.3 g, 38.92%) as an off-white solid. LCMS (ES) m/z=214.1 [M+H]⁺. ¹H-NMR (400 MHz, DMSO-d₆): 1.17 (t, J=7.0 Hz, 3H), 3.86 (d, J=6.0 Hz, 2H), 4.09 (q, J=6.9 Hz, 2H), 6.15 (s, 1H), 6.54 (d, J=8.4 Hz, 2H), 7.07 (d, J=8.4 Hz, 2H).

Step 2: To a solution of ethyl (4-chlorophenyl)glycinate (1.0 g, 4.67 mmol, 1 equiv) in methanol (20 mL), was added 2-chloroacetaldehyde (6.7 mL, 46.73 mmol, 10 equiv) followed by acetic acid (0.5 mL) and stirred for 15 min at rt. NaCNBH₃ (1.17 g, 18.69 mmol, 4 equiv) was added at 0° C. and then the reaction mixture was allowed to stir at rt for 16 h. After completion of the reaction, the reaction mixture was concentrated under reduced pressure to obtain crude product. The crude product was dissolved in ethyl acetate (200 mL) and washed with water (100 mL) and brine solution (100 m L), dried over anhydrous sodium sulfate, filtered and concentrated under reduced pressure to give the crude product. The crude product was purified by silica gel column chromatography by using 8% ethylacetate in n-Hexane as an eluent to afford ethyl N-(2-chloroethyl)-N-(4-chlorophenyl) glycinate (0.9 g, 70.31%) as a pale yellow liquid. LCMS (ES) m/z=276.0 [M+H]⁺. ¹H-NMR (400 MHz, DMSO-d₆): 1.17 (t, J=7.0 Hz, 3H), 3.67-3.71 (m, 4H), 4.09 (q, J=7.0 Hz, 2H), 4.21 (s, 2H), 6.63 (d, J=8.8 Hz, 2H), 7.17 (d, J=9.2 Hz, 2H).

Step 3: Ethyl N-(2-chloroethyl)-N-(4-chlorophenyl)glycinate (0.35 g, 1.26 mmol, 1 equiv) and tert-butyl (3-aminobicyclo[1.1.1]pentan-1-yl)carbamate (0.25 g, 1.26 mmol, 1 equiv) were charged to a sealed tube and diisopropylethylamine (0.87 mL, 5.05 mmol, 4 equiv) was added. The mixture was then heated at 100° C. for 16 h. After that time the reaction mixture was concentrated under reduced pressure to give the crude product. The crude material was purified by silica gel column chromatography by using 35% ethyl acetate in n-Hexane as an eluent to afford tert-butyl (3-(4-(4-chlorophenyl)-2-oxopiperazin-1-yl)bicyclo[1.1.1]pentan-1-yl)carbamate (0.16 g, 16.33%) as a light yellow colour solid. LCMS (ES) m/z=392.1 [M+H]⁺. ¹H-NMR (400 MHz, DMSO-d₆): 1.36 (s, 9H), 2.21 (s, 6H), 3.35 (s, 2H), 3.42 (s, 2H), 3.7 (s, 2H), 6.9 (d, J=8.4 Hz, 2H), 7.22 (d, J=8.4 Hz, 2H), 7.5 (s, 1H).

Step 4: To a solution of tert-butyl (3-(4-(4-chlorophenyl)-2-oxopiperazin-1-yl)bicyclo[1.1.1]pentan-1-yl)carbamate (0.16 g, 0.41 mmol, 1 equiv) in DCM (5 mL) at 0° C. was added 3 mL of 4M HCl in 1,4-dioxane and the mixture was stirred at rt for 4 h. The reaction mixture was concentrated to afford crude product. The crude product was washed with dry n-pentane (50 mL) and dried under high vacuum to afford 1-(3-aminobicyclo[1.1.1]pentan-1-yl)-4-(4-chlorophenyl)piperazin-2-one hydrochloride (0.133 g crude) which was taken to the next step without further purification. LCMS (ES) m/z=292.1 [M+H]⁺.

Step 5: To a solution of 2-(4-chlorophenoxy)acetic acid (0.11 g, 0.59 mmol, 1.5 equiv) in DCM (5 mL) at 0° C. was added triethylamine (0.22 mL, 1.58 mmol, 4 equiv) followed by T3P® (50 wt % in EtOAc) (0.47 mL, 0.79 mmol, 2 equiv) and the mixture was stirred at 0° C. for 10 min. To this reaction mixture was added 1-(3-aminobicyclo[1.1.1]pentan-1-yl)-4-(4-chlorophenyl)piperazin-2-one hydrochloride (0.13 g, 0.39 mmol, 1 equiv) in DCM (2 mL) and triethylamine (0.055 mL, 0.39 mmol, 1 equiv) slowly at 0° C. and the reaction was stirred at rt for 16 h. The reaction mixture was diluted with water (50 mL) and extracted with DCM (2×50 mL). The combined organic layer was washed with saturated aqueous NaHCO₃ solution (50 mL) and then dried over anhydrous Na₂SO₄, filtered and evaporated under vacuum. The crude material was purified by column chromatography twice using an eluent of 50% EtOAc in hexane and again it was purified by preparative HPLC (Analytical conditions: Column: Inertsil ODS 3V (250 mm×4.6 mm×5 mic); Mobile phase(A): 0.1% Ammonia in water; Mobile phase (B): CAN; Flow rate: 1.0 mL/min (40:60)) to afford 2-(4-chlorophenoxy)-N-(3-(4-(4-chlorophenyl)-2-oxopiperazin-1-yl)bicyclo[1.1.1]pentan-1-yl)acetamide (0.017 g, 9.4%) as a white solid. LCMS (ES) m/z=460.3 [M+H]⁺. ¹H-NMR (400 MHz, DMSO-d₆): 2.34 (s, 6H), 3.37-3.43 (m, 4H), 3.71 (s, 2H), 4.42 (s, 2H), 6.91 (d, J=9.2 Hz, 2H), 6.95 (d, J=9.2 Hz, 2H), 7.22 (d, J=8.8 Hz, 2H), 7.32 (d, J=8.8 Hz, 2H), 8.71 (s, 1H).

TABLE 11

| | | | LCMS | |
|---|---|---|---|---|
| | | | m/z | ¹H-NMR (400 MHz, |
| Cmpd # | Structure | Name | [M + H]⁺ | DMSO-d₆) |
| 5c | 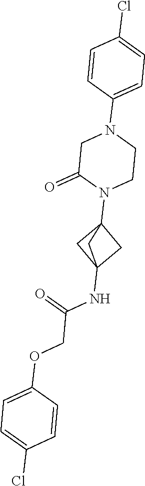 [SMILES: O=C(COc1ccc(Cl)cc1)NC12CC(N3CCN(c4ccc(Cl)cc4)CC3=O)(C1)C2] | 2-(4-chlorophenoxy)- N-(3-(4-(4- chlorophenyl)-2- oxopiperazin-1- yl)bicyclo[1.1.1]pentan- 1-yl)acetamide | 460.3 | 2.34 (s, 6 H), 3.37-3.43 (m, 4 H), 3.71 (s, 2 H), 4.42 (s, 2 H), 6.91 (d, J = 9.2 Hz, 2 H), 6.95 (d, J = 9.2 Hz, 2 H), 7.22 (d, J = 8.8 Hz, 2 H), 7.32 (d, J = 8.8 Hz, 2 H), 8.71 (s, 1 H). |

### Example 6a and Example XXIV

### 2-(4-chlorophenoxy)-N-(3-(3-(4-chlorophenoxy)pyrrolidin-1-yl)bicyclo[1.1.1]pentan-1-yl)acetamide

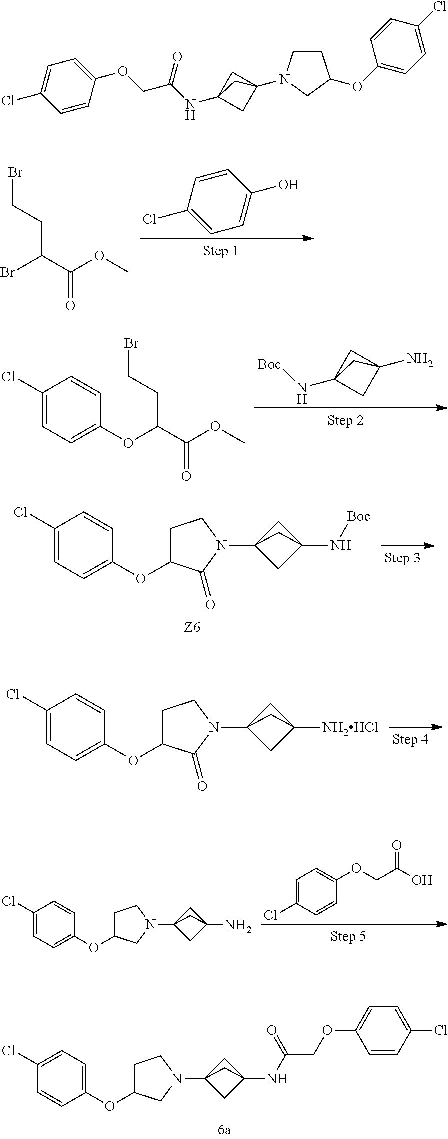

Step 1: To a solution of methyl 2,4-dibromobutanoate (1.2 g, 1 equiv) in DMF (15 mL) was added 4-chlorophenol (0.59 g, 1 equiv) at rt followed by K₂CO₃ (0.636 g, 1 equiv) and the reaction was stirred at 60 ⁰0 for 3 h. The reaction mixture was then allowed to warm to rt. Water (5 mL) was added and the mixture was extracted with EtOAc (3×50 mL). The combined EtOAc extracts were dried over anhydrous Na₂SO₄, filtered and distilled under vacuum. The crude material was purified by flash column chromatography eluting the product at 10% EtOAc in hexane to provide methyl 4-bromo-2-(4-chlorophenoxy)butanoate as a gum (1 g). ¹H NMR (400 MHz, CDCl₃) δ ppm 2.3-2.5 (m, 2H), 3.55-3.64 (m, 2H), 3.76 (s, 3H), 4.83-4.85 (m, 1H), 6.86 (d, J=3.2 Hz, 2H), 7.23-7.25 (m, 2H).

Step 2: Methyl 4-bromo-2-(4-chlorophenoxy)butanoate (0.3 g, 1.01 mmol, 1 equiv) and tert-butyl (3-aminobicyclo[1.1.1]pentan-1-yl)carbamate (0.2 g, 1.01 mmol, 1 equiv) were charged to a sealed tube and Et₃N (0.6 mL) was added. The mixture was then heated at 100° using an oil bath for 1 h. The reaction mixture was diluted with water (50 mL) and extracted with EtOAc (2×50 mL). The combined EtOAc extracts were dried over anhydrous Na₂SO₄, filtered and evaporated under vacuum. The crude material was purified by column chromatography using an eluent of 45% EtOAc in hexane to obtain the desired product tert-butyl (3-(3-(4-chlorophenoxy)-2-oxopyrrolidin-1-yl)bicyclo[1.1.1]pentan-1-yl)carbamate as an off-white solid (0.1 g, 25.6%). LCMS (ES) m/z=337.1 [M+H]⁺ (loss of tert-butyl group). ¹H NMR (400 MHz, DMSO-d₆) δ ppm 1.44 (s, 9H), 2.04-2.18 (m, 1H), 2.39 (s, 6H), 2.46-2.54 (m, 1H), 3.29-3.35 (m, 1H), 3.42-3.47 (m, 1H), 4.77 (t, J=7.2 Hz, 1H), 4.94 (bs, 1H), 6.98 (d, J=9.2 Hz, 2H), 7.21 (d, J=8.8 Hz, 2H).

Step 3: To a solution of tert-butyl (3-(3-(4-chlorophenoxy)-2-oxopyrrolidin-1-yl)bicyclo[1.1.1]pentan-1-yl)carbamate (0.1 g, 0.25 mmol) in DCM (5 mL) at 0° C. was added 2 mL of 4 M HCl in 1,4-dioxane and the mixture was stirred at rt for 16 h. The reaction mixture was concentrated to give 1-(3-aminobicyclo[1.1.1]pentan-1-yl)-3-(4-chlorophenoxy)pyrrolidin-2-one, hydrochloride (crude yield 0.08 g, 96.3%), which was taken to the next step without further purification. LCMS (ES) m/z=293 [M+H]⁺. ¹H NMR (400 MHz, DMSO-d₆) δ ppm 1.89-1.90 (m, 1H), 2.18-2.19 (m, 1H), 2.28-2.33 (m, 6H), 4.98 (t, J=7.2 Hz, 1H), 7.02 (d, J=8.8 Hz, 2H), 7.3 (d, J=9.6 Hz, 1H), 8.79 (s, 3H).

Step 4: To a solution of 1-(3-aminobicyclo[1.1.1]pentan-1-yl)-3-(4-chlorophenoxy)pyrrolidin-2-one hydrochloride (0.08 g, 0.24 mmol, 1 equiv) in THF (5 mL) was added BH₃.Me₂S (0.06 mL, 0.61 mmol, 2.5 equiv) at 0° C. The reaction mixture was then allowed to stir at rt for 16 h. The reaction mixture was quenched with MeOH (1 mL) at 0° C. and stirred for 30 min, and concentrated under reduced pressure to obtain the crude product. This crude material was then dissolved in DCM (50 mL) and washed with saturated Na₂HCO₃ solution. The organic phase was dried over anhydrous Na₂SO₄, filtered and evaporated under vacuum to yield 3-(3-(4-chlorophenoxy)pyrrolidin-1-yl)bicyclo[1.1.1]pentan-1-amine (0.06 g, crude yield), which was used for the next step without further purification. LCMS (ES) m/z=279 [M+H]⁺.

Step 5: To a solution of 2-(4-chlorophenoxy)acetic acid (0.06 g, 0.23 mmol, 1.5 equiv) in DCM (5 mL) at 0° C. was added triethylamine (0.15 mL, 1.07 mmol, 5 equiv) followed by T3P® (50 wt % in EtOAc) (0.25 mL, 0.43 mmol, 2 equiv). The mixture was stirred at 0° C. for 10 min, at which time, T3P® (50 wt. % in ethyl acetate) (1.12 g, 1.768 mmol, 2 equiv) in dichloromethane (10 mL) was added at 0° C. and stirred for 10 min. To this reaction mixture was added 3-(3-(4-chlorophenoxy)pyrrolidin-1-yl)bicyclo[1.1.1]pentan-1-amine (0.06 g) in DCM (1 mL) slowly at 0° C. and the reaction was stirred at rt for 16 h. The reaction mixture was diluted with water (50 mL) and extracted with DCM (2×50 mL). The combined organic layer was washed with saturated aqueous NaHCO₃ solution (50 mL) and then dried over anhydrous Na₂SO₄, filtered and evaporated under reduced pressure. The crude product was purified by preparative TLC using 40% EtOAc in hexanes. After final drying, the desired product 2-(4-chlorophenoxy)-N-(3-(3-(4-chlorophenoxy)pyrrolidin-1-yl)bicyclo[1.1.1]pentan-1-yl)acetamide was obtained as a white solid (0.0108 g, 11.2%). LCMS (ES) m/z=447.3 [M+H]⁺. ¹H NMR (400 MHz, DMSO-d₆) δ ppm 1.76-1.77 (m, 1.5H), 1.95 (s, 6H), 2.2-2.27 (m, 1.5H), 2.58-2.61 (m, 1H), 2.65-2.69 (m, 1H), 2.84 (m, 1H), 4.4 (s, 2H), 4.85 (bs, 1H), 6.88 (d, J=8.8 Hz, 2H), 6.94 (d, J=8.8 Hz, 2H), 7.28 (d, J=8.4 Hz, 2H), 7.32 (d, J=8.8 Hz, 2H), 8.61 (s, 1H).

### Example 6b

### (S)-2-(4-chlorophenoxy)-N-(3-(3-(4-chlorophenoxy)pyrrolidin-1-yl)bicyclo[1.1.1]pentan-1-yl)acetamide

### Example 6c

### (R)-2-(4-chlorophenoxy)-N-(3-(3-(4-chlorophenoxy)pyrrolidin-1-yl)bicyclo[1.1.1]pentan-1-yl)acetamide

Separation of the enantiomers of Example 6b and 6c by chiral prep HPLC purification:

Example 6a was subjected to chiral prep HPLC to obtain Example 6b and Example 6c by using the following conditions: Column: CHIRALPAK 1C (100 mm×4.6 mm×3 mic), mobile phase: Hexane: IPA (85:15) with 0.1% DEA.

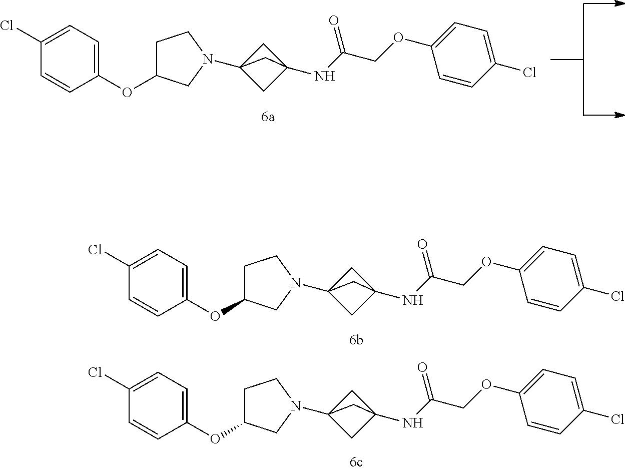

Comparing the observed VCD and IR spectra of example 6b with the calculated spectra of the modeled (R)-structure, the absolute configuration of example 6b was assigned as (S)-. Comparing the observed VCD and IR spectra of example 6c with the calculated spectra of the modeled (R)-structure, the absolute configuration of example 6c was assigned as (R)-.

The Compounds of Examples 6d to 6e were prepared generally according to the procedure described above for Example 6a-6c.

TABLE 12

| | | | LCMS | |
|---|---|---|---|---|
| | | | m/z | ¹H-NMR (400 MHz, |
| Cmpd # | Structure | Name | [M + H]⁺ | DMSO-d₆) |
| 6a | 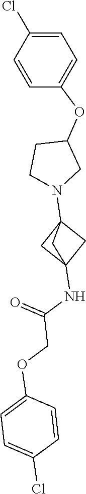 [SMILES: O=C(COc1ccc(Cl)cc1)NC12CC(N3CCC(Oc4ccc(Cl)cc4)C3)(C1)C2] | 2-(4-chlorophenoxy)-N-(3- (3-(4- chlorophenoxy)pyrrolidin- 1-yl)bicyclo[1.1.1]pentan- 1-yl)acetamide | 447.3 | 1.76-1.77 (m, 1.5 H), 1.95 (s, 6 H), 2.2-2.27 (m, 1.5 H), 2.58-2.61 (m, 1 H), 2.65-2.69 (m, 1 H), 2.84 (m, 1 H), 4.4 (s, 2 H), 4.85 (bs, 1 H), 6.88 (d, J = 8.8 Hz, 2 H), 6.94 (d, J = 8.8 Hz, 2 H), 7.28 (d, J = 8.4 Hz, 2 H), 7.32 (d, J = 8.8 Hz, 2 H), 8.61 (s, 1 H). |
| 6b | 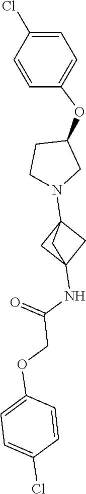 [SMILES: O=C(COc1ccc(Cl)cc1)NC12CC(N3CC[C@@H](Oc4ccc(Cl)cc4)C3)(C1)C2] | (R)-2-(4-chlorophenoxy)- N-(3-(3-(4- chlorophenoxy)pyrrolidin- 1-yl)bicyclo[1.1.1]pentan- 1-yl)acetamide | 447.3 | 1.76-1.75 (m, 1 H), 1.95 (s, 6 H), 2.24-2.1 (m, 1 H), 2.68-2.48 (m, 3 H), 2.87-2.83 (m, 1 H), 4.39 (s, 2 H), 4.84 (s, 1 H), 6.88 (d, J = 7.2 Hz, 2 H), 6.94 (d, J = 8.4 Hz, 2 H), 7.28 (d, J = 9.2 Hz, 2 H), 7.31 (d, J = 8.8 Hz, 2 H), 8.61 (s, 1 H). |
| 6c | 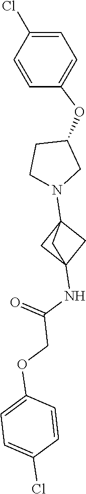 [SMILES: O=C(COc1ccc(Cl)cc1)NC12CC(N3CC[C@H](Oc4ccc(Cl)cc4)C3)(C1)C2] | (S)-2-(4-chlorophenoxy)- N-(3-(3-(4- chlorophenoxy)pyrrolidin- 1-yl)bicyclo[1.1.1]pentan- 1-yl)acetamide | 447.3 | 1.76-1.75 (m, 1 H), 1.95 (s, 6 H), 2.24-2.1 (m, 1 H), 2.68-2.48 (m, 3 H), 2.87-2.83 (m, 1 H), 4.39 (s, 2 H), 4.84 (s, 1 H), 6.88 (d, J = 7.2 Hz, 2 H), 6.94 (d, J = 8.4 Hz, 2 H), 7.28 (d, J = 9.2 Hz, 2 H), 7.31 (d, J = 8.8 Hz, 2 H), 8.61 (s, 1 H). |
| 6d | 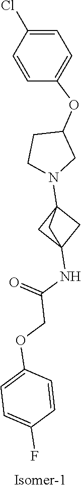 [SMILES: O=C(COc1ccc(F)cc1)NC12CC(N3CCC(Oc4ccc(Cl)cc4)C3)(C1)C2] | N-(3-(3-(4- chlorophenoxy)pyrrolidin- 1-yl)bicyclo[1.1.1]pentan- 1-yl)-2-(4- fluorophenoxy)acetamide | 431.3 | 1.73-1.77 (m, 1 H), 1.95 (s, 6 H), 2.20-2.30 (m, 1 H), 2.53 (d, J = 10.0 Hz, 1 H), 2.65-2.70 (m, 2 H), 2.83-2.88 (m, 1 H), 4.37 (s, 2 H), 4.84 (bs, 1 H), 6.88 (d, J = 8.8 Hz, 2 H), 6.92-6.95 (m, 2 H), 7.10 (t, J = 8.8 Hz, 2 H), 7.28 (d, J = 8.8 Hz, 2 H), 8.59 (s, 1 H). |
| 6e | 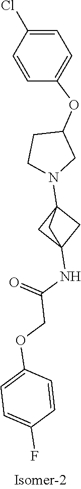 [SMILES: O=C(COc1ccc(F)cc1)NC12CC(N3CCC(Oc4ccc(Cl)cc4)C3)(C1)C2] | N-(3-(3-(4- chlorophenoxy)pyrrolidin- 1-yl)bicyclo[1.1.1]pentan- 1-yl)-2-(4- fluorophenoxy)acetamide | 431.3 | 1.73-1.77 (m, 1 H), 1.95 (s, 6 H), 2.20-2.26 (m, 1 H), 2.58-2.70 (m, 3 H), 2.83-2.88 (m, 1 H), 4.37 (s, 2 H), 4.84 (bs, 1 H), 6.88 (d, J = 8.8 Hz, 2 H), 6.92-6.95 (m, 2 H), 7.10 (t, J = 8.8 Hz, 2 H), 7.28 (d, J = 8.8 Hz, 2 H), 8.59 (s, 1 H). |
| 6d & e | 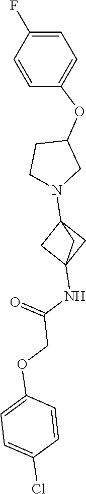 [SMILES: O=C(COc1ccc(Cl)cc1)NC12CC(N3CCC(Oc4ccc(F)cc4)C3)(C1)C2] | 2-(4-chlorophenoxy)-N-(3- (3-(4- fluorophenoxy)pyrrolidin-1- yl)bicyclo[1.1.1]pentan-1- yl)acetamide | 431 | 1.74-1.77 (m, 1 H), 1.96 (s, 6 H), 2.13-2.26 (m, 1 H), 2.40-2.50 (m, 1 H), 2.58-2.60 (m, 1 H), 2.65- 2.70 (m, 1 H), 2.84- 2.88 (m, 1 H), 4.40 (s, 2 H), 4.82-4.85 (m, 1 H), 6.85-6.89 (m, 2 H), 6.95 (d, J = 8.8 Hz, 2 H), 7.08 (t, J = 8.8 Hz, 2 H), 7.32 (d, J = 8.8 Hz, 2 H), 8.61 (s, 1 H). |

### Example 6f and 6d

### N-(3-(3-(3-chloro-4-fluorophenoxy)pyrrolidin-1-yl)bicyclo[1.1.1]pentan-1-yl)-2-(4-chlorophenoxy)acetamide

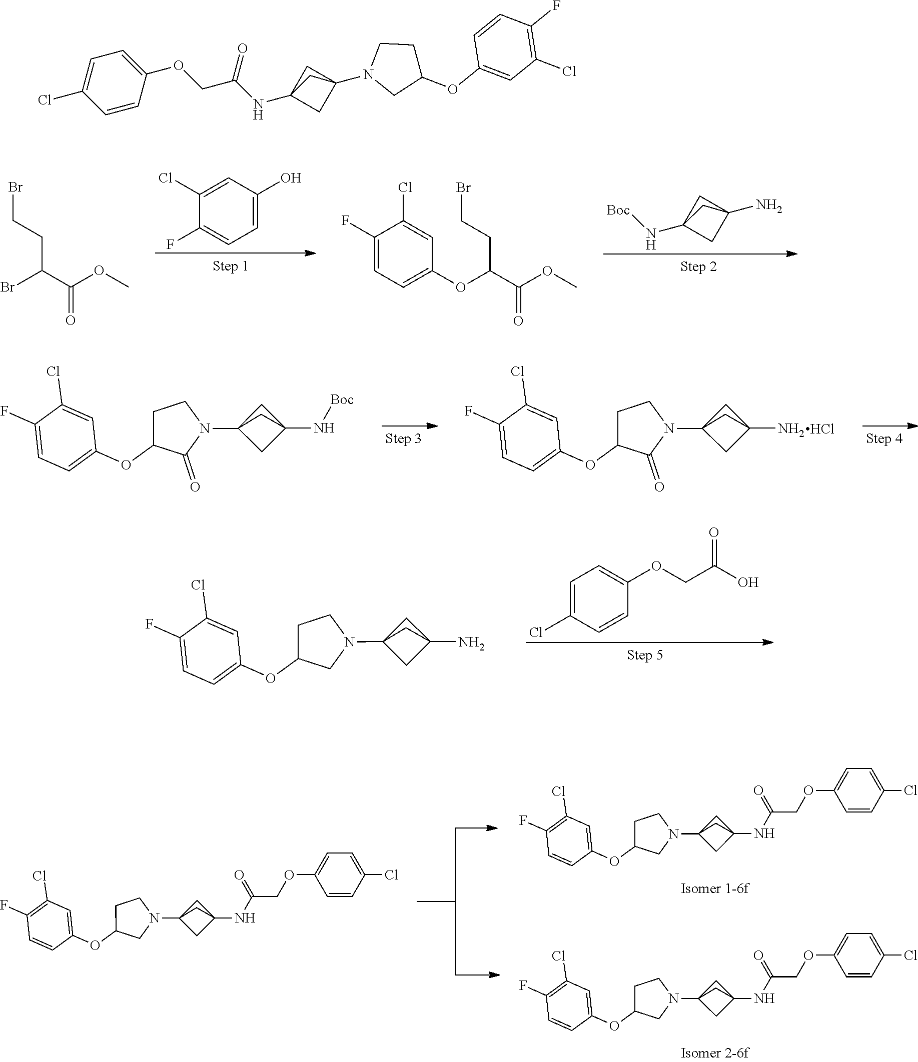

Step 1: To a solution of methyl 3-chloro-4-fluorophenol (2.0 g, 13.65 mmol, 1.0 equiv.) in DMF (50 mL) was added K₂CO₃ (1.88 g, 13.65 mmol, 1.0 equiv.) at 0° C., stirred for 10 mins and then methyl 2,4-dibromobutanoate (1.9 mL, 13.65 mmol, 1.0 equiv.) was added and the reaction was stirred at 60 ⁰0 for 3 h. After consumption of the starting material (TLC, 10% ethyl acetate in hexane), the reaction mixture was diluted with ice cold water (100 mL) and was extracted with EtOAc (2×100 mL). The combined EtOAc extracts were dried over anhydrous Na₂SO₄, filtered and distilled under vacuum. The crude material was purified by flash column chromatography eluting the product at 2-5% EtOAc in hexane to afford methyl 4-bromo-2-(3-chloro-4-fluorophenoxy)butanoate as a light pink liquid (2.76 g, 62.0% yield). ¹H NMR (400 MHz, DMSO-d₆) δ ppm 2.34-2.43 (m, 2H), 3.58-3.68 (m, 5H), 4.98-5.04 (m, 1H), 6.92-6.96 (m, 1H), 7.18-7.21 (m, 1H), 7.29-7.40 (m, 1H).

Step 2: To a solution of tert-butyl (3-aminobicyclo[1.1.1]pentan-1-yl)carbamate (0.8 g, 4.03 mmol, 1.0 equiv.) in Et₃N (2.27 mL, 16.12 mmol, 4.0 equiv.) was added methyl 4-bromo-2-(3-chloro-4-fluorophenoxy)butanoate (1.57 g, 4.84 mmol, 1.2 equiv) at 0° C. in a sealed tube and the mixture was then heated at 100° C. using an oil bath for 1 h. (Note: The reaction was carried out by dividing 0.8 g into 4 batches). After the consumption of the starting material (TLC, 50% ethyl acetate in hexane), the reaction mixture was diluted with ethyl acetate (200 mL) and was washed with water (2×20 mL). The combined EtOAc extracts were dried over anhydrous Na₂SO₄, filtered and evaporated under vacuum. The crude material was purified by column chromatography using an eluent of 60% EtOAc in hexane to obtain the desired product tert-butyl (3-(3-(3-chloro-4-fluorophenoxy)-2-oxopyrrolidin-1-yl)bicyclo[1.1.1]pentan-1-yl)carbamate as a colorless liquid (0.91 g, 55.1%). LCMS (ES) m/z=411[M+H]⁺, 355.0 [M+H]⁺(loss of tert-butyl group). ¹H NMR (400 MHz, DMSO-d₆) δ ppm 1.44 (s, 9H), 2.09-2.18 (m, 1H), 2.39 (s, 6H), 2.46-2.54 (m, 1H), 3.29-3.35 (m, 1H), 3.42-3.47 (m, 1H), 4.73 (t, J=7.4 Hz, 1H), 4.96 (bs, 1H), 6.92-6.95 (m, 1H), 7.00-7.04 (m, 1H), 7.10-7.12 (m, 1H).

Step 3: To a solution of tert-butyl (3-(3-(3-chloro-4-fluorophenoxy)-2-oxopyrrolidin-1-yl)bicyclo[1.1.1]pentan-1-yl)carbamate (0.91 g, 2.21 mmol, 1.0 equiv.) in DCM (20 mL) at 0° C. was added 10 mL of 4 M HCl in 1,4-dioxane and the mixture was stirred at rt for 3 h. After the consumption of the starting material (TLC, 5% MeOH in DCM), the reaction mixture was concentrated and the crude was triturated with n-pentane (2×10 mL) and dried under high vacuum to obtain 1-(3-aminobicyclo[1.1.1]pentan-1-yl)-3-(3-chloro-4-fluorophenoxy)pyrrolidin-2-one hydrochloride (crude yield 0.58 g, 85.2%), which was taken to the next step without further purification. LCMS (ES) m/z=311.1 [M+H]⁺. ¹H NMR (400 MHz, DMSO-d₆) δ ppm 1.87-1.96 (m, 1H), 2.30 (q, J=7.0 Hz, 6H), 2.55-2.58 (m, 1H), 3.25-3.31 (m, 1H), 3.39 (t, J=8.0 Hz, 1H), 5.02 (t, J=7.6 Hz, 1H), 6.99-7.01 (m, 1H), 7.26-7.34 (m, 2H), 8.79 (bs, 3H).

Step 4: To a solution of 1-(3-aminobicyclo[1.1.1]pentan-1-yl)-3-(3-chloro-4-fluorophenoxy)pyrrolidin-2-one hydrochloride (0.75 g, 2.16 mmol, 1.0 equiv.) in THF (10 mL) was added BH₃.Me₂S (0.63 mL, 6.69 mmol, 3.1 equiv.) at 0° C. and the reaction was stirred for 40 h. (Note: 0.75 g was divided into 2 batches and the reaction was performed. 1.5 equiv. of BH₃.Me₂S was added, stirred for 16 h, monitored the progress of the reaction, added again 0.8 equiv. of BH₃.Me₂S, stirred for 8 h and again 0.8 equiv. of BH₃.Me₂S was added and the reaction was stirred for 16 h.). Then the reaction mixture was cooled to 0° C., quenched with MeOH (5 mL), stirred for 30 min, and concentrated under reduced pressure to obtain the crude product. This crude material was triturated with n-pentane (50 mL) and dried under high vacuum to yield 3-(3-(3-chloro-4-fluorophenoxy)pyrrolidin-1-yl)bicyclo[1.1.1]pentan-1-amine as an off-white solid (0.56 g, crude yield), which was used for the next step without further purification. LCMS (ES) m/z=297.1 [M+H]⁺.

Step 5: To a solution of 2-(4-chlorophenoxy)acetic acid (0.42 g, 0.23 mmol, 1.2 equiv.) in DCM (5 mL) at 0° C. was added triethylamine (1.06 mL, 7.52 mmol, 4.0 equiv.), stirred for 10 mins, followed by addition of T3P® (50 wt % in EtOAc) (2.25 mL, 3.76 mmol, 2 equiv). The mixture was stirred at 0° C. for 10 min and then 3-(3-(3-chloro-4-fluorophenoxy)pyrrolidin-1-yl)bicyclo[1.1.1]pentan-1-amine (0.56 g, 1.88 mmol, 1.0 equiv.) in DCM (5 mL) was slowly added at 0° C. and the reaction was stirred at rt for 16 h. After the consumption of the starting material (TLC, 70% ethyl acetate in hexane), the reaction mixture was diluted with DCM (150 mL) and washed with saturated NaHCO₃ (2×10 mL) and water (2×10 mL). The combined organic layer was dried over anhydrous Na₂SO₄, filtered and evaporated under reduced pressure. The crude material was purified by column chromatography using an eluent of 70-80% EtOAc in hexane to obtain the desired product N-(3-(3-(3-chloro-4-fluorophenoxy)pyrrolidin-1-yl)bicyclo[1.1.1]pentan-1-yl)-2-(4-chlorophenoxy)acetamide as a white solid (0.21 g, 24.1%). Racemic product was further submitted for chiral prep HPLC to separate the isomers by using the following analytical conditions: [Column: CHIRALPAK IC (100 mm×4.6 mm×3 mic); Flow rate: 1.0 mL/min; Mobile phase: n-Hexane:IPA with 0.1% DEA (85:15)]. Fractions containing product were evaporated separately under reduced pressure, washed with n-pentane (10 mL) and dried under high vacuum.

Isomer 1 (6f, Single Unknown Stereochemistry):

Recovery: 0.055 g (as off white solid). LCMS (ES) m/z=465.3 [M+H]⁺. ¹H NMR (400 MHz, DMSO-d₆) δ ppm 1.76 (bs, 1H), 1.95 (s, 6H), 2.21-2.23 (m, 1H), 2.58-2.68 (m, 3H), 2.82-2.88 (m, 1H), 4.40 (s, 2H), 4.87 (bs, 1H), 6.88 (s, 1H), 6.94 (d, J=8.4 Hz, 2H), 7.08 (s, 1H), 7.27-7.32 (m, 3H), 8.62 (s, 1H). Chiral HPLC purity: 100.0% at 220 nm ; % ee: 100.0

Isomer 2 (6g, Single Unknown Stereochemistry):

Recovery: 0.025 g (as off white solid). LCMS (ES) m/z=465.3 [M+H]⁺. ¹H NMR (400 MHz, DMSO-d₆) δ ppm 1.74 (bs, 1H), 1.95 (s, 6H), 2.21-2.22 (m, 1H), 2.58-2.68 (m, 3H), 2.82-2.88 (m, 1H), 4.39 (s, 2H), 4.86 (bs, 1H), 6.87 (s, 1H), 6.94 (d, J=7.6 Hz, 2H), 7.07 (s, 1H), 7.26-7.32 (m, 3H), 8.60 (s, 1H). Chiral HPLC purity: 100.0% at 225 nm; % ee: 100.0%.

TABLE 13

| | | | LCMS m/z | ¹H-NMR (400 MHz, |
|---|---|---|---|---|
| Cmpd # | Structure | Name | [M + H]⁺ | DMSO-d₆) |
| 6f | 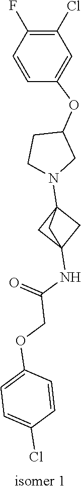 [SMILES: O=C(COc1ccc(Cl)cc1)NC12CC(N3CCC(Oc4ccc(F)c(Cl)c4)C3)(C1)C2] | N-(3-(3-(3-chloro-4- fluorophenoxy)pyrrolidin-1- yl)bicyclo[1.1.1]pentan-1-yl)-2- (4-chlorophenoxy)acetamide | 465.3 | 1.76 (bs, 1 H), 1.95 (s, 6 H), 2.21-2.23 (m, 1 H), 2.58-2.68 (m, 3 H), 2.82- 2.88 (m, 1 H), 4.40 (s, 2 H), 4.87 (bs, 1 H), 6.88 (s, 1 H), 6.94 (d, J = 8.4 Hz, 2 H), 7.08 (s, 1 H), 7.27- 7.32 (m, 3 H), 8.62 (s, 1 H). |
| 6g | 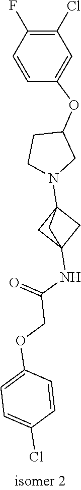 [SMILES: O=C(COc1ccc(Cl)cc1)NC12CC(N3CCC(Oc4ccc(F)c(Cl)c4)C3)(C1)C2] | N-(3-(3-(3-chloro-4- fluorophenoxy)pyrrolidin-1- yl)bicyclo[1.1.1]pentan-1-yl)-2- (4-chlorophenoxy)acetamide | 465.3 | 1.74 (bs, 1 H), 1.95 (s, 6 H), 2.21-2.22 (m, 1 H), 2.58-2.68 (m, 3 H), 2.82- 2.88 (m, 1 H), 4.39 (s, 2 H), 4.86 (bs, 1 H), 6.87 (s, 1 H), 6.94 (d, J = 7.6 Hz, 2 H), 7.07 (s, 1 H), 7.26- 7.32 (m, 3 H), 8.60 (s, 1 H). |

### Example 6h

### N-(3-(3-(4-chlorophenoxy)pyrrolidin-1-yl)bicyclo[1.1.1]pentan-1-yl)-2-((5-chloropyridin-2-yl)oxy)acetamide

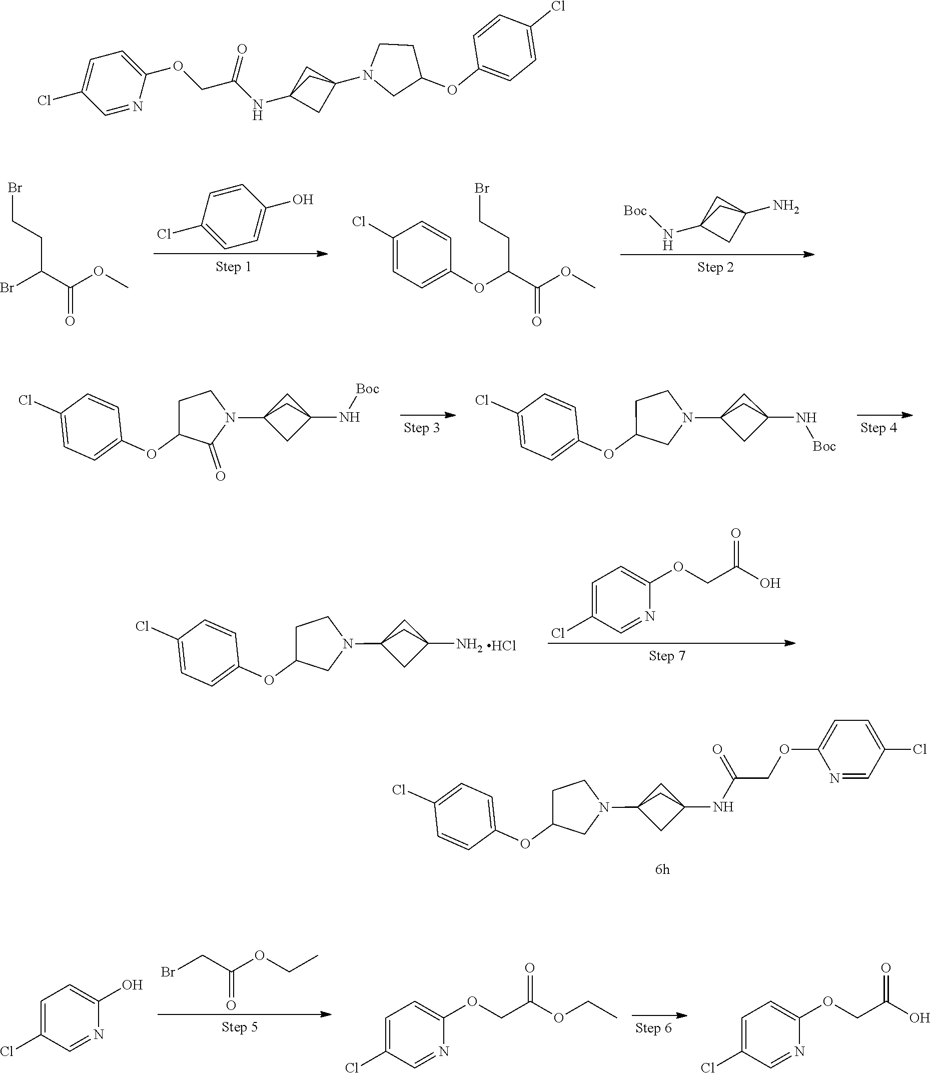

Step 1: To a solution of 4-chlorophenol (3.9 g, 30.46 mmol, 1.0 equiv.) in DMF (180 mL), K₂CO₃ (4.2 g, 30.46 mmol, 1.0 equiv) was added followed by the addition of methyl-2,4-dibromobutanoate (4.26 mL, 30.46 mmol, 1.0 equiv.) at room temperature and the reaction was stirred at 60° C. for 3 h (Note: Reaction was performed by dividing 3.9 g into 3 batches.). After the completion of the reaction (TLC, 10% EtOAc in hexane), the reaction mixture was then allowed to come to room temperature. Water (200 mL) was added and the mixture was extracted with EtOAc (3×100 mL). The combined EtOAc extracts were dried over anhydrous Na₂SO₄, filtered and distilled under vacuum. The crude material was purified by flash column chromatography eluting the product at 5% EtOAc in hexane to provide methyl 4-bromo-2-(4-chlorophenoxy)butanoate as a colorless liquid (5.8 g, 62.0% yield). ¹H NMR (400 MHz, CDCl₃) δ ppm 2.42-2.52 (m, 2H), 3.52-3.60 (m, 2H), 3.76 (s, 3H), 4.82-4.85 (m, 1H), 6.85 (d, J=8.4 Hz, 2H), 7.26 (d, J=8.0 Hz, 2H).

Step 2: To a solution of tert-butyl (3-aminobicyclo[1.1.1]pentan-1-yl)carbamate (3.64 g, 18.38 mmol, 1.0 equiv) in Et₃N (10.5 mL, 73.52 mmol, 4.0 equiv.) in a sealed tube was added methyl 4-bromo-2-(4-chlorophenoxy)butanoate (5.67 g, 18.38 mmol, 1.0 equiv.) and the mixture was heated at 100° C. for 1 h. (Note: Reaction was performed in multiple batches like 0.52 g×7). After completion of the reaction (TLC, 50% EtOAc in hexane), the reaction mixture was diluted with water (100 mL) and extracted with DCM (2×100 mL). The combined EtOAc extracts were dried over anhydrous Na₂SO₄, filtered and evaporated under vacuum. The crude material was purified by column chromatography using an eluent of 35% EtOAc in hexane to obtain the desired product tert-butyl (3-(3-(4-chlorophenoxy)-2-oxopyrrolidin-1-yl)bicyclo[1.1.1]pentan-1-yl)carbamate as an off-white solid (3.0 g, 42.0% yield). LCMS (ES) m/z=393.1 [M+H]⁺. ¹H NMR (400 MHz, DMSO-d₆) δ ppm 1.44 (s, 9H), 2.11-2.18 (m, 1H), 2.39 (s, 6H), 2.48-2.51 (m, 1H), 3.32-3.35 (m, 1H), 3.43-3.47 (m, 1H), 4.77 (t, J=7.0 Hz, 1H), 4.95 (s, 1H), 6.98 (d, J=6.8 Hz, 2H), 7.21 (d, J=7.2 Hz, 2H).

Step 3: To a solution of tert-butyl (3-(3-(4-chlorophenoxy)-2-oxopyrrolidin-1-yl)bicyclo[1.1.1]pentan-1-yl)carbamate (1.8 g, 4.58 mmol, 1.0 equiv.) in THF (30 mL) was added BH₃.Me₂S (0.87 mL, 9.16 mmol, 3 equiv) at 0° C. (Note: 1.8 g was divided into 2 batches and the reaction was performed). After completion of the reaction (TLC, 50% EtOAc in hexane), the reaction mixture was quenched with methanol at 0° C. and stirred for 2 h. The reaction mixture was concentrated under reduced pressure to give crude product, which was diluted with water (200 mL) and extracted with DCM (3×100 mL). The combined organic layer was washed with brine solution (100 mL) and dried over anhydrous Na₂SO₄, filtered and concentrated to obtain the crude product. The crude material was purified by silica gel column chromatography using 55% EtOAc in hexane as an eluent to yield tert-butyl (3-(3-(4-chlorophenoxy)pyrrolidin-1-yl)bicyclo[1.1.1]pentan-1-yl)carbamate as an off-white solid (0.8 g, 46.24% yield). LCMS (ES) m/z=379.1 [M+H]⁺. ¹H NMR (400 MHz, DMSO-d₆) δ ppm 1.44 (s, 9H), 1.91 (bs, 1H), 2.03 (s, 6H), 2.17-2.32 (m, 1H), 2.60-2.61 (m, 1H), 2.76-2.84 (m, 2H), 2.95 (bs, 1H), 4.78 (s, 1H), 4.89 (s, 1H), 6.76 (d, J=8.8 Hz, 2H), 7.21 (d, J=8.8 Hz, 2H).

Step 4: To a solution of tert-butyl (3-(3-(4-chlorophenoxy)pyrrolidin-1-yl)bicyclo[1.1.1]pentan-1-yl)carbamate (0.8 g, 2.11 mmol, 1.0 equiv.) in DCM (10 mL) at 0° C. was added 5 mL of 4 M HCl in 1,4-dioxane and the mixture was stirred at room temperature for 16 h. After completion of the reaction (TLC, 5% MeOH in DCM), the reaction mixture was concentrated to obtain 3-(3-(4-chlorophenoxy)pyrrolidin-1-yl)bicyclo[1.1.1]pentan-1-amine hydrochloride (crude yield 0.65 g, 98.48% yield), which was taken to the next step without further purification. LCMS (ES) m/z=279.1 [M+H]⁺. ¹H NMR (400 MHz, DMSO-d₆) δ ppm 2.09 (bs, 1H), 2.24 (s, 6H), 3.30-3.67 (m, 5H), 5.10 (s, 1H), 6.96 (d, J=8.8 Hz, 2H), 7.34 (d, J=8.8 Hz, 2H), 9.10 (s, 3H).

Step 5: To a solution of 5-chloropyridin-2-ol (2.0 g, 15.44 mmol, 1.0 equiv.) in DMF (20 mL) was added silver carbonate (5.96 g, 21.61 mmol, 1.4 equiv.) at 0° C., stirred for 10 min and then ethyl 2-bromoacetate (2.56 mL, 23.16 mmol, 1.5 equiv.) was added and the mixture was heated at 80° C. for 2 h. After the completion of the reaction (TLC, 20% EtOAc in hexane), the reaction mixture was filtered through a Celite® bed, and washed the Celite® bed with ethyl acetate (100 mL). The filtrate was evaporated under vacuum. The crude material was purified by column chromatography using an eluent of 10-15% EtOAc in hexane to obtain the desired product ethyl 2-((5-chloropyridin-2-yl)oxy)acetate as a colorless liquid (0.67 g, 20.0% yield). LCMS (ES) m/z=216.1 [M+H]^{+ 1}H NMR (400 MHz, DMSO-d₆) δ ppm 1.15 (t, J=7.0 Hz, 3H), 4.10 (q, J=6.9 Hz, 2H), 4.87 (s, 2H), 6.97 (d, J=8.8 Hz, 1H), 7.83 (d, J=8.4 Hz, 1H), 8.16 (s, 1H).

Step 6: To a solution of ethyl 2-((5-chloropyridin-2-yl)oxy)acetate (0.67 g, 3.11 mmol, 1.0 equiv.) in THF (4 mL) and water (4 mL) was added lithium hydroxide monohydrate (1.3 g, 31.1 mmol, 10.0 equiv.) at 0° C. and the mixture was stirred at room temperature for 6 h. After the completion of the reaction (TLC, 5% MeOH in DCM), the reaction mixture was evaporated under vacuum and the aqueous layer was extracted with ethyl acetate (2×10 mL). Then the aqueous layer was cooled to 0° C. and acidified with concentrated HCl (pH adjusted to 1) and was extracted with ethyl acetate (2×50 mL). The combined organic layer was dried over anhydrous Na₂SO₄, filtered and concentrated. The crude material was triturated with n-pentane (2×5 mL) and dried under high vacuum to obtain the desired product 2-((5-chloropyridin-2-yl)oxy)acetic acid as an off-white solid (0.43 g, 73.9% yield). LCMS (ES) m/z=188.0 [M+H]⁺. ¹H NMR (400 MHz, DMSO-d₆) δ ppm 4.79 (s, 2H), 6.94 (d, J=8.8 Hz, 1H), 7.82 (dd, J=8.8 Hz, 2.4 Hz, 1H), 8.162-8.168 (m, 1H), 12.85 (bs, 1H).

Step 7: To a solution of 2-((5-chloropyridin-2-yl)oxy)acetic acid (0.054 g, 0.28 mmol, 1.5 equiv.) in DCM (6 mL) at 0° C. was added triethylamine (0.11 mL, 0.76 mmol, 4.0 equiv), stirred for 10 mins and T3P® (50 wt % in EtOAc) (0.23 mL, 0.38 mmol, 2.0 equiv.) was added. The reaction mixture was stirred at 0° C. for 10 min and then 3-(3-(4-chlorophenoxy)pyrrolidin-1-yl)bicyclo[1.1.1]pentan-1-amine hydrochloride (0.06 g, 0.19 mmol, 1.0 equiv.) (which was neutralized with triethyl amine in DCM) was added at 0° C. and the reaction was stirred at room temperature for 2 h. After the consumption of the starting material (TLC, 5% MeOH in DCM), the reaction mixture was diluted with DCM (100 mL) and was washed with saturated aqueous NaHCO₃ solution (2×10 mL) and water (2×10 mL). The combined organic layer was dried over anhydrous Na₂SO₄, filtered and evaporated under reduced pressure to get the crude. The crude product was purified by silica gel column chromatography using 6-7% MeOH in DCM as an eluent to afford the desired product N-(3-(3-(4-chlorophenoxy)pyrrolidin-1-yl)bicyclo[1.1.1]pentan-1-yl)-2-((5-chloropyridin-2-yl)oxy)acetamide as an off-white solid (0.06 g, 70.6% yield).

LCMS (ES) m/z=448.3 [M+H]⁺. ¹H NMR (400 MHz, DMSO-d₆) δ ppm 1.73-1.76 (m, 1H), 1.95 (s, 6H), 2.17-2.26 (m, 1H), 2.56-2.69 (m, 3H), 2.82-2.86 (m, 1H), 4.63 (s, 2H), 4.82-4.84 (m, 1H), 6.86-6.93 (s, 3H), 7.28 (d, J=9.2 Hz, 2H), 7.81 (dd, J=8.8 Hz, 6.4 Hz, 1H), 8.14 (d, J=2.0 Hz, 1H), 8.55 (s, 1H).

### Example 6i

### N-(3-(3-(4-chlorophenoxy)pyrrolidin-1-yl)bicyclo[1.1.1]pentan-1-yl)-2-(4-(trifluoromethyl)phenoxy)acetamide

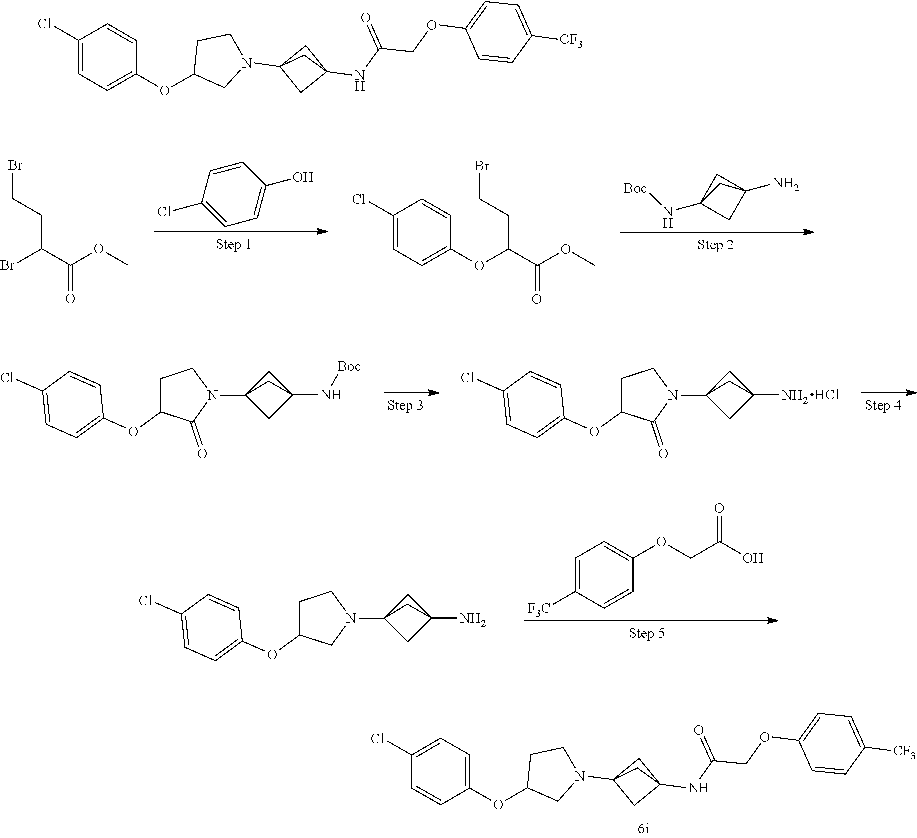

Step 1: To a solution of methyl 2,4-dibromobutanoate (2 g, 7.69 mmol, 1 equiv) in DMF (60 mL) was added 4-chlorophenol (0.98 g, 7.69 mmol, 1 equiv) at rt followed by K₂CO₃ (1.06 g, 7.69 mmol, 1 equiv) and the reaction was stirred at 60° C. for 3 h. The reaction mixture was then allowed to come to rt, ice cold water (100 mL) was added and the mixture was extracted with EtOAc (2×100 mL). The combined EtOAc extracts were dried over anhydrous Na₂SO₄, filtered and distilled under vacuum. The crude material was purified by flash column chromatography eluting the product at 3.7% EtOAc in hexane to afford methyl 4-bromo-2-(4-chlorophenoxy)butanoate as a gum (1.3 g, 56%). LCMS (ES) m/z=308 [M+H]⁺, ¹H NMR (400 MHz, CDCl₃) δ ppm 2.44-2.52 (m, 2H), 3.48-3.58 (m, 2H), 3.76 (s, 3H), 4.83-4.85 (m, 1H), 6.84-6.86 (m, 2H), 7.23-7.25 (m, 2H).

Step 2: Note:—This reaction was performed in two batches, each batch with 0.4 g, ie 2×0.4 g=0.8 g]. Methyl 4-bromo-2-(4-chlorophenoxy)butanoate (0.62 g, 2.02 mmol, 1 equiv) and tert-butyl (3-aminobicyclo[1.1.1]pentan-1-yl)carbamate (0.4 g, 2.02 mmol, 1 equiv) were charged to a sealed tube and Et₃N (1.1 mL, 8.08 mmol, 4 equiv) was added. The reaction mixtures were then heated at 100° C. using an oil bath for 1.5 h. After completion, the reaction mixtures were cooled to room temperature, diluted with water (15 mL) and extracted with EtOAc (2×50 mL). The combined EtOAc extracts were dried over anhydrous Na₂SO₄, filtered and evaporated under vacuum. The crude material was purified by column chromatography using an eluent of 35% EtOAc in hexane to obtain the desired product tert-butyl (3-(3-(4-chlorophenoxy)-2-oxopyrrolidin-1-yl)bicyclo[1.1.1]pentan-1-yl)carbamate as an off-white solid (0.7 g, 70%). LCMS (ES) m/z=393.1 [M+H]⁺. ¹H NMR (400 MHz, CDCl3-d₆) δ ppm 1.44 (s, 9H), 2.13-2.16 (m, 1H), 2.39 (s, 6H), 2.45-2.50 (m, 1H), 3.31-3.33 (m, 1H), 3.43-3.45 (m, 1H), 4.76-4.79 (m, 1H), 6.82 (bs, 1H), 6.98 (d, J=8.8 Hz, 2H), 7.21 (d, J=8.4 Hz, 2H).

Step 3: To a solution of tert-butyl (3-(3-(4-chlorophenoxy)-2-oxopyrrolidin-1-yl)bicyclo[1.1.1]pentan-1-yl)carbamate (0.7 g, 1.78 mmol, 1 equiv) in DCM (15 mL) at 0° C. was added 10 mL of 4 M HCl in 1,4-dioxane and the mixture was stirred at rt for 12 h. The reaction mixture was concentrated to give 1-(3-aminobicyclo[1.1.1]pentan-1-yl)-3-(4-chlorophenoxy)pyrrolidin-2-one hydrochloride (0.5 g, crude), which was taken to the next step without further purification. LCMS (ES) m/z=293.1 [M+H]⁺. ¹H NMR (400 MHz, DMSO-d₆) δ ppm 1.89-1.92 (m, 2H), 2.31 (s, 6H), 3.28-3.30 (m, 1H), 3.36-3.39 (m, 1H), 4.96-5.00 (m, 1H), 7.02 (d, J=8.8 Hz, 2H), 7.31 (d, J=8.4 Hz, 2H), 8.71-8.87 (s, 3H).

Step 4: To a solution of 1-(3-aminobicyclo[1.1.1]pentan-1-yl)-3-(4-chlorophenoxy)pyrrolidin-2-one hydrochloride (0.5 g, 1.52 mmol, 1 equiv) in THF (10 mL) was added BH₃.Me₂S (0.4 mL, 4.5 mmol, 3 equiv) at 0° C. (Note: BH₃.Me₂S was added at 3 interval points of time. First 1.5 equivalents were added and stirred for 16 h and again 0.75 equivalents were added and stirred for 8 h. After that time reaction progress was monitored by LCMS, which showed the presence of starting material and again 0.75 equivalents were added and stirred for 8 h.) After completion of the reaction, the reaction mixture was quenched with MeOH (3 mL) at 0° C., stirred for 30 min, and concentrated under reduced pressure to obtain the crude product. This crude material was washed with n-pentane (15 mL) and dried under vacuum to yield 3-(3-(4-chlorophenoxy)pyrrolidin-1-yl)bicyclo[1.1.1]pentan-1-amine (0.6 g, crude), which was used for the next step without further purification. LCMS (ES) m/z=279 [M+H]⁺.

Step 5: To a solution of 2-(4-(trifluoromethyl)phenoxy)acetic acid (0.56 g, 2.58 mmol, 1.2 equiv) in DCM (10 mL) at 0° C. was added triethylamine (1.2 mL, 8.60 mmol, 4 equiv) followed by T3P® (50 wt % in EtOAc) (2.5 mL, 4.30 mmol, 2 equiv). The mixture was stirred at 0° C. for 10 min, at which time, this reaction mixture was added: 3-(3-(4-chlorophenoxy)pyrrolidin-1-yl)bicyclo[1.1.1]pentan-1-amine (0.6 g, 2.15 mmol, 1 equiv) in DCM (10 mL) slowly at 0° C., and the reaction was stirred at rt for 12 h. The reaction mixture was diluted with water (10 mL) and extracted with DCM (2×20 mL). The combined organic layer was washed with saturated aqueous NaHCO₃ solution (15 mL) and then dried over anhydrous Na₂SO₄, filtered and evaporated under reduced pressure. The crude product was purified by preparative TLC using 50% EtOAc in hexanes. After final drying, the desired product N-(3-(3-(4-chlorophenoxy)pyrrolidin-1-yl)bicyclo[1.1.1]pentan-1-yl)-2-(4-(trifluoromethyl)phenoxy)acetamide was obtained as a white solid (0.097 g, 19%). LCMS (ES) m/z=481.4 [M+H]⁺. ¹H NMR (400 MHz, DMSO-d₆) δ ppm 1.73-1.77 (m, 1H), 1.95 (s, 6H), 2.12-2.26 (m, 1H), 2.58-2.70 (m, 3H), 2.84-2.88 (m, 1H), 4.51 (s, 2H), 4.83-4.86 (m, 1H), 6.88 (d, J=8.8 Hz, 2H), 7.09 (d, J=8.8 Hz, 2H), 7.28 (d, J=8.8 Hz, 2 H), 7.64 (d, J=8.4 Hz, 2H), 8.67 (s, 1H).

The Compounds of Examples 6j -6o were prepared generally according to the procedure described above for Examples 6h and 6i.

TABLE 14

| Cmpd | | | LCMS m/z | ¹H-NMR (400 MHz, |
|---|---|---|---|---|
| # | Structure | Name | [M + H]⁺ | DMSO-d₆) |
| 6h | 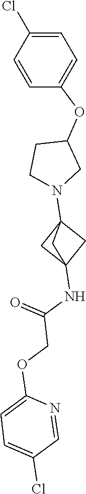 [SMILES: O=C(COc1ccc(Cl)cn1)NC12CC(N3CCC(Oc4ccc(Cl)cc4)C3)(C1)C2] | N-(3-(3-(4-chlorophenoxy)pyrrolidin-1- yl)bicyclo[1.1.1]pentan-1-yl)-2-((5- chloropyridin-2-yl)oxy)acetamide | 448.3 | 1.73-1.76 (m, 1 H), 1.95 (s, 6 H), 2.17- 2.26 (m, 1 H), 2.56- 2.69 (m, 3 H), 2.82- 2.86 (m, 1 H), 4.63 (s, 2 H), 4.82-4.84 (m, 1 H), 6.86-6.93 (s, 3 H), 7.28 (d, J = 9.2 Hz, 2 H), 7.81 (dd, J = 8.8 Hz, 6.4 Hz, 1 H), 8.14 (d, J = 2.0 Hz, 1 H), 8.55 (s, 1 H). |
| 6i | 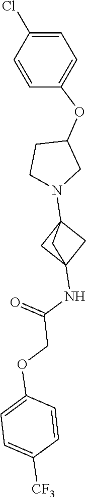 [SMILES: O=C(COc1ccc(C(F)(F)F)cc1)NC12CC(N3CCC(Oc4ccc(Cl)cc4)C3)(C1)C2] | N-(3-(3-(4-chlorophenoxy)pyrrolidin-1- yl)bicyclo[1.1.1]pentan-1-yl)-2-(4- (trifluoromethyl)phenoxy)acetamide | 481.4 | 1.73-1.77 (m, 1 H), 1.95 (s, 6 H), 2.12- 2.26 (m, 1 H), 2.58- 2.70 (m, 3 H), 2.84- 2.88 (m, 1 H), 4.51 (s, 2 H), 4.83-4.86 (m, 1 H), 6.88 (d, J = 8.8 Hz, 2 H), 7.09 (d, J = 8.8 Hz, 2 H), 7.28 (d, J = 8.8 Hz, 2 H), 7.64 (d, J = 8.4 Hz, 2 H), 8.67 (s, 1 H). |
| 6j | 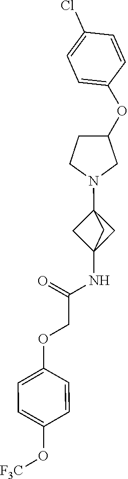 [SMILES: O=C(COc1ccc(OC(F)(F)F)cc1)NC12CC(N3CCC(Oc4ccc(Cl)cc4)C3)(C1)C2] | N-(3-(3-(4-chlorophenoxy)pyrrolidin-1- yl)bicyclo[1.1.1]pentan-1-yl)-2-(4- (trifluoromethoxy)phenoxy)acetamide | 497.4 | 1.77 (s, 1 H), 1.95 (s, 6 H), 2.23 (m, 2 H), 2.61-2.58 (m, 1 H), 2.65 (m, 1 H), 2.86 (m, 1 H), 4.42 (s, 2 H), 7.85 (s, 1 H), 6.88 (d, J = 7.2 Hz, 2 H), 7.00 (d, J = 6.4 Hz, 2 H),7.28 (d, J = 7.6 Hz, 4 H), 8.62 (s, 1 H). |
| 6k | 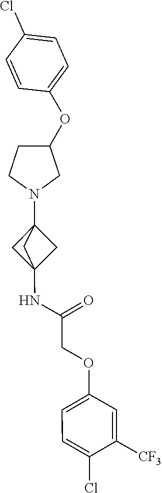 [SMILES: O=C(COc1ccc(Cl)c(C(F)(F)F)c1)NC12CC(N3CCC(Oc4ccc(Cl)cc4)C3)(C1)C2] | 2-(4-chloro-3-(trifluoromethyl)phenoxy)- N-(3-(3-(4-chlorophenoxy)pyrrolidin-1- yl)bicyclo[1.1.1]pentan-1-yl)acetamide | 515.4 | 1.74-1.77 (m, 1 H), 1.96 (s, 6 H), 2.22- 2.25 (m, 1 H), 2.61- 2.69 (m, 3 H), 2.84- 2.88 (m, 1 H), 4.52 (s, 2 H), 4.85 (bs, 1 H), 6.88 (d, J = 8.8 Hz, 2 H), 7.24-7.29 (m, 3 H), 7.39 (s, 1 H), 7.62 (d, J = 9.2 Hz, 1 H), 8.67 (s, 1 H). |
| 6l | 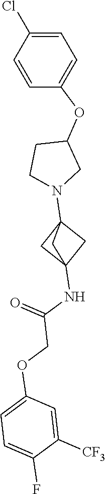 [SMILES: O=C(COc1ccc(F)c(C(F)(F)F)c1)NC12CC(N3CCC(Oc4ccc(Cl)cc4)C3)(C1)C2] | N-(3-(3-(4-chlorophenoxy)pyrrolidin-1- yl)bicyclo[1.1.1]pentan-1-yl)-2-(4-fluoro- 3-(trifluoromethyl)phenoxy)acetamide | 499.0 | 1.75-1.77 (m, 1 H), 1.96 (s, 6 H), 2.20- 2.27 (m, 1 H), 2.58- 2.71 (m, 3 H), 2.84- 2.88 (m, 1 H), 4.49 (s, 2 H), 4.84 (bs, 1 H), 6.88 (d, J = 8.8 Hz, 2 H), 7.27-7.29 (m, 4 H), 7.43 (t, J = 9.6 Hz, 1 H), 8.65 (s, 1 H). |
| 6m | 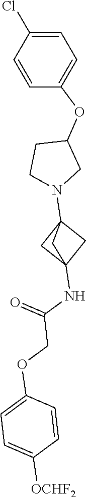 [SMILES: O=C(COc1ccc(OC(F)F)cc1)NC12CC(N3CCC(Oc4ccc(Cl)cc4)C3)(C1)C2] | N-(3-(3-(4-chlorophenoxy)pyrrolidin-1- yl)bicyclo[1.1.1]pentan-1-yl)-2-(4- (difluoromethoxy)phenoxy)acetamide | 479.4 | 1.95-2.06 (m, 1 H), 2.13 (s, 6 H), 2.25- 2.23 (m, 1 H), 2.60- 2.66 (m, 1 H), 2.82- 2.90 (m, 2 H), 2.94- 2.98 (m, 1 H), 4.43 (s, 2 H), 4.88-4.89 (m, 1 H), 6.48-6.86 (m, 4 H), 6.97-6.99 (m, 2 H), 7.08 (d, J = 8.8 Hz, 2 H), 7.23 (d, J = 8.8 Hz, 2 H). |
| 6n | 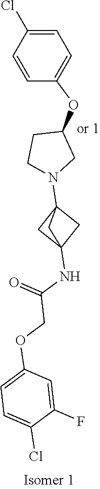 [SMILES: O=C(COc1ccc(Cl)c(F)c1)NC12CC(N3CC[C@@H](Oc4ccc(Cl)cc4)C3)(C1)C2] | 2-(4-chloro-3-fluorophenoxy)-N-(3-(3-(4- chlorophenoxy)pyrrolidin-1- yl)bicyclo[1.1.1]pentan-1-yl)acetamide | 465.0 | 1.76-1.77 (m, 1 H), 1.95 (s, 6 H), 2.20- 2.25 (m, 2 H), 2.58- 2.60 (m, 1 H), 2.65- 2.68 (m, 1 H), 2.84- 2.88 (m, 1 H), 4.44 (s, 2 H), 4.84 (s, 1 H), 6.82 (d, J = 8.8 Hz, 1 H), 6.88 (d, J = 9.2 Hz, 2 H), 7.02- 7.05 (m, 1 H), 7.28 (d, J = 8.8 Hz, 2 H), 7.46 (d, J = 8.8 Hz, 1 H), 8.62 (s, 1 H) |
| 6o | 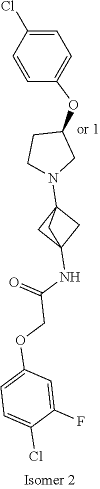 [SMILES: O=C(COc1ccc(Cl)c(F)c1)NC12CC(N3CC[C@@H](Oc4ccc(Cl)cc4)C3)(C1)C2] | 2-(4-chloro-3-fluorophenoxy)-N-(3-(3-(4- chlorophenoxy)pyrrolidin-1- yl)bicyclo[1.1.1]pentan-1-yl)acetamide | 465.0 | 1.75-1.77 (m, 1 H), 1.95 (s, 6 H), 2.20- 2.30 (m, 2 H), 2.58- 2.60 (m, 1 H), 2.65- 2.68 (m, 1 H), 2.84- 2.88 (m, 1 H), 4.44 (s, 2 H), 4.84 (s, 1 H), 6.82 (d, J = 8.8 Hz, 1 H), 6.86 (d, J = 8.8 Hz, 2 H), 7.02- 7.05 (m, 1 H), 7.28 (d, J = 8.8 Hz, 2 H), 7.46 (d, J = 8.8 Hz, 1 H), 8.62 (s, 1 H). |

### Example 6p

### 2-(4-chlorophenoxy)-N-(3-(3-(4-(trifluoromethyl)phenoxy)pyrrolidin-1-yl)bicyclo[1.1.1]pentan-1-yl)acetamide

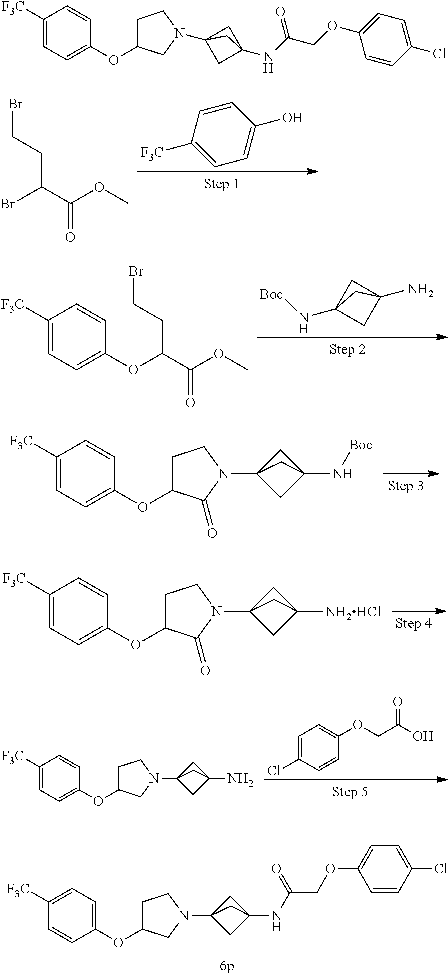

Step 1: To a solution of methyl 2,4-dibromobutanoate (1 g, 3.84 mmol, 1 equiv) in DMF (30 mL) was added 4-(trifluoromethyl)phenol (0.49 g, 3.84 mmol, 1 equiv) at rt followed by K₂CO₃ (0.53 g, 3.84 mmol, 1 equiv) and the reaction was stirred at 60° C. for 3 h. The reaction mixture was then allowed to come to rt. Ice cold water (100 mL) was added and the mixture was extracted with EtOAc (2×100 mL). The combined EtOAc extracts were dried over anhydrous Na₂SO₄, filtered and distilled under vacuum. The crude material was purified by flash column chromatography eluting the product at 3.9% EtOAc in hexane to provide methyl 4-bromo-2-(4-(trifluoromethyl)phenoxy)butanoate as a gum (0.8 g, 61%). LCMS (ES) m/z=342.9 [M+H]⁺, ¹H NMR (400 MHz, CDCl₃) δ ppm 2.36-2.44 (m, 2H), 3.59-3.68 (m, 2H), 3.69 (s, 3H), 5.08-5.11 (m, 1H), 7.10 (d, J=8.4 Hz, 2H), 7.65 (d, J=8.4 Hz, 2H).

Step 2: [Note:—This reaction was performed in two batches, each batch with 0.23 g, ie 2×0.23 g=0.46 g]. To a solution of methyl 4-bromo-2-(4-(trifluoromethyl)phenoxy)butanoate (0.39 g, 1.16 mmol, 1 equiv) and tert-butyl (3-aminobicyclo[1.1.1]pentan-1-yl)carbamate (0.23 g, 1.16 mmol, 1 equiv) charged to a sealed tube was added Et₃N (0.64 mL, 4.64 mmol, 4 equiv). The mixture was then heated at 100° C. using an oil bath for 1.5 h. After completion, the reaction mixtures were cooled to room temperature, diluted with water (15 mL) and extracted with EtOAc (2×50 mL). The combined EtOAc extracts were dried over anhydrous Na₂SO₄, filtered and evaporated under vacuum. The crude material was purified by column chromatography using an eluent of 36% EtOAc in hexane to obtain the desired product tert-butyl (3-(2-oxo-3-(4-(trifluoromethyl)phenoxy)pyrrolidin-1-yl)bicyclo[1.1.1]pentan-1-yl)carbamate as an off-white solid (0.5 g, 55%). LCMS (ES) m/z=371.1 [M+H]⁺ (loss of tert-butyl group).¹H NMR (400 MHz, CDCl3-d₆) δ ppm 1.26-1.28 (m, 1H), 1.44 (s, 9H), 2.16-2.20 (m, 1H), 2.40 (s, 6H), 2.53 (bs, 1H), 3.34-3.36 (m, 1H), 3.47-3.52 (m, 1H), 4.88-4.90 (m, 1H), 7.11 (d, J=7.6 Hz, 2H), 7.53 (d, J=8.4 Hz, 2H).

Step 3: To a solution of tert-butyl (3-(2-oxo-3-(4-(trifluoromethyl)phenoxy)pyrrolidin-1-yl)bicyclo[1.1.1]pentan-1-yl)carbamate (0.5 g, 1.17 mmol, 1 equiv) in DCM (15 mL) at 0° C. was added 10 mL of 4 M HCl in 1,4-dioxane and the mixture was stirred at rt for 12 h. The reaction mixture was concentrated to obtain 1-(3-aminobicyclo[1.1.1]pentan-1-yl)-3-(4-(trifluoromethyl)phenoxy)pyrrolidin-2-one hydrochloride (0.4 g, crude), which was taken to the next step without further purification. LCMS (ES) m/z=327.1 [M+H]⁺. ¹H NMR (400 MHz, DMSO-d₆) δ ppm 2.34 (m, 6H), 3.30-3.41 (m, 4H), 5.13-5.16 (m, 1H), 7.18 (d, J=8.4 Hz, 2H), 7.63 (d, J=8 Hz, 2H), 8.74-8.86 (s, 3H).

Step 4: To a solution of 1-(3-aminobicyclo[1.1.1]pentan-1-yl)-3-(4-(trifluoromethyl)phenoxy)pyrrolidin-2-one hydrochloride (0.4 g, 1.10 mmol, 1 equiv) in THF (10 mL) was added BH₃.Me₂S (0.31 mL, 3.30 mmol, 3 equiv) at 0° C. (Note: BH₃.Me₂S was added at 3 interval points of time: first 1.5 equivalents were added and stirred for 16 h and again 0.75 equivalents were added and stirred for 8 h. After that time, the reaction progress was monitored by LCMS, which showed the presence of starting material and again 0.75 equivalents were added and stirred for 8 h.). After completion of the reaction, the reaction mixture was quenched with MeOH (3 mL) at 0° C., stirred for 30 min, and concentrated under reduced pressure to obtain the crude product. This crude material was washed with n-pentane (15 mL) and dried under vacuum to afford 3-(3-(4-(trifluoromethyl)phenoxy)pyrrolidin-1-yl)bicyclo[1.1.1]pentan-1-amine (0.27 g, crude), which was used for the next step without further purification. LCMS (ES) m/z=313.1 [M+H]⁺.

Step 5: To a solution of 2-(4-chlorophenoxy)acetic acid (0.19 g, 1.03 mmol, 1.2 equiv) in DCM (10 mL) at 0° C. was added triethylamine (0.36 mL, 2.59 mmol, 3 equiv) followed by T3P® (50 wt % in EtOAc) (1.03 mL, 1.72 mmol, 2 equiv). The mixture was stirred at 0° C. for 10 min, at which time, this reaction mixture was added: 3-(3-(4-(trifluoromethyl)phenoxy)pyrrolidin-1-yl)bicyclo[1.1.1]pentan-1-amine (0.27 g, 0.86 mmol, 1 equiv) in DCM (10 mL), slowly at 0° C. and the reaction was stirred at rt for 12 h. The reaction mixture was diluted with water (10 mL) and extracted with DCM (2×20 mL). The combined organic layer was washed with saturated aqueous NaHCO₃ solution (15 mL) and then dried over anhydrous Na₂SO₄, filtered and evaporated under reduced pressure. The crude product was purified by preparative TLC using 50% EtOAc in hexanes. After final drying, the desired product 2-(4-chlorophenoxy)-N-(3-(3-(4-(trifluoromethyl)phenoxy)pyrrolidin-1-yl)bicyclo[1.1.1]pentan-1-yl)acetamide was obtained as a white solid (0.087 g, 21%). LCMS (ES) m/z=481.4 [M+H]⁺. ¹H NMR (400 MHz, CD₃OD) δ ppm 1.93-2.02 (m, 1H), 2.13 (s, 6H), 2.31-2.39 (m, 1H), 2.64-2.68 (m, 1H), 2.85-2.92 (m, 2H), 2.98-3.02 (m, 1H), 4.48 (s, 2H), 4.97-5.00 (m, 1H), 6.95 (d, J=9.2 Hz, 2H), 7.01 (d, J=8.4 Hz, 2H), 7.27 (d, J=9.2 Hz, 2H), 7.56 (d, J=8.4 Hz, 2H).

TABLE 15

| | | | LCMS | |
|---|---|---|---|---|
| | | | m/z | ¹H-NMR *(400 MHz, |
| Cmpd # | Structure | Name | [M + H]⁺ | CD₃OD) |
| 6p | 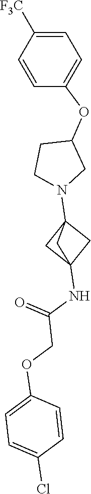 [SMILES: O=C(COc1ccc(Cl)cc1)NC12CC(N3CCC(Oc4ccc(C(F)(F)F)cc4)C3)(C1)C2] | 2-(4-chlorophenoxy)-N-(3-(3-(4- (trifluoromethyl)phenoxy) pyrrolidin-1-yl) bicyclo[1.1.1]pentan-1- yl)acetamide | 481.4 | 1.93-2.02 (m, 1 H), 2.13 (s, 6 H), 2.31-2.39 (m, 1 H), 2.64-2.68 (m, 1 H), 2.85-2.92 (m, 2 H), 2.98- 3.02 (m, 1 H), 4.48 (s, 2 H), 4.97-5.00 (m, 1 H), 6.95 (d, J = 9.2 Hz, 2 H), 7.01 (d, J = 8.4 Hz, 2 H), 7.27 (d, J = 9.2 Hz, 2 H), 7.56 (d, J = 8.4 Hz, 2 H). |

### Example 6q

### 2-(4-chloro-3-fluorophenoxy)-N-(3-(3-(4-chlorophenoxy)pyrrolidin-1-yl)bicyclo[1.1.1]pentan-1-yl)acetamide

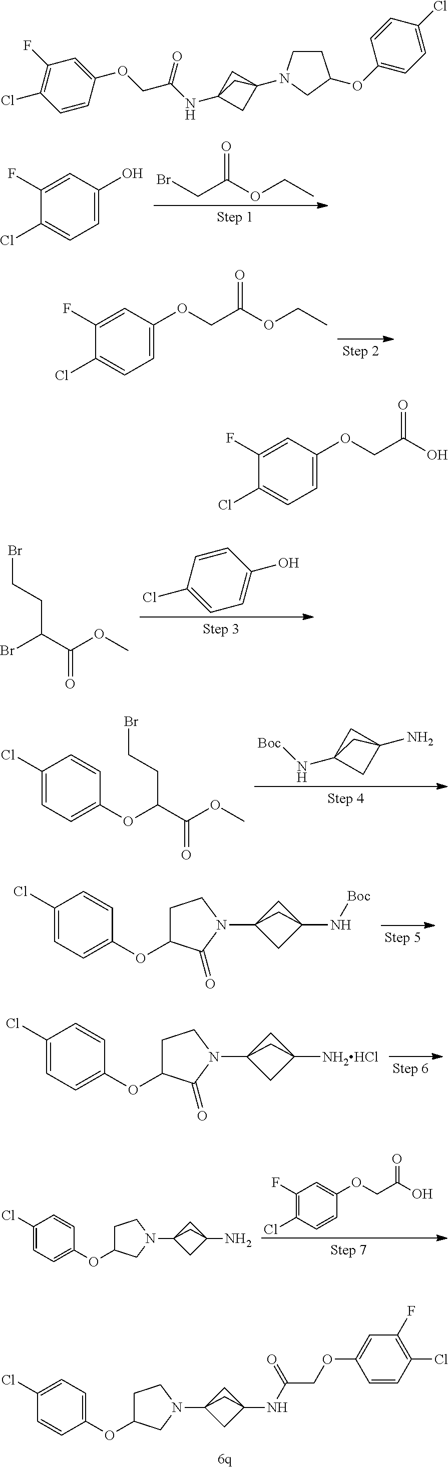

Step 1: To a solution of 4-chloro-3-fluorophenol (3 g, 20.47 mmol, 1 equiv.) in DMF (20 mL) was added K₂CO₃ (8.47 g, 61.41 mmol, 3 equiv.) followed by the addition of ethyl 2-bromoacetate (6.83 g, 40.94 mmol, 2 equiv) dropwise at 0° C. The reaction mixture was allowed to stir at 80° C. for 16 h. The resulting mixture was allowed to warm to 25° C. (rt). After consumption of the starting material (TLC, 10% EtOAc in Hexane), the reaction mixture was filtered through a Büchner funnel and the filtrate was concentrated under reduced pressure. The crude product was purified by flash column chromatography (Combiflash®) using a silica gel column (5% ethyl acetate in hexane) to obtain the title compound ethyl 2-(4-chloro-3-fluorophenoxy)acetate (3.8 g, as a colorless liquid. LCMS (ES) m/z=232 [M+H]⁺. ¹H NMR (400 MHz, CDCl₃) δ ppm 1.30-1.27 (m, 3H), 4.29-4.23 (m, 2H), 4.60 (s, 2H), 6.65-6.63 (m, 1H), 6.73-6.70 (m, 1H), 7.29-7.25 (m, 1H).

Step 2: To a solution of ethyl 2-(4-chloro-3-fluorophenoxy) acetate (3.8 g 16.334 mmol, 1 equiv.) in a mixture of THF (15 mL) and water (15 mL) was added LiOH.H₂O (6.85 g, 41.95 mmol, 10 equiv.) at 0° C. and the resulting mixture was stirred at room temperature for 3 h. After consumption of the starting material (TLC, 5% methanol in DCM), THF was removed under reduced pressure and the residue was diluted with water (20 mL), and washed with Et₂O (2×15 mL) to remove un-reacted ethyl 2-bromoacetate. The aqueous layer was acidified with 1 N HCl up to pH ˜2 at 0° C. The product was extracted with EtOAc (2×30 mL). The combined organic layers were dried over anhydrous sodium sulfate, filtered and concentrated under reduced pressure to obtain the title compound 2-(4-chloro-3-fluorophenoxy)acetic acid (0.720 g, 21% yield) as a white solid. LCMS (ES) m/z=203.0 [M+H]⁺, ¹H NMR (400 MHz, DMSO-d₆) δ ppm 4.71 (s, 2H), 6.81-6.78 (m, 1H), 7.07-7.03 (m, 1H), 7.47-7.43 (t, J=9.2 Hz, 1H), 13.06 (s, 1H).

Step 3: To a solution of methyl 2,4-dibromobutanoate (4.01 g, 0.015 mmol, 1 equiv) in DMF (50 mL) was added 4-chlorophenol (2.0 g, 0.015 mmol, 1 equiv) at rt followed by K₂CO₃ (2.1 g, 0.015 mmol, 1 equiv) and the reaction was stirred at 60° C. for 3 h. The reaction mixture was then allowed to come to rt. Water (5 mL) was added and the mixture was extracted with EtOAc (3×100 mL). The combined EtOAc extracts were dried over anhydrous Na₂SO₄, filtered and distilled under vacuum. The crude material was purified by flash column chromatography (10% EtOAc in hexane) to afford methyl 4-bromo-2-(4-chlorophenoxy)butanoate as a colorless liquid (2.6 g). ¹H NMR (400 MHz, CDCl₃) δ ppm 2.42-2.51 (m, 2H), 3.58-3.62 (m, 2H), 3.76 (s, 3H), 4.82-4.85 (m, 1H), 6.85 (d, J=8.8 Hz, 2H), 7.26 (d, J=7.2 Hz, 2H).

Step 4: Methyl 4-bromo-2-(4-chlorophenoxy)butanoate (0.4 g, 1.31 mmol, 1 equiv) and tert-butyl (3-aminobicyclo[1.1.1]pentan-1-yl)carbamate (0.26 g, 1.31 mmol, 1 equiv) were charged to a sealed tube and Et₃N (0.73 mL) was added. The mixture was then heated at 100° C. for 1 h. The reaction mixture was diluted with cold water (150 mL) and extracted with EtOAc (2×200 mL). The combined EtOAc extracts were dried over anhydrous Na₂SO₄, filtered and evaporated under vacuum. The crude material was purified by column chromatography using an eluent of 50% EtOAc in hexane to obtain the desired product tert-butyl (3-(3-(4-chlorophenoxy)-2-oxopyrrolidin-1-yl)bicyclo[1.1.1]pentan-1-yl)carbamate as an off-white solid (2.2 g, 47.41%) (Reaction was performed in multiple batches: 0.26 g×9). LCMS (ES) m/z=337.1 [M+H]⁺ (loss of tert-butyl group). ¹H NMR (400 MHz, DMSO-d₆) δ ppm 1.36 (s, 9H), 1.83-1.92 (m, 1H), 2.16-2.22 (m, 6H), 3.23-3.25 (m, 1H), 3.32-3.37 (m, 1H), 4.96 (t, J=8.0 Hz, 1H), 7.02 (d, J=9.2 Hz, 2H), 7.3 (d, J=8.8 Hz, 2H), 7.55 (s, 1H). Note: One proton was merged with solvent peak.

Step 5: To a solution of tert-butyl (3-(3-(4-chlorophenoxy)-2-oxopyrrolidin-1-yl)bicyclo[1.1.1]pentan-1-yl)carbamate (2.6 g, 5.59 mmol) in DCM (30 mL) at 0° C. was added 25 mL of 4 M HCl in 1,4-dioxane and the mixture was stirred at rt for 16 h. The reaction mixture was concentrated to give 1-(3-aminobicyclo[1.1.1]pentan-1-yl)-3-(4-chlorophenoxy)pyrrolidin-2-one, hydrochloride (crude yield 1.8 g, 82.9%), which was taken to the next step without further purification. LCMS (ES) m/z=293 [M+H]⁺. ¹H NMR (400 MHz, DMSO-d₆) δ ppm 1.87-1.94 (m, 1H), 2.28-2.33 (m, 6H), 3.32-3.38 (m, 2H), 4.99 (t, J=7.6 Hz, 1H), 7.02 (d, J=8.8 Hz, 2H), 7.31 (d, J=8.4 Hz, 2H), 8.76 (bs, 3H). Note: One proton was merged with solvent peak.

Step 6: To a solution of 1-(3-aminobicyclo[1.1.1]pentan-1-yl)-3-(4-chlorophenoxy)pyrrolidin-2-one hydrochloride (0.34 g, 1.036 mmol, 1 equiv) in THF (4 mL) was added BH₃.Me₂S (0.3 mL, 0.61 mmol, 3 equiv) at 0° C. (Note: BH₃.Me₂S was added at 3 interval points of time: first 1.5 equivalents were added and stirred for 16 h and again 0.75 equivalents were added and stirred for 8 h. After that time reaction progress was monitored by LCMS, which showed the presence of starting material and again 0.75 equivalents were added and stirred for 8 h.). After completion, the reaction mixture was quenched with methanol at 0° C., stirred for 30 min, and then evaporated under reduced pressure to obtain the crude product, which was washed with dry n-pentane (50 mL) and dried to yield 3-(3-(4-chlorophenoxy)pyrrolidin-1-yl)bicyclo[1.1.1]pentan-1-amine (Crude yield 1.4 g, 87%) (Reaction was performed in multiple batches: 0.34g×5), which was used for the next step without further purification. LCMS (ES) m/z=279 [M+H]⁺.

Step 7: To a solution of 2-(4-chloro-3-fluorophenoxy)acetic acid (0.062 g, 0.303 mmol, 1.2 equiv) in DCM (20 mL) at 0° C. was added triethylamine (0.1 mL, 0.718 mmol, 3 equiv) followed by T3P® (50 wt % in EtOAc) (0.2 mL, 0.380 mmol, 1.5 equiv) and the mixture was stirred at 0° C. for 10 min. To this reaction mixture was added 3-(3-(4-chlorophenoxy)pyrrolidin-1-yl)bicyclo[1.1.1]pentan-1-amine (0.08 g, 0.253 mmol, 1 equiv) in DCM (10 mL) slowly at 0° C. and the reaction was stirred at rt for 16 h. The reaction mixture was diluted with water (20 mL) and extracted with DCM (2×20 mL). The combined organic layer was washed with saturated aqueous NaHCO₃ solution (10 mL) and then dried over anhydrous Na₂SO₄, filtered and evaporated under reduced pressure. The crude material was purified by column chromatography twice by using an eluent of 3% MeOH in DCM to afford 2-(4-chloro-3-fluorophenoxy)-N-(3-(3-(4-chlorophenoxy)pyrrolidin-1-yl)bicyclo[1.1.1]pentan-1-yl)acetamide (0.025 g, 18.79%) as a white solid. LCMS (ES) m/z=465.3 [M+H]⁺. ¹H NMR (400 MHz, DMSO-d₆) δ ppm 1.73-1.77 (m, 1H),1.95 (s, 6H), 2.21-2.30 (m, 1H), 2.58-2.68 (m, 3H), 2.84-2.88 (m, 1H), 4.44 (s, 2H), 4.84 (s, 1H), 6.82 (d, J=9.2 Hz, 1H), 6.88 (d, J=8.8 Hz, 2H), 7.02-7.05 (m, 1H), 7.28 (d, J=8.8 Hz, 2H), 7.46 (t, J=8.8 Hz, 1H), 8.62 (s, 1H).

The Compound of Example 6r was prepared generally according to the procedure described above for Example 6q.

TABLE 16

| | | | LCMS | |
|---|---|---|---|---|
| | | | m/z | ¹H-NMR (400 MHz, |
| Cmpd # | Structure | Name | [M + H]⁺ | DMSO-d₆) |
| 6q | 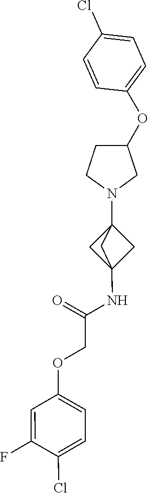 [SMILES: O=C(COc1ccc(Cl)c(F)c1)NC12CC(N3CCC(Oc4ccc(Cl)cc4)C3)(C1)C2] | 2-(4-chloro-3-fluorophenoxy)-N-(3- (3-(4-chlorophenoxy)pyrrolidin-1- yl)bicyclo[1.1.1]pentan-1- yl)acetamide | 465.3 | 1.73-1.77 (m, 1 H), 1.95 (s, 6 H), 2.21-2.30 (m, 1 H), 2.58-2.68 (m, 3 H), 2.84-2.88 (m, 1 H), 4.44 (s, 2 H), 4.84 (s, 1 H), 6.82 (d, J = 9.2 Hz, 1 H), 6.88 (d, J = 8.8 Hz, 2 H), 7.02-7.05 (m, 1 H), 7.28 (d, J = 8.8 Hz, 2 H), 7.46 (t, J = 8.8 Hz, 1 H), 8.62 (s, 1 H). |
| 6r | 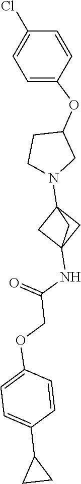 [SMILES: O=C(COc1ccc(C2CC2)cc1)NC12CC(N3CCC(Oc4ccc(Cl)cc4)C3)(C1)C2] | N-(3-(3-(4- chlorophenoxy)pyrrolidin-1- yl)bicyclo[1.1.1]pentan-1-yl)-2-(4- cyclopropylphenoxy)acetamide | 453.4 | 0.54-0.56 (m, 2 H), 0.84- 0.86 (m, 2 H), 1.74-1.84 (m, 2 H), 1.95 (s, 6 H), 2.20- 2.30 (m, 1 H), 2.53-2.70 (m, 3 H), 2.84-2.88 (m, 1 H), 4.33 (s, 2 H), 4.85 (bs, 1 H), 6.80 (d, J = 8.4 Hz, 2 H), 6.88 (d, J = 8.8 Hz, 2 H), 6.97 (d, J = 8.4 Hz, 2 H), 7.28 (d, J = 8.8 Hz, 2 H), 8.54 (s, 1 H). |

### Example 6s

### 2-(4-chlorophenoxy)-N-(3-(3-(pyridin-4-yloxy)pyrrolidin-1-yl)bicyclo[1.1.1]pentan-1-yl)acetamide

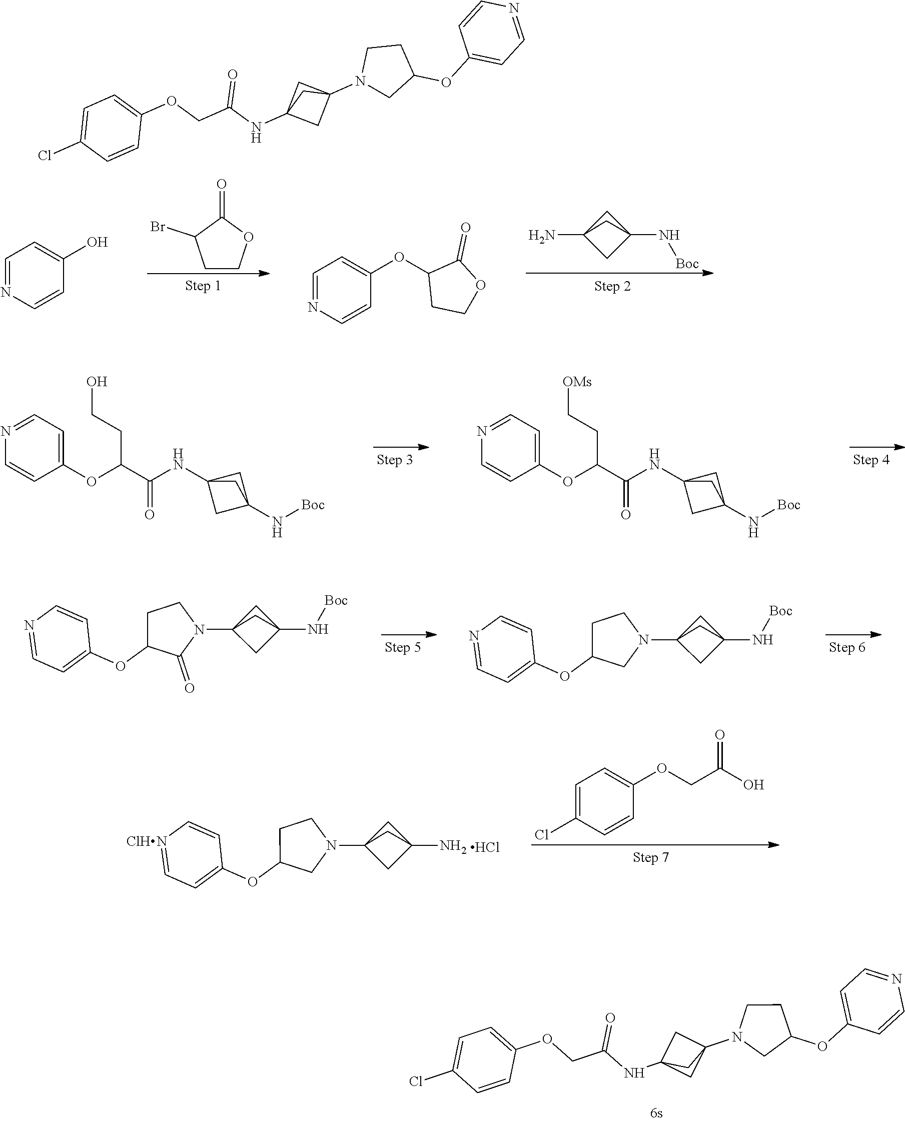

Step 1: To a solution of pyridin-4-ol (2.0 g, 21.03 mmol, 1.0 equiv.) in DMF (30 mL) was added Ag₂CO₃ (8.7 g, 31.54 mmol, 1.5 equiv.) at 0° C., stirred for 5 mins and then 3-bromodihydrofuran-2(3H)-one (2.3 mL, 25.23 mmol, 1.2 equiv.) was added and the reaction was stirred at 60° C. for 2 h. After the consumption of the starting material (TLC, 5% MeOH in DCM), the reaction mixture was filtered through a Celite® bed and washed with EtOAc (200 mL). The organic layer was washed with ice cold water (2×20 mL), dried over anhydrous Na₂SO₄, filtered and distilled under vacuum. The crude material was purified by flash column chromatography (5-6% MeOH in DCM) to afford 3-(pyridin-4-yloxy)dihydrofuran-2(3H)-one as a brown liquid (1.18 g, 31.4% yield). LCMS (ES) m/z=180.0 [M+H]⁺, ¹H NMR (400 MHz, CDCl₃) δ ppm 2.45-2.55 (m, 1H), 2.72-2.80 (m, 1H), 4.33-4.42 (m, 1H), 4.52-4.57 (m, 1H), 5.05 (t, J=7.8 Hz, 1H), 6.95-6.97(m, 2H), 8.47-8.49 (m, 2H).

Step 2: To a solution of 3-(pyridin-4-yloxy)dihydrofuran-2(3H)-one (0.99 g, 5.54 mmol, 1.0 equiv.) in toluene (15.0 mL) was added tert-butyl (3-aminobicyclo[1.1.1]pentan-1-yl)carbamate (1.1 g, 5.54 mmol, 1.0 equiv) at room temperature and the mixture was then heated to 110° C. using an oil bath for 16 h. After the consumption of the starting material (TLC, 5% MeOH in DCM), the reaction mixture was concentrated under vacuum. The crude material was purified by column chromatography using an eluent of 5-7% MeOH in DCM to obtain the desired product tert-butyl (3-(4-hydroxy-2-(pyridin-4-yloxy)butanamido)bicyclo[1.1.1]pentan-1-yl)carbamate as a pale yellow liquid (1.2 g, 57.4% yield). LCMS (ES) m/z=378.1 [M+H]⁺. ¹H NMR (400 MHz, DMSO-d₆) δ ppm 1.34 (s, 9H), 1.88-1.89 (m, 2H), 2.06 (s, 6H), 3.46-3.48 (m, 2H), 4.60 (bs, 1H), 4.71 (bs, 1H), 6.46 (s, 2H), 7.44 (bs, 1H), 8.36 (s, 2H), 8.73 (s, 1H).

Step 3: To a solution of tert-butyl (3-(4-hydroxy-2-(pyridin-4-yloxy)butanamido)bicyclo[1.1.1]pentan-1-yl)carbamate (1.2 g, 3.17 mmol, 1.0 equiv.) in DCM (20 mL) at 0° C. was added triethylamine (1.33 mL, 9.51 mmol, 3.0 equiv.), stirred for 10 mins and then methanesulfonyl chloride (0.5 mL, 6.36 mmol, 2.0 equiv.) was added and the mixture was stirred at room temperature for 3 h. After the consumption of the starting material (TLC, 5% MeOH in DCM), the reaction mixture was cooled to 0° C. and quenched with ice. It was extracted with DCM (2×80 mL). Combined organic layer was dried over anhydrous Na₂SO₄, filtered and distilled under vacuum to afford 4-((3-((tert-butoxycarbonyl)amino)bicyclo[1.1.1]pentan-1-yl)amino)-4-oxo-3-(pyridin-4-yloxy)butyl methanesulfonate (1.6 g, crude), which was taken to the next step without further purification. LCMS (ES) m/z=456.1 [M+H]⁺.

Step 4: To a solution of 4-((3-((tert-butoxycarbonyl)amino)bicyclo[1.1.1]pentan-1-yl)amino)-4-oxo-3-(pyridin-4-yloxy)butyl methanesulfonate (1.6 g, 3.51 mmol, 1.0 equiv.) in THF (20 mL) was added 60% sodium hydride in mineral oil (0.17 g, 4.21 mmol, 1.2 equiv.) at 0° C. and the reaction was stirred at room temperature for 16 h. After the consumption of the starting material (TLC, 5% MeOH in DCM), the reaction mixture was cooled to 0° C., quenched with ice and was extracted with ethyl acetate (2×100 mL). The combined organic layer was dried over anhydrous Na₂SO₄, filtered and distilled under reduced pressure to obtain the crude product. The crude material was purified by column chromatography using an eluent of 5-7% MeOH in DCM to obtain tert-butyl (3-(2-oxo-3-(pyridin-4-yloxy)pyrrolidin-1-yl)bicyclo[1.1.1]pentan-1-yl)carbamate as an off-white solid (0.73 g, 45.3% yield). LCMS (ES) m/z=360.1 [M+H]⁺. ¹H NMR (400 MHz, CDCl3) δ ppm 1.44 (s, 9H), 2.16-2.22 (m, 1H), 2.39 (s, 6H), 2.54-2.56 (m, 1H), 3.33-3.39 (m, 1H), 3.45-3.48 (m, 1H), 4.91-4.95 (m, 2H), 6.95-6.96 (m, 2H), 8.42-8.43 (m, 2H).

Step 5: To a solution of tert-butyl (3-(2-oxo-3-(pyridin-4-yloxy)pyrrolidin-1-yl)bicyclo[1.1.1]pentan-1-yl)carbamate (0.73 g, 2.03 mmol, 1.0 equiv.) in THF (15 mL) was added borane dimethyl sulfide complex (0.39 mL, 4.06 mmol, 2.0 equiv.) at 0° C. and the reaction was stirred at room temperature for 16 h. After the consumption of the starting material (TLC, 5% MeOH in DCM), the reaction mixture was cooled to 0° C., quenched with methanol, stirred for 1 h and distilled under reduced pressure to obtain the crude product. The crude material was purified by column chromatography using an eluent of 5-7% MeOH in DCM to obtain tert-butyl (3-(3-(pyridin-4-yloxy)pyrrolidin-1-yl)bicyclo[1.1.1]pentan-1-yl)carbamate as a pale yellow sticky solid (0.1 g, 14.2% yield). LCMS (ES) m/z=346.2 [M+H]⁺.

Step 6: To a solution of tert-butyl (3-(3-(pyridin-4-yloxy)pyrrolidin-1-yl)bicyclo[1.1.1]pentan-1-yl)carbamate (0.1 g, 0.29 mmol, 1.0 equiv.) in DCM (5 mL) was added 4M HCl in 1,4-dioxane (2.0 mL) at 0° C. and the reaction was stirred at room temperature for 24 h. After the consumption of the starting material (TLC, 10% MeOH in DCM), the reaction mixture was concentrated under reduced pressure to obtain the crude product. The crude material was triturated with n-pentane (2×5 mL) to obtain 3-(3-(pyridin-4-yloxy)pyrrolidin-1-yl)bicyclo[1.1.1]pentan-1-amine dihydrochloride as an off-white solid (0.09 g, crude). LCMS (ES) m/z=246.3 [M+H]⁺.

Step 7: To a solution of 2-(4-chlorophenoxy)acetic acid (0.079 g, 0.42 mmol, 1.5 equiv.) in DCM (8 mL) at 0° C. was added triethylamine (0.19 mL, 1.40 mmol, 5.0 equiv.), stirred for 10 mins, followed by addition of T3P® (50 wt % in EtOAc) (0.5 mL, 0.85 mmol, 3.0 equiv). The mixture was stirred at 0° C. for 10 min and then 3-(3-(pyridin-4-yloxy)pyrrolidin-1-yl)bicyclo[1.1.1]pentan-1-amine dihydrochloride (0.09 g, 0.28 mmol, 1.0 equiv.) (which was neutralized with triethylamine in DCM (3 mL)) was slowly added at 0° C. and the reaction was stirred at room temperature for 16 h. After the consumption of the starting material (TLC, 5% MeOH in DCM), the reaction mixture was diluted with DCM (100 mL) and was washed with saturated NaHCO₃ (2×5 mL) and water (2×5 mL). The combined organic layer was dried over anhydrous Na₂SO₄, filtered and evaporated under reduced pressure. The crude material was purified by column chromatography using an eluent of 5-7% MeOH in DCM to obtain the desired product. It was further purified by Prep HPLC [(Analytical condition: Column: lnertsil ODS 3V (250 mm×4.6 mm×5 mic); Mobile phase (A): 0.1% ammonia in water; Mobile phase (B): Acetonitrile; Flow rate: 1.0 mL/min)] to afford the desired product 2-(4-chlorophenoxy)-N-(3-(3-(pyridin-4-yloxy)pyrrolidin-1-yl)bicyclo[1.1.1]pentan-1-yl)acetamide as an off-white solid (0.033 g, 28.2% yield). LCMS (ES) m/z=414.38 [M+H]⁺. ¹H NMR (400 MHz, DMSO-d₆) δ ppm 1.76-1.79 (m, 1H), 1.96 (s, 6H), 2.23-2.30 (m, 1H), 2.48 (s, 1H), 2.61-2.72 (m, 2H), 2.86-2.90 (m, 1H), 4.40 (s, 2H), 4.96 (bs, 1H), 6.88 (d, J=4.8 Hz, 2H), 6.94 (d, J=8.0 Hz, 2H), 7.31 (d, J=8.4 Hz, 2H), 8.34 (d, J=5.6 Hz, 2H), 8.61 (s, 1H).

Examples 6t and 6u were prepared generally accordingly to the procedure described above for 6s.

TABLE 18

| | | | LCMS | |
|---|---|---|---|---|
| | | | m/z | ¹H-NMR (400 MHz, |
| Cmpd # | Structure | Name | [M + H]⁺ | DMSO-d₆) |
| 6s | 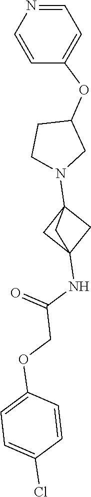 [SMILES: O=C(COc1ccc(Cl)cc1)NC12CC(N3CCC(Oc4ccncc4)C3)(C1)C2] | 2-(4-chlorophenoxy)-N-(3-(3- (pyridin-4-yloxy)pyrrolidin-1- yl)bicyclo[1.1.1]pentan-1- yl)acetamide | 414.38 | 1.76-1.79 (m, 1 H), 1.96 (s, 6 H), 2.23-2.30 (m, 1 H), 2.48 (s, 1 H), 2.61- 2.72 (m, 2 H), 2.86-2.90 (m, 1 H), 4.40 (s, 2 H), 4.96 (bs, 1 H), 6.88 (d, J = 4.8 Hz, 2 H), 6.94 (d, J = 8.0 Hz, 2 H), 7.31 (d, J = 8.4 Hz, 2 H), 8.34 (d, J = 5.6 Hz, 2 H), 8.61 (s, 1 H). |
| 6t | 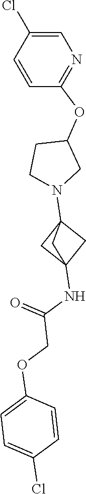 [SMILES: O=C(COc1ccc(Cl)cc1)NC12CC(N3CCC(Oc4ccc(Cl)cn4)C3)(C1)C2] | 2-(4-chlorophenoxy)-N-(3-(3-((5- chloropyridin-2-yl)oxy)pyrrolidin-1- yl)bicyclo[1.1.1]pentan-1- yl)acetamide | 448.0 | 1.77-1.88 (m, 1 H), 1.94 (s, 6 H), 2.18-2.26 (m, 1 H), 2.59-2.71 (m, 3 H), 2.84-2.87 (m, 1 H), 4.39 (s, 2 H), 5.29 (s, 1 H), 6.82 (d, J = 8.8 Hz, 1 H), 6.94 (d, J = 8.8 Hz, 2 H), 7.31 (d, J = 8.8 Hz, 2 H), 7.76 (d, J = 7.2 Hz, 1 H), 8.17 (s, 1 H), 8.61 (s, 1 H) |
| 6u | 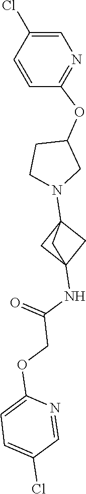 [SMILES: O=C(COc1ccc(Cl)cn1)NC12CC(N3CCC(Oc4ccc(Cl)cn4)C3)(C1)C2] | 2-((5-chloropyridin-2-yl)oxy)-N-(3- (3-((5-chloropyridin-2- yl)oxy)pyrrolidin-1- yl)bicyclo[1.1.1]pentan-1- yl)acetamide | 449.0 | 1.76-1.80 (m, 1 H), 1.92 (s, 6 H), 2.17-2.26 (m, 1 H), 2.58-2.70 (m, 3 H), 2.82-2.86 (m, 1 H), 4.63 (s, 2 H), 5.29 (s, 1 H), 6.82 (d, J = 8.8 Hz, 1 H), 6.92 (d, J = 8.4 Hz, 1 H), 7.74-7.82 (m, 2 H), 8.15- 8.17 (m, 2 H), 8.55 (s, 1 H). |

### Examples 6v and 6w

### N-(3-(3-(4-chloro-3-fluorophenoxy)pyrrolidin-1-yl)bicyclo[1.1.1]pentan-1-yl)-2-(4-chlorophenoxy)acetamide

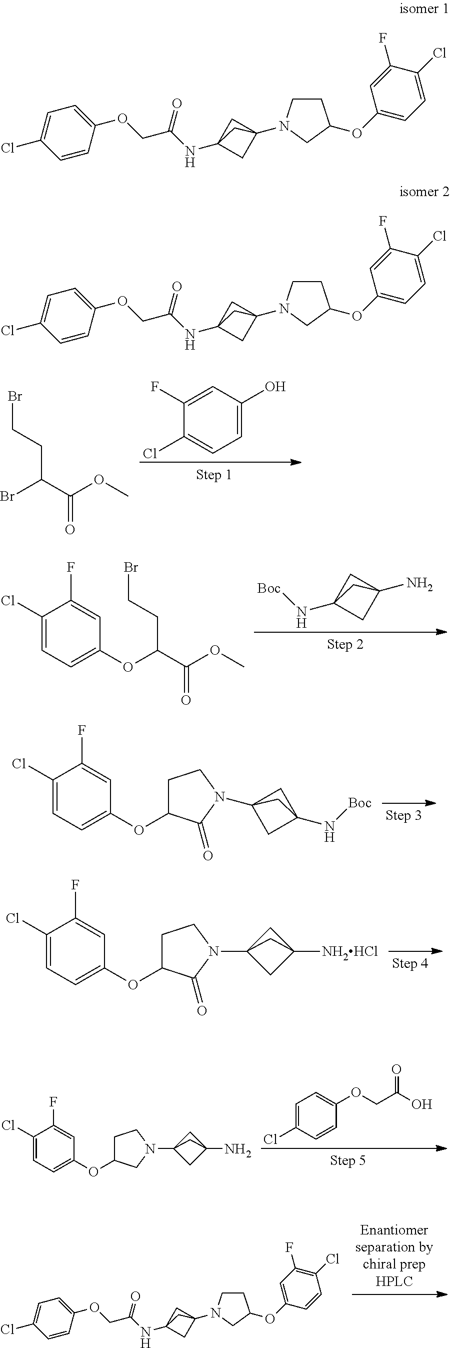

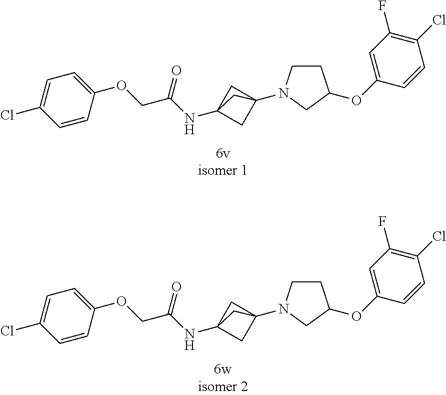 [SMILES: O=C(COc1ccc(Cl)cc1)NC12CC(N3CCC(Oc4ccc(Cl)c(F)c4)C3)(C1)C2 ; O=C(COc1ccc(Cl)cc1)NC12CC(N3CCC(Oc4ccc(Cl)c(F)c4)C3)(C1)C2]

Step 1: To a solution of 4-chloro-3-fluorophenol (1.0 g, 6.82 mmol, 1.0 equiv.) in DMF (20 mL) was added K₂CO₃ (0.94 g, 6.82 mmol, 1.0 equiv.) at 0° C., stirred for 10 mins and then methyl 2,4-dibromobutanoate (0.96 mL, 6.82 mmol, 1.0 equiv.) was added and the reaction was stirred at 60° C. for 3 h. After the consumption of the starting material (TLC, 10% ethyl acetate in hexane), the reaction mixture was diluted with ice cold water (50 mL) and was extracted with EtOAc (2×100 mL). The combined EtOAc extracts were dried over anhydrous Na₂SO₄, filtered and distilled under vacuum. The crude material was purified by flash column chromatography (2-5% EtOAc in hexane) to afford methyl 4-bromo-2-(4-chloro-3-fluorophenoxy)butanoate as a colorless liquid (1.18 g, crude). LCMS (ES) m/z=324.0 [M+H]⁺,

Step 2: To a solution of tert-butyl (3-aminobicyclo[1.1.1]pentan-1-yl)carbamate (0.6 g, 3.03 mmol, 1.0 equiv.) in Et₃N (1.7 mL, 12.12 mmol, 4.0 equiv.) was added methyl 4-bromo-2-(4-chloro-3-fluorophenoxy)butanoate (1.18 g, 3.63 mmol, 1.2 equiv) at room temperature in a sealed tube and the mixture was then heated at 100° C. using an oil bath for 1 h. (Note: The reaction was carried out by dividing 0.6 g into 3 batches). After the consumption of the starting material (TLC, 70% ethyl acetate in hexane), the reaction mixture was diluted with ethyl acetate (100 mL) and was washed with water (2×20 mL). The combined EtOAc extracts were dried over anhydrous Na₂SO₄, filtered and evaporated under vacuum. The crude material was purified by column chromatography using an eluent of 60-70% EtOAc in hexane to obtain the desired product tert-butyl (3-(3-(4-chloro-3-fluorophenoxy)-2-oxopyrrolidin-1-yl)bicyclo[1.1.1]pentan-1-yl)carbamate as an off-white solid (0.48 g, 40.0%). LCMS (ES) m/z=411.3 [M+H]⁺

Step 3: To a solution of tert-butyl (3-(3-(4-chloro-3-fluorophenoxy)-2-oxopyrrolidin-1-yl)bicyclo[1.1.1]pentan-1-yl)carbamate (0.48 g, 1.17 mmol, 1.0 equiv.) in DCM (10 mL) at 0° C. was added 5 mL of 4 M HCl in 1,4-dioxane and the mixture was stirred at room temperature for 16 h. After the consumption of the starting material (TLC, 70% ethyl acetate in hexane), the reaction mixture was concentrated and the crude was triturated with n-pentane (2×10 mL) and dried under high vacuum to afford 1-(3-aminobicyclo[1.1.1]pentan-1-yl)-3-(4-chloro-3-fluorophenoxy)pyrrolidin-2-one hydrochloride (0.37 g, 91.3% yield), which was taken to the next step without further purification. LCMS (ES) m/z=311.1 [M+H]⁺. ¹H NMR (400 MHz, DMSO-d₆) δ ppm 1.91 (bs, 1H), 2.31 (s, 6H), 2.50-2.60 (m, 3H), 5.05 (t, J=7.6 Hz, 1H), 6.88 (d, J=8.8 Hz, 1H), 7.14 (d, J=10.8 Hz, 1H), 7.41-7.50 (m, 1H), 8.81-8.98 (m, 3H).

Step 4: To a solution of 1-(3-aminobicyclo[1.1.1]pentan-1-yl)-3-(4-chloro-3-fluorophenoxy)pyrrolidin-2-one hydrochloride (0.37 g, 1.06 mmol, 1.0 equiv.) in THF (10 mL) was added BH₃.Me₂S (0.31 mL, 3.30 mmol, 3.1 equiv.) at 0° C. and the reaction was stirred for 40 h. (Note: 1.5 equiv. of BH₃.Me₂S complex was added initially, stirred for 16 h, monitored the progress of the reaction by LCMS, added again 0.8 equiv. of BH₃.Me₂S, stirred for 8 h, monitored the progress of the reaction by LCMS and again 0.8 equiv. of BH₃.Me₂S was added and the reaction was stirred for 16 h.) After the consumption of the starting material (TLC, 5% MeOH in DCM), the reaction mixture was cooled to 0° C., quenched with MeOH (5 mL), stirred for 30 min, and concentrated under reduced pressure to obtain the crude product. This crude material was triturated with n-pentane (2×5 mL) and dried under high vacuum to yield 3-(3-(4-chloro-3-fluorophenoxy)pyrrolidin-1-yl)bicyclo[1.1.1]pentan-1-amine as an off-white solid (0.4 g, crude yield), which was used for the next step without further purification. LCMS (ES) m/z=297.1 [M+H]⁺

Step 5: To a solution of 2-(4-chlorophenoxy)acetic acid (0.30 g, 1.62 mmol, 1.2 equiv.) in DCM (10 mL) at 0° C. was added triethylamine (0.75 mL, 5.40 mmol, 4.0 equiv.), stirred for 10 mins, followed by addition of T3P® (50 wt. % in EtOAc) (1.62 mL, 2.70 mmol, 2 equiv). The mixture was stirred at 0° C. for 10 min and then 3-(3-(4-chloro-3-fluorophenoxy)pyrrolidin-1-yl)bicyclo[1.1.1]pentan-1-amine (0.4 g, 1.35 mmol, 1.0 equiv.) in DCM (5 mL) was slowly added at 0° C. and the reaction was stirred at room temperature for 16 h. After the consumption of the starting material (TLC, 70% ethyl acetate in hexane), the reaction mixture was diluted with DCM (200 mL) and was washed with saturated NaHCO₃ (2×20 mL) and water (2×20 mL). The combined organic layer was dried over anhydrous Na₂SO₄, filtered and evaporated under reduced pressure. The crude material was purified by column chromatography using an eluent of 65-80% EtOAc in hexane to obtain the desired product N-(3-(3-(4-chloro-3-fluorophenoxy)pyrrolidin-1-yl)bicyclo[1.1.1]pentan-1-yl)-2-(4-chlorophenoxy)acetamide as an off-white solid (0.16 g, 25.4%).

The racemate product from step 5 was submitted for chiral prep HPLC to separate the isomers by using the following analytical conditions: [Column: CHIRALPAK IC (100 mm×4.6 mm×3 mic); Flow rate: 1.0 mL/min; Mobile phase: n-Hexane: IPA with 0.1% DEA (65:35).] Fractions containing the two isomers were separately evaporated under reduced pressure, triturated with n-pentane (10 mL, HPLC grade) and dried under high vacuum.

### Example 6v

### Isomer 1 (Single Unknown Stereochemistry)

Recovery: 0.021 g (gum). LCMS (ES) m/z=465.3 [M+H]⁺. ¹H NMR (400 MHz, DMSO-d₆) δ ppm 1.74-1.77 (m, 1H), 1.95 (s, 6H), 2.22-2.30 (m, 1H), 2.59-2.69 (m, 3H), 2.84-2.88 (m, 1H), 4.40 (s, 2H), 4.88 (bs, 1H), 6.76 (d, J=9.2 Hz, 1H), 6.94-7.00 (m, 3H), 7.31 (d, J=7.6 Hz, 2H), 7.42 (t, J=8.8 Hz, 1H), 8.61 (s, 1H). Chiral HPLC purity: 100.0% at 225 nm; % ee: 100.0%

### Example 6w

### Isomer 2 (Single Unknown Stereochemistry)

Recovery: 0.025 g (gum). LCMS (ES) m/z=465.3 [M+H]⁺. ¹H NMR (400 MHz, DMSO-d₆) δ ppm 1.76-1.77 (m, 1H), 1.95 (s, 6H), 2.24-2.25 (m, 1H), 2.59-2.69 (m, 3H), 2.85-2.90 (m, 1H), 4.40 (s, 2H), 4.89 (bs, 1H), 6.76 (d, J=9.2 Hz, 1H), 6.94-7.00 (m, 3H), 7.31 (d, J=7.6 Hz, 2H), 7.42 (t, J=8.6 Hz, 1H), 8.61 (s, 1H). Chiral HPLC purity: 100.0% at 225 nm; % ee: 100.0%.

TABLE 17

| | | | LCMS m/z | ¹H-NMR (400 MHz, |
|---|---|---|---|---|
| Cmpd # | Structure | Name | [M + H]⁺ | DMSO-d₆) |
| 6v (Isomer 1) | 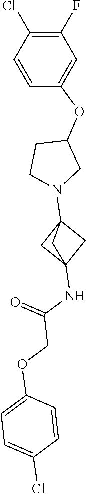 [SMILES: O=C(COc1ccc(Cl)cc1)NC12CC(N3CCC(Oc4ccc(Cl)c(F)c4)C3)(C1)C2] | N-(3-(3-(4-chloro-3- fluorophenoxy)pyrrolidin- 1-yl)bicyclo[1.1.1]pentan- 1-yl)-2-(4- chlorophenoxy)acetamide | 465.3 | 1.74-1.77 (m, 1 H), 1.95 (s, 6 H), 2.22-2.30 (m, 1 H), 2.59-2.69 (m, 3 H), 2.84-2.88 (m, 1 H), 4.40 (s, 2 H), 4.88 (bs, 1 H), 6.76 (d, J = 9.2 Hz, 1 H), 6.94-7.00 (m, 3 H), 7.31 (d, J = 7.6 Hz, 2 H), 7.42 (t, J = 8.8 Hz, 1 H), 8.61 (s, 1 H). |
| 6w (Isomer 2) | 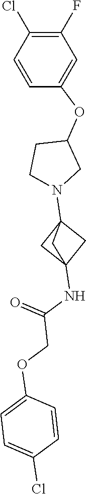 [SMILES: O=C(COc1ccc(Cl)cc1)NC12CC(N3CCC(Oc4ccc(Cl)c(F)c4)C3)(C1)C2] | N-(3-(3-(4-chloro-3- fluorophenoxy)pyrrolidin-1- yl)bicyclo[1.1.1]pentan- 1-yl)-2-(4- chlorophenoxy)acetamide | 465.3 | 1.76-1.77 (m, 1 H), 1.95 (s, 6 H), 2.24-2.25 (m, 1 H), 2.59-2.69 (m, 3 H), 2.85-2.90 (m, 1 H), 4.40 (s, 2 H), 4.89 (bs, 1 H), 6.76 (d, J = 9.2 Hz, 1 H), 6.94-7.00 (m, 3 H), 7.31 (d, J = 7.6 Hz, 2 H), 7.42 (t, J = 8.6 Hz, 1 H), 8.61 (s, 1 H). |

### Example 6x

### N-(3-(3-(bicyclo[4.2.0]octa-1,3,5-trien-3-yloxy)pyrrolidin-1-yl)bicyclo[1.1.1]pentan-1-yl)-2-(4-chlorophenoxy)acetamide

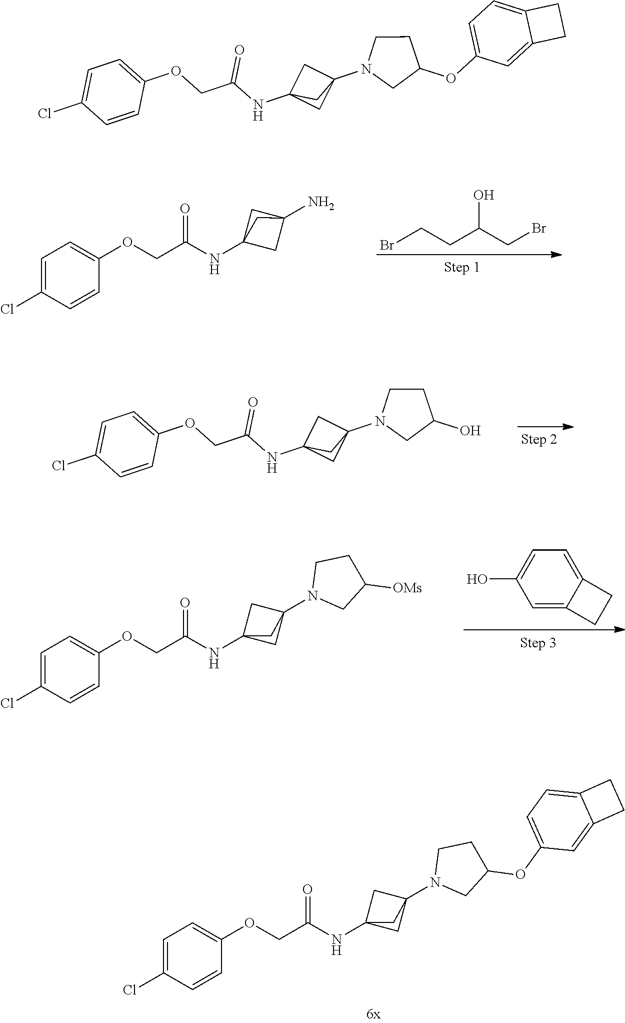

Step 1: To a solution of N-(3-aminobicyclo[1.1.1]pentan-1-yl)-2-(4-chlorophenoxy)acetamide (3.0 g, 11 mmol, 1.0 equiv) in dimethylacetamide (30 mL) was added N,N-Di-isopropylethylamine (9.6 mL, 55 mmol, 5.0 equiv.) and 1,4-dibromobutan-2-ol (2.61 mL, 22 mmol, 2.0 equiv) at room temperature. The reaction mixture was maintained at 80° C. for 1 h. After that, the reaction mixture was cooled to room temperature, quenched with crushed ice (150 mL), and extracted with EtOAc (3×150 mL). The combined organic layer was washed with cold water (100 mL), dried over anhydrous sodium sulfate, filtered and concentrated under reduced pressure. The crude product was purified by flash column chromatography (neutral alumina column) using 0.1% to 5% methanol in DCM as eluent to obtain 2-(4-chlorophenoxy)-N-(3-(3-hydroxypyrrolidin-1-yl)bicyclo[1.1.1]pentan-1-yl)acetamide (1.5 g, 13.2% yield, 3.0 g scale reactions with 3 batches (9.0 g) were done) as a viscous oil. LCMS (ES) m/z=337.1 [M+H]⁺. ¹H NMR (400 MHz, DMSO-d₆) δ ppm 1.49-1.52 (m, 1H), 1.88-1.96 (m, 7H), 2.27-2.31 (m, 1H), 2.38-2.48 (m, 1H), 2.54-2.59 (m, 1H), 2.64-2.68 (m, 1H), 4.15-4.16 (m, 1H), 4.39 (s, 2H), 4.63 (d, J=4.4 Hz, 1H), 6.94 (d, J=8.8 Hz, 2H), 7.31 (d, J=8.8 Hz, 2H), 8.58 (s, 1H).

Step 2: To a solution of 2-(4-chlorophenoxy)-N-(3-(3-hydroxypyrrolidin-1-yl)bicyclo[1.1.1]pentan-1-yl)acetamide (0.20 g, 0.59 mmol, 1.0 equiv) in DCM (50 mL) was added TEA (0.24 mL, 1.7 mmol, 3.0 equiv.) at 0° C. After 10 minutes at the same temperature, mesyl chloride (0.055 mL, 0.71 mmol, 1.2 equiv) was added at 0° C. and reaction mixture was maintained for 2 h at room temperature. The reaction mixture was diluted with DCM (150 mL), and washed with 10% aq. NaHCO₃ solution (2×50 mL) followed by water (50 mL). The organic layer was dried over anhydrous sodium sulfate, filtered and concentrated to afford 1-(3-(2-(4-chlorophenoxy)acetamido)bicyclo[1.1.1]pentan-1-yl)pyrrolidin-3-yl methanesulfonate (0.22 g, crude) as a yellow solid. It was used in the next step without further purification. LCMS (ES) m/z=415.0 [M+H]⁺. ¹H NMR (400 MHz, DMSO-d₆) δ ppm 1.87-1.93 (m, 1H), 1.96 (s, 6H), 2.15-2.23 (m, 1H), 2.39-2.48 (m, 1H), 2.70-2.78 (m, 3H), 3.15 (s, 3H), 4.40 (s, 2H), 5.11-5.14 (m, 1H), 6.95 (d, J=9.2 Hz, 2H), 7.32 (d, J=9.2 Hz, 2H), 8.62 (s, 1H).

Step 3: To a solution of bicyclo[4.2.0]octa-1,3,5-trien-3-ol (0.07 g, 0.57 mmol, 1.2 equiv.) in acetonitrile (10 mL) was added Cs₂CO₃ (0.4 g, 1.2 mmol, 2.5 equiv) and 1-(3-(2-(4-chlorophenoxy)acetamido)bicyclo[1.1.1]pentan-1-yl)pyrrolidin-3-yl methanesulfonate (0.2 g, 0.48 mmol, 1.0 equiv) at room temperature. The reaction mixture was maintained at 85° C. for 2 h under microwave irradiation. Then the reaction mixture was cooled to room temperature, diluted with EtOAc (150 mL), washed with cold water (2×50 mL), and the organic layer was dried over anhydrous sodium sulfate, filtered and concentrated under reduced pressure to obtain the crude product. The crude mass was purified by flash column chromatography using 0.1% to 5% methanol in DCM as eluent and further re-purified by prep HPLC [Analytical conditions: Column: lnertsil ODS 3V (250 mm×4.6 mm×5 mic). Mobile phase (A): 0.1% Ammonia in water, Mobile phase (B): Acetonitrile, T/% B: 0/10, 2/10, 8/80, 13/80, 14/80, 15/10, Flow rate: 1.0 mL/min (35:65), compound RT: 16.15 minutes] to afford N-(3-(3-(bicyclo[4.2.0]octa-1,3,5-trien-3-yloxy)pyrrolidin-1-yl)bicyclo[1.1.1]pentan-1-yl)-2-(4-chlorophenoxy)acetamide (0.06 g, 28.4% yield) as a white solid. LCMS (ES) m/z=439 [M+H]⁺. ¹H NMR (400 MHz, DMSO-d₆) δ ppm 1.73-1.76 (m, 1H), 1.94 (s, 6H), 2.14-2.22 (m, 1H), 2.55-2.68 (m, 3H), 2.82-2.86 (m, 1H), 3.02 (d, J=5.2 Hz, 4H), 4.39 (s, 2H), 4.78 (s, 1H), 6.64 (d, J=7.2 Hz, 2H), 6.93 (t, J=8.8 Hz, 3H), 7.31 (d, J=8.0 Hz, 2H), 8.60 (s, 1H).

TABLE 18B

| | | | LCMS m/z | ¹H-NMR (400 MHz, |
|---|---|---|---|---|
| Cmpd # | Structure | Name | [M + H]⁺ | DMSO-d₆) |
| 6x | 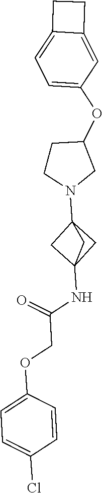 [SMILES: O=C(COc1ccc(Cl)cc1)NC12CC(N3CCC(Oc4ccc5c(c4)CC5)C3)(C1)C2] | N-(3-(3- (bicyclo[4.2.0] octa-1,3,5-trien-3- yloxy)pyrrolidin-1- yl)bicyclo[1.1.1] pentan-1-yl)-2-(4- chlorophenoxy) acetamide | 439 | 1.73-1.76 (m, 1 H), 1.94 (s, 6 H), 2.14-2.22 (m, 1 H), 2.55-2.68 (m, 3 H), 2.82-2.86 (m, 1 H), 3.02 (d, J = 5.2 Hz, 4 H), 4.39 (s, 2 H), 4.78 (s, 1 H), 6.64 (d, J = 7.2 Hz, 2 H), 6.93 (t, J = 8.8 Hz, 3 H), 7.31 (d, J = 8.0 Hz, 2 H), 8.60 (s, 1 H). |

### Example 7a and 7b

### (S)-2-(4-chlorophenoxy)-N-(3-(4-(4-chlorophenoxy)-2-oxopyrrolidin-1-yl)bicyclo[1.1.1]pentan-1-yl)acetamide and (S)-2-(4-chlorophenoxy)-N-(3-(3-(4-chlorophenoxy)-2-oxopyrrolidin-1-yl)bicyclo[1.1.1]pentan-1-yl)acetamide

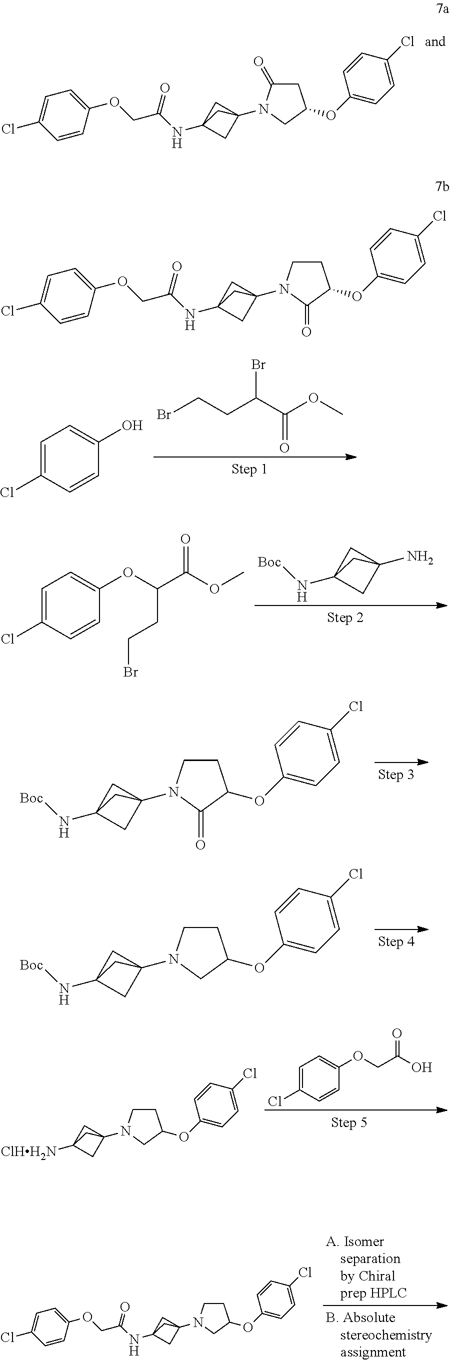

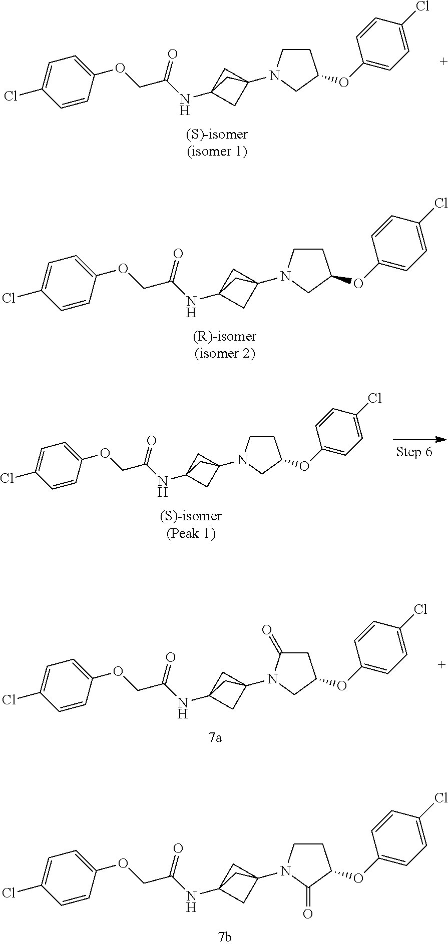 [SMILES: O=C(COc1ccc(Cl)cc1)NC12CC(N3CC[C@H](Oc4ccc(Cl)cc4)C3)(C1)C2 ; O=C(COc1ccc(Cl)cc1)NC12CC(N3CC[C@@H](Oc4ccc(Cl)cc4)C3)(C1)C2 ; O=C(COc1ccc(Cl)cc1)NC12CC(N3CC[C@H](Oc4ccc(Cl)cc4)C3)(C1)C2 ; O=C(COc1ccc(Cl)cc1)NC12CC(N3C[C@@H](Oc4ccc(Cl)cc4)CC3=O)(C1)C2 ; O=C(COc1ccc(Cl)cc1)NC12CC(N3CC[C@H](Oc4ccc(Cl)cc4)C3=O)(C1)C2]

Step 1: To a solution of chlorophenol (10.0 g, 78.12 mmol, 1.0 equiv.) in DMF (160 mL), K₂CO₃ (10.8 g, 78.12 mmol, 1.0 equiv.) was added followed by the addition of methyl-2,4-dibromobutanoate (11.0 mL, 78.12 mmol, 1.0 equiv.) at 0° C. and the reaction was stirred at 60° C. for 2 h. After completion of the reaction (TLC, 10% EtOAc in hexane), the reaction mixture was allowed to come to room temperature and was diluted with ice cold water (300 mL) and the mixture was extracted with EtOAc (3×150 mL). The combined EtOAc extracts were dried over anhydrous Na₂SO₄, filtered and distilled under reduced pressure. The crude material was purified by flash column chromatography eluting the product at 5% EtOAc in hexane to afford methyl 4-bromo-2-(4-chlorophenoxy)butanoate as a colourless liquid (12.9 g, 53.75% yield). ¹H NMR (400 MHz, CDCl₃) δ ppm 2.42-2.48 (m, 2H), 3.59-3.60 (m, 2H), 3.76 (s, 3H), 4.82-4.84 (m, 1H), 6.85 (d, J=6.8 Hz, 2H), 7.24 (d, J=9.2 Hz, 2H).

Step 2: To a solution of tert-butyl (3-aminobicyclo[1.1.1]pentan-1-yl)carbamate (6.2 g, 31.31 mmol, 1.0 equiv) in Et₃N (17.46 mL, 125.25 mmol, 4.0 equiv.) was added methyl 4-bromo-2-(4-chlorophenoxy)butanoate (9.6 g, 31.31 mmol, 1.0 equiv.). The mixture was heated at 100 ⁰0 for 2 h. After completion of the reaction (TLC, 50% EtOAc in hexane), the reaction mixture was diluted with water (100 mL) and extracted with DCM (2×300 mL). The combined extracts were dried over anhydrous Na₂SO₄, filtered and evaporated under vacuum. The crude material was purified by column chromatography using an eluent of 45% EtOAc in hexane to obtain the desired product tert-butyl (3-(3-(4-chlorophenoxy)-2-oxopyrrolidin-1-yl)bicyclo[1.1.1]pentan-1-yl)carbamate as an off-white solid (6.0 g, 48.78% yield). ¹H NMR (400 MHz, DMSO-d₆) δ ppm 1.44 (s, 9H), 2.11-2.15 (m, 1H), 2.39 (s, 6H), 2.49 (bs, 1H), 3.31-3.33 (m, 1H), 3.42-3.44 (m, 1H), 4.77 (t, J=6.6 Hz, 1H), 4.94 (s, 1H), 6.96 (d, J=7.6 Hz, 2H), 7.21 (d, J=7.6 Hz, 2H).

Step 3: To a solution of tert-butyl (3-(3-(4-chlorophenoxy)-2-oxopyrrolidin-1-yl)bicyclo[1.1.1]pentan-1-yl)carbamate (6.0 g, 15.26 mmol, 1.0 equiv.) in THF (20 mL) was added BH₃S(CH₃)₂ (2.9 mL, 30.53 mmol, 2.0 equiv.) at 0° C. (Note: 6.0 g was divided into 2 batches and the reaction was performed.) The reaction stirred at room temperature for 24 h. After completion of the reaction (TLC, 50% EtOAc in hexane), the reaction mixture was quenched with methanol at 0° C. and stirred for 2 h. The reaction mixture was then evaporated under reduced pressure to obtain the crude product, which was diluted with water (200 mL) and extracted with DCM (3×100 mL). The combined organic layer was washed with brine solution (100 mL) and dried over anhydrous Na₂SO₄, filtered and concentrated to obtain the crude which was purified by silica gel column chromatography using 55-60% EtOAc in hexane as an eluent to yield tert-butyl (3-(3-(4-chlorophenoxy)pyrrolidin-1-yl)bicyclo[1.1.1]pentan-1-yl)carbamate as an off-white solid (3.0 g, 52.0% yield). LCMS (ES) m/z=379.1 [M+H]⁺. ¹H NMR (400 MHz, DMSO-d₆) δ ppm 1.43 (s, 9H), 2.02 (s, 7H), 2.26-2.32 (m, 1H), 2.60 (s, 1H), 2.76-2.82 (m, 2H), 2.90 (s, 1H), 4.78 (s, 1H), 4.91 (s, 1H), 6.76 (d, J=6.8 Hz, 2H), 7.20 (d, J=7.2 Hz, 2H).

Step 4: To a solution of tert-butyl (3-(3-(4-chlorophenoxy)pyrrolidin-1-yl)bicyclo[1.1.1]pentan-1-yl)carbamate (3.0 g, 7.91 mmol, 1.0 equiv.) in DCM (20 mL) at 0° C. was added 15 mL of 4 M HCl solution in 1,4-dioxane and the mixture was stirred at room temperature for 16 h. After completion of the reaction (TLC, 50% EtOAc in hexane), the reaction mixture was concentrated to afford the crude product. It was triturated with dry diethyl ether (2×30 mL) and the solid was dried under high vacuum to afford 3-(3-(4-chlorophenoxy)pyrrolidin-1-yl)bicyclo[1.1.1]pentan-1-amine hydrochloride (2.2 g, crude), which was taken to the next step without further purification. LCMS (ES) m/z=279.0 [M+H]⁺. ¹H NMR (400 MHz, DMSO-d₆) δ ppm 2.08 (s, 1H), 2.24 (s, 6H), 3.40-3.50 (m, 5H), 5.10 (s, 1H), 6.97 (d, J=8.4 Hz, 2H), 7.34 (d, J=8.4 Hz, 2H), 9.07 (s, 3H).

Step 5: To a solution of 2-(4-chlorophenoxy)acetic acid (1.18 g, 6.35 mmol, 2.0 equiv.) in DCM (20 mL) at 0° C. was added triethylamine (2.21 mL, 15387 mmol, 5.0 equiv), followed by the addition of T3P® (50 wt % in EtOAc, 3.78 mL, 6.35 mmol, 2.0 equiv.). The reaction mixture was stirred at 0° C. for 5 min and then 3-(3-(4-chlorophenoxy)pyrrolidin-1-yl)bicyclo[1.1.1]pentan-1-amine hydrochloride (1.0 g, 3.17 mmol, 1.0 equiv.) (which was neutralized with triethylamine (1.0 equiv.) in DCM) was added at 0° C. and the reaction was stirred at room temperature for 16 h. After the consumption of the starting material (TLC, 50% EtOAc in hexane), the reaction mixture was diluted with water (100 mL) and was extracted with DCM (2×100 mL). The combined organic layer was washed with saturated aqueous NaHCO₃ solution (100 mL). The combined organic layer was dried over anhydrous Na₂SO₄, filtered and evaporated under reduced pressure to obtain the crude. The crude product was purified by silica gel column chromatography using 65% EtOAc in hexane as an eluent to afford the desired product 2-(4-chlorophenoxy)-N-(3-(3-(4-chlorophenoxy)pyrrolidin-1-yl)bicyclo[1.1.1]pentan-1-yl)acetamide (1.1 g, 71.0% yield) as an off-white solid. LCMS (ES) m/z=447.4 [M+H]⁺. ¹H NMR (400 MHz, DMSO-d₆) δ ppm 1.74-1.77 (m, 1H), 1.95 (s, 6H), 2.18-2.30 (m, 1H), 2.48-2.70 (m, 3H), 2.84-2.86 (m, 1H), 4.40 (s, 2H), 4.83-4.84 (m, 1H), 6.88 (d, J=8.8 Hz, 2H), 6.94 (d, J=9.2 Hz, 2H), 7.28 (d, J=8.8 Hz, 2H), 7.31(d, J=9.2 Hz, 2H), 8.60 (s, 1H).

The racemic product from step 5 was taken forward for isomer separation by chiral prep HPLC. [Analytical conditions:Column:CHIRALPAK IC (100 mm×4.6 mm×3 mic); Mobile phase:n-hexane:IPA with 0.1% DEA (85:15); Flow rate: 1.0 mL/min).] Fractions containing the separated isomers were concentrated under reduced pressure. The solid obtained was triturated with HPLC grade n-hexane (200 mL) and dried under high vacuum.

Based on VCD analysis Isomer 1 was assigned as (S)-2-(4-chlorophenoxy)-N-(3-(3-(4-chlorophenoxy)pyrrolidin-1-yl)bicyclo[1.1.1]pentan-1-yl)acetamide and Isomer 2 as (R)-2-(4-chlorophenoxy)-N-(3-(3-(4-chlorophenoxy)pyrrolidin-1-yl)bicyclo[1.1.1]pentan-1-yl)acetamide.

### (S)-2-(4-chlorophenoxy)-N-(3-(3-(4-chlorophenoxy)pyrrolidin-1-yl)bicyclo[1.1.1]pentan-1-yl)acetamide

Recovery: 0.34 g (off-white solid). LCMS (ES) m/z=447.0 [M+H]⁺. ¹H NMR (400 MHz, DMSO-d₆) δ ppm 1.74-1.77 (m, 1H), 1.95 (s, 6H), 2.18-2.30 (m, 1H), 2.48-2.70 (m, 3H), 2.84-2.86 (m, 1H), 4.40 (s, 2H), 4.83-4.84 (m, 1H), 6.88 (d, J=8.8 Hz, 2H), 6.94 (d, J=9.2 Hz, 2H), 7.28 (d, J=8.8 Hz, 2H), 7.31(d, J=9.2 Hz, 2H), 8.60 (s, 1H).

Chiral HPLC purity: 100% at RT 12.719 min. % ee: 100%

### (R)-2-(4-chlorophenoxy)-N-(3-(3-(4-chlorophenoxy)pyrrolidin-1-yl)bicyclo[1.1.1]pentan-1-yl)acetamide

Recovery: 0.37 g (off-white solid). LCMS (ES) m/z=447.0 [M+H]⁺. ¹H NMR (400 MHz, DMSO-d₆) δ ppm 1.73-1.77 (m, 1H), 1.95 (s, 6H), 2.12-2.26 (m, 1H), 2.65-2.70 (m, 3H), 2.84-2.88 (m, 1H), 4.40 (s, 2H), 4.84 (bs, 1H), 6.88 (d, J=8.8 Hz, 2H), 6.94 (d, J=8.8 Hz, 2H), 7.28 (d, J=8.8 Hz, 2H), 7.31(d, J=8.8 Hz, 2H), 8.60 (s, 1H). Chiral HPLC. purity: 100% at RT 15.67 min; % ee: 100%.

Step 6: To a solution of (S)-2-(4-chlorophenoxy)-N-(3-(3-(4-chlorophenoxy)pyrrolidin-1-yl)bicyclo[1.1.1]pentan-1-yl)acetamide (0.07 g, 0.15 mmol, 1.0 equiv.) in ethyl acetate (5.0 mL) was added ruthenium (IV) oxide monohydrate (0.012 g, 0.078 mmol, 0.5 equiv.) at 0° C., and then 10% aqueous sodium periodate solution (0.16 g, 0.78 mmol, 5.0 equiv.) was added and the reaction was stirred at room temperature for 2 h. After the consumption of the starting material (TLC, 50% ethyl acetate in hexane), the reaction mixture was diluted with EtOAc (100 mL) and was washed with water (2×10 mL). The combined EtOAc extracts were dried over anhydrous Na₂SO₄, filtered and distilled under reduced pressure. The crude material was purified by preparative TLC using 40% EtOAc in hexane (eluted twice) as an eluent. Both the products were isolated separately to afford (S)-2-(4-chlorophenoxy)-N-(3-(4-(4-chlorophenoxy)-2-oxopyrrolidin-1-yl)bicyclo[1.1.1]pentan-1-yl)acetamide (compound 7a, (0.032 g, 44.4% yield) and (S)-2-(4-chlorophenoxy)-N-(3-(3-(4-chlorophenoxy)-2-oxopyrrolidin-1-yl)bicyclo[1.1.1]pentan-1-yl)acetamide (compound 7b, 0.035 g, 48.6% yield) as off-white solid.

(S)-2-(4-chlorophenoxy)-N-(3-(4-(4-chlorophenoxy)-2-oxopyrrolidin-1-yl)bicyclo[1.1.1]pentan-1-yl)acetamide 7a:_LCMS (ES) m/z=461.0 [M+H]⁺. ¹H NMR (400 MHz, DMSO-d₆) δ -ppm 2.29 (s, 6H), 2.81-2.87 (m, 2H), 3.33 (d, J=11.2 Hz, 1H), 3.75-3.80 (m, 1H), 4.41 (s, 2H), 4.99 (bs, 1H), 6.94 (t, J=7.8 Hz, 4H), 7.31 (d, J=8.4 Hz, 4H), 8.70 (s, 1H). Chiral HPLC purity: 99.77% at 280 nm. % ee: 99.54%.

(S)-2-(4-chlorophenoxy)-N-(3-(3-(4-chlorophenoxy)-2-oxopyrrolidin-1-yl)bicyclo[1.1.1]pentan-1-yl)acetamide 7b: LCMS (ES) m/z=461.0 [M+H]⁺. ¹H NMR (400 MHz, DMSO-d₆) δ ppm 1.85-1.90 (m, 2H), 2.31 (s, 6H), 3.34-3.36 (m, 2H), 4.42 (s, 2H), 4.95-4.96 (m, 1H), 6.95 (d, J=8.8 Hz, 2H), 7.01 (d, J=8.8 Hz, 2H), 7.31 (t, J=9.2 Hz, 4H), 8.72 (s, 1H). Chiral HPLC purity: 100.0% at 280 nm. % ee: 100.0%

The ruthenium (IV) oxide oxidation reaction was performed with (R)-2-(4-chlorophenoxy)-N-(3-(3-(4-chlorophenoxy)pyrrolidin-1-yl)bicyclo[1.1.1]pentan-1-yl)acetamide as described above and the products 7c and 7d were isolated by preparative TLC purification.

The Compounds of Examples 7c to 7d were prepared generally according to the procedure described above for Example 7a and 7b.

TABLE 19

| Cmpd | | | LCMS m/z | ¹H-NMR (400 MHz, |
|---|---|---|---|---|
| # | Structure | Name | [M + H]⁺ | DMSO-d₆) |
| 7a | 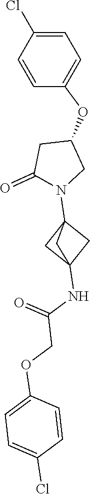 [SMILES: O=C(COc1ccc(Cl)cc1)NC12CC(N3C[C@@H](Oc4ccc(Cl)cc4)CC3=O)(C1)C2] | (S)-2-(4-chlorophenoxy)- N-(3-(4-(4- chlorophenoxy)-2- oxopyrrolidin-1- yl)bicyclo[1.1.1]pentan-1- yl)acetamide | 461.0 | 2.29 (s, 6 H), 2.81-2.87 (m, 2 H), 3.33 (d, J = 11.2 Hz, 1 H), 3.75-3.80 (m, 1 H), 4.41 (s, 2 H), 4.99 (bs, 1 H), 6.94 (t, J = 7.8 Hz, 4 H), 7.31 (d, J = 8.4 Hz, 4 H), 8.70 (s, 1 H). |
| 7b | 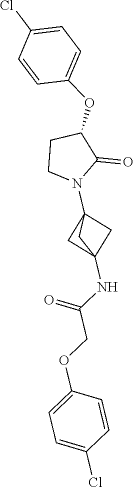 [SMILES: O=C(COc1ccc(Cl)cc1)NC12CC(N3CC[C@H](Oc4ccc(Cl)cc4)C3=O)(C1)C2] | (S)-2-(4-chlorophenoxy)- N-(3-(3-(4- chlorophenoxy)-2- oxopyrrolidin-1- yl)bicyclo[1.1.1]pentan-1- yl)acetamide | 461.0 | 1.85-1.90 (m, 2 H), 2.31 (s, 6 H), 3.34-3.36 (m, 2 H), 4.42 (s, 2 H), 4.95- 4.96 (m, 1 H), 6.95 (d, J = 8.8 Hz, 2 H), 7.01 (d, J = 8.8 Hz, 2 H), 7.31 (t, J = 9.2 Hz, 4 H), 8.72 (s, 1 H). |
| 7c | 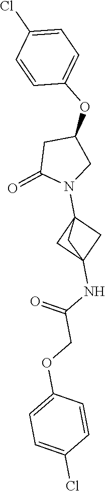 [SMILES: O=C(COc1ccc(Cl)cc1)NC12CC(N3C[C@H](Oc4ccc(Cl)cc4)CC3=O)(C1)C2] | (R)-2-(4-chlorophenoxy)- N-(3-(4-(4- chlorophenoxy)-2- oxopyrrolidin-1- yl)bicyclo[1.1.1]pentan-1- yl)acetamide | 461.4 | 2.29 (s, 6 H), 2.81-2.87 (m, 2 H), 3.33 (d, J = 11.2 Hz, 1 H), 3.75-3.79 (m, 1 H), 4.41 (s, 2 H), 4.99 (bs, 1 H), 6.94 (t, J = 8.0 Hz, 4 H), 7.31 (d, J = 8.8 Hz, 4 H), 8.70 (s, 1 H). |
| 7d | 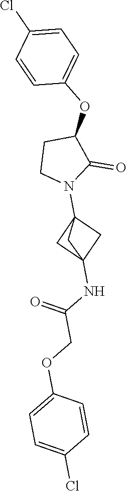 [SMILES: O=C(COc1ccc(Cl)cc1)NC12CC(N3CC[C@@H](Oc4ccc(Cl)cc4)C3=O)(C1)C2] | (R)-2-(4-chlorophenoxy)- N-(3-(3-(4- chlorophenoxy)-2- oxopyrrolidin-1- yl)bicyclo[1.1.1]pentan-1- yl)acetamide | 461.4 | 1.85-1.90 (m, 2 H), 2.31 (s, 6 H), 3.34-3.39 (m, 2 H), 4.42 (s, 2 H), 4.97 (t, J = 7.4 Hz, 1 H), 6.95 (d, J = 8.8 Hz, 2 H), 7.01 (d, J = 8.4 Hz, 2 H), 7.31 (t, J = 9.0 Hz, 4 H), 8.73 (s, 1 H). |

### Example 8a

### 2-(4-chlorophenoxy)-N-(3-(4-(4-chlorophenoxy)piperidin-1-yl)bicyclo[1.1.1]pentan-1-yl)acetamide

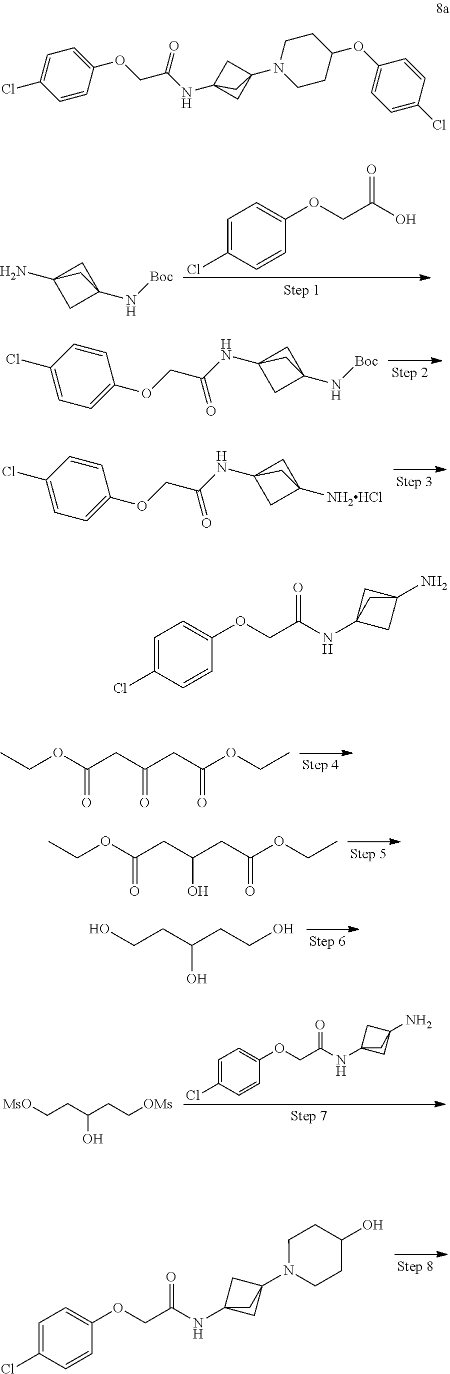

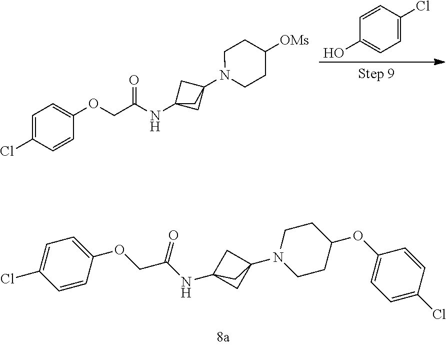 [SMILES: CS(=O)(=O)OC1CCN(C23CC(NC(=O)COc4ccc(Cl)cc4)(C2)C3)CC1 ; Oc1ccc(Cl)cc1 ; O=C(COc1ccc(Cl)cc1)NC12CC(N3CCC(Oc4ccc(Cl)cc4)CC3)(C1)C2]

Step 1: To a solution of 2-(4-chlorophenoxy)acetic acid (33.87 g, 181.57 mmol, 1.2 equiv) in DCM (300 mL) at 0° C. was added triethylamine (63.35 mL, 453.93 mmol, 3 equiv) and T3P® (50 wt. % in ethyl acetate) (135.1 mL, 226.96 mmol, 1.5 equiv) and was stirred for 10 minutes at 0° C. After that tert-butyl (3-aminobicyclo[1.1.1]pentan-1-yl)carbamate (30.0 g, 151.31 mmol, 1 equiv) was added to the reaction mixture and the reaction mixture was stirred at room temperature for 16 hours. After completion (monitored by TLC), the reaction mixture was diluted with water (200 mL) and extracted with DCM (2×300 mL). The combined organic extract was washed with aqueous saturated sodium bicarbonate solution (200 mL), and the organic layer was filtered and concentrated under reduced pressure to obtain the crude product. Following the same procedure, another 30 g batch reaction of tert-butyl (3-aminobicyclo[1.1.1]pentan-1-yl)carbamate was performed to obtain the final combined yield of 108 g of tert-butyl (3-(2-(4-chlorophenoxy)acetamido)bicyclo[1.1.1]pentan-1-yl)carbamate (97.24% yield) as an off-white solid. LCMS (ES) m/z=311.1 [M+H]⁺ (t-butyl cleavage mass was observed). ¹H NMR (400 MHz, DMSO-d₆) β ppm 1.35 (s, 9H), 2.11 (s, 6H), 4.39 (s, 2H), 6.94 (d, J=8.8 Hz, 2H), 7.31 (d, J=8.8 Hz, 2H), 7.46 (bs, 1H), 8.60 (s, 1H).

Step 2: To a solution of tert-butyl (3-(2-(4-chlorophenoxy)acetamido)bicyclo[1.1.1]pentan-1-yl)carbamate (27 g, 73.57 mmol, 1 equiv) in DCM (400 mL) was added a solution of 4 M HCl in 1,4-dioxane (90 mL) at 0° C. The reaction mixture was allowed to warm and stir at room temperature for 12 h. After consumption of the starting material (TLC, 5% Methanol in DCM), DCM was evaporated under reduced pressure. The obtained solid was triturated with diethyl ether (300 mL) and dried under high vacuum to obtain N-(3-aminobicyclo[1.1.1]pentan-1-yl)-2-(4-chlorophenoxy)acetamide hydrochloride. Following the same procedure, another 3 batches were performed to afford a total of 84 g (94.52% yield) of N-(3-aminobicyclo[1.1.1]pentan-1-yl)-2-(4-chlorophenoxy)acetamide hydrochloride as an off-white solid. ¹H NMR (400 MHz, DMSO-d₆) δ ppm 2.22 (s, 6H), 4.44 (s, 2H), 6.95 (d, J=8.8 Hz, 2H), 7.32 (d, J=9.2 Hz, 2H), 8.87 (s, 1H), 9.0 (bs, 3H).

Step 3: N-(3-aminobicyclo[1.1.1]pentan-1-yl)-2-(4-chlorophenoxy)acetamide hydrochloride (10.0 g, 33.16 mmol, 1 equiv) was added to an aqueous sodium bicarbonate solution (5.57 g, 66.30 mmol, 2 equiv. in 100 mL of water) and stirred at room temperature for 1 h. The reaction mixture was extracted with DCM (2×250 mL). The combined organic extract was washed with water (100 mL) and brine solution (50 mL). The organic phase was dried over anhydrous sodium sulfate, filtered and concentrated under reduced pressure to obtain the crude N-(3-aminobicyclo[1.1.1]pentan-1-yl)-2-(4-chlorophenoxy)acetamide (18 g, crude, 10.0 g scale reactions in 2 batches (20.0 g) were performed). 8 g of the crude material was further purified by reverse phase purification: [Column: C18, Mobile phase (A): 0.1% ammonia in water, Mobile phase (B): Acetonitrile] to yield N-(3-aminobicyclo[1.1.1]pentan-1-yl)-2-(4-chlorophenoxy)acetamide (6.6 g, 50.7% yield) as a white solid. LCMS (ES) m/z=250.2 [M+H]⁺ (loss of —NH₂). ¹H NMR (400 MHz, DMSO-d₆) δ ppm 1.91 (s, 6H), 2.10 (s, 2H), 4.38 (s, 2H), 6.94 (d, J=8.8 Hz, 2H), 7.31 (d, J=8.8 Hz, 2H), 8.47 (s, 1H).

Step 4: To a solution of diethyl-3-oxopentanedioate (5.0 g, 24 mmol, 1.0 equiv) in ethanol (50 mL) was added sodium borohydride (0.93 g, 24 mmol, 1.0 equiv.) at 0° C. in portions over a period of 15 minutes and the reaction mixture was maintained for 10 minutes at room temperature. The reaction mixture was quenched with saturated aqueous solution of NH₄Cl (30 mL) at 0° C., extracted with DCM (2×250 mL), dried over anhydrous sodium sulfate, filtered and concentrated under reduced pressure to afford diethyl-3-hydroxypentanedioate (3.5 g, crude) as a viscous liquid. LCMS (ES) m/z=205.0 [M+H]⁺. ¹H NMR (400 MHz, DMSO-d₆) δ ppm 1.16 (t, J=7.0 Hz, 6H), 2.31-2.38 (m, 2H), 2.43-2.48(m, 2H), 4.03 (q, J=7.2 Hz, 4H), 4.20-4.25 (m, 1H), 5.01 (d, J=6.4 Hz, 1H).

Step 5: To a solution of diethyl 3-hydroxypentanedioate (3.0 g, 14 mmol, 1.0 equiv) in dry THF (100 mL) under N₂ atmosphere was added 1 M Lithium aluminiumhydride solution in THF (58.7 mL, 58 mmol, 4.0 equiv.) at 0° C. slowly dropwise over a period of 30 minutes. The reaction mixture was stirred for 16 h at room temperature. The reaction was quenched with aqueous 1 N NaOH solution (20 mL) at 0° C., diluted with DCM (150 mL), and filtered through a Celite® bed. The Celite® bed was washed with a solution of 10% methanol in DCM (2×100 mL). The filtrate was dried over anhydrous sodium sulfate, filtered and concentrated under reduced pressure to afford pentane-1,3,5-triol (1.4 g, 86.5% yield) as a viscous liquid. ¹H NMR (400 MHz, DMSO-d₆) δ ppm 1.40-1.49 (m, 4H), 3.35 (s, 1H), 3.40-3.48 (m, 4H).

Step 6: To a solution of pentane-1,3,5-triol (1.4 g, 11 mmol, 1.0 equi) in pyridine (6 mL) was added mesyl chloride (1.89 mL, 24 mmol, 2.1 equiv) at 0° C. over a period of 30 minutes. The reaction mixture was stirred for 2 h at room temperature. The reaction was quenched with aqueous 2 N HCl solution (50 mL) at 0° C. and extracted with DCM (2×100 mL). The organic layer was washed with aq. 2 N HCl solution (2×50 mL), water (50 mL), 10% aq. NaHCO₃ (50 mL), dried over anhydrous sodium sulfate, filtered and concentrated to afford 3-hydroxypentane-1,5-diyl dimethanesulfonate (1.2 g, crude) as a viscous liquid. LCMS (ES) m/z=277.0 [M+H]⁺.

Step 7: To a solution of N-(3-aminobicyclo[1.1.1]pentan-1-yl)-2-(4-chlorophenoxy)acetamide (0.5 g, 1.8 mmol, 1.0 equiv) in dimethylacetamide (10 mL) was added N,N-Di-isopropylethylamine (1.57 mL, 9 mmol, 5.0 equi) and 3-hydroxypentane-1,5-diyl dimethanesulfonate (1.0 g, 3.6 mmol, 2.0 equiv.) at room temperature. The reaction mixture was maintained at 80° C. for 1.5 h under microwave irradiation. It was then cooled to room temperature and quenched with crushed ice (100 mL) and extracted with DCM (2×100 mL). The combined organic extract was washed with cold water (25 mL), dried over anhydrous sodium sulfate, filtered and concentrated under reduced pressure. The crude product was purified by flash chromatography (neutral alumina column) using 0.1% to 10% methanol in DCM as an eluent to obtain 2-(4-chlorophenoxy)-N-(3-(4-hydroxypiperidin-1-yl)bicyclo[1.1.1]pentan-1-yl)acetamide (0.3 g, 47.6% yield) as a gum. LCMS (ES) m/z=351.0 [M+H]⁺.

Step 8: To a solution of 2-(4-chlorophenoxy)-N-(3-(4-hydroxypiperidin-1-yl)bicyclo[1.1.1]pentan-1-yl)acetamide (0.20 g, 0.57 mmol, 1.0 equiv) in DCM (30 mL) was added TEA (0.24 mL, 1.7 mmol, 3.0 equiv) at 0° C. and stirred for 30 minutes at the same temperature. Then mesyl chloride (0.052 mL, 0.68 mmol, 1.2 equiv) was added at 0° C. and the reaction mixture was stirred for 3 h at room temperature. The reaction mixture was diluted with DCM (100 mL) and washed with 10% aq. NaHCO₃ solution (2×25 mL) and water (2×25 mL). The separated organic layer was dried over anhydrous sodium sulfate, filtered and concentrated under reduced pressure to afford 1-(3-(2-(4-chlorophenoxy)acetamido)bicyclo[1.1.1]pentan-1-yl)piperidin-4-yl methanesulfonate (0.22 g, crude) as a gum. LCMS (ES) m/z=429.1 [M+H]⁺. It was taken to next step without further purification.

Step 9: To a solution of 4-chlorophenol (0.05 g, 0.39 mmol, 1.0 equiv) in DMF (10 mL) was added K₂CO₃ (0.16 g, 1.1 mmol, 3.0 equiv) and 1-(3-(2-(4-chlorophenoxy)acetamido)bicyclo[1.1.1]pentan-1-yl)piperidin-4-yl methanesulfonate (0.2 g, 0.46 mmol, 1.2 equiv) at room temperature. The reaction mixture was stirred at 80° C. for 16 h, after which the reaction mixture was cooled to room temperature and quenched with crushed ice (50 mL). The aqueous was extracted with DCM (2×100 mL) and the combined organic layers were washed with cold water (25 mL), dried over anhydrous sodium sulfate, filtered and concentrated under reduced pressure to obtain the crude product. It was purified by flash chromatography using 0.1% to 10% methanol in DCM as an eluent and further re-purified by prep HPLC [Analytical conditions: Column: XBRIDGE. Mobile phase (A): 0.1% Ammonia in water, Mobile phase (B): Acetonitrile, T/% B: 0/10, 2/10, 8/80, 13/80, 14/80, 15/10, compound RT: 9.60 minutes] to obtain 2-(4-chlorophenoxy)-N-(3-(4-(4-chlorophenoxy)piperidin-1-yl)bicyclo[1.1.1]pentan-1-yl)acetamide (8.0 mg, 4.4% yield) as a white solid. LCMS (ES) m/z=461 [M+H]⁺. ¹H NMR (400 MHz, DMSO-d₆) δ ppm 1.55-1.60 (m, 2H), 1.88-1.91 (m, 2H), 1.94 (s, 6H), 2.22 (t, J=9.4 Hz, 2H), 2.64 (bs, 2H), 4.32 (bs, 1H), 4.40 (s, 2H), 6.94 (d, J=8.8 Hz, 4H), 7.27 (d, J=8.8 Hz, 2H), 7.31 (d, J=8.8 Hz, 2H), 8.60 (s, 1H).

TABLE 20

| | | | LCMS m/z | ¹H-NMR (400 MHz, |
|---|---|---|---|---|
| Cmpd # | Structure | Name | [M + H]⁺ | DMSO-d₆) |
| 8a | 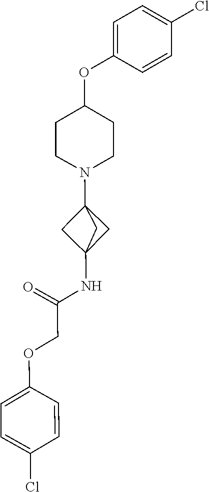 [SMILES: O=C(COc1ccc(Cl)cc1)NC12CC(N3CCC(Oc4ccc(Cl)cc4)CC3)(C1)C2] | 2-(4-chlorophenoxy)-N- (3-(4-(4- chlorophenoxy) piperidin-1- yl)bicyclo[1.1.1]pentan- 1-yl)acetamide | 461.4 | 1.55-1.60 (m, 2 H), 1.88- 1.91 (m, 2 H), 1.94 (s, 6 H), 2.22 (t, J = 9.4 Hz, 2 H), 2.64 (bs, 2 H), 4.32 (bs, 1 H), 4.40 (s, 2 H), 6.94 (d, J = 8.8 Hz, 4 H), 7.27 (d, J = 8.8 Hz, 2 H), 7.31 (d, J = 8.8 Hz, 2 H), 8.60 (s, 1 H). |

### Assay Example 1

### ATF4 Cell Based Assay

The ATF4 reporter assay measures the effect of Thapsigargin induced cellular stress on ATF4 expression. For this reporter assay, a stable cell line was created by transfecting SH-SY5Y cells with a plasmid containing the NanoLuc® luciferase gene fused to the 5′-UTR of ATF4, under the control of the CMV promoter. The ATF4 5′-UTR contains two open reading frames which mediate the cellular stress-dependent translation of the reporter gene. Clones stably expressing the reporter construct were isolated and selected based on the luminescence response to thapsigargin and inhibition of this signal by test compounds. Briefly, SH-SY5Y-ATF4-NanoLuc cells were challenged with Thapsigargin for 14-18 hours to determine the stress effect with or without test compounds.

Cells were propagated in growth media consisting of 90% DMEM F12 (InVitrogen #11320-033), 10% Fetal Bovine Serum (Gibco #10438-026), 5mM Glutamax (Gibco #35050-061), 5 mM Hepes, (Gibco #15630-080), and 0.5 mg/ml Geneticin (Gibco #10131-027). Cells were prepared for the assay by removing all media from cells, washing the plated cells with phosphate buffered saline, and detached by adding a solution comprised of 10% Tryple express solution (InVitrogen12604-021) and 90% enzyme-free cell dissociation buffer HANKS base (Gibco 13150-016). The trypsin was deactivated by adding assay media comprised of 90% phenol-red free DMEM F12 (InVitrogen, 11039), 10% Fetal Bovine Serum (Gibco #10438-026),(5 mM Glutamax (Gibco #35050-061), 5 mM Hepes, (Gibco #15630-080), and 0.5 mg/ml Geneticin (Gibco #10131-027). Suspended cells were spun down at 300 g for 5 min, the supernatant was removed and the cell pellet was suspended in warm media (30-37° C.) comprised as above but without 10% Fetal Bovine Serum to a concentration of 1 e6 cells/ml.

Assay plates were prepared by adding 250 nL of compound stock solution in 100% DMSO to each well, followed by dispensing 20 microliters/well cell suspension to deliver 15-20 k cell/well. Cells were incubated for 1 hour at 37° C. Then, 5 μL of 1.5 μM or 1 μM of Thapsigargin (final concentration: 200-300 nM) was added to each well of cells. Assay plates containing cells were incubated for 14-18 hours at 37° C.

The measurement of luciferase produced by the ATF4 constructs was measured as follows. Aliquots of the Nano-Glo reagent (Nano-Glo® Luciferase Assay Substrate, Promega, N113, Nano-Glo® Luciferase Assay Buffer, Promega, N112 (parts of Nano-Glo® Luciferase Assay System, N1150) were brought to room temperature, the substrate and buffer were mixed according to manufacturer's instructions. The cell plates were equilibrated to room temperature. 25 microliters/well of the mixed Nano-Glo reagent were dispensed into assay wells and pulse spun to settle contents and the plate was sealed with film. The plates were incubated at room temperature for 1 hour before detecting luminescence on an EnVision® plate reader.

### Formulation Example 1

### Capsule Composition

An oral dosage form for administering the present invention is produced by filing a standard two piece hard gelatin capsule with the ingredients in the proportions shown in Formulation Table 21, below.

Formulation Table 21

| INGREDIENTS | AMOUNTS | |
|---|---|---|
| 2-(4-chlorophenoxy)-N-(3-(3-(4-chlorophenyl)-2- | 7 | mg |
| oxoimidazolidin-1-yl)bicyclo[1.1.1]pentan-1-yl)acetamide | | |
| (Compound of Example 1a) | | |
| Lactose | 53 | mg |
| Talc | 16 | mg |
| Magnesium Stearate | 4 | mg |

### Formulation Example 2

### Injectable Parenteral Composition

An injectable form for administering the present invention is produced by stirring 1.7% by weight of N-(3-(2-(4-chlorophenoxy)acetamido)bicyclo[1.1.1]pentan-1-yl)-2-(4-chlorophenyl)cyclopropane-1-carboxamide (Compound of Example 2b) in 10% by volume propylene glycol in water.

### Formulation Example 3

### Tablet Composition

The sucrose, calcium sulfate dihydrate and an ATF4 pathway inhibitor as shown in Formulation Table 22 below, are mixed and granulated in the proportions shown with a 10% gelatin solution. The wet granules are screened, dried, mixed with the starch, talc and stearic acid, screened and compressed into a tablet.

Formulation Table 22

| INGREDIENTS | AMOUNTS | |
|---|---|---|
| 2-(4-chlorophenoxy)-N-(3-(3-(4-chlorophenoxy)pyrrolidin- | 12 | mg |
| 1-yl)bicyclo[1.1.1]pentan-1-yl)acetamide | | |
| (Compound of Example 6a) | | |
| calcium sulfate dihydrate | 30 | mg |
| Sucrose | 4 | mg |
| Starch | 2 | mg |
| Talc | 1 | mg |
| stearic acid | 0.5 | mg |

### Biological Activity

Compounds of the invention are tested for activity against ATF4 translation in the above assay.

The compounds of Examples I to IV were tested generally according to the above ATF4 cell based assay and in a set of two or more experimental runs exhibited an average ATF4 pathway inhibitory activity (IC₅₀)<125 nM.

The compounds of Examples I to XXIII were tested generally according to the above ATF4 cell based assay and in a set of two or more experimental runs exhibited an average ATF4 pathway inhibitory activity (IC₅₀)<325 nM.

The compound of Example III was tested generally according to the above ATF4 cell based assay and in a set of two or more experimental runs exhibited an average ATF4 pathway inhibitory activity (IC₅₀) of 8.89 nM.

The compound of Example VI was tested generally according to the above ATF4 cell based assay and in a set of two or more experimental runs exhibited an average ATF4 pathway inhibitory activity (IC₅₀) of 4.1 nM.

The compound of Example IX was tested generally according to the above ATF4 cell based assay and in a set of two or more experimental runs exhibited an average ATF4 pathway inhibitory activity (IC₅₀) of 300.05 nM.

The compound of Example XII was tested generally according to the above ATF4 cell based assay and in a set of two or more experimental runs exhibited an average ATF4 pathway inhibitory activity (IC₅₀) of 25.2 nM.

The compound of Example XVI was tested generally according to the above ATF4 cell based assay and in a set of two or more experimental runs exhibited an average ATF4 pathway inhibitory activity (IC₅₀) of 13 nM.

The compound of Example XXI was tested generally according to the above ATF4 cell based assay and in a set of two or more experimental runs exhibited an average ATF4 pathway inhibitory activity (IC₅₀) of 33 nM.

The compound of Example XXII was tested generally according to the above ATF4 cell based assay and in a set of two or more experimental runs exhibited an average ATF4 pathway inhibitory activity (IC₅₀) of 1.3 nM.

The compound of Example XXIV and 6a was tested generally according to the above ATF4 cell based assay and in a set of two or more experimental runs exhibited an average ATF4 pathway inhibitory activity (IC₅₀) of 7.3 nM.

The compounds of Examples 1a to 1v were tested generally according to the above ATF4 cell based assay and in at least one set of two or more experimental runs exhibited an average ATF4 pathway inhibitory activity (IC₅₀) from 0.6 to 384 nM.

The compound of Example 1a was tested generally according to the above ATF4 cell based assay and in at least one set of two or more experimental runs exhibited an average ATF4 pathway inhibitory activity (IC₅₀) of 2.1 nM.

The compound of Example 1g was tested generally according to the above ATF4 cell based assay and in at least one set of two or more experimental runs exhibited an average ATF4 pathway inhibitory activity (IC₅₀) of 17 nM.

The compound of Example 1k was tested generally according to the above ATF4 cell based assay and in at least one set of two or more experimental runs exhibited an average ATF4 pathway inhibitory activity (IC₅₀) of 34 nM.

The compound of Example 1l was tested generally according to the above ATF4 cell based assay and in at least one set of two or more experimental runs exhibited an average ATF4 pathway inhibitory activity (IC₅₀) of 1.6 nM.

The compound of Example 1m was tested generally according to the above ATF4 cell based assay and in at least one set of two or more experimental runs exhibited an average ATF4 pathway inhibitory activity (IC₅₀) of 0.6 nM.

The compound of Example 1o was tested generally according to the above ATF4 cell based assay and in at least one set of two or more experimental runs exhibited an average ATF4 pathway inhibitory activity (IC₅₀) of 10.3 nM.

The compound of Example 1r was tested generally according to the above ATF4 cell based assay and in at least one set of two or more experimental runs exhibited an average ATF4 pathway inhibitory activity (IC₅₀) of 1.8 nM.

The compound of Example 1t was tested generally according to the above ATF4 cell based assay and in at least one set of two or more experimental runs exhibited an average ATF4 pathway inhibitory activity (IC₅₀) of 51.7 nM.

The compound of Example 1v was tested generally according to the above ATF4 cell based assay and in at least one set of two or more experimental runs exhibited an average ATF4 pathway inhibitory activity (IC₅₀) of 23.2 nM.

The compounds of Examples 2a to 2c were tested generally according to the above ATF4 cell based assay and in at least one set of two or more experimental runs exhibited an average ATF4 pathway inhibitory activity (IC₅₀) from 12.3 to 27.4 nM.

The compounds of Examples 3a and 3b were tested generally according to the above ATF4 cell based assay and in at least one set of two or more experimental runs exhibited an average ATF4 pathway inhibitory activity (IC₅₀) from 3.6 to 13.6 nM.

The compounds of Examples 4a to 4e were tested generally according to the above ATF4 cell based assay and in at least one set of two or more experimental runs exhibited an average ATF4 pathway inhibitory activity (IC₅₀) from 4.7 to 326 nM.

The compound of Example 4d was tested generally according to the above ATF4 cell based assay and in at least one set of two or more experimental runs exhibited an average ATF4 pathway inhibitory activity (IC₅₀) of 12.2 nM.

The compounds of Examples 5a to 5c were tested generally according to the above ATF4 cell based assay and in at least one set of two or more experimental runs exhibited an average ATF4 pathway inhibitory activity (IC₅₀) from 36 to 2097 nM.

The compounds of Examples 6a to 6x were tested generally according to the above ATF4 cell based assay and in at least one set of two or more experimental runs exhibited an average ATF4 pathway inhibitory activity (IC₅₀) from 1.11 to 210 nM.

The compound of Example 6c was tested generally according to the above ATF4 cell based assay and in at least one set of two or more experimental runs exhibited an average ATF4 pathway inhibitory activity (IC₅₀) of 6.7 nM.

The compound of Example 6e was tested generally according to the above ATF4 cell based assay and in at least one set of two or more experimental runs exhibited an average ATF4 pathway inhibitory activity (IC₅₀) of 20 nM.

The compound of Example 6i was tested generally according to the above ATF4 cell based assay and in at least one set of two or more experimental runs exhibited an average ATF4 pathway inhibitory activity (IC₅₀) of 6.9 nM.

The compound of Example 6k was tested generally according to the above ATF4 cell based assay and in at least one set of two or more experimental runs exhibited an average ATF4 pathway inhibitory activity (IC₅₀) of 24.57 nM.

The compound of Example 6o was tested generally according to the above ATF4 cell based assay and in at least one set of two or more experimental runs exhibited an average ATF4 pathway inhibitory activity (IC₅₀) of 5.7 nM.

The compound of Example 6q was tested generally according to the above ATF4 cell based assay and in at least one set of two or more experimental runs exhibited an average ATF4 pathway inhibitory activity (IC₅₀) of 1.1 nM.

The compound of Example 6v was tested generally according to the above ATF4 cell based assay and in at least one set of two or more experimental runs exhibited an average ATF4 pathway inhibitory activity (IC₅₀) of 9.9 nM.

The compounds of Examples 7a to 7c were tested generally according to the above ATF4 cell based assay and in at least one set of two or more experimental runs exhibited an average ATF4 pathway inhibitory activity (IC₅₀) from 1.2 to 65.8 nM.

The compound of Example 8a was tested generally according to the above ATF4 cell based assay and in at least one set of two or more experimental runs exhibited an average ATF4 pathway inhibitory activity (IC₅₀) of 5.1 nM.

### REFERENCES

1. Wek R C, Jiang H-Y, Anthony T G. Coping with stress: eIF2 kinases and translational control. Biochem. Soc. Trans. 2006 February; 34(Pt I):7-11.
2. Hinnebusch A G, Lorsch J R. The mechanism of eukaryotic translation initiation: new insights and challenges. Cold Spring Harb Perspect Biol. 2012; 4(10): a011544.
3. Krishnamoorthy T, Pavitt G D, Zhang F, Dever T E, Hinnebusch A G. Tight binding of the phosphorylated alpha subunit of initiation factor 2 (eIF2alpha) to the regulatory subunits of guanine nucleotide exchange factor eIF2B is required for inhibition of translation initiation. Mol Cell Biol. 2001 August; 21(15):5018-30.
4. Hinnebusch A G. Translational regulation of GCN4 and the general amino acid control of yeast. Annu. Rev. Microbiol. 2005; 59:407-50.
5. Jackson R J, Hellen C U T, Pestova T V. The mechanism of eukaryotic translation initiation and principles of its regulation. Nat Rev Mol Cell Biol. 2010 February I; I I(2):113-27.
6. Harding H P, Novoa I, Zhang Y, Zeng H, Wek R, Schapira M, et al. Regulated translation initiation controls stress-induced gene expression in mammalian cells. Mol. Cell. 2000 November; 6(5):1099-108.
7. Palam L R, Baird T D, Wek R C. Phosphorylation of eIF2 facilitates ribosomal bypass of an inhibitory upstream ORF to enhance CHOP translation. Journal of Biological Chemistry. 2011 April I; 286(13):10939-49.
8. Vattem K M, Wek R C. Reinitiation involving upstream ORFs regulates ATF4 mRNA translation in mammalian cells. Proc Natl Acad Sci USA. 2004 Aug. 3; 101(31):11269-74.
9. Ma Y, Brewer J W, Diehl J A, Hendershot L M. Two distinct stress signaling pathways converge upon the CHOP promoter during the mammalian unfolded protein response. J. Mol. Biol. 2002 May 17; 318(5):1351-65.
10. Pavitt G D, Ron D. New insights into translational regulation in the endoplasmic reticulum unfolded protein response. Cold Spring Harb Perspect Biol. 2012 Jun.; 4(6): a012278.
11. Ron D, Walter P. Signal integration in the endoplasmic reticulum unfolded protein response. Nat Rev Mol Cell Biol. 2007 July; 8(7):519-29.
12. Gardner B M, Walter P. Unfolded proteins are Irel-activating ligands that directly induce the unfolded protein response. Science. 2011 Sep. 30; 333(6051):1891-4.
13. Harding H P, Zhang Y, Bertolotti A, Zeng H, Ron D. Perk is essential for translational regulation and cell survival during the unfolded protein response. Mol Cell. 2000 May; 5(5):897-904.
14. Walter P, Ron D. The unfolded protein response: from stress pathway to homeostatic regulation. Science. 2011 Nov. 25; 334(6059):1081-6.
15. Tabas I, Ron D. Integrating the mechanisms of apoptosis induced by endoplasmic reticulum stress. Nat Cell Biol. 2011 March l; 13(3):184-90.
16. Shore G C G, Papa F R F, Oakes S A S. Signaling cell death from the endoplasmic reticulum stress response. Current Opinion in Cell Biology. 2011 April I; 23(2):143-9.
17. Bi M, Naczki C, Koritzinsky M, Fels D, 174 WO 2014/144952 PC T/US2014/029568 Blais J, Hu N, Harking H, Novoa I, Varia M, Raleigh J, Scheuner D, Kaufman R J, Bell J, Ron D, Wouters B G, Koumenis C. 2005. ER stress-regulated translation increases tolerance to extreme hypoxia and promotes tumor growth. EMBO J. 24:3470-3481.
18. Bobrovnikova-Marjon E, Pytel D, Vaites L P, Singh N, Koretzky G A, Diehl J A. 2010. PERK promotes cancer cell proliferation and tumor growth by limiting oxidative DNA damage. Oncogene 29: 3881-3895.
19. Avivar-Valderas A, Bobrovnikova-Marjon E, Diehl A, Nagi C, Debnath J, Aguirre-Guiso J A 2011. PERK integrates autophagy and oxidative stress responses to promote survival during extracellular matrix detachment. Mol Cel Biol 31:3616-3629.
20. Axten J M., Medina J. R., Feng Y., Shu A., Romeril S. P. et al. 2012. Discovery of 7-methy-5(I-([3-10 (trifluoromethyl)phenyl]acetyl)-2,3-dihydro-IH-indo1-5yl)-7H-pyrrolo [2,3-d]pyrimidin-4 amine (GSK2606414), a potent and selective first-in class inhibitor of protein kinase R (PKR)-like endplasmic reticulum kinase (PERK). J. Med. Chem. 55(16):7193-7207
21. Ye J. Kumanova M., Hart L. S., Sloane K., Zhang H. et al. 2010. The GCN2-ATF4 pathway is critical for tumour cell survival and proliferation in response to nutrient deprivation. EMBO J. 29: 2082-2096.
22. Moreno J A, Radford H, Peretti D, Steinert J R, Verity N, Martin M G, Halliday M, Morgan J, Dinsdale D, Ortori C A, Barrett D A, Tsaytler P, Bertolotti A, Willis A E, Bushell M, Mallucci G R. 2012. Sustained translational repression by eIF2n-P mediates prion neurodegeneration. Nature 485:507-511.
23. Pavitt G D and Proud C G. 2009. Protein synthesis and its control in neuronal cells with a focus on vanishing white matter disease. Biochem Soc Trans 37:1298-20 1310.
24. Costa-Mattioli M. Gobert D., Harding H., Herdy B. Azzi M., Bruno M. et al, 2005. Translational control of hippocampal synaptic plasticity and memory by the eIF2n kinase GCN2. Nature 436:1166-1173.
25. Costa-Mattioli M., Gobert D., Stern E., Garnache K., Colina R I, Cuello C., Sossin W., Kaufman R., Pelletier J., Rosenblum et al. 2007. eIF2n phosphorylation bidirectionally regulates the switch from short to long term synaptic plasticity and memory. Cell 129: 195-206.
26. Zhu P. J, Huan W., Kalikulov D., Yoo J. W., Placzek A. N. , Stoica L, Zhou H. , Bell J. C., Frielander M. J., Krnjevic K., Noebels J. L., Costa-Mattioli M. 2011. Suppression of PKR promotes network excitability and enhanced cognition by interferon-7-mediated disinhibition. Cell 147: 1384-1396.
27. Borck G., Shin B. S., Stiller B., et al 2012. eIF2y mutation that disrupts eIF2 complex integrity links intellectual disability to impaired translation 30 initiation. Mol Cell 48:1-6.
28. Zeenko V. V., Wang C, Majumder M, Komar A. A., Snider M. D., Merrick W. C., Kaufman R. J. and Hatzoglou M. (2008). An efficient in vitro translation system from mammalian cell lacking translational inhibition caused by eIF2 phosphorylation. RNA 14: 593-602.
29. Mikami S., Masutani M., Sonenber N., Yokoyama S. And Imataka H. 175 WO 2014/144952 PC T/US2014/029568 2006. An efficient mammalian cell-free translation system supplemented with translation factors. Protein Expr. Purif. 46:348-357.

While the preferred embodiments of the invention are illustrated by the above, it is to be understood that the invention is not limited to the precise instructions herein disclosed and that the right to all modifications coming within the scope of the following claims is reserved.

1. A compound represented by the following Formula (IIIQ): 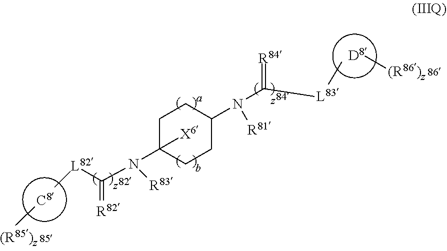
wherein:
L^{82′} is selected from: a bond, —NH—, —O—, —S—, —S(O)—, —S(O)₂—, cycloalkyl, —O-cycloalkyl, cycloalkyl-O—, —NH-cycloalkyl, cycloalkyl-NH—, azetidinyl, —O-azetidinyl, azetidinyl-O—, —N-azetidinyl, azetidinyl-N—, substituted or unsubstituted C₁₋₆alkylene and substituted or unsubstituted C₁₋₆heteroalkylene, or L^{82′} is taken together with R^{83′} to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—, or, L^{82′} is taken together with an R^{85′} substituent adjacent to the point of attachment of L^{82′} to C^{8′} to form a cycloalkyl ring, a heterocycloalkyl ring, or heteroaryl ring fused to C^{8′};
L^{83′} is selected from: cycloalkyl, —O-cycloalkyl, cycloalkyl-O—, —NH-cycloalkyl, cycloalkyl-NH—, azetidinyl, —O-azetidinyl, azetidinyl-O—, —N-azetidinyl, azetidinyl-N—, or L^{83′} and R^{81′} are taken together to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—, or, L^{83′} is taken together with an R^{86′} substituent adjacent to the point of attachment of L^{83′} to D^{8′} to form a cycloalkyl ring, a heterocycloalkyl ring, or heteroaryl ring fused to D^{8′};
R^{81′} is selected from: hydrogen, C₁₋₆alkyl, substituted C₁₋₆alkyl, and heterocycloalkyl, or R^{81′} is taken together with L^{83′} to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
R^{83′} is selected from: hydrogen, C₁₋₆alkyl, substituted C₁₋₆alkyl, and heterocycloalkyl, or R^{83′} is taken together with L^{82′} to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
R^{85′} is selected from: fluoro, chloro, bromo, iodo, —OCH₃, —OCH₂Ph, —C(O)Ph, —CF₃, —CN, —S(O)CH₃, —OH, —NH₂, —COOH, —CONH₂, —NO₂, —C(O)CH₃, —C≡CH, —CH₂C≡CH, —SCH₃, —SO₃H, —SO₂NH₂, —NHC(O)NH₂, —NHC(O)H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or, two adjacent R^{85′} substituents can combine to form a cycloalkyl ring, a heterocycloalkyl ring, or heteroaryl ring fused to C^{8′}, or, an R^{85′} substituent adjacent to the point of attachment of L^{82′} to C^{8′} can combine with L^{82′} to form a cycloalkyl ring, a heterocycloalkyl ring, or heteroaryl ring fused to C^{8′};
R^{86′} is selected from: fluoro, chloro, bromo, iodo, —OCH₃, —OCH₂Ph, —C(O)Ph, —CF₃, —CN, —S(O)CH₃, —OH, —NH₂, —COOH, —CONH₂, —NO₂, —C(O)CH₃, —C≡CH, —CH₂C≡CH, —SCH₃, —SO₃H, —SO₂NH₂, —NHC(O)NH₂, —NHC(O)H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or, two adjacent R^{86′} substituents can combine to form a cycloalkyl ring, a heterocycloalkyl ring, or heteroaryl ring fused to D^{8′}, or, an R^{86′} substituent adjacent to the point of attachment of L^{83′} to D^{8′} can combine with L^{83′} to form a cycloalkyl ring, a heterocycloalkyl ring, or heteroaryl ring fused to D^{8′};
R^{82′} and R^{84′} are independently NR^{88′}, O, CH₂, or S;
R^{88′} is selected from: hydrogen, C₁₋₆alkyl and C₁₋₆alkyl substituted 1 to 6 times by fluoro;
a and b are independently 0 or 1;
C^{8′} and D^{8′} are independently phenyl or pyridyl;
X^{6′} is C₁₋₃alkylene or C₁₋₃alkylene substituted 1 to 3 times by fluoro;
Z^{82′} and z^{84′} are independently 0 or 1; and
Z^{85′} and z^{86′} are independently an integer from 0 to 5;
or a pharmaceutically acceptable salt thereof.

2. The compound of claim 1 represented by Formula (IVQ): 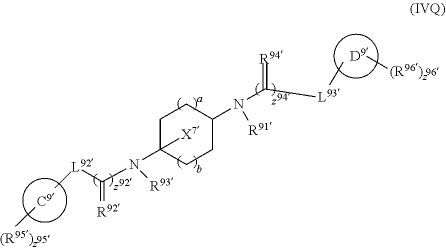
wherein:
L^{92′} is selected from: a bond, —NH—, —O—, —S—, —S(O)—, —S(O)₂—, substituted or unsubstituted C₁₋₆alkylene and substituted or unsubstituted C₁₋₆heteroalkylene;
L^{93′} is selected from: cycloalkyl, —O-cycloalkyl, and cycloalkyl-O—, azetidinyl, —O-azetidinyl, azetidinyl-O—, or L^{93′} is taken together with R^{91′} to form: heterocycloalkyl, heterocycloalkyl-O—, oxoheterocycloalkyl, or oxoheterocycloalkyl-O—, or, L^{93′} is taken together with an R^{96′} substituent adjacent to the point of attachment of L^{93′} to form a cycloalkyl ring, a heterocycloalkyl ring, or heteroaryl ring;
R^{91′} is selected from: hydrogen, C₁₋₆alkyl, substituted C₁₋₆alkyl, and heterocycloalkyl, or R^{91′} is taken together with L^{93′} to form: heterocycloalkyl, heterocycloalkyl-O—, oxoheterocycloalkyl, or oxoheterocycloalkyl-O—;
R^{93′} is selected from: hydrogen, C₁₋₆alkyl, substituted C₁₋₆alkyl, and heterocycloalkyl;
R^{95′} is selected from: fluoro, chloro, bromo, iodo, —OCH₃, —OCH₂Ph, —C(O)Ph, —CF₃, —CN, —S(O)CH₃, —OH, —NH₂, —COOH, —CONH₂, —NO₂, —C(O)CH₃, —C≡CH, —CH₂C≡CH, —SCH₃, —SO₃H, —SO₂NH₂, —NHC(O)NH₂, —NHC(O)H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{96′} is selected from: fluoro, chloro, bromo, iodo, —OCH₃, —OCH₂Ph, —C(O)Ph, —CF₃, —CN, —S(O)CH₃, —OH, —NH₂, —COOH, —CONH₂, —NO₂, —C(O)CH₃, —C≡CH, —CH₂C≡CH, —SCH₃, —SO₃H, —SO₂NH₂, —NHC(O)NH₂, —NHC(O)H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or, two adjacent R^{96′} substituents can combine to form a cycloalkyl ring, a heterocycloalkyl ring, or heteroaryl ring fused to D^{9′}, or, an R^{96′} substituent adjacent to the point of attachment of L^{93′} to D^{9′} can combine with L^{93′} to form a cycloalkyl ring, a heterocycloalkyl ring, or heteroaryl ring fused to D^{9′};
R^{92′} and R^{94′} are independently NR^{98′}, O, or S;
R^{98′} is selected from: hydrogen, C₁₋₆alkyl and C₁₋₆alkyl substituted 1 to 6 times by fluoro;
a and b are independently 0 or 1;
C^{9′} and D^{9′} are independently phenyl or pyridyl;
X^{7′} is C₁₋₃alkylene or C₁₋₃alkylene substituted 1 to 3 times by fluoro;
Z^{92′} and z^{94′} are independently 0 or 1; and
Z^{95′} and z^{96′} are independently an integer from 0 to 5;
or a pharmaceutically acceptable salt thereof.

3. The compound of claim 1 represented by Formula (VQ): 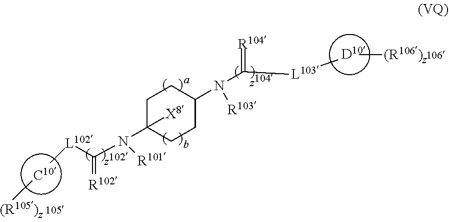
wherein:
L^{102′} is selected from: a bond, —CH₂—, —NH—, CH₂-O—, —O—CH₂—, cyclopropyl, —O-cyclopropyl, cyclopropyl-O—, —NH-cyclopropyl, cyclopropyl-NH—, azetidinyl, —O-azetidinyl, azetidinyl-O—, —N-azetidinyl, azetidinyl-N—, —O—CH₂—CH₂—, and —CH₂—CH₂—O—, or L^{102′} is taken together with R^{101′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—, or, L^{102′} is taken together with an R^{105′} substituent adjacent to the point of attachment of L^{102′} to form a heterocycloalkyl ring;
L^{103′} is selected from: cyclopropyl, —O-cyclopropyl, cyclopropyl-O—, —NH-cyclopropyl, cyclopropyl-NH—, azetidinyl, —O-azetidinyl, azetidinyl-O—, —N-azetidinyl, azetidinyl-N—, or L^{103′} is taken together with R^{103′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—, or, L^{103′} is taken together with an R^{106′} substituent adjacent to the point of attachment of L^{103′} to form a heterocycloalkyl ring;
R^{101′} is selected from: hydrogen, C₁₋₆alkyl, substituted C₁₋₆alkyl, and oxetanyl, or R¹⁰¹ is taken together with L^{102′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
R^{103′} is selected from: hydrogen, C₁₋₆alkyl, substituted C₁₋₆alkyl, and oxetanyl, or R^{103′} is taken together with L^{103′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
R^{105′} is selected from: methyl, cyclopropyl, —OCF₃, fluoro, chloro, —SCH₃, —OCH₃, —OCHF₂, and —CF₃, or, an R^{105′} substituent adjacent to the point of attachment of L^{102′} to C^{10′} can combine with L^{102′} to form a heterocycloalkyl ring fused to C^{10′};
R^{106′} is selected from: methyl, cyclopropyl, —OCF₃, fluoro, chloro, —SCH₃, —OCH₃, —OCHF₂, and —CF₃, or, an R^{106′} substituent adjacent to the point of attachment of L^{103′} to D^{10′} can combine with L^{103′} to form a heterocycloalkyl ring fused to D^{10′};
R^{102′} and R^{104′} are O;
a and b are independently 0 or 1;
C^{10′} and D^{10′} are independently phenyl or pyridyl;
X^{8′} is selected from —CH₂— and —CH₂—CH₂—;
Z^{102′} and z^{104′} are independently 0 or 1; and
Z^{105′} and z^{106′} are independently an integer from 0 to 5;
or a pharmaceutically acceptable salt thereof.

4. The compound of claim 1 represented by Formula (VIQ): 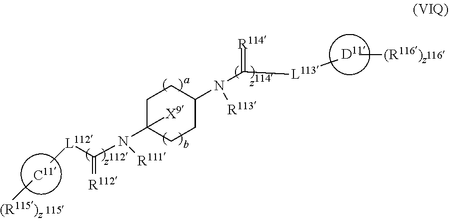
wherein:
L^{112′} is selected from: a bond, —CH₂—, —NH—, CH₂—O—, —O—CH₂—, —O—CH₂—CH₂—, and —CH₂—CH₂—O—;
L^{113′} is selected from: cyclopropyl, —O-cyclopropyl, cyclopropyl-O—, azetidinyl, —O-azetidinyl, azetidinyl-O—, or L^{113′} is taken together with R^{113′} to form: imidazolidinyl, azetidinyl, azetidinyl-O—, piperidinyl, piperidinyl-O—, piperazinyl, piperazinyl-O—, oxopiperazinyl, oxopiperazinyl-O—, pyrrolidinyl, pyrrolidinyl-O—, oxopyrrolidinyl, or oxopyrrolidinyl-O—, or, L^{113′} is taken together with an R^{116′} substituent adjacent to the point of attachment of L^{113′} to form a heterocycloalkyl ring;
R^{113′} is selected from: hydrogen, C₁₋₆alkyl, substituted C₁₋₆alkyl, and oxetanyl or R^{113′} is taken together with L^{113′} to form: imidazolidinyl, azetidinyl, azetidinyl-O—, piperidinyl, piperidinyl-O—, piperazinyl, piperazinyl-O—, oxopiperazinyl, oxopiperazinyl-O—, pyrrolidinyl, pyrrolidinyl-O—, oxopyrrolidinyl, or oxopyrrolidinyl-O—;
R^{111′} is selected from: hydrogen, C₁₋₆alkyl, substituted C₁₋₆alkyl, and oxetanyl;
R^{115′} is selected from: methyl, cyclopropyl, —OCF₃, fluoro, chloro, —SCH₃, —OCH₃, —OCHF₂, and —CF₃;
R^{116′} is selected from: methyl, cyclopropyl, —OCF₃, fluoro, chloro, —SCH₃, —OCH₃, —OCHF₂, and —CF₃, or, an R^{116′} substituent adjacent to the point of attachment of L^{113′} to D^{11′} can combine with L^{113′} to form a heterocycloalkyl ring fused to D^{11′};
R^{112′} and R^{114′} are O;
a and b are independently 0 or 1;
C^{11′} and D^{11′} are independently phenyl or pyridyl;
X^{9′} is selected from —CH₂— and —CH₂—CH₂—;
Z^{112′} and z^{114′} are independently 0 or 1; and
Z^{115′}and z^{116′} are independently an integer from 0 to 5;
or a pharmaceutically acceptable salt thereof.

5. The compound of claim 1 represented by Formula (VIIQ): 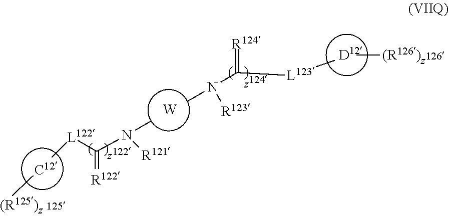
wherein:
W is selected from bicyclopentanyl and bicyclohexanyl;
L^{122′} is selected from: a bond, —CH₂—, —NH—, CH₂—O—, —O—CH₂—, cyclopropyl, —O-cyclopropyl, cyclopropyl-O—, —NH-cyclopropyl, cyclopropyl-NH—, azetidinyl, —O-azetidinyl, azetidinyl-O—, —N-azetidinyl, azetidinyl-N—, —O—CH₂—CH₂—, and —CH₂—CH₂—O—, or L^{122′} is taken together with R^{121′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—, or, L^{122′} is taken together with an R^{125′} substituent adjacent to the point of attachment of L^{122′} to form a cyclohexyl ring, a cyclobutyl ring, or a tetrahydro-pyran ring;
L^{123′} is selected from: cyclopropyl, azetidinyl, —O-azetidinyl, azetidinyl-O—, —N-azetidinyl, azetidinyl-N—, or L^{123′} is taken together with R^{123′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—, or, L^{123′} is taken together with an R^{126′} substituent adjacent to the point of attachment of L^{123′} to form a cyclohexyl ring, a cyclobutyl ring, or a tetrahydro-pyran ring;
R^{121′} is selected from: hydrogen, C₁₋₆alkyl, substituted C₁₋₆alkyl, and oxetanyl, or R^{121′} is taken together with L^{122′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
R^{123′} is hydrogen or R^{123′} is taken together with L^{123′} to form: imidazolidinyl, azetidinyl, azetidinyl-O—, azetidinyl-N—, azetidinyl-CH₂—, piperidinyl, piperidinyl-O—, piperidinyl-N—, piperidinyl-CH₂—, piperazinyl, piperazinyl-O—, piperazinyl-N—, piperazinyl-CH₂—, oxopiperazinyl, oxopiperazinyl-O—, oxopiperazinyl-N—, oxopiperazinyl-CH₂—, pyrrolidinyl, pyrrolidinyl-O—, pyrrolidinyl-N—, pyrrolidinyl-CH₂—, oxopyrrolidinyl, oxopyrrolidinyl-O—, oxopyrrolidinyl-N—, or oxopyrrolidinyl-CH₂—;
R¹²⁵ is selected from: methyl, cyclopropyl, —OCF₃, fluoro, chloro, —SCH₃, —OCH₃, —OCHF₂, and —CF₃, or, an R^{125′} substituent adjacent to the point of attachment of L^{122′} to C^{12′} can combine with L^{122′} to form a cyclohexyl ring, a cyclobutyl ring, or a tetrahydro-pyran ring fused to C^{12′};
R^{126′} is selected from: methyl, cyclopropyl, —OCF₃, fluoro, chloro, —SCH₃, —OCH₃, —OCHF₂, and —CF₃, or, an R^{126′} substituent adjacent to the point of attachment of L^{123′} to D^{12′} can combine with L^{123′} to form a cyclohexyl ring, a cyclobutyl ring, or a tetrahydro-pyran ring fused to D^{12′};
R^{122′} and R^{124′} are O;
C^{12′} and D^{12′} are independently phenyl or pyridyl;
Z^{122′} and z^{124′} are independently 0 or 1; and
Z^{125′} and z^{126′} are independently an integer from 0 to 3;
or a salt thereof including a pharmaceutically acceptable salt thereof.

6. The compound of claim 1 represented by Formula (VIIIQ): 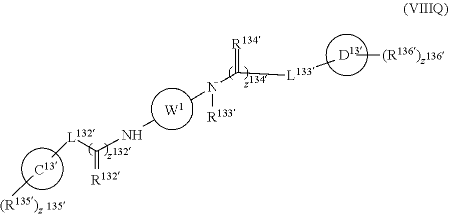
wherein:
W¹ is selected from bicyclopentanyl and bicyclohexanyl;
L^{132′} is selected from: a bond, —CH₂—, —NH—, CH₂—O—, —O—CH₂—, —O—CH₂—CH₂—, and —CH₂—CH₂—O—;
L^{133′} is selected from: cyclopropyl, —O-cyclopropyl, cyclopropyl-O—, azetidinyl, —O-azetidinyl, azetidinyl-O—, or L^{133′} is taken together with R^{133′} to form: imidazolidinyl, azetidinyl, azetidinyl-O—, piperidinyl, piperidinyl-O—, piperazinyl, piperazinyl-O—, oxopiperazinyl, oxopiperazinyl-O—, pyrrolidinyl, pyrrolidinyl-O—, oxopyrrolidinyl, or oxopyrrolidinyl-O— or, L^{133′} is taken together with an R^{136′} substituent adjacent to the point of attachment of L^{133′} to form a cyclohexyl ring, a cyclobutyl ring, or a tetrahydro-pyran ring;
R^{133′} is hydrogen or R^{133′} is taken together with L^{133′} to form: imidazolidinyl, azetidinyl, azetidinyl-O—, piperidinyl, piperidinyl-O—, piperazinyl, piperazinyl-O—, oxopiperazinyl, oxopiperazinyl-O—, pyrrolidinyl, pyrrolidinyl-O—, oxopyrrolidinyl, or oxopyrrolidinyl-O—;
R¹³⁵ is selected from: methyl, cyclopropyl, —OCF₃, fluoro, chloro, —SCH₃, —OCH₃, —OCHF₂, and —CF₃;
R^{136′} is selected from: methyl, cyclopropyl, —OCF₃, fluoro, chloro, —SCH₃, —OCH₃, —OCHF₂, and —CF₃, or, an R^{136′} substituent adjacent to the point of attachment of L^{133′} to D^{13′} can combine with L^{133′} to form a cyclohexyl ring, a cyclobutyl ring, or a tetrahydro-pyran ring fused to D^{13′};
R^{132′} and R^{134′} are O;
C^{13′} and D^{13′} are each independently phenyl or pyridyl;
Z^{132′} and z^{134′} are each independently 0 or 1; and
Z^{135′} and z^{136′} are each independently an integer from 0 to 3;
or a salt thereof including a pharmaceutically acceptable salt thereof.

7. The compound of claim 1 selected from:
2-(4-chlorophenoxy)-N-(3-(3-(4-chlorophenyl)-2-oxoimidazolidin-1-yl)bicyclo[1.1.1]pentan-1-yl)acetamide;
2-(4-chlorophenoxy)-N-(3-(3-(4-fluorophenyl)-2-oxoimidazolidin-1-yl)bicyclo[1.1.1]pentan-1-yl)acetamide;
N-(3-(3-(4-chlorophenyI)-2-oxoimidazolidin-1-yl)bicyclo[1.1.1]pentan-1-yl)-2-(4-fluorophenoxy)acetamide;
N-(3-(3-(4-chloro-2-methylphenyl)-2-oxoimidazolidin-1-yl)bicyclo[1.1.1]pentan-1-yl)-2-(4-chlorophenoxy)acetamide;
N-(3-(3-(4-chlorophenyl)-2-oxoimidazolidin-1-yl)bicyclo[1.1.1]pentan-1-yl)-2-(4-cyclopropylphenoxy)acetamide;
2-(4-chlorophenoxy)-N-(3-(3-(5-chloropyridin-2-yl)-2-oxoimidazolidin-1-yl)bicyclo[1.1.1]pentan-1-yl)acetamide;
2-(3-chlorophenoxy)-N-(3-(3-(4-chlorophenyl)-2-oxoimidazolidin-1-yl)bicyclo[1.1.1]pentan-1-yl)acetamide;
N-(3-(3-(4-chlorophenyl)-2-oxoimidazolidin-1-yl)bicyclo[1.1.1]pentan-1-yl)-2-(4-(trifluoromethoxy)phenoxy)acetamide;
2-(4-chlorophenoxy)-N-(3-(3-(4-chlorophenyl)-2-oxoimidazolidin-1-yl)bicyclo[1.1.1]pentan-1-yl)acetamide;
2-(4-chloro-3-(trifluoromethyl)phenoxy)-N-(3-(3-(4-chlorophenyI)-2-oxoimidazolidin-1-yl)bicyclo[1.1.1]pentan-1-yl)acetamide;
2-(4-chlorophenoxy)-N-(3-(3-(3-chlorophenyl)-2-oxoimidazolidin-1-yl)bicyclo[1.1.1]pentan-1-yl)acetamide;
N-(3-(2-(4-chlorophenoxy)acetamido)bicyclo[1.1.1]pentan-1-yl)-2-(4-chlorophenyl)cyclopropane-1-carboxamide;
N-(4-(2-(4-chlorophenoxy)acetamido)bicyclo[2.1.1]hexan-1-yl)-2-(4-chlorophenyl)cyclopropane-1-carboxamide;
2-(4-chlorophenoxy)-N-(3-(2-(4-chlorophenoxy)acetamido)bicyclo[1.1.1]pentan-1-yl)cyclopropane-1-carboxamide;
2-(4-chlorophenoxy)-N-(3-((1-(4-chlorophenyl)azetidin-3-yl)amino)bicyclo[1.1.1]pentan-1-yl)acetamide;
2-(4-chlorophenoxy)-N-(3-(3-(4-chlorophenoxy)azetidin-1-yl)bicyclo[1.1.1]pentan-1-yl)acetamide;
2-(4-chlorophenoxy)-N-(3-(2-((5,6,7,8-tetrahydronaphthalen-2-yl)oxy)acetamido)bicyclo[1.1.1]pentan-1-yl)acetamide;
5-chloro-N-(3-(2-(4-chlorophenoxy)acetamido)bicyclo[1.1.1]pentan-1-yl)-2,3-dihydrobenzofuran-2-carboxamide;
2-(bicyclo[4.2.0]octa-1(6),2,4-trien-3-yloxy)-N-(3-(2-(4-chlorophenoxy)acetamido)bicyclo[1.1.1]pentan-1-yl)acetamide;
2-(4-chlorophenoxy)-N-(3-(2-(chroman-6-yloxy)acetamido)bicyclo[1.1.1]pentan-1-yl)acetamide;
2-(4-chlorophenoxy)-N-(3-(4-(4-chlorophenyl)piperidin-1-yl)bicyclo[1.1.1]pentan-1-yl)acetamide;
2-(4-chlorophenoxy)-N-(3-(4-(4-chlorophenyl)piperazin-1-yl)bicyclo[1.1.1]pentan-1-yl)acetamide;
2-(bicyclo[4.2.0]octa-1,3,5-trien-3-yloxy)-N-(4-(2-(4-chlorophenoxy)acetamido)bicyclo[2.2.1]heptan-1-yl)acetamide;
2-(4-chlorophenoxy)-N-(3-(3-(4-chlorophenoxy)pyrrolidin-1-yl)bicyclo[1.1.1]pentan-1-yl)acetamide;
(S)-2-(4-chlorophenoxy)-N-(3-(3-(4-chlorophenoxy)pyrrolidin-1-yl)bicyclo[1.1.1]pentan-1-yl)acetamide;
(R)-2-(4-chlorophenoxy)-N-(3-(3-(4-chlorophenoxy)pyrrolidin-1-yl)bicyclo[1.1.1]pentan-1-yl)acetamide;
N-(3-(3-(4-chlorophenoxy)pyrrolidin-1-yl)bicyclo[1.1.1]pentan-1-yl)-2-(4-fluorophenoxy)acetamide isomer 1;
N-(3-(3-(4-chlorophenoxy)pyrrolidin-1-yl)bicyclo[1.1.1]pentan-1-yl)-2-(4-fluorophenoxy)acetamide isomer 2;
2-(4-chlorophenoxy)-N-(3-(3-(4-fluorophenoxy)pyrrolidin-1-yl)bicyclo[1.1.1]pentan-1-yl)acetamide;
N-(3-(3-(3-chloro-4-fluorophenoxy)pyrrolidin-1-yl)bicyclo[1.1.1]pentan-1-yl)-2-(4-chlorophenoxy)acetamide isomer 1;
N-(3-(3-(3-chloro-4-fluorophenoxy)pyrrolidin-1-yl)bicyclo[1.1.1]pentan-1-yl)-2-(4-chlorophenoxy)acetamide isomer 2;
N-(3-(3-(4-chlorophenoxy)pyrrolidin-1-yl)bicyclo[1.1.1]pentan-1-yl)-2-((5-chloropyridin-2-yl)oxy)acetamide;
N-(3-(3-(4-chlorophenoxy)pyrrolidin-1-yl)bicyclo[1.1.1]pentan-1-yl)-2-(4-(trifluoromethyl)phenoxy)acetamide;
N-(3-(3-(4-chlorophenoxy)pyrrolidin-1-yl)bicyclo[1.1.1]pentan-1-yl)-2-(4-(trifluoromethoxy)phenoxy)acetamide;
2-(4-chloro-3-(trifluoromethyl)phenoxy)-N-(3-(3-(4-chlorophenoxy)pyrrolidin-1-yl)bicyclo[1.1.1]pentan-1-yl)acetamide;
2-(4-chlorophenoxy)-N-(3-(3-(4-(trifluoromethyl)phenoxy)pyrrolidin-1-yl)bicyclo[1.1.1]pentan-1-yl)acetamide;
2-(4-chloro-3-fluorophenoxy)-N-(3-(3-(4-chlorophenoxy)pyrrolidin-1-yl)bicyclo[1.1.1]pentan-1-yl)acetamide;
N-(3-(3-(4-chlorophenoxy)pyrrolidin-1-yl)bicyclo[1.1.1]pentan-1-yl)-2-(4-cyclopropylphenoxy)acetamide;
N-(3-(3-(4-chloro-3-fluorophenoxy)pyrrolidin-1-yl)bicyclo[1.1.1]pentan-1-yl)-2-(4-chlorophenoxy)acetamide isomer 1;
N-(3-(3-(4-chloro-3-fluorophenoxy)pyrrolidin-1-yl)bicyclo[1.1.1]pentan-1-yl)-2-(4-chlorophenoxy)acetamide isomer 2;
2-(4-chlorophenoxy)-N-(3-(3-(pyridin-4-yloxy)pyrrolidin-1-yl)bicyclo[1.1.1]pentan-1-yl)acetamide;
1-(3-((2-(4-chlorophenoxy)ethyl)amino)bicyclo[1.1.1]pentan-1-yl)-3-(4-chlorophenyl)imidazolidin-2-one;
2-(4-chloro-3-fluorophenoxy)-N-(3-(3-(4-chlorophenyl)-2-oxoimidazolidin-1-yl)bicyclo[1.1.1]pentan-1-yl)acetamide;
N-(3-(3-(4-chlorophenyl)-2-oxoimidazolidin-1-yl)bicyclo[1.1.1]pentan-1-yl)-2-(4-methoxyphenoxy)acetamide;
2-(3-chloro-4-fluorophenoxy)-N-(3-(3-(4-chlorophenyl)-2-oxoimidazolidin-1-yl)bicyclo[1.1.1]pentan-1-yl)acetamide;
N-(3-(3-(4-chloro-3-(trifluoromethyl)phenyl)-2-oxoimidazolidin-1-yl)bicyclo[1.1.1]pentan-1-yl)-2-(4-chlorophenoxy)acetamide;
2-(4-chlorophenoxy)-N-(3-(3-(4-fluoro-3-(trifluoromethyl)phenyl)-2-oxoimidazolidin-1-yl)bicyclo[1.1.1]pentan-1-yl)acetamide;
N-(3-(5-chloroisoindolin-2-yl)bicyclo[1.1.1]pentan-1-yl)-2-(4-chlorophenoxy)acetamide;
N-(3-(3-(4-chlorophenyl)-2-oxoimidazolidin-1-yl)bicyclo[1.1.1]pentan-1-yl)-2-((5-chloropyridin-2-yl)oxy)acetamide;
2-(4-chlorophenoxy)-N-(3-(2-oxo-3-(4-(trifluoromethyl)phenyl)imidazolidin-1-yl)bicyclo[1.1.1]pentan-1-yl)acetamide;
N-(3-(3-(4-chlorophenyl)-2-oxoimidazolidin-1-yl)bicyclo[1.1.1]pentan-1-yl)-2-(4-fluoro-3-(trifluoromethyl)phenoxy)acetamide;
N-(3-(3-(4-chlorophenoxy)pyrrolidin-1-yl)bicyclo[1.1.1]pentan-1-yl)-2-(4-fluoro-3-(trifluoromethyl)phenoxy)acetamide;
N-(3-(3-(4-chlorophenoxy)pyrrolidin-1-yl)bicyclo[1.1.1]pentan-1-yl)-2-(4-(difluoromethoxy)phenoxy)acetamide;
2-(4-chloro-3-fluorophenoxy)-N-(3-(3-(4-chlorophenoxy)pyrrolidin-1-yl)bicyclo[1.1.1]pentan-1-yl)acetamide isomer 1;
(R)-2-(4-chloro-3-fluorophenoxy)-N-(3-(3-(4-chlorophenoxy)pyrrolid in-1-yl)bicyclo[1.1.1]pentan-1-yl)acetamide isomer 2;
2-(4-chlorophenoxy)-N-(3-(3-((5-chloropyridin-2-yl)oxy)pyrrolidin-1-yl)bicyclo[1.1.1]pentan-1-yl)acetamide;
2-((5-chloropyridin-2-yl)oxy)-N-(3-(3-((5-chloropyridin-2-yl)oxy)pyrrolidin-1-yl)bicyclo[1.1.1]pentan-1-yl)acetamide;
2-(4-chlorophenoxy)-N-(3-(3-(4-methoxyphenyl)-2-oxoimidazolidin-1-yl)bicyclo[1.1.1]pentan-1-yl)acetamide;
N-(3-(3-(4-chloro-2-fluorophenyl)-2-oxoimidazolidin-1-yl)bicyclo[1.1.1]pentan-1-yl)-2-(4-chlorophenoxy)acetamide;
N-(3-(3-(bicyclo[4.2.0]octa-1,3,5-trien-3-yloxy)pyrrolidin-1-yl)bicyclo[1.1.1]pentan-1-yl)-2-(4-chlorophenoxy)acetamide;
(S)-2-(4-chlorophenoxy)-N-(3-(4-(4-chlorophenoxy)-2-oxopyrrolidin-1-yl)bicyclo[1.1.1]pentan-1-yl)acetamide;
(S)-2-(4-chlorophenoxy)-N-(3-(3-(4-chlorophenoxy)-2-oxopyrrolidin-1-yl)bicyclo[1.1.1]pentan-1-yl)acetamide;
(R)-2-(4-chlorophenoxy)-N-(3-(4-(4-chlorophenoxy)-2-oxopyrrolidin-1-yl)bicyclo[1.1.1]pentan-1-yl)acetamide;
(R)-2-(4-chlorophenoxy)-N-(3-(3-(4-chlorophenoxy)-2-oxopyrrolidin-1-yl)bicyclo[1.1.1]pentan-1-yl)acetamide;
2-(4-chlorophenoxy)-N-(3-(3-(4-(methylthio)phenyl)-2-oxoimidazolidin-1-yl)bicyclo[1.1.1]pentan-1-yl)acetamide; and
2-(4-chlorophenoxy)-N-(3-(4-(4-chlorophenoxy)piperidin-1-yl)bicyclo[1.1.1]pentan-1-yl)acetamide;
or a pharmaceutically acceptable salt thereof.

8. A pharmaceutical composition comprising the compound according to claim 1 or a pharmaceutically acceptable salt thereof and a pharmaceutically acceptable excipient.

9. A method of treating a disease selected from: cancer, pre-cancerous syndromes, Alzheimer's disease, spinal cord injury, traumatic brain injury, ischemic stroke, stroke, diabetes, Parkinson disease, Huntington's disease, Creutzfeldt-Jakob Disease, prion diseases, progressive supranuclear palsy, amyotrophic lateral sclerosis, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementia, cognitive impairment, atherosclerosis, ocular diseases, neurological disorders, pain, in organ transplantation and arrhythmias, in a human in need thereof, which comprises administering to such human a therapeutically effective amount of the compound as described in claim 1 or a pharmaceutically acceptable salt thereof.

10. (canceled)

11. A method of treating a disease selected from: cancer, pre-cancerous syndromes, Alzheimer's disease, spinal cord injury, traumatic brain injury, ischemic stroke, stroke, diabetes, Parkinson disease, Huntington's disease, Creutzfeldt-Jakob Disease, and related prion diseases, progressive supranuclear palsy, amyotrophic lateral sclerosis, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementia, cognitive impairment, atherosclerosis, ocular diseases, neurological disorders, pain, in organ transplantation and arrhythmias in a human in need thereof, which comprises administering to such human a therapeutically effective amount of a compound of claim 7 or a pharmaceutically acceptable salt thereof.

12.-13. (canceled)

14. The method according to claim 11 wherein said cancer is selected from: brain (gliomas), glioblastomas, astrocytomas, glioblastoma multiforme, Bannayan-Zonana syndrome, Cowden disease, Lhermitte-Duclos disease, breast, colon, head and neck, kidney, lung, liver, melanoma, ovarian, pancreatic, adenocarcinoma, ductal adenocarcinoma, adenosquamous carcinoma, acinar cell carcinoma, glucagonoma, insulinoma, prostate, sarcoma and thyroid.

15. (canceled)

16. A method of inhibiting the ATF4 pathway in a human in need thereof, which comprises administering to such human a therapeutically effective amount of the compound as described in claim 1 or a pharmaceutically acceptable salt thereof.

17. (canceled)

18. A method of treating cancer in a human in need thereof, which comprises: administering to such human a therapeutically effective amount of
a) the compound as described in claim 1 or a pharmaceutically acceptable salt thereof; and
b) at least one anti-neoplastic agent.

19. (canceled)

20. A pharmaceutical combination comprising:
a) the compound as described in claim 1 or a pharmaceutically acceptable salt thereof; and
b) at least one anti-neoplastic agent.

21. (canceled)

22. The method according to claim 9 wherein said cancer is selected from: breast cancer, inflammatory breast cancer, ductal carcinoma, lobular carcinoma, colon cancer, pancreatic cancer, insulinomas, adenocarcinoma, ductal adenocarcinoma, adenosquamous carcinoma, acinar cell carcinoma, glucagonoma, skin cancer, melanoma, metastatic melanoma, lung cancer, small cell lung cancer, non-small cell lung cancer, squamous cell carcinoma, adenocarcinoma, large cell carcinoma, brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, head and neck, kidney, liver, melanoma, ovarian, pancreatic, adenocarcinoma, ductal adenocarcinoma, adenosquamous carcinoma, acinar cell carcinoma, glucagonoma, insulinoma, prostate, sarcoma, osteosarcoma, giant cell tumor of bone, thyroid,
lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocytic leukemia, promyelocytic leukemia, erythroleukemia,
malignant lymphoma, hodgkins lymphoma, non-hodgkins lymphoma, lymphoblastic T cell lymphoma, Burkitt's lymphoma, follicular lymphoma,
neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), neuroendocrine cancers and testicular cancer.

23. (canceled)

24. A process for preparing a pharmaceutical composition containing a pharmaceutically acceptable excipient and an effective amount of a compound as described in claim 1 or a pharmaceutically acceptable salt thereof, which process comprises bringing the compound or a pharmaceutically acceptable salt thereof into association with a pharmaceutically acceptable excipient.

25. The method according to claim 9 wherein said pre-cancerous syndrome is selected from: cervical intraepithelial neoplasia, monoclonal gammapathy of unknown significance (MGUS), myelodysplastic syndrome, aplastic anemia, cervical lesions, skin nevi (pre-melanoma), prostatic intraepithleial (intraductal) neoplasia (PIN), Ductal Carcinoma in situ (DCIS), colon polyps and severe hepatitis or cirrhosis.

26. (canceled)

27. A method of treating ocular diseases in a human in need thereof, which comprises administering to such human a therapeutically effective amount of a compound as described in claim 1 or a pharmaceutically acceptable salt thereof.

28. The method according to claim 27 wherein the ocular disease is selected from: rubeosis irides; neovascular glaucoma; pterygium; vascularized glaucoma filtering blebs; conjunctival papilloma; choroidal neovascularization associated with age-related macular degeneration (AMD), myopia, prior uveitis, trauma, or idiopathic; macular edema; retinal neovascularization due to diabetes; age-related macular degeneration (AMD); macular degeneration (AMD); ocular ischemic syndrome from carotid artery disease; ophthalmic or retinal artery occlusion; sickle cell retinopathy; retinopathy of prematurity; Eale's Disease; and VonHippel-Lindau syndrome.

29. The method according to claim 27 wherein the ocular disease is selected from: age-related macular degeneration (AMD) and macular degeneration.

30. A method of treating neurodegeneration in a human in need thereof, which comprises administering to such human a therapeutically effective amount of the compound as described in claim 1 or a pharmaceutically acceptable salt thereof.

31. A method of preventing organ damage during the transportation of organs for transplantation, which comprises adding the compound as described in claim 1 or a pharmaceutically acceptable salt thereof, to a solution housing the organ during transportation.

32.-39. (canceled)
